SEQUENCE LISTING (481 sequences)

SEQ ID NO: 1 - protein, 1368 aa, Streptococcus pyogenes [sequence omitted]

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 84 aa, Bacillus subtilis
MTNLSDIIEKETGKQLVIQESILMLPEEVEEVIGNKPESDILVHTAYDESTDENVMLLTSDAPEYKPWALVIQDSNGENKIKML

SEQ ID NO: 4 - protein, 9 aa, Human immunodeficiency virus 1
RKKRRQRRR

SEQ ID NO: 5 - protein, 12 aa, Human immunodeficiency virus 1
RQARRNRRRRWR

SEQ ID NO: 6 - protein, 7 aa, unidentified
PKKKRKV

SEQ ID NO: 7 - protein, 16 aa, unidentified
KRPAATKKAGQAKKKK

SEQ ID NO: 8 - protein, 9 aa, unidentified
PAAKRVKLD

SEQ ID NO: 9 - protein, 157 aa, synthetic construct
GDAHTGSSGSEFGGGSGGGSGGGSEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGGGSGGGSKLNGSSGSELDK

SEQ ID NO: 10 - protein, 161 aa, synthetic construct
GDAHTGSSGSEFGGGSGGGSGGGSEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGGGSGGGSKLNGSSGSELDKKDEL

SEQ ID NO: 11 - protein, 4 aa, unidentified
KDEL

SEQ ID NO: 12 - protein, 9 aa, Human immunodeficiency virus 1
RKKRRQRRR

SEQ ID NO: 13 - protein, 36 aa, synthetic construct
HHHHHHGMGAAGRKKRRQRRRPPAGTSVSLKKKRKV

SEQ ID NO: 14 - protein, 9 aa, unidentified
LAGLIDADG

SEQ ID NO: 15 - DNA, 21 bp, Homo sapiens
  source 1..21: mol_type = genomic DNA
cttgagcgtgctgaacagcgc

SEQ ID NO: 16 - DNA, 21 bp, Homo sapiens
  source 1..21: mol_type = genomic DNA
agagactccagttgccagcgg

SEQ ID NO: 17 - DNA, 21 bp, Homo sapiens
  source 1..21: mol_type = genomic DNA
agcgccacaagcttccggact

SEQ ID NO: 18 - DNA, 21 bp, Homo sapiens
  source 1..21: mol_type = genomic DNA
gggagatcatccacaaagccc

SEQ ID NO: 19 - DNA, 21 bp, Homo sapiens
  source 1..21: mol_type = genomic DNA
aattccgctggcaactggagt

SEQ ID NO: 20 - DNA, 21 bp, Homo sapiens
  source 1..21: mol_type = genomic DNA
tggatgatctcccgaaactga

SEQ ID NO: 21 - DNA, 21 bp, Homo sapiens
  source 1..21: mol_type = genomic DNA
cagtgtgtatcggtgcatggt

SEQ ID NO: 22 - DNA, 21 bp, Homo sapiens
  source 1..21: mol_type = genomic DNA
aacctactaatgggatcattc

SEQ ID NO: 23 - DNA, 21 bp, Homo sapiens
  source 1..21: mol_type = genomic DNA
tgtcgatgagggctttgtgga

SEQ ID NO: 24 - DNA, 21 bp, Homo sapiens
  source 1..21: mol_type = genomic DNA
tcctcagtttcgggagatcat

SEQ ID NO: 25 - DNA, 21 bp, Homo sapiens
  source 1..21: mol_type = genomic DNA
cagggtggcctggatgtttct

SEQ ID NO: 26 - DNA, 21 bp, Homo sapiens
  source 1..21: mol_type = genomic DNA
aaaccatgcaccgatacacac

SEQ ID NO: 27 - DNA, 21 bp, Homo sapiens
  source 1..21: mol_type = genomic DNA
tatttgagcaatatgcggaaa

SEQ ID NO: 28 - DNA, 21 bp, Homo sapiens
  source 1..21: mol_type = genomic DNA
ttccagtgagttgtactgaag

SEQ ID NO: 29 - DNA, 21 bp, Homo sapiens
  source 1..21: mol_type = genomic DNA
cgcatattgctcaaatacaaa

SEQ ID NO: 30 - DNA, 21 bp, Homo sapiens
  source 1..21: mol_type = genomic DNA
agcaatatgcggaaagctgtg

SEQ ID NO: 31 - DNA, 21 bp, Homo sapiens
  source 1..21: mol_type = genomic DNA
aagttgcgtgtgtctgtttcc

SEQ ID NO: 32 - DNA, 21 bp, Homo sapiens
  source 1..21: mol_type = genomic DNA
ttctggctttccagggtggcc

SEQ ID NO: 33 - DNA, 21 bp, Homo sapiens
  source 1..21: mol_type = genomic DNA
ataagattgtcccattcatca

SEQ ID NO: 34 - DNA, 20 bp, Homo sapiens
  source 1..20: mol_type = genomic DNA
cacgcaactttaaattccgc

SEQ ID NO: 35 - DNA, 20 bp, Homo sapiens
  source 1..20: mol_type = genomic DNA
tcaactggtgtcgagaagtc

SEQ ID NO: 36 - DNA, 21 bp, Homo sapiens
  source 1..21: mol_type = genomic DNA
tcctttgcaacatcctcagaa

SEQ ID NO: 37 - DNA, 20 bp, Homo sapiens
  source 1..20: mol_type = genomic DNA
ggcgctgttcagcacgctca

SEQ ID NO: 38 - DNA, 21 bp, Homo sapiens
  source 1..21: mol_type = genomic DNA
aacaaattcctgagatttgag

SEQ ID NO: 39 - DNA, 20 bp, Homo sapiens
  source 1..20: mol_type = genomic DNA
tcgagaagtccggaagcttg

SEQ ID NO: 40 - DNA, 20 bp, Homo sapiens
  source 1..20: mol_type = genomic DNA
tgaagtccacttcgggccat

SEQ ID NO: 41 - DNA, 20 bp, Homo sapiens
  source 1..20: mol_type = genomic DNA
tcagtacatgtggggcgttc

SEQ ID NO: 42 - DNA, 20 bp, Homo sapiens
  source 1..20: mol_type = genomic DNA
ttccagtgtgtatcggtgca

SEQ ID NO: 43 - DNA, 20 bp, Homo sapiens
  source 1..20: mol_type = genomic DNA
cggggctttgctatgatact

SEQ ID NO: 44 - DNA, 21 bp, Homo sapiens
  source 1..21: mol_type = genomic DNA
aaatgatgaatgatcccatta

SEQ ID NO: 45 - DNA, 20 bp, Homo sapiens
  source 1..20: mol_type = genomic DNA
gttgtactgaagtccacttc

SEQ ID NO: 46 - DNA, 20 bp, Homo sapiens
  source 1..20: mol_type = genomic DNA
tggacttcagtacaactcac

SEQ ID NO: 47 - DNA, 20 bp, Homo sapiens
  source 1..20: mol_type = genomic DNA
agttgtactgaagtccactt

SEQ ID NO: 48 - DNA, 20 bp, Homo sapiens
  source 1..20: mol_type = genomic DNA
cctcatcgacagaaacatcc

SEQ ID NO: 49 - DNA, 20 bp, Homo sapiens
  source 1..20: mol_type = genomic DNA
gcggaaagctgtgaagatac

SEQ ID NO: 50 - DNA, 20 bp, Homo sapiens
  source 1..20: mol_type = genomic DNA
ttttcagcgccacaagcttc

SEQ ID NO: 51 - DNA, 20 bp, Homo sapiens
  source 1..20: mol_type = genomic DNA
ctgaagtccacttcgggcca

SEQ ID NO: 52 - DNA, 20 bp, Homo sapiens
  source 1..20: mol_type = genomic DNA
gccacttctcagtacatgtg

SEQ ID NO: 53 - DNA, 20 bp, Homo sapiens
  source 1..20: mol_type = genomic DNA
tgatgaatgatcccattagt

SEQ ID NO: 54 - DNA, 20 bp, Homo sapiens
  source 1..20: mol_type = genomic DNA
cctggatgtttctgtcgatg

SEQ ID NO: 55 - DNA, 20 bp, Homo sapiens
  source 1..20: mol_type = genomic DNA
gccccacatgtactgagaag

SEQ ID NO: 56 - DNA, 20 bp, Homo sapiens
  source 1..20: mol_type = genomic DNA
tgcggaaagctgtgaagata

SEQ ID NO: 57 - DNA, 20 bp, Homo sapiens
  source 1..20: mol_type = genomic DNA
ctggatgtttctgtcgatga

SEQ ID NO: 58 - DNA, 20 bp, Homo sapiens
  source 1..20: mol_type = genomic DNA
ttctgtcgatgagggctttg

SEQ ID NO: 59 - DNA, 20 bp, Homo sapiens
  source 1..20: mol_type = genomic DNA
atgccacttctcagtacatg

SEQ ID NO: 60 - DNA, 20 bp, Homo sapiens
  source 1..20: mol_type = genomic DNA
gggcgttcaggacacagact

SEQ ID NO: 61 - DNA, 20 bp, Homo sapiens
  source 1..20: mol_type = genomic DNA
ttgctcaaatacaaagcctg

SEQ ID NO: 62 - DNA, 20 bp, Homo sapiens
  source 1..20: mol_type = genomic DNA
atgggacaatcttatcaaaa

SEQ ID NO: 63 - DNA, 20 bp, Homo sapiens
  source 1..20: mol_type = genomic DNA
cacagacttggtactgagga

SEQ ID NO: 64 - DNA, 20 bp, Homo sapiens
  source 1..20: mol_type = genomic DNA
ggcttccacagacacaccca

SEQ ID NO: 65 - DNA, 20 bp, Homo sapiens
  source 1..20: mol_type = genomic DNA
tctcaggaatttgttgaaac

SEQ ID NO: 66 - DNA, 20 bp, Homo sapiens
  source 1..20: mol_type = genomic DNA
tggacttcagtacaactcac

SEQ ID NO: 67 - DNA, 20 bp, Homo sapiens
  source 1..20: mol_type = genomic DNA
gccccacatgtactgagaag

SEQ ID NO: 68 - DNA, 20 bp, Homo sapiens
  source 1..20: mol_type = genomic DNA
gggcgttcaggacacagact

SEQ ID NO: 69 - DNA, 20 bp, Homo sapiens
  source 1..20: mol_type = genomic DNA
tctcaggaatttgttgaaac

SEQ ID NO: 70 - RNA, 21 nt, synthetic construct
  source 1..21: mol_type = other RNA
cttgagcgtgctgaacagcgc

SEQ ID NO: 71 - RNA, 21 nt, synthetic construct
  source 1..21: mol_type = other RNA
agagactccagttgccagcgg

SEQ ID NO: 72 - RNA, 21 nt, synthetic construct
  source 1..21: mol_type = other RNA
agcgccacaagcttccggact

SEQ ID NO: 73 - RNA, 21 nt, synthetic construct
  source 1..21: mol_type = other RNA
gggagatcatccacaaagccc

SEQ ID NO: 74 - RNA, 21 nt, synthetic construct
  source 1..21: mol_type = other RNA
aattccgctggcaactggagt

SEQ ID NO: 75 - RNA, 21 nt, synthetic construct
  source 1..21: mol_type = other RNA
tggatgatctcccgaaactga

SEQ ID NO: 76 - RNA, 21 nt, synthetic construct
  source 1..21: mol_type = other RNA
cagtgtgtatcggtgcatggt

SEQ ID NO: 77 - RNA, 21 nt, synthetic construct
  source 1..21: mol_type = other RNA
aacctactaatgggatcattc

SEQ ID NO: 78 - RNA, 21 nt, synthetic construct
  source 1..21: mol_type = other RNA
tgtcgatgagggctttgtgga

SEQ ID NO: 79 - RNA, 21 nt, synthetic construct
  source 1..21: mol_type = other RNA
tcctcagtttcgggagatcat

SEQ ID NO: 80 - RNA, 21 nt, synthetic construct
  source 1..21: mol_type = other RNA
cagggtggcctggatgtttct

SEQ ID NO: 81 - RNA, 21 nt, synthetic construct
  source 1..21: mol_type = other RNA
aaaccatgcaccgatacacac

SEQ ID NO: 82 - RNA, 21 nt, synthetic construct
  source 1..21: mol_type = other RNA
tatttgagcaatatgcggaaa

SEQ ID NO: 83 - RNA, 21 nt, synthetic construct
  source 1..21: mol_type = other RNA
ttccagtgagttgtactgaag

SEQ ID NO: 84 - RNA, 21 nt, synthetic construct
  source 1..21: mol_type = other RNA
cgcatattgctcaaatacaaa

SEQ ID NO: 85 - RNA, 21 nt, synthetic construct
  source 1..21: mol_type = other RNA
agcaatatgcggaaagctgtg

SEQ ID NO: 86 - RNA, 21 nt, synthetic construct
  source 1..21: mol_type = other RNA
aagttgcgtgtgtctgtttcc

SEQ ID NO: 87 - RNA, 21 nt, synthetic construct
  source 1..21: mol_type = other RNA
ttctggctttccagggtggcc

SEQ ID NO: 88 - RNA, 21 nt, synthetic construct
  source 1..21: mol_type = other RNA
ataagattgtcccattcatca

SEQ ID NO: 89 - RNA, 20 nt, synthetic construct
  source 1..20: mol_type = other RNA
cacgcaactttaaattccgc

SEQ ID NO: 90 - RNA, 20 nt, synthetic construct
  source 1..20: mol_type = other RNA
tcaactggtgtcgagaagtc

SEQ ID NO: 91 - RNA, 21 nt, synthetic construct
  source 1..21: mol_type = other RNA
tcctttgcaacatcctcagaa

SEQ ID NO: 92 - RNA, 20 nt, synthetic construct
  source 1..20: mol_type = other RNA
ggcgctgttcagcacgctca

SEQ ID NO: 93 - RNA, 21 nt, synthetic construct
  source 1..21: mol_type = other RNA
aacaaattcctgagatttgag

SEQ ID NO: 94 - RNA, 20 nt, synthetic construct
  source 1..20: mol_type = other RNA
tcgagaagtccggaagcttg

SEQ ID NO: 95 - RNA, 20 nt, synthetic construct
  source 1..20: mol_type = other RNA
tgaagtccacttcgggccat

SEQ ID NO: 96 - RNA, 20 nt, synthetic construct
  source 1..20: mol_type = other RNA
tcagtacatgtggggcgttc

SEQ ID NO: 97 - RNA, 20 nt, synthetic construct
  source 1..20: mol_type = other RNA
ttccagtgtgtatcggtgca

SEQ ID NO: 98 - RNA, 20 nt, synthetic construct
  source 1..20: mol_type = other RNA
cggggctttgctatgatact

SEQ ID NO: 99 - RNA, 21 nt, synthetic construct
  source 1..21: mol_type = other RNA
aaatgatgaatgatcccatta

SEQ ID NO: 100 - RNA, 20 nt, synthetic construct
  source 1..20: mol_type = other RNA
gttgtactgaagtccacttc

SEQ ID NO: 101 - RNA, 20 nt, synthetic construct
  source 1..20: mol_type = other RNA
tggacttcagtacaactcac

SEQ ID NO: 102 - RNA, 20 nt, synthetic construct
  source 1..20: mol_type = other RNA
agttgtactgaagtccactt

SEQ ID NO: 103 - RNA, 20 nt, synthetic construct
  source 1..20: mol_type = other RNA
cctcatcgacagaaacatcc

SEQ ID NO: 104 - RNA, 20 nt, synthetic construct
  source 1..20: mol_type = other RNA
gcggaaagctgtgaagatac

SEQ ID NO: 105 - RNA, 20 nt, synthetic construct
  source 1..20: mol_type = other RNA
ttttcagcgccacaagcttc

SEQ ID NO: 106 - RNA, 20 nt, synthetic construct
  source 1..20: mol_type = other RNA
ctgaagtccacttcgggcca

SEQ ID NO: 107 - RNA, 20 nt, synthetic construct
  source 1..20: mol_type = other RNA
gccacttctcagtacatgtg

SEQ ID NO: 108 - RNA, 20 nt, synthetic construct
  source 1..20: mol_type = other RNA
tgatgaatgatcccattagt

SEQ ID NO: 109 - RNA, 20 nt, synthetic construct
  source 1..20: mol_type = other RNA
cctggatgtttctgtcgatg

SEQ ID NO: 110 - RNA, 20 nt, synthetic construct
  source 1..20: mol_type = other RNA
gccccacatgtactgagaag

SEQ ID NO: 111 - RNA, 20 nt, synthetic construct
  source 1..20: mol_type = other RNA
tgcggaaagctgtgaagata

SEQ ID NO: 112 - RNA, 20 nt, synthetic construct
  source 1..20: mol_type = other RNA
ctggatgtttctgtcgatga

SEQ ID NO: 113 - RNA, 20 nt, synthetic construct
  source 1..20: mol_type = other RNA
ttctgtcgatgagggctttg

SEQ ID NO: 114 - RNA, 20 nt, synthetic construct
  source 1..20: mol_type = other RNA
atgccacttctcagtacatg

SEQ ID NO: 115 - RNA, 20 nt, synthetic construct
  source 1..20: mol_type = other RNA
gggcgttcaggacacagact

SEQ ID NO: 116 - RNA, 20 nt, synthetic construct
  source 1..20: mol_type = other RNA
ttgctcaaatacaaagcctg

SEQ ID NO: 117 - RNA, 20 nt, synthetic construct
  source 1..20: mol_type = other RNA
atgggacaatcttatcaaaa

SEQ ID NO: 118 - RNA, 20 nt, synthetic construct
  source 1..20: mol_type = other RNA
cacagacttggtactgagga

SEQ ID NO: 119 - RNA, 20 nt, synthetic construct
  source 1..20: mol_type = other RNA
ggcttccacagacacaccca

SEQ ID NO: 120 - RNA, 20 nt, synthetic construct
  source 1..20: mol_type = other RNA
tctcaggaatttgttgaaac

SEQ ID NO: 121 - RNA, 20 nt, synthetic construct
  source 1..20: mol_type = other RNA
tggacttcagtacaactcac

SEQ ID NO: 122 - RNA, 20 nt, synthetic construct
  source 1..20: mol_type = other RNA
gccccacatgtactgagaag

SEQ ID NO: 123 - RNA, 20 nt, synthetic construct
  source 1..20: mol_type = other RNA
gggcgttcaggacacagact

SEQ ID NO: 124 - RNA, 20 nt, synthetic construct
  source 1..20: mol_type = other RNA
tctcaggaatttgttgaaac

SEQ ID NO: 125 - DNA, 41 bp, synthetic construct
  source 1..41: mol_type = other DNA
taatttctactcttgtagatcttgagcgtgctgaacagcgc

SEQ ID NO: 126 - DNA, 41 bp, synthetic construct
  source 1..41: mol_type = other DNA
taatttctactcttgtagatagagactccagttgccagcgg

SEQ ID NO: 127 - DNA, 41 bp, synthetic construct
  source 1..41: mol_type = other DNA
taatttctactcttgtagatagcgccacaagcttccggact

SEQ ID NO: 128 - DNA, 41 bp, synthetic construct
  source 1..41: mol_type = other DNA
taatttctactcttgtagatgggagatcatccacaaagccc

SEQ ID NO: 129 - DNA, 41 bp, synthetic construct
  source 1..41: mol_type = other DNA
taatttctactcttgtagataattccgctggcaactggagt

SEQ ID NO: 130 - DNA, 41 bp, synthetic construct
  source 1..41: mol_type = other DNA
taatttctactcttgtagattggatgatctcccgaaactga

SEQ ID NO: 131 - DNA, 41 bp, synthetic construct
  source 1..41: mol_type = other DNA
taatttctactcttgtagatcagtgtgtatcggtgcatggt

SEQ ID NO: 132 - DNA, 41 bp, synthetic construct
  source 1..41: mol_type = other DNA
taatttctactcttgtagataacctactaatgggatcattc

SEQ ID NO: 133 - DNA, 41 bp, synthetic construct
  source 1..41: mol_type = other DNA
taatttctactcttgtagattgtcgatgagggctttgtgga

SEQ ID NO: 134 - DNA, 41 bp, synthetic construct
  source 1..41: mol_type = other DNA
taatttctactcttgtagattcctcagtttcgggagatcat

SEQ ID NO: 135 - DNA, 41 bp, synthetic construct
  source 1..41: mol_type = other DNA
taatttctactcttgtagatcagggtggcctggatgtttct

SEQ ID NO: 136 - DNA, 41 bp, synthetic construct
  source 1..41: mol_type = other DNA
taatttctactcttgtagataaaccatgcaccgatacacac

SEQ ID NO: 137 - DNA, 41 bp, synthetic construct
  source 1..41: mol_type = other DNA
taatttctactcttgtagattatttgagcaatatgcggaaa

SEQ ID NO: 138 - DNA, 41 bp, synthetic construct
  source 1..41: mol_type = other DNA
taatttctactcttgtagatttccagtgagttgtactgaag

SEQ ID NO: 139 - DNA, 41 bp, synthetic construct
  source 1..41: mol_type = other DNA
taatttctactcttgtagatcgcatattgctcaaatacaaa

SEQ ID NO: 140 - DNA, 41 bp, synthetic construct
  source 1..41: mol_type = other DNA
taatttctactcttgtagatagcaatatgcggaaagctgtg

SEQ ID NO: 141 - DNA, 41 bp, synthetic construct
  source 1..41: mol_type = other DNA
taatttctactcttgtagataagttgcgtgtgtctgtttcc

SEQ ID NO: 142 - DNA, 41 bp, synthetic construct
  source 1..41: mol_type = other DNA
taatttctactcttgtagatttctggctttccagggtggcc

SEQ ID NO: 143 - DNA, 41 bp, synthetic construct
  source 1..41: mol_type = other DNA
taatttctactcttgtagatataagattgtcccattcatca

SEQ ID NO: 144 - DNA, 100 bp, synthetic construct
  source 1..100: mol_type = other DNA
cacgcaactttaaattccgcgttttagagctagaaatagcaagttaaaataaggctagtccgttatcaacttgaaaaagtggcaccgagtcggtgctttt

SEQ ID NO: 145 - DNA, 100 bp, synthetic construct
  source 1..100: mol_type = other DNA
tcaactggtgtcgagaagtcgttttagagctagaaatagcaagttaaaataaggctagtccgttatcaacttgaaaaagtggcaccgagtcggtgctttt

SEQ ID NO: 146 - DNA, 41 bp, synthetic construct
  source 1..41: mol_type = other DNA
taatttctactcttgtagattcctttgcaacatcctcagaa

SEQ ID NO: 147 - DNA, 100 bp, synthetic construct
  source 1..100: mol_type = other DNA
ggcgctgttcagcacgctcagttttagagctagaaatagcaagttaaaataaggctagtccgttatcaacttgaaaaagtggcaccgagtcggtgctttt

SEQ ID NO: 148 - DNA, 41 bp, synthetic construct
  source 1..41: mol_type = other DNA
taatttctactcttgtagataacaaattcctgagatttgag

SEQ ID NO: 149 - DNA, 100 bp, synthetic construct
  source 1..100: mol_type = other DNA
tcgagaagtccggaagcttggttttagagctagaaatagcaagttaaaataaggctagtccgttatcaacttgaaaaagtggcaccgagtcggtgctttt

SEQ ID NO: 150 - DNA, 100 bp, synthetic construct
  source 1..100: mol_type = other DNA
tgaagtccacttcgggccatgttttagagctagaaatagcaagttaaaataaggctagtccgttatcaacttgaaaaagtggcaccgagtcggtgctttt

SEQ ID NO: 151 - DNA, 100 bp, synthetic construct
  source 1..100: mol_type = other DNA
tcagtacatgtggggcgttcgttttagagctagaaatagcaagttaaaataaggctagtccgttatcaacttgaaaaagtggcaccgagtcggtgctttt

SEQ ID NO: 152 - DNA, 100 bp, synthetic construct
  source 1..100: mol_type = other DNA
ttccagtgtgtatcggtgcagttttagagctagaaatagcaagttaaaataaggctagtccgttatcaacttgaaaaagtggcaccgagtcggtgctttt

SEQ ID NO: 153 - DNA, 100 bp, synthetic construct
  source 1..100: mol_type = other DNA
cggggctttgctatgatactgttttagagctagaaatagcaagttaaaataaggctagtccgttatcaacttgaaaaagtggcaccgagtcggtgctttt

SEQ ID NO: 154 - DNA, 41 bp, synthetic construct
  source 1..41: mol_type = other DNA
taatttctactcttgtagataaatgatgaatgatcccatta

SEQ ID NO: 155 - DNA, 100 bp, synthetic construct
  source 1..100: mol_type = other DNA
gttgtactgaagtccacttcgttttagagctagaaatagcaagttaaaataaggctagtccgttatcaacttgaaaaagtggcaccgagtcggtgctttt

SEQ ID NO: 156 - DNA, 100 bp, synthetic construct
  source 1..100: mol_type = other DNA
tggacttcagtacaactcacgttttagagctagaaatagcaagttaaaataaggctagtccgttatcaacttgaaaaagtggcaccgagtcggtgctttt

SEQ ID NO: 157 - DNA, 100 bp, synthetic construct
  source 1..100: mol_type = other DNA
agttgtactgaagtccacttgttttagagctagaaatagcaagttaaaataaggctagtccgttatcaacttgaaaaagtggcaccgagtcggtgctttt

SEQ ID NO: 158 - DNA, 100 bp, synthetic construct
  source 1..100: mol_type = other DNA
cctcatcgacagaaacatccgttttagagctagaaatagcaagttaaaataaggctagtccgttatcaacttgaaaaagtggcaccgagtcggtgctttt

SEQ ID NO: 159 - DNA, 100 bp, synthetic construct
  source 1..100: mol_type = other DNA
gcggaaagctgtgaagatacgttttagagctagaaatagcaagttaaaataaggctagtccgttatcaacttgaaaaagtggcaccgagtcggtgctttt

SEQ ID NO: 160 - DNA, 100 bp, synthetic construct
  source 1..100: mol_type = other DNA
ttttcagcgccacaagcttcgttttagagctagaaatagcaagttaaaataaggctagtccgttatcaacttgaaaaagtggcaccgagtcggtgctttt

SEQ ID NO: 161 - DNA, 100 bp, synthetic construct
  source 1..100: mol_type = other DNA
ctgaagtccacttcgggccagttttagagctagaaatagcaagttaaaataaggctagtccgttatcaacttgaaaaagtggcaccgagtcggtgctttt

SEQ ID NO: 162 - DNA, 100 bp, synthetic construct
  source 1..100: mol_type = other DNA
gccacttctcagtacatgtggttttagagctagaaatagcaagttaaaataaggctagtccgttatcaacttgaaaaagtggcaccgagtcggtgctttt

SEQ ID NO: 163 - DNA, 100 bp, synthetic construct
  source 1..100: mol_type = other DNA
tgatgaatgatcccattagtgttttagagctagaaatagcaagttaaaataaggctagtccgttatcaacttgaaaaagtggcaccgagtcggtgctttt

SEQ ID NO: 164 - DNA, 100 bp, synthetic construct
  source 1..100: mol_type = other DNA
cctggatgtttctgtcgatggttttagagctagaaatagcaagttaaaataaggctagtccgttatcaacttgaaaaagtggcaccgagtcggtgctttt

SEQ ID NO: 165 - DNA, 100 bp, synthetic construct
  source 1..100: mol_type = other DNA
gccccacatgtactgagaaggttttagagctagaaatagcaagttaaaataaggctagtccgttatcaacttgaaaaagtggcaccgagtcggtgctttt

SEQ ID NO: 166 - DNA, 100 bp, synthetic construct
  source 1..100: mol_type = other DNA
tgcggaaagctgtgaagatagttttagagctagaaatagcaagttaaaataaggctagtccgttatcaacttgaaaaagtggcaccgagtcggtgctttt

SEQ ID NO: 167 - DNA, 100 bp, synthetic construct
  source 1..100: mol_type = other DNA
ctggatgtttctgtcgatgagttttagagctagaaatagcaagttaaaataaggctagtccgttatcaacttgaaaaagtggcaccgagtcggtgctttt

SEQ ID NO: 168 - DNA, 100 bp, synthetic construct
  source 1..100: mol_type = other DNA
ttctgtcgatgagggctttggttttagagctagaaatagcaagttaaaataaggctagtccgttatcaacttgaaaaagtggcaccgagtcggtgctttt

SEQ ID NO: 169 - DNA, 100 bp, synthetic construct
  source 1..100: mol_type = other DNA
atgccacttctcagtacatggttttagagctagaaatagcaagttaaaataaggctagtccgttatcaacttgaaaaagtggcaccgagtcggtgctttt

SEQ ID NO: 170 - DNA, 100 bp, synthetic construct
  source 1..100: mol_type = other DNA
gggcgttcaggacacagactgttttagagctagaaatagcaagttaaaataaggctagtccgttatcaacttgaaaaagtggcaccgagtcggtgctttt

SEQ ID NO: 171 - DNA, 100 bp, synthetic construct
  source 1..100: mol_type = other DNA
ttgctcaaatacaaagcctggttttagagctagaaatagcaagttaaaataaggctagtccgttatcaacttgaaaaagtggcaccgagtcggtgctttt

SEQ ID NO: 172 - DNA, 100 bp, synthetic construct
  source 1..100: mol_type = other DNA
atgggacaatcttatcaaaagttttagagctagaaatagcaagttaaaataaggctagtccgttatcaacttgaaaaagtggcaccgagtcggtgctttt

SEQ ID NO: 173 - DNA, 100 bp, synthetic construct
  source 1..100: mol_type = other DNA
cacagacttggtactgaggagttttagagctagaaatagcaagttaaaataaggctagtccgttatcaacttgaaaaagtggcaccgagtcggtgctttt

SEQ ID NO: 174 - DNA, 100 bp, synthetic construct
  source 1..100: mol_type = other DNA
ggcttccacagacacacccagttttagagctagaaatagcaagttaaaataaggctagtccgttatcaacttgaaaaagtggcaccgagtcggtgctttt

SEQ ID NO: 175 - DNA, 100 bp, synthetic construct
  source 1..100: mol_type = other DNA
tctcaggaatttgttgaaacgttttagagctagaaatagcaagttaaaataaggctagtccgttatcaacttgaaaaagtggcaccgagtcggtgctttt

SEQ ID NO: 176 - DNA, 100 bp, synthetic construct; identical to SEQ ID NO: 156
  source 1..100: mol_type = other DNA

SEQ ID NO: 177 - DNA, 100 bp, synthetic construct; identical to SEQ ID NO: 165
  source 1..100: mol_type = other DNA

SEQ ID NO: 178 - DNA, 100 bp, synthetic construct; identical to SEQ ID NO: 170
  source 1..100: mol_type = other DNA

SEQ ID NO: 179 - DNA, 100 bp, synthetic construct; identical to SEQ ID NO: 175
  source 1..100: mol_type = other DNA

SEQ ID NO: 180 - DNA, 20 bp, Homo sapiens
  source 1..20: mol_type = genomic DNA
tccgttcgcacgccgattac

SEQ ID NO: 181 - DNA, 21 bp, Homo sapiens
  source 1..21: mol_type = genomic DNA
acctggatggcagccgcgaga

SEQ ID NO: 182 - DNA, 20 bp, Homo sapiens
  source 1..20: mol_type = genomic DNA
gccggtaatcggcgtgcgaa

SEQ ID NO: 183 - DNA, 20 bp, Homo sapiens
  source 1..20: mol_type = genomic DNA
gcgcgtgatgcgccggtaat

SEQ ID NO: 184 - DNA, 21 bp, Homo sapiens
  source 1..21: mol_type = genomic DNA
gcccttagcgtgaagatggcc

SEQ ID NO: 185 - DNA, 21 bp, Homo sapiens
  source 1..21: mol_type = genomic DNA
aggccggccgctttcacctgg

SEQ ID NO: 186 - DNA, 20 bp, Homo sapiens
  source 1..20: mol_type = genomic DNA
cggcgtgcgaacggaatgtg

SEQ ID NO: 187 - DNA, 20 bp, Homo sapiens
  source 1..20: mol_type = genomic DNA
cgggcgctgccggtcaaatc

SEQ ID NO: 188 - DNA, 20 bp, Homo sapiens
  source 1..20: mol_type = genomic DNA
aagtgcacgcggatgctcgt

SEQ ID NO: 189 - DNA, 20 bp, Homo sapiens
  source 1..20: mol_type = genomic DNA
ggacgcctgcggattctact

SEQ ID NO: 190 - DNA, 20 bp, Homo sapiens
  source 1..20: mol_type = genomic DNA
tgtgcggaagtgcgtgtcgc

SEQ ID NO: 191 - DNA, 20 bp, Homo sapiens
  source 1..20: mol_type = genomic DNA
gggcgaaaaagcagttccgc

SEQ ID NO: 192 - DNA, 20 bp, Homo sapiens
  source 1..20: mol_type = genomic DNA
gacgcctgcggattctactg

SEQ ID NO: 193 - DNA, 20 bp, Homo sapiens
  source 1..20: mol_type = genomic DNA
tggtgcgcgacagccgccag

SEQ ID NO: 194 - DNA, 20 bp, Homo sapiens
  source 1..20: mol_type = genomic DNA
aaagtgcacgcggatgctcg

SEQ ID NO: 195 - DNA, 20 bp, Homo sapiens
  source 1..20: mol_type = genomic DNA
tggacgcctgcggattctac

SEQ ID NO: 196 - DNA, 20 bp, Homo sapiens
  source 1..20: mol_type = genomic DNA
atccgcgtgcactttcaggc

SEQ ID NO: 197 - DNA, 20 bp, Homo sapiens
  source 1..20: mol_type = genomic DNA
agtagaatccgcaggcgtcc

SEQ ID NO: 198 - DNA, 20 bp, Homo sapiens
  source 1..20: mol_type = genomic DNA
cgaggccatcttcacgctaa

SEQ ID NO: 199 - DNA, 20 bp, Homo sapiens
  source 1..20: mol_type = genomic DNA
cgctgccggtcaaatctgga

SEQ ID NO: 200 - DNA, 20 bp, Homo sapiens
  source 1..20: mol_type = genomic DNA
tttcgcccttagcgtgaaga

SEQ ID NO: 201 - DNA, 20 bp, Homo sapiens
  source 1..20: mol_type = genomic DNA
cgaaaaagcagttccgctgg

SEQ ID NO: 202 - DNA, 20 bp, Homo sapiens
  source 1..20: mol_type = genomic DNA
gtgcgccccgtgcacgctca

SEQ ID NO: 203 - DNA, 20 bp, Homo sapiens
  source 1..20: mol_type = genomic DNA
tgcggaagtgcgtgtcgccg

SEQ ID NO: 204 - DNA, 20 bp, Homo sapiens
  source 1..20: mol_type = genomic DNA
gcggaagtgcgtgtcgccgg

SEQ ID NO: 205 - DNA, 20 bp, Homo sapiens
  source 1..20: mol_type = genomic DNA
gggcccccagtagaatccgc

SEQ ID NO: 206 - DNA, 20 bp, Homo sapiens
  source 1..20: mol_type = genomic DNA
cgctggcgcgcgtgatgcgc

SEQ ID NO: 207 - DNA, 20 bp, Homo sapiens
  source 1..20: mol_type = genomic DNA
tcaggtagtcgcggaggacg

SEQ ID NO: 208 - DNA, 20 bp, Homo sapiens
  source 1..20: mol_type = genomic DNA
gagcatccgcgtgcactttc

SEQ ID NO: 209 - DNA, 20 bp, Homo sapiens
  source 1..20: mol_type = genomic DNA
ctcaggtagtcgcggaggac

SEQ ID NO: 210 - DNA, 20 bp, Homo sapiens
  source 1..20: mol_type = genomic DNA
ccgaggccatcttcacgcta

SEQ ID NO: 211 - DNA, 20 bp, Homo sapiens
  source 1..20: mol_type = genomic DNA
cagcgcatcgtggccaccgt

SEQ ID NO: 212 - DNA, 20 bp, Homo sapiens
  source 1..20: mol_type = genomic DNA
cgtgcgccccgtgcacgctc

SEQ ID NO: 213 - DNA, 20 bp, Homo sapiens
  source 1..20: mol_type = genomic DNA
agcgcgctcctggacgcctg

SEQ ID NO: 214 - DNA, 20 bp, Homo sapiens
  source 1..20: mol_type = genomic DNA
ggatgcgagccaggttctcg

SEQ ID NO: 215 - DNA, 20 bp, Homo sapiens
  source 1..20: mol_type = genomic DNA
tgcgccccgtgcacgctcag

SEQ ID NO: 216 - DNA, 20 bp, Homo sapiens
  source 1..20: mol_type = genomic DNA
caccgtgggccgcgagaacc

SEQ ID NO: 217 - DNA, 20 bp, Homo sapiens
  source 1..20: mol_type = genomic DNA
gcgtgcacggggcgcacgag

SEQ ID NO: 218 - DNA, 20 bp, Homo sapiens
  source 1..20: mol_type = genomic DNA
gcggatgctcgtgggtcccg

SEQ ID NO: 219 - DNA, 20 bp, Homo sapiens
  source 1..20: mol_type = genomic DNA
gctgccggtcaaatctggaa

SEQ ID NO: 220 - DNA, 20 bp, Homo sapiens
  source 1..20: mol_type = genomic DNA
ggccggcctgaaagtgcacg

SEQ ID NO: 221 - DNA, 20 bp, Homo sapiens
  source 1..20: mol_type = genomic DNA
gctcaggtagtcgcggagga

SEQ ID NO: 222 - DNA, 20 bp, Homo sapiens
  source 1..20: mol_type = genomic DNA
tcaggccggccgctttcacc

SEQ ID NO: 223 - DNA, 20 bp, Homo sapiens
  source 1..20: mol_type = genomic DNA
taggatggtagcacacaacc

SEQ ID NO: 224 - DNA, 20 bp, Homo sapiens
  source 1..20: mol_type = genomic DNA
cggtggccacgatgcgctgg

SEQ ID NO: 225 - DNA, 20 bp, Homo sapiens
  source 1..20: mol_type = genomic DNA
aggaaggttctggccgccgt

SEQ ID NO: 226 - DNA, 20 bp, Homo sapiens
  source 1..20: mol_type = genomic DNA
gtcgcggaggacggggttga

SEQ ID NO: 227 - DNA, 20 bp, Homo sapiens
  source 1..20: mol_type = genomic DNA
caggttctcgcggcccacgg

SEQ ID NO: 228 - DNA, 20 bp, Homo sapiens
  source 1..20: mol_type = genomic DNA
gtgtgctaccatcctacaga

SEQ ID NO: 229 - DNA, 20 bp, Homo sapiens
  source 1..20: mol_type = genomic DNA
cttagcgtgaagatggcctc

SEQ ID NO: 230 - DNA, 20 bp, Homo sapiens
  source 1..20: mol_type = genomic DNA
aggagctcaggtagtcgcgg

SEQ ID NO: 231 - DNA, 20 bp, Homo sapiens
  source 1..20: mol_type = genomic DNA
cgctgcgccagcgccgcgtg

SEQ ID NO: 232 - DNA, 20 bp, Homo sapiens
  source 1..20: mol_type = genomic DNA
cggcgccgcgccgcatgctg

SEQ ID NO: 233 - DNA, 20 bp, Homo sapiens
  source 1..20: mol_type = genomic DNA
agccaggttctcgcggccca

SEQ ID NO: 234 - DNA, 20 bp, Homo sapiens
  source 1..20: mol_type = genomic DNA
gctggcggctgtcgcgcacc

SEQ ID NO: 235 - DNA, 20 bp, Homo sapiens
  source 1..20: mol_type = genomic DNA
cggctgcgcgccgagcccgt

SEQ ID NO: 236 - DNA, 20 bp, Homo sapiens
  source 1..20: mol_type = genomic DNA
gtgcggaagtgcgtgtcgcc

SEQ ID NO: 237 - DNA, 20 bp, Homo sapiens
  source 1..20: mol_type = genomic DNA
agaggcagtcgaagctctcg

SEQ ID NO: 238 - DNA, 20 bp, Homo sapiens
  source 1..20: mol_type = genomic DNA
tcgcggaggacggggttgag

SEQ ID NO: 239 - DNA, 20 bp, Homo sapiens
  source 1..20: mol_type = genomic DNA
agtgcgtgtcgccgggggcc

SEQ ID NO: 240 - DNA, 20 bp, Homo sapiens
  source 1..20: mol_type = genomic DNA
ctgccggtcaaatctggaag

SEQ ID NO: 241 - DNA, 20 bp, Homo sapiens
  source 1..20: mol_type = genomic DNA
gcggctgtcgcgcaccagga

SEQ ID NO: 242 - DNA, 20 bp, Homo sapiens
  source 1..20: mol_type = genomic DNA
agctctcgcggctgccatcc

SEQ ID NO: 243 - DNA, 20 bp, Homo sapiens
  source 1..20: mol_type = genomic DNA
gccggccgctttcacctgga

SEQ ID NO: 244 - DNA, 20 bp, Homo sapiens
  source 1..20: mol_type = genomic DNA
gatggtagcacacaaccagg

SEQ ID NO: 245 - DNA, 20 bp, Homo sapiens
  source 1..20: mol_type = genomic DNA
gtgctaccatcctacagaag

SEQ ID NO: 246 - DNA, 20 bp, Homo sapiens
  source 1..20: mol_type = genomic DNA
ctgcattgtcggctgccacc

SEQ ID NO: 247 - DNA, 20 bp, Homo sapiens
  source 1..20: mol_type = genomic DNA
cgagcccgtgggcaccttcc

SEQ ID NO: 248 - DNA, 20 bp, Homo sapiens
  source 1..20: mol_type = genomic DNA
cgactgcctcttcgagctgc

SEQ ID NO: 249 - DNA, 20 bp, Homo sapiens
  source 1..20: mol_type = genomic DNA
ccacggtggccacgatgcgc

SEQ ID NO: 250 - DNA, 20 bp, Homo sapiens
  source 1..20: mol_type = genomic DNA
agtcgcggaggacggggttg

SEQ ID NO: 251 - DNA, 20 bp, Homo sapiens
  source 1..20: mol_type = genomic DNA
cggtcaaatctggaagggga

SEQ ID NO: 252 - DNA, 20 bp, Homo sapiens
  source 1..20: mol_type = genomic DNA
tgtgctaccatcctacagaa

SEQ ID NO: 253 - DNA, 20 bp, Homo sapiens
  source 1..20: mol_type = genomic DNA
gcggccgtgccccgcggtcc

SEQ ID NO: 254 - DNA, 20 bp, Homo sapiens
  source 1..20: mol_type = genomic DNA
cagcagcagagccccgacgg

SEQ ID NO: 255 - DNA, 20 bp, Homo sapiens
  source 1..20: mol_type = genomic DNA
ccagcgcatcgtggccaccg

SEQ ID NO: 256 - DNA, 20 bp, Homo sapiens
  source 1..20: mol_type = genomic DNA
ccgtcggggctctgctgctg

SEQ ID NO: 257 - DNA, 20 bp, Homo sapiens
  source 1..20: mol_type = genomic DNA
gcgcaccaggaaggtgccca

SEQ ID NO: 258 - DNA, 20 bp, Homo sapiens
  source 1..20: mol_type = genomic DNA
ggcggcgccgcgccgcatgc

SEQ ID NO: 259 - DNA, 20 bp, Homo sapiens
  source 1..20: mol_type = genomic DNA
tgggggcccctgagcgtgca

SEQ ID NO: 260 - DNA, 20 bp, Homo sapiens
  source 1..20: mol_type = genomic DNA
gcgcccgcggccgtgccccg

SEQ ID NO: 261 - DNA, 20 bp, Homo sapiens
  source 1..20: mol_type = genomic DNA
ggctgccatccaggtgaaag

SEQ ID NO: 262 - DNA, 20 bp, Homo sapiens
  source 1..20: mol_type = genomic DNA
cgcggaggacggggttgagg

SEQ ID NO: 263 - DNA, 20 bp, Homo sapiens
  source 1..20: mol_type = genomic DNA
gggcccccagtagaatccgc

SEQ ID NO: 264 - RNA, 20 nt, synthetic construct
  source 1..20: mol_type = other RNA
tccgttcgcacgccgattac

SEQ ID NO: 265 - RNA, 21 nt, synthetic construct
  source 1..21: mol_type = other RNA
acctggatggcagccgcgaga

SEQ ID NO: 266 - RNA, 20 nt, synthetic construct
  source 1..20: mol_type = other RNA
gccggtaatcggcgtgcgaa

SEQ ID NO: 267 - RNA, 20 nt, synthetic construct
  source 1..20: mol_type = other RNA
gcgcgtgatgcgccggtaat

SEQ ID NO: 268 - RNA, 21 nt, synthetic construct
  source 1..21: mol_type = other RNA
gcccttagcgtgaagatggcc

SEQ ID NO: 269 - RNA, 21 nt, synthetic construct
  source 1..21: mol_type = other RNA
aggccggccgctttcacctgg

SEQ ID NO: 270 - RNA, 20 nt, synthetic construct
  source 1..20: mol_type = other RNA
cggcgtgcgaacggaatgtg

SEQ ID NO: 271 - RNA, 20 nt, synthetic construct
  source 1..20: mol_type = other RNA
cgggcgctgccggtcaaatc

SEQ ID NO: 272 - RNA, 20 nt, synthetic construct
  source 1..20: mol_type = other RNA
aagtgcacgcggatgctcgt

SEQ ID NO: 273 - RNA, 20 nt, synthetic construct
  source 1..20: mol_type = other RNA
ggacgcctgcggattctact

SEQ ID NO: 274 - RNA, 20 nt, synthetic construct
  source 1..20: mol_type = other RNA
tgtgcggaagtgcgtgtcgc

SEQ ID NO: 275 - RNA, 20 nt, synthetic construct
  source 1..20: mol_type = other RNA
gggcgaaaaagcagttccgc

SEQ ID NO: 276 - RNA, 20 nt, synthetic construct
  source 1..20: mol_type = other RNA
gacgcctgcggattctactg

SEQ ID NO: 277 - RNA, 20 nt, synthetic construct
  source 1..20: mol_type = other RNA
tggtgcgcgacagccgccag

SEQ ID NO: 278 - RNA, 20 nt, synthetic construct
  source 1..20: mol_type = other RNA
aaagtgcacgcggatgctcg

SEQ ID NO: 279 - RNA, 20 nt, synthetic construct
  source 1..20: mol_type = other RNA
tggacgcctgcggattctac

SEQ ID NO: 280 - RNA, 20 nt, synthetic construct
  source 1..20: mol_type = other RNA
atccgcgtgcactttcaggc

SEQ ID NO: 281 - RNA, 20 nt, synthetic construct
  source 1..20: mol_type = other RNA
agtagaatccgcaggcgtcc

SEQ ID NO: 282 - RNA, 20 nt, synthetic construct
  source 1..20: mol_type = other RNA
cgaggccatcttcacgctaa

SEQ ID NO: 283 - RNA, 20 nt, synthetic construct
  source 1..20: mol_type = other RNA
cgctgccggtcaaatctgga

SEQ ID NO: 284 - RNA, 20 nt, synthetic construct
  source 1..20: mol_type = other RNA
tttcgcccttagcgtgaaga

SEQ ID NO: 285 - RNA, 20 nt, synthetic construct
  source 1..20: mol_type = other RNA
cgaaaaagcagttccgctgg

SEQ ID NO: 286 - RNA, 20 nt, synthetic construct
  source 1..20: mol_type = other RNA
gtgcgccccgtgcacgctca

SEQ ID NO: 287 - RNA, 20 nt, synthetic construct
  source 1..20: mol_type = other RNA
tgcggaagtgcgtgtcgccg

SEQ ID NO: 288 - RNA, 20 nt, synthetic construct
  source 1..20: mol_type = other RNA
gcggaagtgcgtgtcgccgg

SEQ ID NO: 289 - RNA, 20 nt, synthetic construct
  source 1..20: mol_type = other RNA
gggcccccagtagaatccgc

SEQ ID NO: 290 - RNA, 20 nt, synthetic construct
  source 1..20: mol_type = other RNA
cgctggcgcgcgtgatgcgc

SEQ ID NO: 291 - RNA, 20 nt, synthetic construct
  source 1..20: mol_type = other RNA
tcaggtagtcgcggaggacg

SEQ ID NO: 292 - RNA, 20 nt, synthetic construct
  source 1..20: mol_type = other RNA
gagcatccgcgtgcactttc

SEQ ID NO: 293 - RNA, 20 nt, synthetic construct
  source 1..20: mol_type = other RNA
ctcaggtagtcgcggaggac

SEQ ID NO: 294 - RNA, 20 nt, synthetic construct
  source 1..20: mol_type = other RNA
ccgaggccatcttcacgcta

SEQ ID NO: 295 - RNA, 20 nt, synthetic construct
  source 1..20: mol_type = other RNA
cagcgcatcgtggccaccgt

SEQ ID NO: 296 - RNA, 20 nt, synthetic construct
  source 1..20: mol_type = other RNA
cgtgcgccccgtgcacgctc

SEQ ID NO: 297 - RNA, 20 nt, synthetic construct
  source 1..20: mol_type = other RNA
agcgcgctcctggacgcctg

SEQ ID NO: 298 - RNA, 20 nt, synthetic construct
  source 1..20: mol_type = other RNA
ggatgcgagccaggttctcg

SEQ ID NO: 299 - RNA, 20 nt, synthetic construct
  source 1..20: mol_type = other RNA
tgcgccccgtgcacgctcag

SEQ ID NO: 300 - RNA, 20 nt, synthetic construct
  source 1..20: mol_type = other RNA
caccgtgggccgcgagaacc

SEQ ID NO: 301 - RNA, 20 nt, synthetic construct
  source 1..20: mol_type = other RNA
gcgtgcacggggcgcacgag

SEQ ID NO: 302 - RNA, 20 nt, synthetic construct
  source 1..20: mol_type = other RNA
gcggatgctcgtgggtcccg

SEQ ID NO: 303 - RNA, 20 nt, synthetic construct
  source 1..20: mol_type = other RNA
gctgccggtcaaatctggaa

SEQ ID NO: 304 - RNA, 20 nt, synthetic construct
  source 1..20: mol_type = other RNA
ggccggcctgaaagtgcacg

SEQ ID NO: 305 - RNA, 20 nt, synthetic construct
  source 1..20: mol_type = other RNA
gctcaggtagtcgcggagga

SEQ ID NO: 306 - RNA, 20 nt, synthetic construct
  source 1..20: mol_type = other RNA
tcaggccggccgctttcacc

SEQ ID NO: 307 - RNA, 20 nt, synthetic construct
  source 1..20: mol_type = other RNA
taggatggtagcacacaacc

SEQ ID NO: 308 - RNA, 20 nt, synthetic construct
  source 1..20: mol_type = other RNA
cggtggccacgatgcgctgg

SEQ ID NO: 309 - RNA, 20 nt, synthetic construct
  source 1..20: mol_type = other RNA
aggaaggttctggccgccgt

SEQ ID NO: 310 - RNA, 20 nt, synthetic construct
  source 1..20: mol_type = other RNA
gtcgcggaggacggggttga

SEQ ID NO: 311 - RNA, 20 nt, synthetic construct
  source 1..20: mol_type = other RNA
caggttctcgcggcccacgg

SEQ ID NO: 312 - RNA, 20 nt, synthetic construct
  source 1..20: mol_type = other RNA
gtgtgctaccatcctacaga

SEQ ID NO: 313 - RNA, 20 nt, synthetic construct
  source 1..20: mol_type = other RNA
cttagcgtgaagatggcctc

SEQ ID NO: 314 - RNA, 20 nt, synthetic construct
  source 1..20: mol_type = other RNA
aggagctcaggtagtcgcgg

SEQ ID NO: 315 - RNA, 20 nt, synthetic construct
  source 1..20: mol_type = other RNA
cgctgcgccagcgccgcgtg

SEQ ID NO: 316 - RNA, 20 nt, synthetic construct
  source 1..20: mol_type = other RNA
cggcgccgcgccgcatgctg

SEQ ID NO: 317 - RNA, 20 nt, synthetic construct
  source 1..20: mol_type = other RNA
agccaggttctcgcggccca

SEQ ID NO: 318 - RNA, 20 nt, synthetic construct
  source 1..20: mol_type = other RNA
gctggcggctgtcgcgcacc

SEQ ID NO: 319 - RNA, 20 nt, synthetic construct
  source 1..20: mol_type = other RNA
cggctgcgcgccgagcccgt

SEQ ID NO: 320 - RNA, 20 nt, synthetic construct
  source 1..20: mol_type = other RNA
gtgcggaagtgcgtgtcgcc

SEQ ID NO: 321 - RNA, 20 nt, synthetic construct
  source 1..20: mol_type = other RNA
agaggcagtcgaagctctcg

SEQ ID NO: 322 - RNA, 20 nt, synthetic construct
  source 1..20: mol_type = other RNA
tcgcggaggacggggttgag

SEQ ID NO: 323 - RNA, 20 nt, synthetic construct
  source 1..20: mol_type = other RNA
agtgcgtgtcgccgggggcc

SEQ ID NO: 324 - RNA, 20 nt, synthetic construct
  source 1..20: mol_type = other RNA
ctgccggtcaaatctggaag

SEQ ID NO: 325 - RNA, 20 nt, synthetic construct
  source 1..20: mol_type = other RNA
gcggctgtcgcgcaccagga

SEQ ID NO: 326 - RNA, 20 nt, synthetic construct
  source 1..20: mol_type = other RNA
agctctcgcggctgccatcc

SEQ ID NO: 327 - RNA, 20 nt, synthetic construct
  source 1..20: mol_type = other RNA
gccggccgctttcacctgga

SEQ ID NO: 328 - RNA, 20 nt, synthetic construct
  source 1..20: mol_type = other RNA
gatggtagcacacaaccagg

SEQ ID NO: 329 - RNA, 20 nt, synthetic construct
  source 1..20: mol_type = other RNA
gtgctaccatcctacagaag

SEQ ID NO: 330 - RNA, 20 nt, synthetic construct
  source 1..20: mol_type = other RNA
ctgcattgtcggctgccacc

SEQ ID NO: 331 - RNA, 20 nt, synthetic construct
  source 1..20: mol_type = other RNA
cgagcccgtgggcaccttcc

SEQ ID NO: 332 - RNA, 20 nt, synthetic construct
  source 1..20: mol_type = other RNA
cgactgcctcttcgagctgc

SEQ ID NO: 333 - RNA, 20 nt, synthetic construct
  source 1..20: mol_type = other RNA
ccacggtggccacgatgcgc

SEQ ID NO: 334 - RNA, 20 nt, synthetic construct
  source 1..20: mol_type = other RNA
agtcgcggaggacggggttg

SEQ ID NO: 335 - RNA, 20 nt, synthetic construct
  source 1..20: mol_type = other RNA
cggtcaaatctggaagggga

SEQ ID NO: 336 - RNA, 20 nt, synthetic construct
  source 1..20: mol_type = other RNA
tgtgctaccatcctacagaa

SEQ ID NO: 337 - RNA, 20 nt, synthetic construct
  source 1..20: mol_type = other RNA
gcggccgtgccccgcggtcc

SEQ ID NO: 338 - RNA, 20 nt, synthetic construct
  source 1..20: mol_type = other RNA
cagcagcagagccccgacgg

SEQ ID NO: 339 - RNA, 20 nt, synthetic construct
  source 1..20: mol_type = other RNA
ccagcgcatcgtggccaccg

SEQ ID NO: 340 - RNA, 20 nt, synthetic construct
  source 1..20: mol_type = other RNA
ccgtcggggctctgctgctg

SEQ ID NO: 341 - RNA, 20 nt, synthetic construct
  source 1..20: mol_type = other RNA
gcgcaccaggaaggtgccca

SEQ ID NO: 342 - RNA, 20 nt, synthetic construct
  source 1..20: mol_type = other RNA
ggcggcgccgcgccgcatgc

SEQ ID NO: 343 - RNA, 20 nt, synthetic construct
  source 1..20: mol_type = other RNA
tgggggcccctgagcgtgca

SEQ ID NO: 344 - RNA, 20 nt, synthetic construct
  source 1..20: mol_type = other RNA
gcgcccgcggccgtgccccg

SEQ ID NO: 345 - RNA, 20 nt, synthetic construct
  source 1..20: mol_type = other RNA
ggctgccatccaggtgaaag

SEQ ID NO: 346 - RNA, 20 nt, synthetic construct
  source 1..20: mol_type = other RNA
cgcggaggacggggttgagg

SEQ ID NO: 347 - RNA, 20 nt, synthetic construct
  source 1..20: mol_type = other RNA
gggcccccagtagaatccgc

SEQ ID NO: 348 - DNA, 100 bp, synthetic construct
  source 1..100: mol_type = other DNA
tccgttcgcacgccgattacgttttagagctagaaatagcaagttaaaataaggctagtccgttatcaacttgaaaaagtggcaccgagtcggtgctttt

SEQ ID NO: 349 - DNA, 41 bp, synthetic construct
  source 1..41: mol_type = other DNA
taatttctactcttgtagatacctggatggcagccgcgaga

SEQ ID NO: 350 - DNA, 100 bp, synthetic construct
  source 1..100: mol_type = other DNA
gccggtaatcggcgtgcgaagttttagagctagaaatagcaagttaaaataaggctagtccgttatcaacttgaaaaagtggcaccgagtcggtgctttt

SEQ ID NO: 351 - DNA, 100 bp, synthetic construct
  source 1..100: mol_type = other DNA
gcgcgtgatgcgccggtaatgttttagagctagaaatagcaagttaaaataaggctagtccgttatcaacttgaaaaagtggcaccgagtcggtgctttt

SEQ ID NO: 352 - DNA, 41 bp, synthetic construct
  source 1..41: mol_type = other DNA
taatttctactcttgtagatgcccttagcgtgaagatggcc

SEQ ID NO: 353 - DNA, 41 bp, synthetic construct
  source 1..41: mol_type = other DNA
taatttctactcttgtagataggccggccgctttcacctgg

SEQ ID NO: 354 - DNA, 100 bp, synthetic construct
  source 1..100: mol_type = other DNA
cggcgtgcgaacggaatgtggttttagagctagaaatagcaagttaaaataaggctagtccgttatcaacttgaaaaagtggcaccgagtcggtgctttt

SEQ ID NO: 355 - DNA, 100 bp, synthetic construct
  source 1..100: mol_type = other DNA
cgggcgctgccggtcaaatcgttttagagctagaaatagcaagttaaaataaggctagtccgttatcaacttgaaaaagtggcaccgagtcggtgctttt

SEQ ID NO: 356 - DNA, 100 bp, synthetic construct
  source 1..100: mol_type = other DNA
aagtgcacgcggatgctcgtgttttagagctagaaatagcaagttaaaataaggctagtccgttatcaacttgaaaaagtggcaccgagtcggtgctttt

SEQ ID NO: 357 - DNA, 100 bp, synthetic construct
  source 1..100: mol_type = other DNA
ggacgcctgcggattctactgttttagagctagaaatagcaagttaaaataaggctagtccgttatcaacttgaaaaagtggcaccgagtcggtgctttt

SEQ ID NO: 358 - DNA, 100 bp, synthetic construct
  source 1..100: mol_type = other DNA
tgtgcggaagtgcgtgtcgcgttttagagctagaaatagcaagttaaaataaggctagtccgttatcaacttgaaaaagtggcaccgagtcggtgctttt

SEQ ID NO: 359 - DNA, 100 bp, synthetic construct
  source 1..100: mol_type = other DNA
gggcgaaaaagcagttccgcgttttagagctagaaatagcaagttaaaataaggctagtccgttatcaacttgaaaaagtggcaccgagtcggtgctttt

SEQ ID NO: 360 - DNA, 100 bp, synthetic construct
  source 1..100: mol_type = other DNA
gacgcctgcggattctactggttttagagctagaaatagcaagttaaaataaggctagtccgttatcaacttgaaaaagtggcaccgagtcggtgctttt

SEQ ID NO: 361 - DNA, 100 bp, synthetic construct
  source 1..100: mol_type = other DNA
tggtgcgcgacagccgccaggttttagagctagaaatagcaagttaaaataaggctagtccgttatcaacttgaaaaagtggcaccgagtcggtgctttt

SEQ ID NO: 362 - DNA, 100 bp, synthetic construct
  source 1..100: mol_type = other DNA
aaagtgcacgcggatgctcggttttagagctagaaatagcaagttaaaataaggctagtccgttatcaacttgaaaaagtggcaccgagtcggtgctttt

SEQ ID NO: 363 - DNA, 100 bp, synthetic construct
  source 1..100: mol_type = other DNA
tggacgcctgcggattctacgttttagagctagaaatagcaagttaaaataaggctagtccgttatcaacttgaaaaagtggcaccgagtcggtgctttt

SEQ ID NO: 364 - DNA, 100 bp, synthetic construct
  source 1..100: mol_type = other DNA
atccgcgtgcactttcaggcgttttagagctagaaatagcaagttaaaataaggctagtccgttatcaacttgaaaaagtggcaccgagtcggtgctttt

SEQ ID NO: 365 - DNA, 100 bp, synthetic construct
  source 1..100: mol_type = other DNA
agtagaatccgcaggcgtccgttttagagctagaaatagcaagttaaaataaggctagtccgttatcaacttgaaaaagtggcaccgagtcggtgctttt

SEQ ID NO: 366 - DNA, 100 bp, synthetic construct
  source 1..100: mol_type = other DNA
cgaggccatcttcacgctaagttttagagctagaaatagcaagttaaaataaggctagtccgttatcaacttgaaaaagtggcaccgagtcggtgctttt

SEQ ID NO: 367 - DNA, 100 bp, synthetic construct
  source 1..100: mol_type = other DNA
cgctgccggtcaaatctggagttttagagctagaaatagcaagttaaaataaggctagtccgttatcaacttgaaaaagtggcaccgagtcggtgctttt

SEQ ID NO: 368 - DNA, 100 bp, synthetic construct
  source 1..100: mol_type = other DNA
tttcgcccttagcgtgaagagttttagagctagaaatagcaagttaaaataaggctagtccgttatcaacttgaaaaagtggcaccgagtcggtgctttt

SEQ ID NO: 369 - DNA, 100 bp, synthetic construct
  source 1..100: mol_type = other DNA
cgaaaaagcagttccgctgggttttagagctagaaatagcaagttaaaataaggctagtccgttatcaacttgaaaaagtggcaccgagtcggtgctttt

SEQ ID NO: 370 - DNA, 100 bp, synthetic construct
  source 1..100: mol_type = other DNA
gtgcgccccgtgcacgctcagttttagagctagaaatagcaagttaaaataaggctagtccgttatcaacttgaaaaagtggcaccgagtcggtgctttt

SEQ ID NO: 371 - DNA, 100 bp, synthetic construct
  source 1..100: mol_type = other DNA
tgcggaagtgcgtgtcgccggttttagagctagaaatagcaagttaaaataaggctagtccgttatcaacttgaaaaagtggcaccgagtcggtgctttt

SEQ ID NO: 372 - DNA, 100 bp, synthetic construct
  source 1..100: mol_type = other DNA
gcggaagtgcgtgtcgccgggttttagagctagaaatagcaagttaaaataaggctagtccgttatcaacttgaaaaagtggcaccgagtcggtgctttt

SEQ ID NO: 373 - DNA, 100 bp, synthetic construct
  source 1..100: mol_type = other DNA
gggcccccagtagaatccgcgttttagagctagaaatagcaagttaaaataaggctagtccgttatcaacttgaaaaagtggcaccgagtcggtgctttt

SEQ ID NO: 374 - DNA, 100 bp, synthetic construct
  source 1..100: mol_type = other DNA
cgctggcgcgcgtgatgcgcgttttagagctagaaatagcaagttaaaataaggctagtccgttatcaacttgaaaaagtggcaccgagtcggtgctttt

SEQ ID NO: 375 - DNA, 100 bp, synthetic construct
  source 1..100: mol_type = other DNA
tcaggtagtcgcggaggacggttttagagctagaaatagcaagttaaaataaggctagtccgttatcaacttgaaaaagtggcaccgagtcggtgctttt

SEQ ID NO: 376 - DNA, 100 bp, synthetic construct
  source 1..100: mol_type = other DNA
gagcatccgcgtgcactttcgttttagagctagaaatagcaagttaaaataaggctagtccgttatcaacttgaaaaagtggcaccgagtcggtgctttt

SEQ ID NO: 377 - DNA, 100 bp, synthetic construct
  source 1..100: mol_type = other DNA
ctcaggtagtcgcggaggacgttttagagctagaaatagcaagttaaaataaggctagtccgttatcaacttgaaaaagtggcaccgagtcggtgctttt

SEQ ID NO: 378 - DNA, 100 bp, synthetic construct
  source 1..100: mol_type = other DNA
ccgaggccatcttcacgctagttttagagctagaaatagcaagttaaaataaggctagtccgttatcaacttgaaaaagtggcaccgagtcggtgctttt

SEQ ID NO: 379 - DNA, 100 bp, synthetic construct
  source 1..100: mol_type = other DNA
cagcgcatcgtggccaccgtgttttagagctagaaatagcaagttaaaataaggctagtccgttatcaacttgaaaaagtggcaccgagtcggtgctttt

SEQ ID NO: 380 - DNA, 100 bp, synthetic construct
  source 1..100: mol_type = other DNA
cgtgcgccccgtgcacgctcgttttagagctagaaatagcaagttaaaataaggctagtccgttatcaacttgaaaaagtggcaccgagtcggtgctttt

SEQ ID NO: 381 - DNA, 100 bp, synthetic construct
  source 1..100: mol_type = other DNA
agcgcgctcctggacgcctggttttagagctagaaatagcaagttaaaataaggctagtccgttatcaacttgaaaaagtggcaccgagtcggtgctttt

SEQ ID NO: 382 - DNA, 100 bp, synthetic construct
  source 1..100: mol_type = other DNA
ggatgcgagccaggttctcggttttagagctagaaatagcaagttaaaataaggctagtccgttatcaacttgaaaaagtggcaccgagtcggtgctttt

SEQ ID NO: 383 - DNA, 100 bp, synthetic construct
  source 1..100: mol_type = other DNA
tgcgccccgtgcacgctcaggttttagagctagaaatagcaagttaaaataaggctagtccgttatcaacttgaaaaagtggcaccgagtcggtgctttt

SEQ ID NO: 384 - DNA, 100 bp, synthetic construct
  source 1..100: mol_type = other DNA
caccgtgggccgcgagaaccgttttagagctagaaatagcaagttaaaataaggctagtccgttatcaacttgaaaaagtggcaccgagtcggtgctttt

SEQ ID NO: 385 - DNA, 100 bp, synthetic construct
  source 1..100: mol_type = other DNA
gcgtgcacggggcgcacgaggttttagagctagaaatagcaagttaaaataaggctagtccgttatcaacttgaaaaagtggcaccgagtcggtgctttt

SEQ ID NO: 386 - DNA, 100 bp, synthetic construct
  source 1..100: mol_type = other DNA
gcggatgctcgtgggtcccggttttagagctagaaatagcaagttaaaataaggctagtccgttatcaacttgaaaaagtggcaccgagtcggtgctttt

SEQ ID NO: 387 - DNA, 100 bp, synthetic construct
  source 1..100: mol_type = other DNA
gctgccggtcaaatctggaagttttagagctagaaatagcaagttaaaataaggctagtccgttatcaacttgaaaaagtggcaccgagtcggtgctttt

SEQ ID NO: 388 - DNA, 100 bp, synthetic construct
  source 1..100: mol_type = other DNA
ggccggcctgaaagtgcacggttttagagctagaaatagcaagttaaaataaggctagtccgttatcaacttgaaaaagtggcaccgagtcggtgctttt

SEQ ID NO: 389 - DNA, 100 bp, synthetic construct
  source 1..100: mol_type = other DNA
gctcaggtagtcgcggaggagttttagagctagaaatagcaagttaaaataaggctagtccgttatcaacttgaaaaagtggcaccgagtcggtgctttt

SEQ ID NO: 390 - DNA, 100 bp, synthetic construct
  source 1..100: mol_type = other DNA
tcaggccggccgctttcaccgttttagagctagaaatagcaagttaaaataaggctagtccgttatcaacttgaaaaagtggcaccgagtcggtgctttt

SEQ ID NO: 391 - DNA, 100 bp, synthetic construct
  source 1..100: mol_type = other DNA
taggatggtagcacacaaccgttttagagctagaaatagcaagttaaaataaggctagtccgttatcaacttgaaaaagtggcaccgagtcggtgctttt

SEQ ID NO: 392 - DNA, 100 bp, synthetic construct
  source 1..100: mol_type = other DNA
cggtggccacgatgcgctgggttttagagctagaaatagcaagttaaaataaggctagtccgttatcaacttgaaaaagtggcaccgagtcggtgctttt

SEQ ID NO: 393 - DNA, 100 bp, synthetic construct
  source 1..100: mol_type = other DNA
aggaaggttctggccgccgtgttttagagctagaaatagcaagttaaaataaggctagtccgttatcaacttgaaaaagtggcaccgagtcggtgctttt

SEQ ID NO: 394 - DNA, 100 bp, synthetic construct
  source 1..100: mol_type = other DNA
gtcgcggaggacggggttgagttttagagctagaaatagcaagttaaaataaggctagtccgttatcaacttgaaaaagtggcaccgagtcggtgctttt

SEQ ID NO: 395 - DNA, 100 bp, synthetic construct
  source 1..100: mol_type = other DNA
caggttctcgcggcccacgggttttagagctagaaatagcaagttaaaataaggctagtccgttatcaacttgaaaaagtggcaccgagtcggtgctttt

SEQ ID NO: 396 - DNA, 100 bp, synthetic construct
  source 1..100: mol_type = other DNA
gtgtgctaccatcctacagagttttagagctagaaatagcaagttaaaataaggctagtccgttatcaacttgaaaaagtggcaccgagtcggtgctttt

SEQ ID NO: 397 - DNA, 100 bp, synthetic construct
  source 1..100: mol_type = other DNA
cttagcgtgaagatggcctcgttttagagctagaaatagcaagttaaaataaggctagtccgttatcaacttgaaaaagtggcaccgagtcggtgctttt

SEQ ID NO: 398 - DNA, 100 bp, synthetic construct
  source 1..100: mol_type = other DNA
aggagctcaggtagtcgcgggttttagagctagaaatagcaagttaaaataaggctagtccgttatcaacttgaaaaagtggcaccgagtcggtgctttt

SEQ ID NO: 399 - DNA, 100 bp, synthetic construct
  source 1..100: mol_type = other DNA
cgctgcgccagcgccgcgtggttttagagctagaaatagcaagttaaaataaggctagtccgttatcaacttgaaaaagtggcaccgagtcggtgctttt

SEQ ID NO: 400 - DNA, 100 bp, synthetic construct
  source 1..100: mol_type = other DNA
cggcgccgcgccgcatgctggttttagagctagaaatagcaagttaaaataaggctagtccgttatcaacttgaaaaagtggcaccgagtcggtgctttt

SEQ ID NO: 401 - DNA, 100 bp, synthetic construct
  source 1..100: mol_type = other DNA
agccaggttctcgcggcccagttttagagctagaaatagcaagttaaaataaggctagtccgttatcaacttgaaaaagtggcaccgagtcggtgctttt

SEQ ID NO: 402 - DNA, 100 bp, synthetic construct
  source 1..100: mol_type = other DNA
gctggcggctgtcgcgcaccgttttagagctagaaatagcaagttaaaataaggctagtccgttatcaacttgaaaaagtggcaccgagtcggtgctttt

SEQ ID NO: 403 - DNA, 100 bp, synthetic construct
  source 1..100: mol_type = other DNA
cggctgcgcgccgagcccgtgttttagagctagaaatagcaagttaaaataaggctagtccgttatcaacttgaaaaagtggcaccgagtcggtgctttt

SEQ ID NO: 404 - DNA, 100 bp, synthetic construct
  source 1..100: mol_type = other DNA
gtgcggaagtgcgtgtcgccgttttagagctagaaatagcaagttaaaataaggctagtccgttatcaacttgaaaaagtggcaccgagtcggtgctttt

SEQ ID NO: 405 - DNA, 100 bp, synthetic construct
  source 1..100: mol_type = other DNA
agaggcagtcgaagctctcggttttagagctagaaatagcaagttaaaataaggctagtccgttatcaacttgaaaaagtggcaccgagtcggtgctttt

SEQ ID NO: 406 - DNA, 100 bp, synthetic construct
  source 1..100: mol_type = other DNA
tcgcggaggacggggttgaggttttagagctagaaatagcaagttaaaataaggctagtccgttatcaacttgaaaaagtggcaccgagtcggtgctttt

SEQ ID NO: 407 - DNA, 100 bp, synthetic construct
  source 1..100: mol_type = other DNA
agtgcgtgtcgccgggggccgttttagagctagaaatagcaagttaaaataaggctagtccgttatcaacttgaaaaagtggcaccgagtcggtgctttt

SEQ ID NO: 408 - DNA, 100 bp, synthetic construct
  source 1..100: mol_type = other DNA
ctgccggtcaaatctggaaggttttagagctagaaatagcaagttaaaataaggctagtccgttatcaacttgaaaaagtggcaccgagtcggtgctttt

SEQ ID NO: 409 - DNA, 100 bp, synthetic construct
  source 1..100: mol_type = other DNA
gcggctgtcgcgcaccaggagttttagagctagaaatagcaagttaaaataaggctagtccgttatcaacttgaaaaagtggcaccgagtcggtgctttt

SEQ ID NO: 410 - DNA, 100 bp, synthetic construct
  source 1..100: mol_type = other DNA
agctctcgcggctgccatccgttttagagctagaaatagcaagttaaaataaggctagtccgttatcaacttgaaaaagtggcaccgagtcggtgctttt

SEQ ID NO: 411 - DNA, 100 bp, synthetic construct
  source 1..100: mol_type = other DNA
gccggccgctttcacctggagttttagagctagaaatagcaagttaaaataaggctagtccgttatcaacttgaaaaagtggcaccgagtcggtgctttt

SEQ ID NO: 412 - DNA, 100 bp, synthetic construct
  source 1..100: mol_type = other DNA
gatggtagcacacaaccagggttttagagctagaaatagcaagttaaaataaggctagtccgttatcaacttgaaaaagtggcaccgagtcggtgctttt

SEQ ID NO: 413 - DNA, 100 bp, synthetic construct
  source 1..100: mol_type = other DNA
gtgctaccatcctacagaaggttttagagctagaaatagcaagttaaaataaggctagtccgttatcaacttgaaaaagtggcaccgagtcggtgctttt

SEQ ID NO: 414 - DNA, 100 bp, synthetic construct
  source 1..100: mol_type = other DNA
ctgcattgtcggctgccaccgttttagagctagaaatagcaagttaaaataaggctagtccgttatcaacttgaaaaagtggcaccgagtcggtgctttt

SEQ ID NO: 415 - DNA, 100 bp, synthetic construct
  source 1..100: mol_type = other DNA
cgagcccgtgggcaccttccgttttagagctagaaatagcaagttaaaataaggctagtccgttatcaacttgaaaaagtggcaccgagtcggtgctttt

SEQ ID NO: 416 - DNA, 100 bp, synthetic construct
  source 1..100: mol_type = other DNA
cgactgcctcttcgagctgcgttttagagctagaaatagcaagttaaaataaggctagtccgttatcaacttgaaaaagtggcaccgagtcggtgctttt

SEQ ID NO: 417 - DNA, 100 bp, synthetic construct
  source 1..100: mol_type = other DNA
ccacggtggccacgatgcgcgttttagagctagaaatagcaagttaaaataaggctagtccgttatcaacttgaaaaagtggcaccgagtcggtgctttt

SEQ ID NO: 418 - DNA, 100 bp, synthetic construct
  source 1..100: mol_type = other DNA
agtcgcggaggacggggttggttttagagctagaaatagcaagttaaaataaggctagtccgttatcaacttgaaaaagtggcaccgagtcggtgctttt

SEQ ID NO: 419 - DNA, 100 bp, synthetic construct
  source 1..100: mol_type = other DNA
cggtcaaatctggaaggggagttttagagctagaaatagcaagttaaaataaggctagtccgttatcaacttgaaaaagtggcaccgagtcggtgctttt

SEQ ID NO: 420 - DNA, 100 bp, synthetic construct
  source 1..100: mol_type = other DNA
tgtgctaccatcctacagaagttttagagctagaaatagcaagttaaaataaggctagtccgttatcaacttgaaaaagtggcaccgagtcggtgctttt

SEQ ID NO: 421 - DNA, 100 bp, synthetic construct
  source 1..100: mol_type = other DNA
gcggccgtgccccgcggtccgttttagagctagaaatagcaagttaaaataaggctagtccgttatcaacttgaaaaagtggcaccgagtcggtgctttt

SEQ ID NO: 422 - DNA, 100 bp, synthetic construct
  source 1..100: mol_type = other DNA
cagcagcagagccccgacgggttttagagctagaaatagcaagttaaaataaggctagtccgttatcaacttgaaaaagtggcaccgagtcggtgctttt

SEQ ID NO: 423 - DNA, 100 bp, synthetic construct
  source 1..100: mol_type = other DNA
ccagcgcatcgtggccaccggttttagagctagaaatagcaagttaaaataaggctagtccgttatcaacttgaaaaagtggcaccgagtcggtgctttt

SEQ ID NO: 424 - DNA, 100 bp, synthetic construct
  source 1..100: mol_type = other DNA
ccgtcggggctctgctgctggttttagagctagaaatagcaagttaaaataaggctagtccgttatcaacttgaaaaagtggcaccgagtcggtgctttt

SEQ ID NO: 425 - DNA, 100 bp, synthetic construct
  source 1..100: mol_type = other DNA
gcgcaccaggaaggtgcccagttttagagctagaaatagcaagttaaaataaggctagtccgttatcaacttgaaaaagtggcaccgagtcggtgctttt

SEQ ID NO: 426 - DNA, 100 bp, synthetic construct
  source 1..100: mol_type = other DNA
ggcggcgccgcgccgcatgcgttttagagctagaaatagcaagttaaaataaggctagtccgttatcaacttgaaaaagtggcaccgagtcggtgctttt

SEQ ID NO: 427 - DNA, 100 bp, synthetic construct
  source 1..100: mol_type = other DNA
tgggggcccctgagcgtgcagttttagagctagaaatagcaagttaaaataaggctagtccgttatcaacttgaaaaagtggcaccgagtcggtgctttt

SEQ ID NO: 428 - DNA, 100 bp, synthetic construct
  source 1..100: mol_type = other DNA
gcgcccgcggccgtgccccggttttagagctagaaatagcaagttaaaataaggctagtccgttatcaacttgaaaaagtggcaccgagtcggtgctttt

SEQ ID NO: 429 - DNA, 100 bp, synthetic construct
  source 1..100: mol_type = other DNA
ggctgccatccaggtgaaaggttttagagctagaaatagcaagttaaaataaggctagtccgttatcaacttgaaaaagtggcaccgagtcggtgctttt

SEQ ID NO: 430 - DNA, 100 bp, synthetic construct
  source 1..100: mol_type = other DNA
cgcggaggacggggttgagggttttagagctagaaatagcaagttaaaataaggctagtccgttatcaacttgaaaaagtggcaccgagtcggtgctttt

SEQ ID NO: 431 - DNA, 100 bp, synthetic construct; identical to SEQ ID NO: 373
  source 1..100: mol_type = other DNA

SEQ ID NO: 432 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 433 - RNA, 16 nt, synthetic construct
  source 1..16: mol_type = other RNA
gttttagagctatgct

SEQ ID NO: 434 - RNA, 20 nt, synthetic construct
  source 1..20: mol_type = other RNA
taatttctactcttgtagat

SEQ ID NO: 435 - RNA, 23 nt, synthetic construct
  source 1..23: mol_type = other RNA
tgatagatgatcccattggtagg

SEQ ID NO: 436 - RNA, 23 nt, synthetic construct
  source 1..23: mol_type = other RNA
tcgtgaagtcaggaagctcgtgg

SEQ ID NO: 437 - RNA, 23 nt, synthetic construct
  source 1..23: mol_type = other RNA
atgcagcttgtcagtacatgtgg

SEQ ID NO: 438 - RNA, 23 nt, synthetic construct
  source 1..23: mol_type = other RNA
ctttgtatttgagcaatatgcgg

SEQ ID NO: 439 - RNA, 23 nt, synthetic construct
  source 1..23: mol_type = other RNA
gcagcttgtcagtacatgtgggg

SEQ ID NO: 440 - RNA, 20 nt, synthetic construct
  source 1..20: mol_type = other RNA
tgatagatgatcccattggt

SEQ ID NO: 441 - RNA, 20 nt, synthetic construct
  source 1..20: mol_type = other RNA
tcgtgaagtcaggaagctcg

SEQ ID NO: 442 - RNA, 20 nt, synthetic construct
  source 1..20: mol_type = other RNA
atgcagcttgtcagtacatg

SEQ ID NO: 443 - RNA, 20 nt, synthetic construct
  source 1..20: mol_type = other RNA
ctttgtatttgagcaatatg

SEQ ID NO: 444 - RNA, 20 nt, synthetic construct
  source 1..20: mol_type = other RNA
gcagcttgtcagtacatgtg

SEQ ID NO: 445 - RNA, 23 nt, synthetic construct
  source 1..23: mol_type = other RNA
gatggtagcacgcaaccaggtgg

SEQ ID NO: 446 - RNA, 23 nt, synthetic construct
  source 1..23: mol_type = other RNA
agccgacaatgcgatctccccgg

SEQ ID NO: 447 - RNA, 23 nt, synthetic construct
  source 1..23: mol_type = other RNA
gatgcgccggtaatcggagtggg

SEQ ID NO: 448 - RNA, 23 nt, synthetic construct
  source 1..23: mol_type = other RNA
tgatgcgccggtaatcggagtgg

SEQ ID NO: 449 - RNA, 23 nt, synthetic construct
  source 1..23: mol_type = other RNA
gcggaagtgagtgtcgccagggg

SEQ ID NO: 450 - RNA, 20 nt, synthetic construct
  source 1..20: mol_type = other RNA
gatggtagcacgcaaccagg

SEQ ID NO: 451 - RNA, 20 nt, synthetic construct
  source 1..20: mol_type = other RNA
agccgacaatgcgatctccc

SEQ ID NO: 452 - RNA, 20 nt, synthetic construct
  source 1..20: mol_type = other RNA
gatgcgccggtaatcggagt

SEQ ID NO: 453 - RNA, 20 nt, synthetic construct
  source 1..20: mol_type = other RNA
tgatgcgccggtaatcggag

SEQ ID NO: 454 - RNA, 20 nt, synthetic construct
  source 1..20: mol_type = other RNA
gcggaagtgagtgtcgccag

SEQ ID NO: 455 - RNA, 36 nt, synthetic construct
  source 1..36: mol_type = other RNA
taggatggtagcacacaaccgttttagagctatgct

SEQ ID NO: 456 - RNA, 36 nt, synthetic construct
  source 1..36: mol_type = other RNA
gatggtagcacacaaccagggttttagagctatgct

SEQ ID NO: 457 - RNA, 36 nt, synthetic construct
  source 1..36: mol_type = other RNA
tgcgccccgtgcacgctcaggttttagagctatgct

SEQ ID NO: 458 - RNA, 36 nt, synthetic construct
  source 1..36: mol_type = other RNA
acgcctgcggattctactgggttttagagctatgct

SEQ ID NO: 459 - RNA, 36 nt, synthetic construct
  source 1..36: mol_type = other RNA
cggcgtgcgaacggaatgtggttttagagctatgct

SEQ ID NO: 460 - RNA, 36 nt, synthetic construct
  source 1..36: mol_type = other RNA
cgaaaaagcagttccgctgggttttagagctatgct

SEQ ID NO: 461 - RNA, 36 nt, synthetic construct
  source 1..36: mol_type = other RNA
tggtgcgcgacagccgccaggttttagagctatgct

SEQ ID NO: 462 - RNA, 36 nt, synthetic construct
  source 1..36: mol_type = other RNA
agcgcgctcctggacgcctggttttagagctatgct

SEQ ID NO: 463 - RNA, 100 nt, synthetic construct
  source 1..100: mol_type = other RNA
taggatggtagcacacaaccgttttagagctagaaatagcaagttaaaataaggctagtccgttatcaacttgaaaaagtggcaccgagtcggtgctttt

SEQ ID NO: 464 - RNA, 100 nt, synthetic construct
  source 1..100: mol_type = other RNA
gatggtagcacacaaccagggttttagagctagaaatagcaagttaaaataaggctagtccgttatcaacttgaaaaagtggcaccgagtcggtgctttt

SEQ ID NO: 465 - RNA, 100 nt, synthetic construct
  source 1..100: mol_type = other RNA
tgcgccccgtgcacgctcaggttttagagctagaaatagcaagttaaaataaggctagtccgttatcaacttgaaaaagtggcaccgagtcggtgctttt

SEQ ID NO: 466 - RNA, 100 nt, synthetic construct
  source 1..100: mol_type = other RNA
acgcctgcggattctactgggttttagagctagaaatagcaagttaaaataaggctagtccgttatcaacttgaaaaagtggcaccgagtcggtgctttt

SEQ ID NO: 467 - RNA, 100 nt, synthetic construct
  source 1..100: mol_type = other RNA
cggcgtgcgaacggaatgtggttttagagctagaaatagcaagttaaaataaggctagtccgttatcaacttgaaaaagtggcaccgagtcggtgctttt

SEQ ID NO: 468 - RNA, 100 nt, synthetic construct
  source 1..100: mol_type = other RNA
cgaaaaagcagttccgctgggttttagagctagaaatagcaagttaaaataaggctagtccgttatcaacttgaaaaagtggcaccgagtcggtgctttt

SEQ ID NO: 469 - RNA, 100 nt, synthetic construct
  source 1..100: mol_type = other RNA
tggtgcgcgacagccgccaggttttagagctagaaatagcaagttaaaataaggctagtccgttatcaacttgaaaaagtggcaccgagtcggtgctttt

SEQ ID NO: 470 - RNA, 100 nt, synthetic construct
  source 1..100: mol_type = other RNA
agcgcgctcctggacgcctggttttagagctagaaatagcaagttaaaataaggctagtccgttatcaacttgaaaaagtggcaccgagtcggtgctttt

SEQ ID NO: 471 - RNA, 36 nt, synthetic construct; identical to SEQ ID NO: 455
  source 1..36: mol_type = other RNA

SEQ ID NO: 472 - RNA, 36 nt, synthetic construct; identical to SEQ ID NO: 456
  source 1..36: mol_type = other RNA

SEQ ID NO: 473 - RNA, 36 nt, synthetic construct; identical to SEQ ID NO: 457
  source 1..36: mol_type = other RNA

SEQ ID NO: 474 - RNA, 36 nt, synthetic construct; identical to SEQ ID NO: 458
  source 1..36: mol_type = other RNA

SEQ ID NO: 475 - RNA, 36 nt, synthetic construct; identical to SEQ ID NO: 459
  source 1..36: mol_type = other RNA

SEQ ID NO: 476 - RNA, 36 nt, synthetic construct; identical to SEQ ID NO: 460
  source 1..36: mol_type = other RNA

SEQ ID NO: 477 - RNA, 36 nt, synthetic construct; identical to SEQ ID NO: 461
  source 1..36: mol_type = other RNA

SEQ ID NO: 478 - RNA, 36 nt, synthetic construct; identical to SEQ ID NO: 462
  source 1..36: mol_type = other RNA

SEQ ID NO: 479 - DNA, 100 bp, synthetic construct
  source 1..100: mol_type = other DNA
acgcctgcggattctactgggttttagagctagaaatagcaagttaaaataaggctagtccgttatcaacttgaaaaagtggcaccgagtcggtgctttt

SEQ ID NO: 480 - RNA, 20 nt, synthetic construct
  source 1..20: mol_type = other RNA
acgcctgcggattctactgg

SEQ ID NO: 481 - RNA, 36 nt, synthetic construct; identical to SEQ ID NO: 458
  source 1..36: mol_type = other RNA

(19) United States
(12) Patent Application Publication
(10) Pub. No.: US 2024/0409923 A1
(43) Pub. Date: Dec. 12, 2024

(54) GUIDE RNAS AND USES THEREOF

(71) Applicant: Spotlight Therapeutics, Hayward, CA (US)

(72) Inventors: Benjamin G. Gowen, San Leandro, CA (US); Mary Janatpour, Castro Valley, CA (US); Kory Melton, Pleasanton, CA (US); Akshay Tambe, Oakland, CA (US); Spencer Wei, Albany, CA (US)

(21) Appl. No.: 18/640,660

(22) Filed: Apr. 19, 2024

Related U.S. Application Data
(63) Continuation of application No. PCT/US2022/078549, filed on Oct. 21, 2022.
(60) Provisional application No. 63/271,065, filed on Oct. 22, 2021.

(51) Int. Cl. C12N 15/11; C12N 9/22; C12N 15/88; C12N 15/90

(52) U.S. Cl. CPC C12N 15/11; C12N 9/22; C12N 15/88; C12N 15/907; C12N 2310/20

(57) ABSTRACT

Provided herein are DNA targeting systems comprising a site-directed modifying polypeptide and a Tumor Necrosis Factor Alpha-induced Protein 3 (TNFAIP3)-specific guide RNA or a Suppressor of Cytokine Signaling 1 (SOCS1)-specific guide RNA, and uses thereof.

### RELATED APPLICATIONS

This application is a continuation of International Application No. PCT/US2022/078549, filed on Oct. 21, 2022, which claims priority to U.S. Provisional Application No. 63/271,065, filed on Oct. 22, 2021. The contents of the foregoing applications are hereby incorporated by reference.

### SEQUENCE LISTING

The instant application contains a Sequence Listing which has been submitted electronically in XML format, and which is hereby incorporated by reference in its entirety. Said XML copy, created on Oct. 21, 2022, is named S106638_1170WO_SL.xml, and is 437,605 bytes in size.

### BACKGROUND OF THE INVENTION

Cancer is a heterogeneous group of malignant diseases responsible for millions of deaths worldwide each year. Despite significant research efforts, many cancers remain incurable, largely due to their progression from a localized disease to a metastatic disease. Moreover, cancer cells have developed means to evade the standard checkpoints of the immune system. Therapies that target immune checkpoints have been explored as cancer therapeutics. However, the effectiveness of such immunomodulatory treatment strategies depends on the ability of the immune system to mount an anti-tumor immune response in the tumor microenvironment (Galon and Bruni. (2019). Nat. Rev. Drug Disc. 18 (3), 197-218; Sharma and Allison. (2015). Science. 348 (6230), 56-61). Accordingly, to generate an effective immune response in the tumor microenvironment, there is a need in the art for agents capable of modulating the tumor microenvironment in a manner that increases tumor immunogenicity.

CRISPR-associated RNA-guided endonucleases, such as Cas9, have become a versatile tool for genome engineering in various cell types and organisms (see, e.g., U.S. Pat. No. 8,697,359). Guided by a guide RNA, such as a dual-RNA complex or a chimeric single-guide RNA, RNA-guided endonucleases (e.g., Cas9) can generate site-specific double-strand breaks (DSBs) or single-stranded breaks (SSBs) within target nucleic acids (e.g., double-stranded DNA (dsDNA), single-stranded DNA (ssDNA), or RNA). When cleavage of a target nucleic acid occurs within a cell (e.g., a eukaryotic cell), the break in the target nucleic acid can be repaired by nonhomologous end joining (NHEJ) or homology directed repair (HDR). In addition, catalytically inactive RNA-guided endonucleases (e.g., Cas9) alone or fused to transcriptional activator or repressor domains can be used to alter transcription levels at sites within target nucleic acids by binding to the target site without cleavage. However, there remains a need to identify genes that can be effectively targeted by CRISPR systems for the treatment of cancer.

### SUMMARY OF THE INVENTION

Provided herein are DNA targeting systems for modifying an immunomodulatory gene capable of influencing the tumor microenvironment. In particular, provided are DNA targeting systems for modifying a TNFAIP3 gene or a SOCS1 gene, and related uses thereof.

The TNFAIP3 gene (also known as, e.g., A20, AISBL, OTUD7C, TNFA1P2) encodes Tumor necrosis factor alpha-induced protein 3, an ubiquitin-editing enzyme that contains both ubiquitin ligase and deubiquitinase activities. The TNFAIP3 protein is involved in immune and inflammatory responses signaled by cytokines and has been shown to inhibit NF-kappa B activation as well as TNF-mediated apoptosis.

The SOCS1 gene (also known as, e.g., JAB, CIS1, SSI1, CISH1, AISIMD, SOCS-1) encodes a member of the STAT-induced STAT inhibitor (SSI) family, also known as the suppressor of cytokine signaling (SOCS) family. The protein encoded by the SOCS1 gene is a cytokine-inducible regulator that is involved in a negative feedback loop that attenuates cytokine signaling. The expression of SOCS1 can be induced by a subset of cytokines, including IL2, IL3 erythropoietin (EPO), CSF2/GM-CSF, and interferon (IFN)-gamma.

As described herein, TNFAIP3 and SOCS1 have been identified as genes that can modulate the tumor microenvironment modulation through inflammatory pathways. Accordingly, provided herein are gRNAs that target a TNFAIP3 gene or a SOCS1 gene, and uses thereof.

In one aspect, provided herein is a method of reducing expression of an immunomodulatory gene in a mammalian cell, the method comprising contacting the mammalian cell with a site-directed modifying polypeptide and a guide RNA (gRNA) targeting the immunomodulatory gene, thereby reducing expression of the immunomodulatory gene in the mammalian cell, wherein the immunomodulatory gene is a Tumor Necrosis Factor Alpha-induced Protein 3 (TNFAIP3) gene or a Suppressor Of Cytokine Signaling 1 (SOCS1) gene.

In some embodiments, the mammalian cell is a mouse cell, a non-human primate cell, or a human cell. In some embodiments, the mammalian cell is a cancer cell, a monocyte, a macrophage (e.g., M1 macrophage, M2 macrophage), an endothelial cell, an epithelial cell, a natural killer cell, a pericyte, a neutrophil, a T cell (e.g., CD8+ T cell, CD4+ T cell), a B cell, a dendritic cell, or a fibroblast. In some embodiments, the mammalian cell is a cancer cell.

In some embodiments, the contacting step comprises delivering to the mammalian cell as a nucleoprotein complex comprising the site-directed modifying polypeptide bound to the gRNA.

In some embodiments, the contacting step comprises delivering to the mammalian cell the site-directed modifying polypeptide and the gRNA separately.

In another aspect, provided herein is a method of reducing expression of an immunomodulatory gene in a mammalian subject, the method comprising administering to the mammalian subject a therapeutically effective amount of a site-directed modifying polypeptide and a guide RNA (gRNA) targeting the immunomodulatory gene, thereby reducing expression of the immunomodulatory gene in the mammalian subject, wherein the immunomodulatory gene is a Tumor Necrosis Factor Alpha-induced Protein 3 (TNFAIP3) gene or a Suppressor Of Cytokine Signaling 1 (SOCS1) gene.

In some embodiments, the method comprises locally administering to the mammalian subject the therapeutically effective amount of the site-directed modifying polypeptide comprising the gRNA targeting the immunomodulatory gene. In some embodiments, the method comprises intratumorally administering to the mammalian subject the therapeutically effective amount of the site-directed modifying polypeptide comprising the gRNA targeting the immunomodulatory gene. In some embodiments, the method comprises peritumorally administering to the mammalian subject the therapeutically effective amount of the site-directed modifying polypeptide comprising the gRNA targeting the immunomodulatory gene.

In some embodiments, the method comprises locally administering a therapeutically effective amount of a site-directed modifying polypeptide and a gRNA targeting the immunomodulatory gene to the mammalian subject. In some embodiments, the method comprises intratumorally administering the site-directed modifying polypeptide and the gRNA to the mammalian subject. In some embodiments, the method comprises peritumorally administering of the site-directed modifying polypeptide and the gRNA to the mammalian subject.

In some embodiments, the method comprises systemically administering to the mammalian subject the therapeutically effective amount of the site-directed modifying polypeptide and the gRNA targeting the immunomodulatory gene.

In some embodiments, the immunomodulatory gene is a human immunomodulatory gene.

In one embodiment, the mammalian subject is a human.

In some embodiments, the mammalian subject has a cancer.

In some embodiments, the method further comprises administering an immune checkpoint blockade agent to the mammalian subject. In one embodiment, the immune checkpoint blockade agent is a modulator (e.g., an inhibitor or an activator) of an immune checkpoint molecule selected from the group consisting of A2AR, B7-H3, B7-H4, BTLA, CD27, CD28, CD40, CD80, CD86, CD122, CD137, CD137-L, CD160, CD226, CGEN-15049, CTLA-4, GITR, GITR-L, GALS, HVEM, ICOS, IDO, KIR, NOX2, OX40, PD-1, PD-L1, PD-L2, PD-L3, PD-L4, SIGLEC7, SIGLEC15, TIGIT, TIM-3, VISTA, 2B4, and LAG-3.

In some embodiments, the immune checkpoint blockade agent is a modulator (e.g., an inhibitor or an activator) of A2AR. In some embodiments, the immune checkpoint blockade agent is a modulator (e.g., an inhibitor or an activator) of B7-H3. In some embodiments, the immune checkpoint blockade agent is a modulator (e.g., an inhibitor or an activator) of B7-H4. In some embodiments, the immune checkpoint blockade agent is a modulator (e.g., an inhibitor or an activator) of BTLA. In some embodiments, the immune checkpoint blockade agent is a modulator (e.g., an activator) of CD27. In some embodiments, the immune checkpoint blockade agent is a modulator (e.g., an activator) of CD28. In some embodiments, the immune checkpoint blockade agent is a modulator (e.g., an inhibitor or an activator) of CD40. In some embodiments, the immune checkpoint blockade agent is a modulator (e.g., an inhibitor or an activator) of CD80. In some embodiments, the immune checkpoint blockade agent is a modulator (e.g., an inhibitor or an activator) of CD86. In some embodiments, the immune checkpoint blockade agent is a modulator (e.g., an inhibitor or an activator) of CD122. In some embodiments, the immune checkpoint blockade agent is a modulator (e.g., an activator) of CD137. In some embodiments, the immune checkpoint blockade agent is a modulator (e.g., an inhibitor or an activator) of CD137-L. In some embodiments, the immune checkpoint blockade agent is a modulator (e.g., an inhibitor or an activator) of CD160. In some embodiments, the immune checkpoint blockade agent is a modulator (e.g., an inhibitor or an activator) of CD226. In some embodiments, the immune checkpoint blockade agent is a modulator (e.g., an inhibitor or an activator) of CGEN-15049. In some embodiments, the immune checkpoint blockade agent is a modulator (e.g., an inhibitor or an activator) of CTLA-4. In some embodiments, the immune checkpoint blockade agent is a modulator (e.g., an inhibitor or an activator) of GITR. In some embodiments, the immune checkpoint blockade agent is a modulator (e.g., an inhibitor or an activator) of GITR-L. In some embodiments, the immune checkpoint blockade agent is a modulator (e.g., an inhibitor or an activator) of GALS. In some embodiments, the immune checkpoint blockade agent is a modulator (e.g., an inhibitor or an activator) of HVEM. In some embodiments, the immune checkpoint blockade agent is a modulator (e.g., an activator) of ICOS. In some embodiments, the immune checkpoint blockade agent is a modulator (e.g., an inhibitor or an activator) of IDO. In some embodiments, the immune checkpoint blockade agent is a modulator (e.g., an inhibitor or an activator) of KIR. In some embodiments, the immune checkpoint blockade agent is a modulator (e.g., an inhibitor or an activator) of NOX2. In some embodiments, the immune checkpoint blockade agent is a modulator (e.g., an activator) of OX40. In some embodiments, the immune checkpoint blockade agent is a modulator (e.g., an inhibitor or an activator) of PD-1. In some embodiments, the immune checkpoint blockade agent is a modulator (e.g., an inhibitor or an activator) of PD-L1. In some embodiments, the immune checkpoint blockade agent is a modulator (e.g., an inhibitor or an activator) of PD-L2. In some embodiments, the immune checkpoint blockade agent is a modulator (e.g., an inhibitor or an activator) of PD-L3. In some embodiments, the immune checkpoint blockade agent is a modulator (e.g., an inhibitor or an activator) of PD-L4. In some embodiments, the immune checkpoint blockade agent is a modulator (e.g., an inhibitor or an activator) of SIGLEC7. In some embodiments, the immune checkpoint blockade agent is a modulator (e.g., an inhibitor or an activator) of SIGLEC15. In some embodiments, the immune checkpoint blockade agent is a modulator (e.g., an inhibitor or an activator) of TIGIT. In some embodiments, the immune checkpoint blockade agent is a modulator (e.g., an inhibitor or an activator) of TIM-3. In some embodiments, the immune checkpoint blockade agent is a modulator (e.g., an inhibitor or an activator) of VISTA. In some embodiments, the immune checkpoint blockade agent is a modulator (e.g., an inhibitor or an activator) of 2B4. In some embodiments, the immune checkpoint blockade agent is a modulator (e.g., an inhibitor or an activator) of LAG-3.

In some embodiments, the gRNA is a single guide RNA (sgRNA). In some embodiments, the gRNA comprises a crRNA and a trRNA. In some embodiments, the gRNA comprises a crRNA (e.g, without a tracrRNA).

In some embodiments, the site-directed modifying polypeptide is an RNA-guided nuclease. In some embodiments, the RNA-guided nuclease is a Class 2 Cas polypeptide. In some embodiments, the Class 2 Cas polypeptide is a Type II Cas polypeptide. In one embodiment, the Type II Cas polypeptide is Cas9. In some embodiments, the Class 2 Cas polypeptide is a Type V Cas polypeptide. In one embodiment, the Type V Cas polypeptide is Cas12 (e.g., Cas12a).

In some embodiments, the site-directed modifying polypeptide is a base editor. In some embodiments, the base editor is a cytosine base editor. In some embodiments, the base editor is an adenine base editor.

In some embodiments, the gRNA is capable of targeting a non-human primate TNFAIP3 gene, a mouse TNFAIP3 gene, and/or a human TNFAIP3 gene.

In some embodiments, the gRNA is capable of targeting a mouse TNFAIP3 gene and a human TNFAIP3 gene.

In some embodiments, the gRNA is capable of targeting a non-human primate TNFAIP3 gene and a human TNFAIP3 gene.

In some embodiments, the gRNA is capable of targeting a non-human primate TNFAIP3 gene, a mouse Tnfaip3 gene and a human TNFAIP3 gene.

In some embodiments, the gRNA targets a region with an TNFAIP3 gene that has 100% homology in the DNA target sequence in humans, non-human primates (e.g., rhesus and/or cynomolgus), and/or mice.

In some embodiments, the gRNA is capable of targeting a non-human primate SOCS1 gene, a mouse SOCS1 gene, and/or a human SOCS1 gene.

In some embodiments, the gRNA is capable of targeting a mouse TNFAIP3 gene and a human SOCS1 gene.

In some embodiments, the gRNA is capable of targeting a non-human primate SOCS1 gene and a human SOCS1 gene.

In some embodiments, the gRNA is capable of targeting a non-human primate SOCS1 gene, a mouse Socs1 gene and a human SOCS1 gene.

In some embodiments, the gRNA targets a region with a SOCS1 gene that has 100% homology in the DNA target sequence in humans, non-human primates (e.g., rhesus and/or cynomolgus), and/or mice.

In some embodiments, the site-directed modifying polypeptide further comprises a cell targeting agent, thereby forming a targeted active gene editing (TAGE) agent. In some embodiments, the cell targeting agent is a ligand, a cell penetrating peptide, or an antigen-binding polypeptide. In some embodiments, the ligand binds to an extracellular cell membrane-bound molecule or protein.

In some embodiments, the antigen binding polypeptide is an antibody, an antigen-binding portion of an antibody, or an antibody mimetic. In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule. In some embodiments, the antigen-binding portion of the antibody is a nanobody, a domain antibody, an scFv, a Fab, a diabody, a BiTE, a diabody, a DART, a minibody, a F(ab′)₂, or an intrabody. In some embodiments, the antibody is an intact antibody or a bispecific antibody. In some embodiments, the antigen binding polypeptide binds to an extracellular cell membrane-bound molecule or protein.

In some embodiments, the immunomodulatory gene is a TNFAIP3 gene (e.g., a human TNFAIP3 gene) and the gRNA targeting the immunomodulatory gene is a gRNA targeting the TNFAIP3 gene.

In some embodiments, the gRNA targets a region of the TNFAIP3 gene that encodes an OTU domain of a human TNFAIP3 protein (e.g., corresponding to amino acid residues 92-263 of UniProt Accession No. P21580); a ubiquitin-interacting subdomain of a human TNFAIP3 protein (e.g., corresponding to amino acid residues 157-159, 190-192, and/or 224-227 of UniProt Accession No. P21580); a TRAF-binding domain of a human TNFAIP3 protein (e.g., corresponding to amino acid residues 58-300 of Uniprot Accession No. P21580); a TNIP1-interaction domain of a human TNFAIP3 protein (e.g., corresponding to amino acid residues 369-775 of Uniprot Accession No. P21580); a RIPK1-interacting domain of a human TNFAIP3 protein (e.g., corresponding to amino acid residues 386-453 of Uniprot Accession No. P21580); a lysosomal localization domain of a human TNFAIP3 protein (e.g., corresponding to amino acid residues 697-790 of Uniprot Accession No. P21580); a zinc-finger domain of a human TNFAIP3 protein (e.g., corresponding to amino acid residues 381-416, 472-507, 515-548, 601-636, 651-686, 710-745, and/or 756-790 of Uniprot Accession No. P21580) of a human TNFAIP3 protein. In some embodiments, the gRNA targeting the TNFAIP3 gene targets a region within exon 2, 3, or 4 of the human TNFAIP3 gene. In some embodiments, the gRNA targets a region within exon 2 of the human TNFAIP3 gene. In some embodiments, the gRNA targets a region within exon 3 of the human TNFAIP3 gene.

In some embodiments, the gRNA targeting the TNFAIP3 gene comprises a polynucleotide sequence set forth in any one of SEQ ID NOs: 70-124.

In some embodiments, the site-directed modifying polypeptide comprises Cas9 and the gRNA targeting the TNFAIP3 gene comprises a polynucleotide sequence set forth in any one of SEQ ID NOs: 89, 90, 92, 94-98, and 100-120.

In some embodiments, the site-directed modifying polypeptide comprises Cas12a and the gRNA targeting the TNFAIP3 gene comprises a polynucleotide sequence set forth in any one of SEQ ID NOs: 70-88, 91, 93, and 99.

In some embodiments, the site-directed modifying polypeptide comprises a base editor and the gRNA targeting the TNFAIP3 gene comprises a polynucleotide sequence set forth in any one of SEQ ID NOs: 121-124.

In some embodiments, the immunomodulatory gene is a SOCS1 gene (e.g., a human SOCS1 gene) and the gRNA is a gRNA targeting the SOCS1 gene.

In some embodiments, the gRNA targets a region of the SOCS1 gene that encodes an SH2 domain of a human SOCS1 protein (e.g., corresponding to amino acid residues 79-174 of Uniprot Accession No. 015524); an SOCS box domain of a human SOCS1 protein (e.g., corresponding to amino acid residues 161-210 of Uniprot Accession No. 015524); a kinase inhibitory region of a human SOCS1 protein (e.g., corresponding to amino acid residues 55-66 of Uniprot Accession No. O15524); an extended SH2 subdomain of a human SOCS1 protein (e.g., corresponding to amino acid residues 67-78 of Uniprot Accession No. 015524); or an elongin BC complex interaction domain of a human SOCS1 protein (e.g., corresponding to amino acid residues 173-182 of Uniprot Accession No. O15524). In some embodiments, the gRNA targeting the SOCS1 gene targets a region within exon 2 of the human SOCS1 gene. The DNA targeting system of claim 79, wherein the gRNA targets a region of the SOCS1 gene that encodes the first six amino acids of the SOCS1 protein. The DNA targeting system of claim 79, wherein the gRNA targets a region of the SOCS1 gene that encodes the first 14 amino acids of the SOCS1 protein.

In some embodiments, the gRNA targeting the SOCS1 gene comprises a polynucleotide sequence corresponding to any one of SEQ ID NOs: 264-347.

In some embodiments, the gRNA targeting the SOCS1 gene comprises a polynucleotide sequence corresponding to any one of SEQ ID NOs: 264-347 and 481.

In some embodiments, the site-directed modifying polypeptide comprises Cas9 and the gRNA targeting the SOCS1 gene comprises a polynucleotide sequence set forth in any one of SEQ ID NOS: 264, 266, 267, or 270-346.

In some embodiments, the site-directed modifying polypeptide comprises Cas9 and the gRNA targeting the SOCS1 gene comprises a polynucleotide sequence set forth in any one of SEQ ID NOs: 264, 266, 267, 270-346, or 481.

In some embodiments, the site-directed modifying polypeptide comprises Cas12 and the gRNA targeting the SOCS1 gene comprises a polynucleotide sequence set forth in any one of SEQ ID NOs: 265, 268, or 269.

In some embodiments, the site-directed modifying polypeptide comprises a base editor and the gRNA targeting the SOCS1 gene comprises a polynucleotide sequence set forth in SEQ ID NO: 347.

In another aspect, provided herein is a DNA targeting system for modifying an immunomodulatory gene, wherein the DNA targeting system comprises a site-directed modifying polypeptide and a guide RNA (gRNA) capable of targeting the immunomodulatory gene, and wherein the immunomodulatory gene is a Tumor Necrosis Factor Alpha-induced Protein 3 (TNFAIP3) gene or a Suppressor Of Cytokine Signaling 1 (SOCS1) gene.

In some embodiments, the gRNA comprises a single guide RNA (sgRNA). In some embodiments, the gRNA comprises a crispr RNA (crRNA) and a trans-activating crispr RNA (trRNA). In some embodiments, the gRNA comprises a crRNA (e.g., without a tracrRNA).

In certain embodiments, provided is a crRNA targeting a TNFAIP3 gene, wherein the crRNA comprises a polynucleotide sequence of any one of SEQ ID NOs: 70-124.

In certain embodiments, provided is a crRNA targeting a SOCS1 gene, wherein the crRNA comprises a polynucleotide sequence of any one of SEQ ID NOs: 264-347. In certain embodiments, provided is a crRNA targeting a SOCS1 gene, wherein the crRNA comprises a polynucleotide sequence of any one of SEQ ID NOs: 264-347 and 481.

In some embodiments, the site-directed modifying polypeptide comprises an RNA-guided nuclease.

In some embodiments, the RNA-guided nuclease is a Class 2 Cas polypeptide. In some embodiments, the Class 2 Cas polypeptide is a Type II Cas polypeptide. In one embodiment, the Type II Cas polypeptide is Cas9.

In some embodiments, the Class 2 Cas polypeptide is a Type V Cas polypeptide. In one embodiment, the Type V Cas polypeptide is Cas 12 (e.g., Cas12a).

In some embodiments, the site-directed modifying polypeptide comprises a base editor. In some embodiments, the base editor is a cytosine base editor. In some embodiments, the base editor is an adenine base editor.

In some embodiments, the site-directed modifying polypeptide further comprises a cell targeting agent, thereby forming a targeted active gene editing (TAGE) agent. In some embodiments, the cell targeting agent comprises a ligand, a cell penetrating peptide, or an antigen-binding polypeptide.

In some embodiments, the ligand binds to an extracellular cell membrane-bound molecule or protein.

In some embodiments, the antigen binding polypeptide is an antibody, an antigen-binding portion of an antibody, or an antibody mimetic. In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule. In some embodiments, the antigen-binding portion of the antibody is a nanobody, a domain antibody, an scFv, a Fab, a diabody, a BiTE, a diabody, a DART, a minibody, a F(ab′)₂, or an intrabody. In some embodiments, the antibody is an intact antibody or a bispecific antibody. In some embodiments, the antigen binding polypeptide binds to an extracellular cell membrane-bound molecule or protein.

In another aspect, provided herein is a DNA targeting system for modifying a Tumor necrosis factor alpha-induced protein 3 (TNFAIP3) gene comprising Cas9 and a guide RNA (gRNA), wherein the gRNA comprises a polynucleotide set forth in any one of SEQ ID NOs: 139, 143-148, 151-191, and 193-194.

In another aspect, provided herein is a DNA targeting system for modifying a Tumor necrosis factor alpha-induced protein 3 (TNFAIP3) gene comprising Cas12 and a guide RNA (gRNA), wherein the gRNA comprises a polynucleotide set forth in any one of SEQ ID NOs: 105-138, 140-142, and 149-150.

In another aspect, provided herein is a DNA targeting system for modifying a Tumor necrosis factor alpha-induced protein 3 (TNFAIP3) gene comprising a base editor and a guide RNA (gRNA), wherein the gRNA comprises a polynucleotide set forth in SEQ ID NO: 192. In some embodiments, the base editor is a cytosine base editor. In some embodiments, the base editor is an adenine base editor.

In some embodiments, the immunomodulatory gene is a TNFAIP3 gene (e.g., a human TNFAIP3 gene) and the gRNA targeting the immunomodulatory gene is a gRNA targeting the TNFAIP3 gene.

In some embodiments, the gRNA targets a region of the TNFAIP3 gene that encodes an OTU domain (e.g., corresponding to amino acid residues 92-263 of UniProt Accession No. P21580), a ubiquitin-interacting subdomain (e.g., corresponding to amino acid residues 157-159, 190-192, and/or 224-227 of UniProt Accession No. P21580), a TRAF-binding domain (e.g., corresponding to amino acid residues 58-300 of Uniprot Accession No. P21580), TNIP1-interaction domain (e.g., corresponding to amino acid residues 369-775 of Uniprot Accession No. P21580), a RIPK1-interacting domain (e.g., corresponding to amino acid residues 386-453 of Uniprot Accession No. P21580); a lysosomal localization domain (e.g., corresponding to amino acid residues 697-790 of Uniprot Accession No. P21580); a zinc-finger domain of the human TNFAIP3 protein (e.g., corresponding to amino acid residues 381-416, 472-507, 515-548, 601-636, 651-686, 710-745, and/or 756-790 of Uniprot Accession No. P21580) of a TNFAIP3 protein. In some embodiments, the gRNA targeting the TNFAIP3 gene targets a region within exon 2, 3, or 4 of the human TNFAIP3 gene.

In some embodiments, the gRNA targeting the TNFAIP3 gene comprises a polynucleotide sequence corresponding to any one of SEQ ID NOs: 70-124.

In some embodiments, the site-directed modifying polypeptide comprises Cas9 and the gRNA targeting the TNFAIP3 gene comprises a polynucleotide sequence set forth in any one of SEQ ID NOs: 89, 90, 92, 94-98, and 100-120.

In some embodiments, the site-directed modifying polypeptide is Cas12a and the gRNA targeting the TNFAIP3 gene comprises a polynucleotide sequence set forth in any one of SEQ ID NOs: 70-88, 91, 93, and 99.

In some embodiments, the site-directed modifying polypeptide is a base editor and the gRNA targeting the TNFAIP3 gene comprises a polynucleotide sequence set forth in any one of SEQ ID NOs: 121-124.

In some embodiments, the immunomodulatory gene is a SOCS1 gene (e.g., a human SOCS1 gene) and the gRNA is a gRNA targeting the SOCS1 gene.

In some embodiments, the gRNA targets a region of the SOCS1 gene that encodes an SH2 domain of a human SOCS1 protein (e.g., corresponding to amino acid residues 79-174 of Uniprot Accession No. 015524); an SOCS box domain of a human SOCS1 protein (e.g., corresponding to amino acid residues 161-210 of Uniprot Accession No. 015524); a kinase inhibitory region of a human SOCS1 protein (e.g., corresponding to amino acid residues 55-66 of Uniprot Accession No. O15524); an extended SH2 subdomain of a human SOCS1 protein (e.g., corresponding to amino acid residues 67-78 of Uniprot Accession No. 015524); or an elongin BC complex interaction domain of a human SOCS1 protein (e.g., corresponding to amino acid residues 173-182 of Uniprot Accession No. O15524). In some embodiments, the gRNA targeting the SOCS1 gene targets a region within exon 2 of the SOCS1 gene. The DNA targeting system of claim 79, wherein the gRNA targets a region of the SOCS1 gene that encodes the first six amino acids of the SOCS1 protein. The DNA targeting system of claim 79, wherein the gRNA targets a region of the SOCS1 gene that encodes the first 14 amino acids of the SOCS1 protein.

In some embodiments, the gRNA targets a site within the first six amino acids (16 nucleotides) from the translational start site of the SOCS1 gene. In some embodiments, the gRNA targets a site within the first 14 amino acids (40 nucleotides) from the translational start site of the SOCS1 gene.

In some embodiments, the gRNA targeting the SOCS1 gene comprises a polynucleotide sequence set forth in any one of SEQ ID NOs: 264-347.

In some embodiments, the gRNA targeting the SOCS1 gene comprises a polynucleotide sequence set forth in any one of SEQ ID NOs: 264-347 and 481.

In some embodiments, the site-directed modifying polypeptide comprises Cas9 and the gRNA targeting the SOCS1 gene comprises a polynucleotide sequence set forth in any one of SEQ ID NOs: 303, 264, 266, 267, and 270-346.

In some embodiments, the site-directed modifying polypeptide comprises Cas9 and the gRNA targeting the SOCS1 gene comprises a polynucleotide sequence set forth in any one of SEQ ID NOs: 303, 264, 266, 267, 270-346, and 481.

In some embodiments, the site-directed modifying polypeptide comprises Cas12 and the gRNA targeting the SOCS1 gene comprises a polynucleotide sequence set forth in any one of SEQ ID NOs: 265, 268, and 269.

In some embodiments, the site-directed modifying polypeptide comprises a base editor and the gRNA targeting the SOCS1 gene comprises a polynucleotide sequence set forth in SEQ ID NO: 347.

In another aspect, provided herein is an isolated polynucleotide encoding any of the DNA targeting systems disclosed herein.

In some embodiments, the DNA targeting system is for modifying a TNFAIP3 gene and the isolated polynucleotide comprises the nucleotide sequence of any one of SEQ ID NOs: 125-179 (e.g., wherein the isolated polynucleotide encodes a TNFAIP3 gRNA).

In some embodiments, the DNA targeting system is for modifying a SOCS1 gene and the isolated polynucleotide comprises the nucleotide sequence of any one of SEQ ID NOs: 348-431 (e.g., wherein the isolated polynucleotide encodes a SOCS1 gRNA).

In some embodiments, the DNA targeting system is for modifying a SOCS1 gene and the isolated polynucleotide comprises the nucleotide sequence of any one of SEQ ID NOs: 348-431 and 479 (e.g., wherein the isolated polynucleotide encodes a SOCS1 gRNA).

In another aspect, provided herein is a vector comprising any isolated polynucleotide disclosed herein.

In another aspect, provided herein is a cell comprising any isolated polynucleotide disclosed herein.

In a further aspect, provided herein is a lipid nanoparticle (LNP) comprising an mRNA encoding a DNA targeting system disclosed herein.

In some embodiments, the gRNA and the site-directed modifying polypeptide of the DNA targeting system are encoded on separate RNA constructs. In some embodiments, the gRNA and the site-directed modifying polypeptide of the DNA targeting system are encoded on the same RNA constructs.

In another aspect, provided herein is a lipid nanoparticle (LNP) comprising an mRNA encoding a gRNA (e.g., capable of targeting the TNFAIP3 gene) comprising a polynucleotide sequence set forth in any one of SEQ ID NOs: 70-124.

In another aspect, provided herein is a lipid nanoparticle (LNP) comprising an mRNA encoding a gRNA (e.g., capable of targeting the SOCS1 gene) comprising a polynucleotide sequence set forth in any one of SEQ ID NOs: 264-347.

In another aspect, provided herein is a lipid nanoparticle (LNP) comprising an mRNA encoding a gRNA (e.g., capable of targeting the SOCS1 gene) comprising a polynucleotide sequence set forth in any one of SEQ ID NOs: 264-347 and 481.

In another aspect, provided herein is a lipid nanoparticle (LNP) comprising a ribonucleoprotein comprising a site-directed modifying polypeptide and a gRNA capable of targeting a TNFAIP3 gene (e.g., a gRNA comprising a polynucleotide sequence set forth in any one of SEQ ID NOs: 70-124).

In another aspect, provided herein is a lipid nanoparticle (LNP) comprising a ribonucleoprotein comprising a site-directed modifying polypeptide and a gRNA capable of targeting a SOCS1 gene (e.g., a gRNA comprising a polynucleotide sequence set forth in any one of SEQ ID NOs: 264-347).

In another aspect, provided herein is a lipid nanoparticle (LNP) comprising a ribonucleoprotein comprising a site-directed modifying polypeptide and a gRNA capable of targeting a SOCS1 gene (e.g., a gRNA comprising a polynucleotide sequence set forth in any one of SEQ ID NOs: 264-347 and 481).

### BRIEF DESCRIPTION OF THE DRAWINGS

FIGS. 1A-1D graphically depict the results of a screen for guide RNAs (gRNA) that target the TNFAIP3 (A20) gene in mouse T cells. FIG. 1A graphically depicts the editing rate (observed indel rate (%)) for a 300 pmol dose of each of the indicated TNFAIP3 gRNAs as compared to a non-targeting gRNA. FIG. 1B graphically depicts the percent editing rate (i.e., indel rate (%)) for each of the indicated TNFAIP3 gRNAs as a function of gRNA dose (pmol). gRNAs shown in black were selected for follow up studies in vivo. FIG. 1C graphically depicts the percent editing rate (i.e., observed indel rate (%); y-axis) for each of the indicated TNFAIP3 gRNAs as a function of Doench score (computational prediction of guide activity; x-axis). The results in FIG. 1C indicate that computational prediction of high activity is not sufficient to identify high efficiency guides. TNFAIP3 gRNAs having an increased editing rate relative to other gRNA candidates assessed, low variance in editing rate and high specificity in editing outcomes, and that fulfilled other biological criteria were selected for further validation. FIG. 1D graphically depicts indel repair outcomes observed following a 300 pmol dose of each of the indicated TNFAIP3 gRNAs.

FIGS. 2A and 2B graphically depict the results of an ex vivo study evaluating Tnfaip3 (A20) gene editing in primary murine T cells. The indel rate (FIG. 2A) and the frameshift rate (FIG. 2B) are shown for each of the indicated gRNAs.

FIG. 3 graphically depicts the results of an experiment showing that the editing (indel) rate does not correlate with computational prediction score (doench score) for murine sgRNA targeting Tnfaip3 (A20).

FIGS. 4A-4D graphically depict the results of a screen for guide RNAs (gRNA) that target the SOCS1 gene in mouse T cells. FIG. 4A graphically depicts the editing rate (observed indel rate (%)) for a 300 pmol dose of each of the indicated SOCS1 gRNAs. FIG. 4B graphically depicts the percent editing rate (i.e., indel rate (%)) for each of the indicated SOCS1 gRNAs as a function of gRNA dose (pmol). gRNAs shown in black were selected for follow up studies in vivo. FIG. 4C graphically depicts the percent editing rate (i.e., observed indel rate (%); y-axis) for each of the indicated SOCS1 gRNAs as a function of Doench score (computational prediction of guide activity; x-axis). The results in FIG. 4C indicate that computational prediction of high activity is not sufficient to identify high efficiency guides. SOCS1 gRNAs having an increased editing rate relative to other gRNA candidates assessed, low variance in editing rate and high specificity in editing outcomes, and that fulfilled other biological criteria were selected for further validation. FIG. 4D graphically depicts indel repair outcomes observed following a 300 pmol dose of each of the indicated SOCS1 gRNAs.

FIGS. 5A and 5B graphically depict the results of an ex vivo study evaluating SOCS1 gene editing in primary murine T cells. The indel rate (FIG. 5A) and the frameshift rate (FIG. 5B) are shown for each of the indicated gRNAs.

FIG. 6 graphically depicts the results of an experiment showing that the editing (indel) rate does not correlate with computational prediction score (doench score) for murine sgRNA targeting SOCS1.

FIG. 7 graphically depicts the results of an in vivo study evaluating the anti-tumor efficacy of A20 (Tnfaip3) and Socs1 targeting TAGE in combination with immune checkpoint blockade. *p<0.05, **p<0.01, *p<0.001, ****p<0.0001, ns not significant, 1-way ANOVA with multiple testing correction.

FIGS. 8A-8C graphically depict the results of an in vivo study evaluating the anenestic and enestic anti-tumor efficacy of Socs1 targeting TAGE in combination with immune checkpoint blockade. FIG. 8A shows enestic tumor volume following treatment with each of the indicated conditions. FIG. 8B shows anenestic tumor volume following treatment with each of the indicated gRNAs. FIG. 8C shows normalized tumor volume (% of average of negative control group) following treatment with each of the indicated gRNAs. **p<0.01, ns not significant, 1-way ANOVA with multiple testing correction.

FIG. 9 graphically depicts the results of a screen for the identification of high efficiency human SOCS1 targeting guides RNAs.

FIG. 10 shows increased expression of CXCL10 mRNA in primary human T cells 3, 4, or 5 days after SOCS1 editing. CXCL10 gene expression was measured by qPCR and normalized to GAPDH as a reference gene. Points show biological replicates (individual electroporation samples), lines show mean±SEM.

### DETAILED DESCRIPTION OF THE INVENTION

### I. Definitions

As used herein, the term “immunomodulatory gene” refers to a gene whose altered expression in a mammal has an effect on the immune system of the mammal, directly or indirectly. For example, the immunomodulatory gene may be a gene that has a role in inflammatory pathways in a mammal, such as a TNFAIP3 gene or a SOCS1 gene.

The term “TNFAIP3,” also known as “Tumor necrosis factor (TNF) alpha-induced protein 3”, “OTU Domain-Containing Protein 7C,” “A20”, “AISBL”, “OTUD7C”, “TNA1P2”, refers to a gene encoding a TNF alpha induced protein 3 from any vertebrate or mammalian source, including, but not limited to human, bovine, chicken, rodent, mouse, rat, porcine, ovine, primate, monkey, and guinea pig, unless specified otherwise. The term also refers to fragments and variants of native TNFAIP3 that maintain at least one in vivo or in vitro activity of a native TNFAIP3. The term encompasses full-length unprocessed precursor forms of TNFAIP3 as well as mature forms resulting from post-translational processing.

The TNFAIP3 gene in humans is located in the human chromosomal region 6q23.3. The nucleotide sequence of the genomic region of human chromosome harboring the TNFAIP3 gene may be found in, for example, the Genome Reference Consortium Human Build 38 (also referred to as Human Genome build 38 or GRCh38) available at GenBank. The nucleotide sequence of the genomic region of human chromosome 6 harboring the TNFAIP3 gene may also be found at, for example, GenBank Accession No. NC_000006.12, corresponding to nucleotides 137866317-137883312) of human chromosome 6. Examples of human TNFAIP3 DNA, mRNA, and amino acid sequences are readily available using publicly available databases, e.g., GenBank, UniProt, and OMIM. Additional information on the human TNFAIP3 gene can be found, for example, at NCBI under Gene ID: 7128.

The TNFAIP3 gene in mice is located in the mouse chromosomal region 10 A3; 10 8.08 cM. The nucleotide sequence of the genomic region of mouse chromosome harboring the TNFAIP3 gene may be found in, for example, the Genome Reference Consortium Mouse Build 39 (also referred to as Mouse Genome build 39 or GRCm39) available at GenBank. The nucleotide sequence of the genomic region of mouse chromosome 10 harboring the TNFAIP3 gene may also be found at, for example, GenBank Accession No. NC_000076.7, corresponding to nucleotides 18876658-18891158 of mouse chromosome 10. Examples of mouse TNFAIP3 DNA, mRNA, and amino acid sequences are readily available using publicly available databases, e.g., GenBank, UniProt, and OMIM. Additional information on the mouse TNFAIP3 gene can be found, for example, at NCBI under Gene ID: 21929.

The term “TNFAIP3” as used herein also refers to naturally occurring DNA sequence variations of the TNFAIP3 gene, such as a single nucleotide polymorphism in the TNFAIP3 gene. Numerous SNPs within the TNFAIP3 gene have been identified and may be found at, for example, at NCBI dbSNP, and in the clinical variant database at NCBI ClinVar under the term “TNFAIP3”.

The term “SOCS1,” also known as “Suppressor of cytokine signaling 1”, “STAT-Induced STAT Inhibitor 1,” “Tec-Interacting Protein 3,” “Cytokine-Inducible SH2 Protein 1,” “JAK Binding Protein,” “SOCS-1,” “JAB,” “CIS1”, “SSI1”, “TIP3”, “CISH1”, “AISIMD,” “SOCS-1,” refers to a gene encoding a STAT-induced STAT inhibitor (SSI) 1, also known as suppressor of cytokine signaling (SOCS) 1, from any vertebrate or mammalian source, including, but not limited to human, bovine, chicken, rodent, mouse, rat, porcine, ovine, primate, monkey, and guinea pig, unless specified otherwise. The term also refers to fragments and variants of native SOCS1 that maintain at least one in vivo or in vitro activity of a native SOCS1. The term encompasses full-length unprocessed precursor forms of SOCS1 as well as mature forms resulting from post-translational processing.

The SOCS1 gene in humans is located in the human chromosomal region 16p13.13. The nucleotide sequence of the genomic region of human chromosome harboring the SOCS1 gene may be found in, for example, the Genome Reference Consortium Human Build 38 (also referred to as Human Genome build 38 or GRCh38) available at GenBank. The nucleotide sequence of the genomic region of human chromosome 16 harboring the SOCS1 gene may also be found at, for example, GenBank Accession No. NC_000016.10, corresponding to nucleotides 11254417-11256204) of human chromosome 16. Examples of human SOCS1 DNA, mRNA, and amino acid sequences are readily available using publicly available databases, e.g., GenBank, UniProt, and OMIM. Additional information on the human SOCS1 gene can be found, for example, at NCBI under Gene ID: 8651.

The SOCS1 gene in mice is located in the mouse chromosomal region 16 A1; 16 5.81 cM. The nucleotide sequence of the genomic region of mouse chromosome harboring the SOCS1 gene may be found in, for example, the Genome Reference Consortium Mouse Build 39 (also referred to as Mouse Genome build 39 or GRCm39) available at GenBank. The nucleotide sequence of the genomic region of mouse chromosome 16 harboring the SOCS1 gene may also be found at, for example, GenBank Accession No. NC_000082.7, corresponding to nucleotides 10601672-10603400 of mouse chromosome 16. Examples of mouse SOCS1 DNA, mRNA, and amino acid sequences are readily available using publicly available databases, e.g., GenBank, UniProt, and OMIM. Additional information on the mouse SOCS1 gene can be found, for example, at NCBI under Gene ID: 12703.

The term “SOCS1” as used herein also refers to naturally occurring DNA sequence variations of the SOCS1 gene, such as a single nucleotide polymorphism in the SOCS1 gene. Numerous SNPs within the SOCS1 gene have been identified and may be found at, for example, at NCBI dbSNP, and in the clinical variant database at NCBI ClinVar under the term “SOCS1”.

As used herein, the term “guide RNA” or “gRNA” refers to an RNA molecule comprising crispr RNA (crRNA) and can further include a trans-activating crispr RNA (tracrRNA). As used herein, the term “crRNA” refers to an RNA molecule having a polynucleotide-targeting guide sequence, a stem sequence, and, optionally, a 5-overhang sequence. As used herein, the term “tracrRNA” refers to an RNA molecule having a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules. As used herein, a gRNA can associate with a site-directed modifying polypeptide (e.g., a RNA-guided nuclease, such as a Cas protein) and aid in targeting the Cas protein to a specific location within a target polynucleotide (e.g., a DNA).

The term “DNA targeting system” refers to a site-directed modifying polypeptide and an RNA molecule (e.g., a guide RNA) capable of directing the site-directed modifying polypeptide to a specific DNA target. In one embodiment, the DNA targeting system is a CRISPR-Cas DNA-targeting system.

As used herein, the term “target site,” refers to a nucleic acid sequence that defines a portion of a nucleic acid to which a binding molecule will bind, provided sufficient conditions for binding exist. In some embodiments, a target site is a nucleic acid sequence to which a nuclease described herein binds and/or that is cleaved by such nuclease. In some embodiments, a target site is a nucleic acid sequence to which a guide RNA described herein binds. In some embodiments, the target site is about 1-10 nucleotides from a protospacer adjacent motif (PAM) that is recognized by the nuclease (e.g., about 1, about 2, about 3, about 4, about 5, about 6, about 7, about 8, about 9, or about 10 nucleotides from the PAM). In specific embodiments the PAM is NGG (e.g., Cas9 PAM). A target site may be single-stranded or double-stranded. If single-stranded, the target site can be located on the sense strand or antisense strand.

As used herein, a “site-directed modifying polypeptide” refers to a protein that is targeted to a specific nucleic acid sequence or set of similar sequences of a polynucleotide chain via recognition of the particular sequence(s) by the modifying polypeptide itself or an associated molecule (e.g., RNA), wherein the polypeptide can modify the polynucleotide chain.

The terms “polypeptide” or “protein”, as used interchangeably herein, refer to any polymeric chain of amino acids. The term “polypeptide” encompasses native or artificial proteins, protein fragments and polypeptide analogs of a protein sequence.

The term “targeted active gene editing” or “TAGE” agent refers to a complex of molecules including a cell targeting agent (such as, but not limited to, an antigen binding polypeptide (e.g., an antibody or an antigen-binding portion thereof), a ligand, a cell penetrating peptide (CPP), or combinations thereof), that specifically binds to an extracellular target molecule (e.g., an extracellular protein or glycan, such as an extracellular protein on the cell surface) displayed on a cell membrane or otherwise promotes cellular internalization, and a site-directed modifying polypeptide (such as, but not limited to, an endonuclease) that recognizes a nucleic acid sequence. The cell targeting agent of a TAGE agent is associated with the site-directed modifying polypeptide such that at least the site-directed modifying polypeptide is internalized by a target cell, e.g., a cell expressing an extracellular molecule bound by the cell targeting agent. An example of a TAGE agent is an active CRISPR targeting or TAGE agent where the site directed polypeptide is a nucleic acid-guided DNA endonuclease (e.g., RNA-guided endonuclease or DNA-guided endonuclease), such as Cas9 or Cas 12. In some embodiments, the TAGE agent includes at least one NLS. Notably, a TAGE agent can target any nucleic acid within a cell, including, but not limited to, a gene.

The term “antigen binding polypeptide” as used herein refers to a protein that binds to a specified target antigen, such as an extracellular cell membrane-bound protein (e.g., a cell surface protein). Examples of an antigen binding polypeptide include an antibody, antigen-binding fragment of an antibody, and an antibody mimetic. In certain embodiments, an antigen-binding polypeptide is an antigen binding peptide.

The term “conjugation moiety” as used herein refers to a moiety that is capable of conjugating two more or more molecules, such as an antigen binding protein, a CPP, or a ligand and a site-directed modifying polypeptide. The term “conjugation,” as used herein, refers to the physical or chemical complexation formed between a molecule (for e.g. an antigen binding protein (e.g., an antibody), CPP, or ligand) and the second molecule (e.g. a site-directed modifying polypeptide, therapeutic agent, drug or a targeting molecule). The chemical complexation constitutes specifically a bond or chemical moiety formed between a functional group of a first molecule (e.g., an antigen binding protein (e.g., an antibody), CPP, or ligand) with a functional group of a second molecule (e.g., a site-directed modifying polypeptide, a therapeutic agent or drug). Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. In one embodiment, conjugation is achieved via a physical association or non-covalent complexation.

As used herein, the term “ligand” refers to a molecule that is capable of specifically binding to another molecule on or in a cell, such as one or more cell surface receptors, and includes molecules such as proteins, hormones, neurotransmitters, cytokines, growth factors, cell adhesion molecules, or nutrients. Generally, a ligand that binds to another specific molecule or molecules. For example, a ligand may bind to a receptor. A site-specific modifying polypeptide (e.g., nuclease) of TAGE agent can be associated with one or more ligands through covalent or non-covalent linkage. Examples of ligands useful herein, or targets bound by ligands, and further description of ligands in general, are disclosed in Bryant & Stow (2005). Traffic, 6 (10), 947-953; Olsnes et al. (2003). Physiological reviews, 83(1), 163-182; and Planque, N. (2006). Cell Communication and Signaling, 4(1), 7, which are incorporated herein by reference.

As used herein, the term “target cell” refers to a cell or population of cells, such as mammalian cells (e.g., human cells), which includes a nucleic acid sequence in which site-directed modification of the nucleic acid is desired (e.g., to produce a genetically-modified cell in vivo or ex vivo). In some instances, a target cell displays on its cell membrane an extracellular molecule (e.g., an extracellular protein such as a receptor or a ligand, or glycan) specifically bound by a cell targeting agent of the TAGE agent.

As used herein, the term “genetically-modified cell” refers to a cell, or an ancestor thereof, in which a DNA sequence has been deliberately modified by a site-directed modifying polypeptide.

As used herein, the term “nucleic acid” refers to a molecule comprising nucleotides, including a polynucleotide, an oligonucleotide, or other DNA or RNA. In one embodiment, a nucleic acid is present in a cell and can be transmitted to progeny of the cell via cell division. In some instances, a nucleic acid is a gene (e.g., an endogenous gene) found within the genome of a cell within its chromosomes. In other instances, a nucleic acid is a mammalian expression vector that has been transfected into a cell. DNA that is incorporated into the genome of a cell using, e.g., transfection methods, is also considered within the scope of a “nucleic acid” as used herein, even if the incorporated DNA is not meant to be transmitted to progeny cells. As used herein, the term “polynucleotide”, refers to a polymer of deoxyribonucleotides or ribonucleotides in either single- or double-stranded form, and unless otherwise stated, encompasses known analogs of natural nucleotides or synthetic (i.e., synthesized) nucleic acids that can function in a similar manner as naturally occurring nucleotides.

As used herein, the term “endosomal escape agent” or “endosomal release agent” refers to an agent (e.g., a peptide) that, when conjugated to a molecule (e.g., a polypeptide, such as a site-directed modifying polypeptide), is capable of promoting release of the molecule from an endosome within a cell. Polypeptides that remain within endosomes can eventually be targeted for degradation or recycling rather than released into the cytoplasm or trafficked to a desired subcellular destination. Accordingly, in some embodiments, a TAGE agent comprises an endosomal escape agent.

As used herein, the term “stably associated” when used in the context of a TAGE agent, refers to the ability of the cell targeting agent and the site-directed modifying polypeptide to complex in such a way that the complex can be internalized into a target cell such that nucleic acid editing can occur within the cell. Examples of ways to determine if a TAGE agent is stably associated include in vitro assays whereby association of the complex is determined following exposure of a cell to the TAGE agent, e.g., by determining whether gene editing occurred using a standard gene editing systems. Examples of such assays are known in the art, such as SDS-PAGE, Western blot, size exclusion chromatography (SEC), and electrophoretic mobility shift assay to determine protein complexes; PCR amplification, direct sequencing (e.g., next-generation sequencing or Sanger sequencing), enzymatic cleavage of a locus with a nuclease (e.g., Celery) of the gene locus to confirm editing; and indirect phenotypic assays that measure the downstream effects of editing a specific gene, such as loss of a protein as measured by Western blot or flow cytometry or generation of a functional protein, as measured by functional assays.

As used herein, the term “modifying a nucleic acid” refers to any modification to a nucleic acid targeted by a site-directed modifying polypeptide. Examples of such modifications include any changes to the amino acid sequence including, but not limited to, any insertion, deletion, or substitution of an amino acid residue in the nucleic acid sequence relative to a reference sequence (e.g., a wild-type or a native sequence). Such amino acid changes may, for example, may lead to a change in expression of a gene (e.g., an increase or decrease in expression) or replacement of a nucleic acid sequence. Modifications of nucleic acids can further include double stranded cleavage, single stranded cleavage, or binding of any RNA-guided endonuclease disclosed herein to a target site. Binding of a RNA-guided endonuclease can inhibit expression of the nucleic acid or can increase expression of any nucleic acid in operable linkage to the nucleic acid comprising the target site.

The term “cell-penetrating peptide” (CPP) refers to a peptide, generally of about 5-60 amino acid residues (e.g., 5-10, 10-15, 15-20, 20-25, 25-30, 30-35, 35-40, 40-45, 45-50, 50-55, or 55-60 amino acid resides) in length, that can facilitate cellular uptake of a conjugated molecule, particularly one or more site-specific modifying polypeptides. A CPP can also be characterized in certain embodiments as being able to facilitate the movement or traversal of a molecular conjugate across/through one or more of a lipid bilayer, micelle, cell membrane, organelle membrane (e.g., nuclear membrane), vesicle membrane, or cell wall. A CPP herein can be cationic, amphipathic, or hydrophobic in certain embodiments. Examples of CPPs useful herein, and further description of CPPs in general, are disclosed in Borrelli, Antonella, et al. Molecules 23.2 (2018): 295; Milletti, Francesca. Drug discovery today 17.15-16 (2012): 850-860, which are incorporated herein by reference. Further, there exists a database of experimentally validated CPPs (CPPsite, Gautam et al., 2012). The CPP of a TAGE agent of the invention can be any known CPP, such as a CPP shown in the CPPsite database.

As used herein, the term “nuclear localization signal” or “NLS” refers to a peptide that, when conjugated to a molecule (e.g., a polypeptide, such as a site-directed modifying polypeptide), is capable of promoting import of the molecule into the cell nucleus by nuclear transport. The NLS can, for example, direct transport of a protein with which it is associated from the cytoplasm of a cell across the nuclear envelope barrier. The NLS is intended to encompass not only the nuclear localization sequence of a particular peptide, but also derivatives thereof that are capable of directing translocation of a cytoplasmic polypeptide across the nuclear envelope barrier. In some embodiments, one or more NLSs (e.g., 1, 2, 3, 4, 5, 6, 7, 8, 2-6, 3-7, 4-8, 5-9, 6-10, 7-10, 8-10 NLSs) can be attached to the N-terminus, the C-terminus, or both the N- and C-termini of the polypeptide of a TAGE agent herein.

The term “TAT-related peptide” as used herein, refers to a CPP that is derived from the transactivator of transcription (TAT) of human immunodeficiency virus. The amino acid sequence of a TAT peptide comprises RKKRRQRRR (SEQ ID NO: 12). Thus, a TAT-related peptide includes any peptide comprising the amino acid sequence of RKKRRQRRR (SEQ ID NO: 12), or an amino acid sequence having conservative amino acid substitutions wherein the peptide is still able to internalize into a cell. In certain embodiments, a TAT-related peptide includes 1, 2, or 3 amino acid substitutions, wherein the TAT-related peptide is able to internalize into a target cell.

As used herein, the term “specifically binds” in the context of an antigen binding polypeptide refers to an antigen-binding polypeptide which recognizes and binds to an antigen present in a sample, but which antigen binding polypeptide does not substantially recognize or bind other molecules in the sample. In one embodiment, an antigen binding polypeptide that specifically binds to an antigen, binds to an antigen with an Kd of at least about 1×10⁻⁴, 1×10⁻⁵, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more as determined by surface plasmon resonance or other approaches known in the art (e.g., filter-binding assay, fluorescence polarization, isothermal titration calorimetry), including those described further herein. In one embodiment, an antigen binding polypeptide specifically binds to an antigen if the antigen binding polypeptide binds to an antigen with an affinity that is at least two-fold greater as determined by surface plasmon resonance than its affinity for a nonspecific antigen. When used in the context of a ligand, the term “specifically binds” refers to the ability of a ligand to recognize and bind to its respective receptor(s). When used in the context of a CPP, the term “specifically binds” refers to the ability of CPPs to translocate a cell's membrane. In some instances, when a CPP(s) and either an antibody or a ligand are combined as a TAGE agent, the TAGE agent may display the specific binding properties of both the antibody or ligand and the CPP(s). For example, in such instances, the antibody or ligand of the TAGE agent may confer specific binding to an extracellular cell surface molecule, such as a cell surface protein, while the CPP(s) confers enhanced ability of the TAGE agent to translocate across a cell membrane.

The term “antibody” is used herein in the broadest sense and encompasses various antibody structures, including but not limited to monoclonal antibodies, polyclonal antibodies, multispecific antibodies (e.g., bispecific antibodies), nanobodies, monobodies, and antibody fragments so long as they exhibit the desired antigen-binding activity.

The term “antibody” includes an immunoglobulin molecule comprising four polypeptide chains, two heavy (H) chains and two light (L) chains inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three complementarity determining regions (CDRs) and four framework (FRs), arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. Thus, the VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

The term “Fc domain” is used to define the C-terminal region of an immunoglobulin heavy chain, which may be generated by papain digestion of an intact antibody. The Fc domain may be a native sequence Fc domain or a variant Fc domain. The Fc domain of an immunoglobulin generally comprises two constant domains, a CH2 domain and a CH3 domain, and optionally comprises a CH4 domain Replacements of amino acid residues in the Fc portion to alter antibody effector function are known in the art (Winter, et al. U.S. Pat. Nos. 5,648,260; 5,624,821). The Fc domain of an antibody mediates several important effector functions e.g. cytokine induction, ADCC, phagocytosis, complement dependent cytotoxicity (CDC) and half-life/clearance rate of antibody and antigen-antibody complexes. In certain embodiments, at least one amino acid residue is altered (e.g., deleted, inserted, or replaced) in the Fc domain of an Fc domain-containing binding protein such that effector functions of the binding protein are altered.

An “intact” or a “full length” antibody, as used herein, refers to an antibody comprising four polypeptide chains, two heavy (H) chains and two light (L) chains. In one embodiment, an intact antibody is an intact IgG antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of one mammalian species, such as a mouse, have been grafted onto human framework sequences. Additional framework region modifications may be made within the human framework sequences. A “humanized form” of an antibody, e.g., a non-human antibody, refers to an antibody that has undergone humanization.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); and multispecific antibodies formed from antibody fragments.

A “multispecific antigen binding polypeptide” or “multispecific antibody” is an antigen binding polypeptide that targets and binds to more than one antigen or epitope. A “bispecific,” “dual-specific” or “bifunctional” antigen binding polypeptide or antibody is a hybrid antigen binding polypeptide or antibody, respectively, having two different antigen binding sites. Bispecific antigen binding polypeptides and antibodies are examples of a multispecific antigen binding polypeptide or a multispecific antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553, Brinkmann and Kontermann. 2017. MABS. 9 (2): 182-212. The two binding sites of a bispecific antigen binding polypeptide or antibody, for example, will bind to two different epitopes, which may reside on the same or different protein targets.

The term “antibody mimetic” or “antibody mimic” refers to a molecule that is not structurally related to an antibody but is capable of specifically binding to an antigen. Examples of antibody mimetics include, but are not limited to, an adnectin (i.e., fibronectin based binding molecules), an affilin, an affimer, an affitin, an alphabody, an affibody, DARPins, an anticalin, an avimer, a fynomer, a Kunitz domain peptide, a monobody, a nanoCLAMP, a nanobody, a unibody, a versabody, an aptamer, and a peptidic molecule all of which employ binding structures that, while they mimic traditional antibody binding, are generated from and function via distinct mechanisms.

Amino acid sequences described herein may include “conservative mutations,” including the substitution, deletion or addition of nucleic acids that alter, add or delete a single amino acid or a small number of amino acids in a coding sequence where the nucleic acid alterations result in the substitution of a chemically similar amino acid. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N) and glutamine (Q); N, Q, serine(S), threonine (T), and tyrosine (Y); K, R, H, D, and E; D, E, N, and Q; alanine (A), valine (V), leucine (L), isoleucine (I), proline (P), phenylalanine (F), tryptophan (W), methionine (M), cysteine (C), and glycine (G); F, W, and Y; H, F, W, and Y; C, S and T; C and A; S and T; C and S; S, T, and Y; V, I, and L; V, I, and T. Other conservative amino acid substitutions are also recognized as valid, depending on the context of the amino acid in question. For example, in some cases, methionine (M) can substitute for lysine (K). In addition, sequences that differ by conservative variations are generally homologous.

The term “isolated” refers to a compound, which can be e.g. an antibody or antibody fragment, that is substantially free of other cellular material. Thus, in some aspects, antibodies provided are isolated antibodies which have been separated from antibodies with a different specificity.

As used herein, an “immune checkpoint” or “immune checkpoint molecule” is a molecule that modulates the immune system. An immune checkpoint molecule can be a stimulatory checkpoint molecule, i.e., turn up a signal, or inhibitory checkpoint molecule, i.e., turn down a signal. In specific embodiments, the immune checkpoint is a protein expressed either by T cells or by antigen presenting cells (APC). Certain types of cancer cells express immune checkpoint proteins to evade immune clearance. In some embodiments, the immune checkpoint is a molecule that regulates T cell activation (e.g., negative co-stimulation). Molecules that regulate T cell co-stimulation and co-inhibition are described, for example, in Chen L, Flies D B. Nature Reviews Immunology. 2013 April;13 (4): 227-42, which is hereby incorporated by reference.

As used herein, an “immune checkpoint blockade” is an agent capable of altering the activity of an immune checkpoint in a subject. In certain embodiments, an immune checkpoint blockade agent alters the function of one or more immune checkpoint molecules including, but not limited to, A2AR, B7-H3, B7-H4, BTLA, CD27, CD28, CD40, CD40L, CD47, CD70, CD80, CD86, CD112/PVRL2, CD122, CD137, CD137-L, CD155/PVR, CD160, CD226, CGEN-15049, CTLA-4, GITR, GITR-L, GALS, HVEM, ICOS, IDO, KIR, NOX2, OX40, OX40L, PD-1, PD-L1, PD-L2, PD-L3, PD-L4, SIGLEC7, SIGLEC15, SIRPa, TIGIT, TIM-3, VISTA, 2B4, or LAG-3. The immune checkpoint blockade agent may be an agonist or an antagonist of the immune checkpoint. In some embodiments, the immune checkpoint blockade agent is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint blockade agent is a small molecule. In some embodiments, the immune checkpoint blockade agent targets a molecule that regulates T cell activation (e.g., negative co-stimulation). In one embodiment, the immune checkpoint blockade agent is an anti-CTLA-4 antibody, an anti-PD-1 antibody, or an anti-PD-L1 antibody. Additional of immune checkpoints and immune checkpoint blockade agents can be found, for example, in Pardoll D M. Nature Reviews Cancer. 2012 April; 12(4): 252-64, which is hereby incorporated by reference.

Additional definitions are described in the sections below.

Various aspects of the invention are described in further detail in the following subsections.

### II. DNA Targeting System for Modifying TNFAIP3 or SOCS1

### Guide RNAs

Provided herein are DNA targeting systems, and related compositions and methods, for modifying an immunomodulatory gene selected from Tumor Necrosis Factor Alpha-Induced Protein 3 (TNFAIP3) gene or a Suppressor of Cytokine Signaling 1 (SOCS1) gene. Each component of the DNA targeting system is described in further detail herein.

### A. DNA Targeting System for Modifying TNFAIP3

In one aspect, provided herein is a DNA targeting system, and related compositions and methods, for modifying a Tumor Necrosis Factor Alpha-Induced Protein 3 (TNFAIP3) gene. In some embodiments, the DNA targeting system includes a site-directed modifying polypeptide and a guide RNA (gRNA) capable of targeting a TNFAIP3 gene. The TNFAIP3 gene may be within a cell, e.g., a cell within a subject, such as a human. The present disclosure also provides methods of using DNA targeting systems for modifying a TNFAIP3 gene, and for treating a subject who would benefit from modifying a TNFAIP3 gene (e.g., in one or more tissues of the subject), such as a subject having a cancer (e.g., a tumor).

In one embodiment, the TNFAIP3 gene is a human TNFAIP3 gene. The human TNFAIP3 gene includes 13 exons and is located at locus 6q23.3 of the human genome. The nucleotide sequence of the genomic region of human chromosome harboring the TNFAIP3 gene may be found in, for example, the Genome Reference Consortium Human Build 38 (also referred to as Human Genome build 38 or GRCh38) available at GenBank. The nucleotide sequence of the genomic region of human chromosome 6 harboring the TNFAIP3 gene may also be found at, for example, GenBank Accession No. NC_000006.12, corresponding to nucleotides 137866317-137883312) of human chromosome 6. Examples of human TNFAIP3 DNA, mRNA, and amino acid sequences are readily available using publicly available databases, e.g., GenBank, UniProt, and OMIM. Additional information on the human TNFAIP3 gene can be found, for example, at NCBI under Gene ID: 7128.

In one embodiment, the TNFAIP3 gene is a mouse TNFAIP3 gene (i.e., Tnfaip3). The mouse TNFAIP3 gene includes 10 exons and is located at locus 10 A3; 10 8.08 cM of the mouse genome. The TNFAIP3 gene in mice is located in the mouse chromosomal region 10 A3; 10 8.08 cM. The nucleotide sequence of the genomic region of mouse chromosome harboring the TNFAIP3 gene may be found in, for example, the Genome Reference Consortium Mouse Build 39 (also referred to as Mouse Genome build 39 or GRCm39) available at GenBank. The nucleotide sequence of the genomic region of mouse chromosome 10 harboring the TNFAIP3 gene may also be found at, for example, GenBank Accession No. NC_000076.7, corresponding to nucleotides 18876658-18891158 of mouse chromosome 10. Examples of mouse TNFAIP3 DNA, mRNA, and amino acid sequences are readily available using publicly available databases, e.g., GenBank, UniProt, and OMIM. Additional information on the mouse TNFAIP3 gene can be found, for example, at NCBI under Gene ID: 21929.

Multiple transcript variants of the human TNFAIP3 gene have been identified. The nucleotide and amino acid sequence of human TNFAIP3 transcript variant 1 can be found in, for example, GenBank Reference Sequence: NM_001270507.2; the nucleotide and amino acid sequence of a human TNFAIP3 transcript variant 2 can be found in, for example, GenBank Reference Sequence: NM_001270508.2; and the nucleotide and amino acid sequence of a human TNFAIP3 transcript variant 3 can be found in, for example, GenBank Reference Sequence: NM_006290.4. All three variants encode the same protein.

There is one major transcriptional isoform of TNFAIP3 (Uniprot Accession No. P21580-1; Genbank Accession No: NP_001257436.1, NP_001257437.1, or NP_006281.1) and eight potential isoforms that have been computationally mapped.

The DNA targeting system herein may be designed to bind and recognize a target region within a TNFAIP3 gene. In certain embodiments, the target region is within an exon or in a region flanking an exon (e.g., within 10, 20, 50, 100, 200, or 500 nucleotides upstream of the 5′ end or downstream of the 3′ end of the exon) of a TNFAIP3 gene. In certain embodiments, the target region is within exons, 2, 3, or 4 f a TNFAIP3 gene.

The TNFAIP3 protein includes several functional domains, including an OTU (ovarian tumour) deubiquitinase domain, a TRAF-binding region, at least three ubiquitin-interacting regions (within the OTU domain), a TNIP1-interacting region, a RIPK1-interacting region, a lysosomal localization domain and at least seven A20-type zinc finger domains. The A20-type zinc fingers mediate the ubiquitin ligase activity of TNFAIP3, while the OTU domain mediates the deubiquitinase activity of TNFAIP3 (Wertz, I. E et al. (2004). Nature, 430(7000), 694-699). In some embodiments, the target region is a region of the TNFAIP3 gene that encodes a catalytic domain or a binding domain of the TNFAIP3 protein.

In one embodiment, the target region is a region of the TNFAIP3 gene that encodes an OTU domain of the human TNFAIP3 protein (e.g., corresponding to amino acid residues 92-263 of Uniprot Accession No. P21580). In one embodiment, the target region is a region of the TNFAIP3 gene that encodes a TRAF-binding region of the human TNFAIP3 protein (e.g., corresponding to amino acid residues 58-300 of Uniprot Accession No. P21580). In one embodiment, the target region is a region of the TNFAIP3 gene that encodes a TNIP1-interacting domain of the human TNFAIP3 protein (e.g., corresponding to amino acid residues 369-775 of Uniprot Accession No. P21580). In one embodiment, the target region is a region of the TNFAIP3 gene that encodes a RIPK1-interacting domain of the human TNFAIP3 protein (e.g., corresponding to amino acid residues 386-453 of Uniprot Accession No. P21580). In one embodiment, the target region is a region of the TNFAIP3 gene that encodes a lysosomal localization domain of the human TNFAIP3 protein (e.g., corresponding to amino acid residues 697-790 of Uniprot Accession No. P21580). In one embodiment, the target region is a region of the TNFAIP3 gene that encodes a zinc-finger domain of the human TNFAIP3 protein (e.g., corresponding to amino acid residues 381-416, 472-507, 515-548, 601-636, 651-686, 710-745, and/or 756-790 of Uniprot Accession No. P21580).

In some embodiment, the DNA-targeting system may be designed to reduce or inhibit expression of the TNFAIP3 gene. For example, in one embodiment, the DNA-targeting system modifies a TNFAIP3 gene in a manner that results in reduced expression of the TNFAIP3 gene.

Alternatively, the DNA-targeting system may be designed to modify a TNFAIP3 gene in a manner that disrupts an activity of the encoded TNFAIP3 protein, such as an enzymatic or a binding activity of a TNFAIP3 protein. For example, in one embodiment, the DNA-targeting system modifies a TNFAIP3 gene in a manner that abrogates an activity of the encoded TNFAIP3 protein (i.e., produces a loss-of-function TNFAIP3 protein), or fragment thereof, without necessarily reducing gene expression levels. In certain embodiments, the DNA targeting system introduces a substitution, such as a missense or nonsense mutation, at a residue required for an activity (e.g., a catalytic activity or a binding activity) of the encoded TNFAIP3 protein.

“Inhibiting” or “reducing” expression or activity of a TNFAIP3 gene includes any level of inhibition of a TNFAIP3 gene, e.g., at least partial suppression of the expression of a TNFAIP3 gene or at least partial suppression of an activity of a TNFAIP3 protein encoded by the TNFAIP3 gene, such as a reduction of expression by at least about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 2%, 4%, 5%, 10%, 15%, 20%, 30%, 40%, 50%, 60%, 70%, 80%, 90%, 95%, or more relative to a control or below the level of detection of TNFAIP3 expression or activity.

In some embodiments, inhibition or reduction of expression of the TNFAIP3 gene is by at least about 0.10%, at least about 0.11%, at least about 0.12%, at least about 0.13%, at least about 0.14%, at least about 0.15%, at least about 0.16%, at least about 0.17%, at least about 0.18%, at least about 0.19%, at least about 0.20%, at least about 0.30%, at least about 0.40%, at least about 0.50%, at least about 0.6%, at least about 0.7%, at least about 0.8%, at least about 0.9%, or at least about 1%, at least about 2%, at least about 3%, at least about 4%, at least about 5%, or at least about 10% relative to a control. In some embodiments, inhibition or reduction of expression of the TNFAIP3 gene is 0.05%-0.10%, 0.05%-0.15%, 0.05%-0.2%, 0.05%-0.4%, 0.05%-0.8%, 0.5%-1%, 0.5%-2%, 0.5%-5%, 0.1%-0.2%, 0.1%-0.4%, 0.1%-0.8%, 0.1%-1%, 0.1%-2%, 0.1%-5%, or 0.1%-10%, relative to a control.

In some embodiments, inhibition or reduction of expression is by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% relative to a control. In some embodiments, inhibition or reduction of expression of the TNFAIP3 gene is 10%-20%, 10%-40%, 10%-60%, 10%-80%, 10%-100%, 15%-30%, 15%-50%, 15%-75%, 15%-100%, 20%-40%, 20%-60%-20%-80%, 20%-100%, 30%-60%, 30%-80%, 30%-100%, 40%-80%, 40%-100%, 50%-80%, 50%-100%, 60%-70%, 60%-80%, 60%-90%, 70%-90%, 70%-100%, 80%-100%, or 90%-100% relative to a control.

In some embodiments, the DNA targeting systems herein are contacted with cells in a population of cells. In such instances, the TNFAIP3 gene may be modified in a portion of cells in the population. The methods herein may modify both copies of the TNFAIP3 gene or a single copy of the TNFAIP3 gene (e.g., leading to haploinsufficiency) in a cell. The percentage of cells that include a modified gene (e.g., a TNFAIP3 gene) may be used to assess the editing rate. For example, the TNFAIP3 gene may be modified in at least about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 2%, 4%, 5%, 10%, 15%, 20%, 30%, 40%, 50%, 60%, 70%, 80%, 90%, 95%, or more cells in the population of cells.

In some embodiments, the methods herein are effective to modify a TNFAIP3 gene in at least about 0.10%, at least about 0.11%, at least about 0.12%, at least about 0.13%, at least about 0.14%, at least about 0.15%, at least about 0.16%, at least about 0.17%, at least about 0.18%, at least about 0.19%, at least about 0.20%, at least about 0.30%, at least about 0.40%, at least about 0.50%, at least about 0.6%, at least about 0.7%, at least about 0.8%, at least about 0.9%, or at least about 1%, at least about 2%, at least about 3%, at least about 4%, at least about 5%, or at least about 10% cells in the population of cells. In some embodiments, the methods herein are effective to modify a TNFAIP3 gene in 0.05%-0.10%, 0.05%-0.15%, 0.05%-0.2%, 0.05%-0.4%, 0.05%-0.8%, 0.5-1%, 0.5%-2%, 0.5%-5%, 0.1%-0.2%, 0.1%-0.4%, 0.1%-0.8%, 0.1%-1%, 0.1%-2%, 0.1%-5%, or 0.1%-10% cells in the population of cells.

In certain embodiments, the methods herein are effective to modify a TNFAIP3 gene in at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of cells in a population of cells. In some embodiments, the methods herein are effective to modify a TNFAIP3 gene in 10%-20%, 10%-40%, 10%-60%, 10%-80%, 10%-100%, 15%-30%, 15%-50%, 15%-75%, 15%-100%, 20%-40%, 20%-60%-20%-80%, 20%-100%, 30%-60%, 30%-80%, 30%-100%, 40%-80%, 40%-100%, 50%-80%, 50%-100%, 60%-70%, 60%-80%, 60%-90%, 70%-90%, 70%-100%, 80%-100%, or 90%-100% of cells in the population of cells.

The methods herein may modify a single copy or both copies of the TNFAIP3 gene in a cell. In some embodiments, both copies of the TNFAIP3 gene are modified in a cell. Alternatively, a single copy of the TNFAIP3 gene is modified in a cell (e.g., leading to haploinsufficiency).

The expression of a TNFAIP3 gene may be assessed based on the level of any variable associated with TNFAIP3 gene expression, e.g., TNFAIP3 mRNA level or TNFAIP3 protein level. The expression of a TNFAIP3 gene may also be assessed indirectly based on, for example, ubiquitin ligase activity, deubiquitinase activity, modulation of a known biological function of TNFAIP3 (e.g. modulation of NF-kB signaling), or downstream pharmacodynamics effect of TNFAIP3 in a tissue sample, such as a tumor sample. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive or non-targeting agent control).

In the methods and compositions herein, the gRNA targeting TNFAIP3 may comprise a gRNA identified in the Examples and as summarized in Table 1. Accordingly, in some embodiments, the gRNA targeting the TNFAIP3 gene comprises a polynucleotide sequence (an RNA sequence, e.g., an gRNA spacer) set forth in any one of SEQ ID NOs: 70-124, or a variant thereof having at least 90%, 95%, 97%, 98%, or 99% sequence identity (e.g., wherein the variant is capable of directing the site-directed modifying polypeptide to the target site). In certain embodiments, provided is a crRNA comprising a polynucleotide sequence of any one of SEQ ID NOs: 70-124. In some embodiments, the gRNA targeting the TNFAIP3 gene targets the DNA sequence of any one of SEQ ID NOs: 15-69, or a variant thereof having at least 90%, 95%, 97%, 98%, or 99% sequence identity. In some embodiments, the gRNA targeting the TNFAIP3 gene is encoded by an isolated polynucleotide comprising the polynucleotide sequence (DNA sequence) of any one of SEQ ID NOs: 125-179, or a variant thereof having at least 90%, 95%, 97%, 98%, or 99% sequence identity, wherein the variant encodes a gRNA capable of directing the site-directed modifying polypeptide to the target site.

In some embodiments, the gRNA comprises the polynucleotide sequence of SEQ ID NO:70. In some embodiments, the gRNA comprises the polynucleotide sequence of SEQ ID NO:71. In some embodiments, the gRNA comprises the polynucleotide sequence of SEQ ID NO:72. In some embodiments, the gRNA comprises the polynucleotide sequence of SEQ ID NO:73. In some embodiments, the gRNA comprises the polynucleotide sequence of SEQ ID NO:74. In some embodiments, the gRNA comprises the polynucleotide sequence of SEQ ID NO:75. In some embodiments, the gRNA comprises the polynucleotide sequence of SEQ ID NO:76. In some embodiments, the gRNA comprises the polynucleotide sequence of SEQ ID NO:77. In some embodiments, the gRNA comprises the polynucleotide sequence of SEQ ID NO:78. In some embodiments, the gRNA comprises the polynucleotide sequence of SEQ ID NO:79. In some embodiments, the gRNA comprises the polynucleotide sequence of SEQ ID NO:80. In some embodiments, the gRNA comprises the polynucleotide sequence of SEQ ID NO:81. In some embodiments, the gRNA comprises the polynucleotide sequence of SEQ ID NO:82. In some embodiments, the gRNA comprises the polynucleotide sequence of SEQ ID NO:83. In some embodiments, the gRNA comprises the polynucleotide sequence of SEQ ID NO:84. In some embodiments, the gRNA comprises the polynucleotide sequence of SEQ ID NO:85. In some embodiments, the gRNA comprises the polynucleotide sequence of SEQ ID NO:86. In some embodiments, the gRNA comprises the polynucleotide sequence of SEQ ID NO:87. In some embodiments, the gRNA comprises the polynucleotide sequence of SEQ ID NO:88. In some embodiments, the gRNA comprises the polynucleotide sequence of SEQ ID NO:89. In some embodiments, the gRNA comprises the polynucleotide sequence of SEQ ID NO:90. In some embodiments, the gRNA comprises the polynucleotide sequence of SEQ ID NO:91. In some embodiments, the gRNA comprises the polynucleotide sequence of SEQ ID NO:92. In some embodiments, the gRNA comprises the polynucleotide sequence of SEQ ID NO:93. In some embodiments, the gRNA comprises the polynucleotide sequence of SEQ ID NO:94. In some embodiments, the gRNA comprises the polynucleotide sequence of SEQ ID NO:95. In some embodiments, the gRNA comprises the polynucleotide sequence of SEQ ID NO:96. In some embodiments, the gRNA comprises the polynucleotide sequence of SEQ ID NO:97. In some embodiments, the gRNA comprises the polynucleotide sequence of SEQ ID NO:98. In some embodiments, the gRNA comprises the polynucleotide sequence of SEQ ID NO:99. In some embodiments, the gRNA comprises the polynucleotide sequence of SEQ ID NO:100. In some embodiments, the gRNA comprises the polynucleotide sequence of SEQ ID NO:101. In some embodiments, the gRNA comprises the polynucleotide sequence of SEQ ID NO:102. In some embodiments, the gRNA comprises the polynucleotide sequence of SEQ ID NO:103. In some embodiments, the gRNA comprises the polynucleotide sequence of SEQ ID NO:104. In some embodiments, the gRNA comprises the polynucleotide sequence of SEQ ID NO:105. In some embodiments, the gRNA comprises the polynucleotide sequence of SEQ ID NO:106. In some embodiments, the gRNA comprises the polynucleotide sequence of SEQ ID NO:107. In some embodiments, the gRNA comprises the polynucleotide sequence of SEQ ID NO:108. In some embodiments, the gRNA comprises the polynucleotide sequence of SEQ ID NO:109. In some embodiments, the gRNA comprises the polynucleotide sequence of SEQ ID NO:110. In some embodiments, the gRNA comprises the polynucleotide sequence of SEQ ID NO:111. In some embodiments, the gRNA comprises the polynucleotide sequence of SEQ ID NO:112. In some embodiments, the gRNA comprises the polynucleotide sequence of SEQ ID NO:113. In some embodiments, the gRNA comprises the polynucleotide sequence of SEQ ID NO:114. In some embodiments, the gRNA comprises the polynucleotide sequence of SEQ ID NO:115. In some embodiments, the gRNA comprises the polynucleotide sequence of SEQ ID NO:116. In some embodiments, the gRNA comprises the polynucleotide sequence of SEQ ID NO:117. In some embodiments, the gRNA comprises the polynucleotide sequence of SEQ ID NO:118. In some embodiments, the gRNA comprises the polynucleotide sequence of SEQ ID NO:119. In some embodiments, the gRNA comprises the polynucleotide sequence of SEQ ID NO:120. In some embodiments, the gRNA comprises the polynucleotide sequence of SEQ ID NO:121. In some embodiments, the gRNA comprises the polynucleotide sequence of SEQ ID NO:122. In some embodiments, the gRNA comprises the polynucleotide sequence of SEQ ID NO:123. In some embodiments, the gRNA comprises the polynucleotide sequence of SEQ ID NO:124.

In some embodiments, the gRNA targeting the TNFAIP3 gene comprises modifications of the gRNA sequence (e.g., modifications of a gRNA sequence provided herein). In some cases, the gRNA (e.g., sgRNA) comprises a first polynucleotide sequence that is complementary to the target nucleic acid and a second polynucleotide sequence that interacts with a Cas polypeptide. In other instances, the gRNA (e.g., sgRNA) comprises one or more modified nucleotides. In some cases, one or more of the nucleotides in the first polynucleotide sequence and/or the second polynucleotide sequence are modified nucleotides.

In some embodiments, the modified nucleotides comprise a modification in a ribose group, a phosphate group, a nucleobase, or a combination thereof. In some instances, the modification in the ribose group comprises a modification at the 2′ position of the ribose group. In some cases, the modification at the 2′ position of the ribose group is selected from the group consisting of 2′-O-methyl, 2′-fluoro, 2′-deoxy, 2′-O-(2-methoxyethyl), and a combination thereof. In other instances, the modification in the phosphate group comprises a phosphorothioate modification. In other embodiments, the modified nucleotides are selected from the group consisting of a 2′-ribo 3′-phosphorothioate(S), 2′-O-methyl (M) nucleotide, a 2′-O-methyl 3′-phosphorothioate (MS) nucleotide, a 2′-O-methyl 3′-thioPACE (MSP) nucleotide, and a combination thereof.

Further, non-base modifiers (such as a C3 spacer propanediol group or a ZEN modifier napthyl-azo group) can be placed at one or both ends of the gRNA without loss of activity and also block exonuclease attack. Accordingly, in some embodiments, the gRNA comprises a C3 spacer propanediol group. In some embodiments, the gRNA comprises a ZEN modifier napthyl-azo group.

The gRNAs of the DNA targeting systems provided herein may be designed for use with a particular site-directed modifying polypeptide, such as a Cas9 polypeptide, a Cas12 polypeptide (e.g., Cas12a), or a base editor.

In one embodiment, the DNA targeting system comprises Cas9 (e.g., spCas9) as the site directed-modifying polypeptide and a guide RNA (gRNA), wherein the gRNA comprises a polynucleotide sequence set forth in any one of SEQ ID NOs: 89, 90, 92, 94-98, and 100-120, or a variant thereof having at least 90%, 95%, 97%, 98%, or 99% sequence identity, wherein the variant is capable of directing the site-directed modifying polypeptide to the target site. In some embodiments, the gRNA targeting the TNFAIP3 gene targets the DNA sequence of any one of SEQ ID NOs: 34, 35, 37, 39-43, and 45-65, or a variant thereof having at least 90%, 95%, 97%, 98%, or 99% sequence identity. In some embodiments, the gRNA targeting the TNFAIP3 gene is encoded by an isolated polynucleotide comprising the polynucleotide sequence (DNA sequence) of any one of SEQ ID NOS: 144, 145, 147, 149-153, and 155-175, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the TNFAIP3 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the TNFAIP3 gene comprises a non-base modifier (such as a C3 spacer propanediol group or a ZEN modifier napthyl-azo group) at one or both ends of the gRNA.

In one embodiment, the DNA targeting system comprises Cas12 (e.g., Cas12a) as the site directed-modifying polypeptide and a guide RNA (gRNA), wherein the at least one gRNA comprises a polynucleotide sequence set forth in any one of SEQ ID NOs: 70-88, 91, 93, and 99, or a variant thereof having at least 90%, 95%, 97%, 98%, or 99% sequence identity, wherein the variant is capable of directing the site-directed modifying polypeptide to the target site. In some embodiments, the gRNA targeting the TNFAIP3 gene targets the DNA sequence of any one of SEQ ID NOs: 15-33, 36, 38, and 44, or a variant thereof having at least 90%, 95%, 97%, 98%, or 99% sequence identity.

In some embodiments, the gRNA targeting the TNFAIP3 gene is encoded by an isolated polynucleotide comprising the polynucleotide sequence (DNA sequence) of any one of SEQ ID NOS: 125-143, 146, 148, and 154, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the TNFAIP3 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the TNFAIP3 gene comprises a non-base modifier (such as a C3 spacer propanediol group or a ZEN modifier napthyl-azo group) at one or both ends of the gRNA.

In one embodiment, the DNA targeting system comprises a base editor (e.g., a cytosine base editor (CBE)) as the site directed-modifying polypeptide and a guide RNA (gRNA), wherein the at least one gRNA comprises the polynucleotide of SEQ ID NO: 121-124, or a variant thereof having at least 90%, 95%, 97%, 98%, or 99% sequence identity, wherein the variant is capable of directing the site-directed modifying polypeptide to the target site. In some embodiments, the gRNA targeting the TNFAIP3 gene targets the DNA sequence of any one of SEQ ID NOs: 66-69, or a variant thereof having at least 90%, 95%, 97%, 98%, or 99% sequence identity. In some embodiments, the gRNA targeting the TNFAIP3 gene is encoded by an isolated polynucleotide comprising the polynucleotide sequence (DNA sequence) of SEQ ID NO: 176-179, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the TNFAIP3 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the TNFAIP3 gene comprises a non-base modifier (such as a C3 spacer propanediol group or a ZEN modifier napthyl-azo group) at one or both ends of the gRNA.

The DNA targeting system may be used to target any exon in TNFAIP3, including any one of exons 2, 3, or 4. In some embodiments, the DNA targeting system comprises a site-directed modifying polypeptide (e.g., Cas9) and gRNA capable of targeting a 5′ region of the TNFAIP3 gene. gRNAs targeting earlier regions of the TNFAIP3 gene may induce a frameshift causing indel that lead to a loss of function protein. Alternatively, the DNA targeting system comprises a site-directed modifying polypeptide (e.g., Cas9) and gRNA capable of targeting a later region of the TNFAIP3 gene.

In some embodiments, the DNA targeting system comprises a site-directed modifying polypeptide (e.g., Cas9, Cas12a, or a base editor) and gRNA capable of targeting a region of the TNFAIP3 gene that encodes an OTU domain of a human TNFAIP3 protein (e.g., corresponding to amino acid residues 92-263 of UniProt Accession No. P21580); a ubiquitin-interacting subdomain of a human TNFAIP3 protein (e.g., corresponding to amino acid residues 157-159, 190-192, and/or 224-227 of UniProt Accession No. P21580); a TRAF-binding domain of a human TNFAIP3 protein (e.g., corresponding to amino acid residues 58-300 of Uniprot Accession No. P21580); a TNIP1-interaction domain of a human TNFAIP3 protein (e.g., corresponding to amino acid residues 369-775 of Uniprot Accession No. P21580); a RIPK1-interacting domain of a human TNFAIP3 protein (e.g., corresponding to amino acid residues 386-453 of Uniprot Accession No. P21580); a lysosomal localization domain of a human TNFAIP3 protein (e.g., corresponding to amino acid residues 697-790 of Uniprot Accession No. P21580); a zinc-finger domain of a human TNFAIP3 protein (e.g., corresponding to amino acid residues 381-416, 472-507, 515-548, 601-636, 651-686, 710-745, and/or 756-790 of Uniprot Accession No. P21580) of a human TNFAIP3 protein.

Further details regarding site-directed modifying polypeptides and gRNAs that may be used in the compositions and methods are provided herein (see, e.g., Table 1). Additionally, in some embodiments, the site-directed modifying polypeptide further comprises a cell targeting agent, thereby forming a targeted active gene editing (TAGE) agent, which are further described in section III.

TABLE 1
Human TNFAIP3 Guide RNAs (gRNAs)

| | | | | | | DNA Sequence | |
|---|---|---|---|---|---|---|---|
| | | Target DNA | SEQ | gRNA spacer | SEQ | encoding gRNA | SEQ |
| gRNA | Cas | Sequence | ID | sequence (5′ | ID | DNA Sequence | ID |
| Name | Enzyme | (5′ to 3′) | NO | to 3′) | NO | (5′ to 3′) | NO |
| 137874943 | Cas12a | CTTGAGCGTG | 15 | CUUGAGCGUGC | 70 | TAATTTCTACTC | 125 |
| rev | | CTGAACAGCG | | UGAACAGCGC | | TTGTAGATCTTG | |
| | | C | | | | AGCGTGCTGAA | |
| | | | | | | CAGCGC | |
| 137874990 | Cas12a | AGAGACTCCA | 16 | AGAGACUCCAG | 71 | TAATTTCTACTC | 126 |
| rev | | GTTGCCAGCG | | UUGCCAGCGG | | TTGTAGATAGA | |
| | | G | | | | GACTCCAGTTG | |
| | | | | | | CCAGCGG | |
| 137871512 | Cas12a | AGCGCCACAA | 17 | AGCGCCACAAG | 72 | TAATTTCTACTC | 127 |
| rev | | GCTTCCGGACT | | CUUCCGGACU | | TTGTAGATAGC | |
| | | | | | | GCCACAAGCTT | |
| | | | | | | CCGGACT | |
| 137871405 | Cas12a | GGGAGATCAT | 18 | GGGAGAUCAUC | 73 | TAATTTCTACTC | 128 |
| forw | | CCACAAAGCC | | CACAAAGCCC | | TTGTAGATGGG | |
| | | C | | | | AGATCATCCAC | |
| | | | | | | AAAGCCC | |
| 137874961 | Cas12a | AATTCCGCTGG | 19 | AAUUCCGCUGG | 74 | TAATTTCTACTC | 129 |
| forw | | CAACTGGAGT | | CAACUGGAGU | | TTGTAGATAATT | |
| | | | | | | CCGCTGGCAAC | |
| | | | | | | TGGAGT | |
| 137871422 | Cas12a | TGGATGATCTC | 20 | UGGAUGAUCUC | 75 | TAATTTCTACTC | 130 |
| rev | | CCGAAACTGA | | CCGAAACUGA | | TTGTAGATTGGA | |
| | | | | | | TGATCTCCCGA | |
| | | | | | | AACTGA | |
| 137871372 | Cas12a | CAGTGTGTATC | 21 | CAGUGUGUAUC | 76 | TAATTTCTACTC | 131 |
| rev | | GGTGCATGGT | | GGUGCAUGGU | | TTGTAGATCAGT | |
| | | | | | | GTGTATCGGTG | |
| | | | | | | CATGGT | |
| 137871315 | Cas12a | AACCTACTAAT | 22 | AACCUACUAAU | 77 | TAATTTCTACTC | 132 |
| forw | | GGGATCATTC | | GGGAUCAUUC | | TTGTAGATAACC | |
| | | | | | | TACTAATGGGAT | |
| | | | | | | CATTC | |
| 137871439 | Cas12a | TGTCGATGAG | 23 | UGUCGAUGAGG | 78 | TAATTTCTACTC | 133 |
| rev | | GGCTTTGTGGA | | GCUUUGUGGA | | TTGTAGATTGTC | |
| | | | | | | GATGAGGGCTT | |
| | | | | | | TGTGGA | |
| 137871394 | Cas12a | TCCTCAGTTTC | 24 | UCCUCAGUUUC | 79 | TAATTTCTACTC | 134 |
| forw | | GGGAGATCAT | | GGGAGAUCAU | | TTGTAGATTCCT | |
| | | | | | | CAGTTTCGGGA | |
| | | | | | | GATCAT | |
| 137871459 | Cas12a | CAGGGTGGCC | 25 | CAGGGUGGCCU | 80 | TAATTTCTACTC | 135 |
| rev | | TGGATGTTTCT | | GGAUGUUUCU | | TTGTAGATCAG | |
| | | | | | | GGTGGCCTGGA | |
| | | | | | | TGTTTCT | |
| 137871345 | Cas12a | AAACCATGCAC | 26 | AAACCAUGCAC | 81 | TAATTTCTACTC | 136 |
| forw | | CGATACACAC | | CGAUACACAC | | TTGTAGATAAAC | |
| | | | | | | CATGCACCGAT | |
| | | | | | | ACACAC | |
| 137871254 | Cas12a | TATTTGAGCAA | 27 | UAUUUGAGCAA | 82 | TAATTTCTACTC | 137 |
| forw | | TATGCGGAAA | | UAUGCGGAAA | | TTGTAGATTATT | |
| | | | | | | TGAGCAATATG | |
| | | | | | | CGGAAA | |
| 137875778 | Cas12a | TTCCAGTGAGT | 28 | UUCCAGUGAGU | 83 | TAATTTCTACTC | 138 |
| rev | | TGTACTGAAG | | UGUACUGAAG | | TTGTAGATTTCC | |
| | | | | | | JAGTGAGTTGTA | |
| | | | | | | CTGAAG | |
| 137871275 | Cas12a | CGCATATTGCT | 29 | CGCAUAUUGCU | 84 | TAATTTCTACTC | 139 |
| rev | | CAAATACAAA | | CAAAUACAAA | | TTGTAGATCGC | |
| | | | | | | ATATTGCTCAAA | |
| | | | | | | TACAAA | |
| 137871260 | Cas12a | AGCAATATGCG | 30 | AGCAAUAUGCG | 85 | TAATTTCTACTC | 140 |
| forw | | GAAAGCTGTG | | GAAAGCUGUG | | TTGTAGATAGCA | |
| | | | | | | ATATGCGGAAA | |
| | | | | | | GCTGTG | |
| 137874963 | Cas12a | AAGTTGCGTGT | 31 | AAGUUGCGUGU | 86 | TAATTTCTACTC | 141 |
| rev | | GTCTGTTTCC | | GUCUGUUUCC | | TTGTAGATAAGT | |
| | | | | | | TGCGTGTGTCT | |
| | | | | | | GTTTCC | |
| 137871470 | Cas12a | TTCTGGCTTTC | 32 | UUCUGGCUUUC | 87 | TAATTTCTACTC | 142 |
| rev | | CAGGGTGGCC | | CAGGGUGGCC | | TTGTAGATTTCT | |
| | | | | | | GGCTTTCCAGG | |
| | | | | | | GTGGCC | |
| 137875717 | Cas12a | ATAAGATTGTC | 33 | AUAAGAUUGUC | 88 | TAATTTCTACTC | 143 |
| rev | | CCATTCATCA | | CCAUUCAUCA | | TTGTAGATATAA | |
| | | | | | | GATTGTCCCATT | |
| | | | | | | CATCA | |
| 137874973 | SpCas9 | CACGCAACTTT | 34 | CACGCAACUUU | 89 | CACGCAACTTTA | 144 |
| forw | | AAATTCCGC | | AAAUUCCGC | | AATTCCGCGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |
| 137871495 | SpCas9 | TCAACTGGTGT | 35 | UCAACUGGUGU | 90 | TCAACTGGTGT | 145 |
| forw | | CGAGAAGTC | | CGAGAAGUC | | CGAGAAGTCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875790 | Cas12a | TCCTTTGCAAC | 36 | UCCUUUGCAAC | 91 | TAATTTCTACTC | 146 |
| forw | | ATCCTCAGAA | | AUCCUCAGAA | | TTGTAGATTCCT | |
| | | | | | | TTGCAACATCCT | |
| | | | | | | CAGAA | |
| 137874941 | SpCas9 | GGCGCTGTTC | 37 | GGCGCUGUUCA | 92 | GGCGCTGTTCA | 147 |
| forw | | AGCACGCTCA | | GCACGCUCA | | GCACGCTCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875009 | Cas12a | AACAAATTCCT | 38 | AACAAAUUCCU | 93 | TAATTTCTACTC | 148 |
| rev | | GAGATTTGAG | | GAGAUUUGAG | | TTGTAGATAACA | |
| | | | | | | AATTCCTGAGAT | |
| | | | | | | TTGAG | |
| 137871505 | SpCas9 | TCGAGAAGTCC | 39 | UCGAGAAGUCC | 94 | TCGAGAAGTCC | 149 |
| forw | | GGAAGCTTG | | GGAAGCUUG | | GGAAGCTTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875739 | SpCas9 | TGAAGTCCACT | 40 | UGAAGUCCACU | 95 | TGAAGTCCACTT | 150 |
| rev | | TCGGGCCAT | | UCGGGCCAU | | CGGGCCATGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137874896 | SpCas9 | TCAGTACATGT | 41 | UCAGUACAUGU | 96 | TCAGTACATGT | 151 |
| forw | | GGGGCGTTC | | GGGGCGUUC | | GGGGCGTTCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871352 | SpCas9 | TTCCAGTGTGT | 42 | UUCCAGUGUGU | 97 | TTCCAGTGTGTA | 152 |
| rev | | ATCGGTGCA | | AUCGGUGCA | | TCGGTGCAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875033 | SpCas9 | CGGGGCTTTG | 43 | CGGGGCUUUG | 98 | CGGGGCTTTGC | 153 |
| forw | | CTATGATACT | | CUAUGAUACU | | TATGATACTGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871347 | Cas12a | AAATGATGAAT | 44 | AAAUGAUGAAU | 99 | TAATTTCTACTC | 154 |
| rev | | GATCCCATTA | | GAUCCCAUUA | | TTGTAGATAAAT | |
| | | | | | | GATGAATGATC | |
| | | | | | | CCATTA | |
| 137875746 | SpCas9 | GTTGTACTGAA | 45 | GUUGUACUGAA | 100 | GTTGTACTGAA | 155 |
| rev | | GTCCACTTC | | GUCCACUUC | | GTCCACTTCGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875773 | SpCas9 | TGGACTTCAGT | 46 | UGGACUUCAGU | 101 | TGGACTTCAGT | 156 |
| forw | | ACAACTCAC | | ACAACUCAC | | ACAACTCACGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875747 | SpCas9 | AGTTGTACTGA | 47 | AGUUGUACUGA | 102 | AGTTGTACTGAA | 157 |
| rev | | AGTCCACTT | | AGUCCACUU | | GTCCACTTGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |
| 137871448 | SpCas9 | CCTCATCGACA | 48 | CCUCAUCGACA | 103 | CCTCATCGACA | 158 |
| forw | | GAAACATCC | | GAAACAUCC | | GAAACATCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871292 | SpCas9 | GCGGAAAGCT | 49 | GCGGAAAGCUG | 104 | GCGGAAAGCTG | 159 |
| forw | | GTGAAGATAC | | UGAAGAUAC | | TGAAGATACGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871494 | SpCas9 | TTTTCAGCGCC | 50 | UUUUCAGCGCC | 105 | TTTTCAGCGCC | 160 |
| rev | | ACAAGCTTC | | ACAAGCUUC | | ACAAGCTTCGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875740 | SpCas9 | CTGAAGTCCAC | 51 | CUGAAGUCCAC | 106 | CTGAAGTCCAC | 161 |
| rev | | TTCGGGCCA | | UUCGGGCCA | | TTCGGGCCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137874888 | SpCas9 | GCCACTTCTCA | 52 | GCCACUUCUCA | 107 | GCCACTTCTCA | 162 |
| forw | | GTACATGTG | | GUACAUGUG | | GTACATGTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871321 | SpCas9 | TGATGAATGAT | 53 | UGAUGAAUGAU | 108 | TGATGAATGATC | 163 |
| rev | | CCCATTAGT | | CCCAUUAGU | | CCATTAGTGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |
| 137871428 | SpCas9 | CCTGGATGTTT | 54 | CCUGGAUGUUU | 109 | CCTGGATGTTT | 164 |
| rev | | CTGTCGATG | | CUGUCGAUG | | CTGTCGATGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137874869 | SpCas9 | GCCCCACATGT | 55 | GCCCCACAUGU | 110 | GCCCCACATGT | 165 |
| rev | | ACTGAGAAG | | ACUGAGAAG | | ACTGAGAAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871291 | SpCas9 | TGCGGAAAGC | 56 | UGCGGAAAGCU | 111 | TGCGGAAAGCT | 166 |
| forw | | TGTGAAGATA | | GUGAAGAUA | | GTGAAGATAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871427 | SpCas9 | CTGGATGTTTC | 57 | CUGGAUGUUUC | 112 | CTGGATGTTTCT | 167 |
| rev | | TGTCGATGA | | UGUCGAUGA | | GTCGATGAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871419 | SpCas9 | TTCTGTCGATG | 58 | UUCUGUCGAUG | 113 | TTCTGTCGATGA | 168 |
| rev | | AGGGCTTTG | | AGGGCUUUG | | GGGCTTTGGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137874886 | SpCas9 | ATGCCACTTCT | 59 | AUGCCACUUCU | 114 | ATGCCACTTCTC | 169 |
| forw | | CAGTACATG | | CAGUACAUG | | AGTACATGGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |
| 137874908 | SpCas9 | GGGCGTTCAG | 60 | GGGCGUUCAG | 115 | GGGCGTTCAGG | 170 |
| Iforw | | GACACAGACT | | GACACAGACU | | ACACAGACTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137871246 | SpCas9 | TTGCTCAAATA | 61 | UUGCUCAAAUA | 116 | TTGCTCAAATAC | 171 |
| rev | | CAAAGCCTG | | CAAAGCCUG | | AAAGCCTGGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875722 | SpCas9 | ATGGGACAATC | 62 | AUGGGACAAUC | 117 | ATGGGACAATC | 172 |
| forw | | TTATCAAAA | | UUAUCAAAA | | TTATCAAAAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | IGGTGCTTTT | |
| 137874920 | SpCas9 | CACAGACTTGG | 63 | CACAGACUUGG | 118 | CACAGACTTGG | 173 |
| forw | | TACTGAGGA | | UACUGAGGA | | TACTGAGGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875743 | SpCas9 | GGCTTCCACA | 64 | GGCUUCCACAG | 119 | GGCTTCCACAG | 174 |
| forw | | GACACACCCA | | ACACACCCA | | ACACACCCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875014 | SpCas9 | TCTCAGGAATT | 65 | UCUCAGGAAUU | 120 | TCTCAGGAATTT | 175 |
| forw | | TGTTGAAAC | | UGUUGAAAC | | GTTGAAACGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |
| 137875773 | BaseEditor | TGGACTTCAGT | 66 | UGGACUUCAGU | 121 | TGGACTTCAGT | 176 |
| forw | | ACAACTCAC | | ACAACUCAC | | ACAACTCACGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875009 | BaseEditor | GCCCCACATGT | 67 | GCCCCACAUGU | 122 | GCCCCACATGT | 177 |
| rev | | ACTGAGAAG | | ACUGAGAAG | | ACTGAGAAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137874908 | BaseEditor | GGGCGTTCAG | 68 | GGGCGUUCAG | 123 | GGGCGTTCAGG | 178 |
| forw | | GACACAGACT | | GACACAGACU | | ACACAGACTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 137875014 | BaseEditor | TCTCAGGAATT | 69 | UCUCAGGAAUU | 124 | TCTCAGGAATTT | 179 |
| forw | | TGTTGAAAC | | UGUUGAAAC | | GTTGAAACGTTT | |
| | | | | | | TAGAGCTAGAA | |
| | | | | | | ATAGCAAGTTAA | |
| | | | | | | AATAAGGCTAG | |
| | | | | | | TCCGTTATCAAC | |
| | | | | | | TTGAAAAAGTG | |
| | | | | | | GCACCGAGTCG | |
| | | | | | | GTGCTTTT | |

### B. DNA Targeting System for Modifying SOCS1

### Guide RNAs

In one aspect, provided herein is a DNA targeting system, and related compositions and methods, for modifying a Suppressor Of Cytokine Signaling 1 (SOCS1) gene. In some embodiments, the DNA targeting system includes a site-directed modifying polypeptide and a guide RNA (gRNA) capable of targeting a SOCS1 gene. The SOCS1 gene may be within a cell, e.g., a cell within a subject, such as a human. The present disclosure also provides methods of using DNA targeting systems for modifying a SOCS1 gene, and for treating a subject who would benefit from modifying a SOCS1 gene (e.g., in one or more tissues of the subject), such as a subject having a cancer (e.g., a tumor).

In one embodiment, the SOCS1 gene is a human SOCS1 gene. The human SOCS1 gene includes two exons and is located at locus 16p13.13 of the human genome. The nucleotide sequence of the genomic region of human chromosome harboring the SOCS1 gene may be found in, for example, the Genome Reference Consortium Human Build 38 (also referred to as Human Genome build 38 or GRCh38) available at GenBank. The nucleotide sequence of the genomic region of human chromosome 16 harboring the SOCS1 gene may also be found at, for example, GenBank Accession No. NC_000016.10, corresponding to nucleotides 11254417-11256204) of human chromosome 16. Examples of human SOCS1 DNA, mRNA, and amino acid sequences are readily available using publicly available databases, e.g., GenBank, UniProt, and OMIM. Additional information on the human SOCS1 gene can be found, for example, at NCBI under Gene ID: 8651.

In one embodiment, the SOCS1 gene is a mouse SOCS1 gene (i.e., Socs1). The mouse SOCS1 gene includes two exons and is located at locus 16 A1; 16 5.81 cM of the mouse genome. The nucleotide sequence of the genomic region of mouse chromosome harboring the SOCS1 gene may be found in, for example, the Genome Reference Consortium Mouse Build 39 (also referred to as Mouse Genome build 39 or GRCm39) available at GenBank. The nucleotide sequence of the genomic region of mouse chromosome 16 harboring the SOCS1 gene may also be found at, for example, GenBank Accession No. NC_000082.7, corresponding to nucleotides 10601672-10603400 of mouse chromosome 16. Examples of mouse SOCS1 DNA, mRNA, and amino acid sequences are readily available using publicly available databases, e.g., GenBank, UniProt, and OMIM. Additional information on the mouse SOCS1 gene can be found, for example, at NCBI under Gene ID: 12703. One transcript variant of the human SOCS1 gene has been identified. The nucleotide and amino acid sequence of human SOCS1 transcript variant 1 can be found in, for example, GenBank Reference Sequence: NM_003745.2.

The DNA targeting system herein may be designed to bind and recognize a target region within a SOCS1 gene. In certain embodiments, the target region is within an exon or in a region flanking an exon (e.g., within 10, 20, 50, 100, 200, or 500 nucleotides upstream of the 5′ end or downstream of the 3′ end of the exon) of a SOCS1 gene. In certain embodiments, the target region is within exon 2 of a SOCS1 gene.

The SOCS1 protein includes several functional domains, including an SH2 (Src homology 2) domain, a SOCS box domain, a kinase inhibitory region, an extended SH2 subdomain, and an elongin BC complex interaction domain. In some embodiments, the target region is a region of the SOCS1 gene that encodes a catalytic domain or a binding domain of the SOCS1 protein. In one embodiment, the target region is a region of the SOCS1 gene that encodes an SH2 domain of the human SOCS1 protein (e.g., corresponding to amino acid residues 79-174 of Uniprot Accession No. O15524). In one embodiment, the target region is a region of the SOCS1 gene that encodes an SOCS box domain of the human SOCS1 protein (e.g., corresponding to amino acid residues 161-210 of Uniprot Accession No. 015524). In one embodiment, the target region is a region of the SOCS1 gene that encodes a kinase inhibitory region of the human SOCS1 protein (e.g., corresponding to amino acid residues 55-66 of Uniprot Accession No. 015524). In one embodiment, the target region is a region of the SOCS1 gene that encodes an extended SH2 subdomain of the human SOCS1 protein (e.g., corresponding to amino acid residues 67-78 of Uniprot Accession No. 015524). In one embodiment, the target region is a region of the SOCS1 gene that encodes an elongin BC complex interaction domain of the human SOCS1 protein (e.g., corresponding to amino acid residues 173-182 of Uniprot Accession No. 015524).

In some embodiment, the DNA-targeting system may be designed to reduce or inhibit expression of the SOCS1 gene. For example, in one embodiment, the DNA-targeting system modifies a SOCS1 gene in a manner that results in reduced expression of the SOCS1 gene.

Alternatively, the DNA-targeting system may be designed to modify a SOCS1 gene in a manner that disrupts an activity of the encoded SOCS1 protein, such as an enzymatic or a binding activity of a SOCS1 protein. For example, in one embodiment, the DNA-targeting system modifies a SOCS1 gene in a manner that abrogates an activity of the encoded SOCS1 protein (i.e., produces a loss-of-function SOCS1 protein), or fragment thereof, without necessarily reducing gene expression levels. In certain embodiments, the DNA targeting system introduces a substitution, such as a missense or nonsense mutation, at a residue required for an activity (e.g., a catalytic activity or a binding activity) of the encoded SOCS1 protein.

“Inhibiting” or “reducing” expression or activity of a SOCS1 gene includes any level of inhibition of a SOCS1 gene, e.g., at least partial suppression of the expression of a SOCS1 gene or at least partial suppression of an activity of a SOCS1 protein encoded by the SOCS1 gene, such as a reduction of expression by at least about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 2%, 4%, 5%, 10%, 15%, 20%, 30%, 40%, 50%, 60%, 70%, 80%, 90%, 95%, or more relative to a control or below the level of detection of SOCS1 expression or activity.

In some embodiments, inhibition or reduction of expression of the SOCS1 gene is by at least about 0.10%, at least about 0.11%, at least about 0.12%, at least about 0.13%, at least about 0.14%, at least about 0.15%, at least about 0.16%, at least about 0.17%, at least about 0.18%, at least about 0.19%, at least about 0.20%, at least about 0.30%, at least about 0.40%, at least about 0.50%, at least about 0.6%, at least about 0.7%, at least about 0.8%, at least about 0.9%, or at least about 1%, at least about 2%, at least about 3%, at least about 4%, at least about 5%, or at least about 10% relative to a control. In some embodiments, inhibition or reduction of expression of the SOCS1 gene is 0.05%-0.10%, 0.05%-0.15%, 0.05%-0.2%, 0.05%-0.4%, 0.05%-0.8%, 0.5%-1%, 0.5%-2%, 0.5%-5%, 0.1%-0.2%, 0.1%-0.4%, 0.1%-0.8%, 0.1%-1%, 0.1%-2%, 0.1%-5%, or 0.1%-10%, relative to a control.

In some embodiments, inhibition or reduction of expression is by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% relative to a control. In some embodiments, inhibition or reduction of expression of the SOCS1 gene is 10%-20%, 10%-40%, 10%-60%, 10%-80%, 10%-100%, 15%-30%, 15%-50%, 15%-75%, 15%-100%, 20%-40%, 20%-60%-20%-80%, 20%-100%, 30%-60%, 30%-80%, 30%-100%, 40%-80%, 40%-100%, 50%-80%, 50%-100%, 60%-70%, 60%-80%, 60%-90%, 70%-90%, 70%-100%, 80%-100%, or 90%-100% relative to a control.

In some embodiments, the DNA targeting systems herein are contacted with cells in a population of cells. In such instances, the SOCS1 gene may be modified in a portion of cells in the population. The methods herein may modify both copies of the SOCS1 gene or a single copy of the SOCS1 gene (e.g., leading to haploinsufficiency) in a cell. The percentage of cells that include a modified gene (e.g., a SOCS1 gene) may be used to assess the editing rate. For example, the SOCS1 gene may be modified in at least about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 2%, 4%, 5%, 10%, 15%, 20%, 30%, 40%, 50%, 60%, 70%, 80%, 90%, 95%, or more cells in the population of cells.

In some embodiments, the methods herein are effective to modify a SOCS1 gene in at least about 0.10%, at least about 0.11%, at least about 0.12%, at least about 0.13%, at least about 0.14%, at least about 0.15%, at least about 0.16%, at least about 0.17%, at least about 0.18%, at least about 0.19%, at least about 0.20%, at least about 0.30%, at least about 0.40%, at least about 0.50%, at least about 0.6%, at least about 0.7%, at least about 0.8%, at least about 0.9%, or at least about 1%, at least about 2%, at least about 3%, at least about 4%, at least about 5%, or at least about 10% cells in the population of cells. In some embodiments, the methods herein are effective to modify a SOCS1 gene in 0.05%-0.10%, 0.05%-0.15%, 0.05%-0.2%, 0.05%-0.4%, 0.05%-0.8%, 0.5-1%, 0.5%-2%, 0.5%-5%, 0.1%-0.2%, 0.1%-0.4%, 0.1%-0.8%, 0.1%-1%, 0.1%-2%, 0.1%-5%, or 0.1%-10% cells in the population of cells.

In certain embodiments, the methods herein are effective to modify a SOCS1 gene in at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of cells in a population of cells. In some embodiments, the methods herein are effective to modify a SOCS1 gene in 10%-20%, 10%-40%, 10%-60%, 10%-80%, 10%-100%, 15%-30%, 15%-50%, 15%-75%, 15%-100%, 20%-40%, 20%-60%-20%-80%, 20%-100%, 30%-60%, 30%-80%, 30%-100%, 40%-80%, 40%-100%, 50%-80%, 50%-100%, 60%-70%, 60%-80%, 60%-90%, 70%-90%, 70%-100%, 80%-100%, or 90%-100% of cells in the population of cells.

The methods herein may modify a single copy or both copies of the SOCS1 gene in a cell. In some embodiments, both copies of the SOCS1 gene are modified in a cell. Alternatively, a single copy of the SOCS1 gene is modified in a cell (e.g., leading to haploinsufficiency).

The expression of a SOCS1 gene may be assessed based on the level of any variable associated with SOCS1 gene expression, e.g., SOCS1 mRNA level or SOCS1 protein level. The expression of a SOCS1 gene may also be assessed indirectly based on, for example, the level of activity of SOCS1 (e.g., measurement of known downstream biological responses such as cytokine expression/signaling) or downstream pharmacodynamics effect of SOCS1 in a tissue sample, such as a tumor sample. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive or non-targeting agent control).

In the methods and compositions herein, the gRNA targeting SOCS1 may comprise a gRNA identified in the Examples and as summarized in Table 2. Accordingly, in some embodiments, the gRNA targeting the SOCS1 gene comprises a polynucleotide sequence (an RNA sequence, e.g., an gRNA spacer) set forth in any one of SEQ ID NOs: 264-347 or 481, or a variant thereof having at least 90%, 95%, 97%, 98%, or 99% sequence identity (e.g., wherein the variant is capable of directing the site-directed modifying polypeptide to the target site). In certain embodiments, provided is a crRNA comprising a polynucleotide sequence of any one of SEQ ID NOs: 264-347 or 481. In some embodiments, the gRNA targeting the SOCS1 gene targets the DNA sequence of any one of SEQ ID NOs: 180-263, or a variant thereof having at least 90%, 95%, 97%, 98%, or 99% sequence identity. In some embodiments, the gRNA targeting the SOCS1 gene is encoded by an isolated polynucleotide comprising the polynucleotide sequence (DNA sequence) of any one of SEQ ID NOs: 348-431 and 479, or a variant thereof having at least 90%, 95%, 97%, 98%, or 99% sequence identity, wherein the variant encodes a gRNA capable of directing the site-directed modifying polypeptide to the target site.

In some embodiments, the gRNA comprises the polynucleotide sequence of SEQ ID NO:264. In some embodiments, the gRNA comprises the polynucleotide sequence of SEQ ID NO:265. In some embodiments, the gRNA comprises the polynucleotide sequence of SEQ ID NO:266. In some embodiments, the gRNA comprises the polynucleotide sequence of SEQ ID NO:267. In some embodiments, the gRNA comprises the polynucleotide sequence of SEQ ID NO:268. In some embodiments, the gRNA comprises the polynucleotide sequence of SEQ ID NO:269. In some embodiments, the gRNA comprises the polynucleotide sequence of SEQ ID NO:270. In some embodiments, the gRNA comprises the polynucleotide sequence of SEQ ID NO:271. In some embodiments, the gRNA comprises the polynucleotide sequence of SEQ ID NO:272. In some embodiments, the gRNA comprises the polynucleotide sequence of SEQ ID NO:273. In some embodiments, the gRNA comprises the polynucleotide sequence of SEQ ID NO:274. In some embodiments, the gRNA comprises the polynucleotide sequence of SEQ ID NO:275. In some embodiments, the gRNA comprises the polynucleotide sequence of SEQ ID NO:276. In some embodiments, the gRNA comprises the polynucleotide sequence of SEQ ID NO:277. In some embodiments, the gRNA comprises the polynucleotide sequence of SEQ ID NO:278. In some embodiments, the gRNA comprises the polynucleotide sequence of SEQ ID NO:279. In some embodiments, the gRNA comprises the polynucleotide sequence of SEQ ID NO:280. In some embodiments, the gRNA comprises the polynucleotide sequence of SEQ ID NO:281. In some embodiments, the gRNA comprises the polynucleotide sequence of SEQ ID NO:282. In some embodiments, the gRNA comprises the polynucleotide sequence of SEQ ID NO:283. In some embodiments, the gRNA comprises the polynucleotide sequence of SEQ ID NO:284. In some embodiments, the gRNA comprises the polynucleotide sequence of SEQ ID NO:285. In some embodiments, the gRNA comprises the polynucleotide sequence of SEQ ID NO:286. In some embodiments, the gRNA comprises the polynucleotide sequence of SEQ ID NO:287. In some embodiments, the gRNA comprises the polynucleotide sequence of SEQ ID NO:288. In some embodiments, the gRNA comprises the polynucleotide sequence of SEQ ID NO:289. In some embodiments, the gRNA comprises the polynucleotide sequence of SEQ ID NO:290. In some embodiments, the gRNA comprises the polynucleotide sequence of SEQ ID NO:291. In some embodiments, the gRNA comprises the polynucleotide sequence of SEQ ID NO:292. In some embodiments, the gRNA comprises the polynucleotide sequence of SEQ ID NO:293. In some embodiments, the gRNA comprises the polynucleotide sequence of SEQ ID NO:294. In some embodiments, the gRNA comprises the polynucleotide sequence of SEQ ID NO:295. In some embodiments, the gRNA comprises the polynucleotide sequence of SEQ ID NO:296. In some embodiments, the gRNA comprises the polynucleotide sequence of SEQ ID NO:297. In some embodiments, the gRNA comprises the polynucleotide sequence of SEQ ID NO:298. In some embodiments, the gRNA comprises the polynucleotide sequence of SEQ ID NO:299. In some embodiments, the gRNA comprises the polynucleotide sequence of SEQ ID NO:300. In some embodiments, the gRNA comprises the polynucleotide sequence of SEQ ID NO:301. In some embodiments, the gRNA comprises the polynucleotide sequence of SEQ ID NO:302. In some embodiments, the gRNA comprises the polynucleotide sequence of SEQ ID NO:303. In some embodiments, the gRNA comprises the polynucleotide sequence of SEQ ID NO:304. In some embodiments, the gRNA comprises the polynucleotide sequence of SEQ ID NO:305. In some embodiments, the gRNA comprises the polynucleotide sequence of SEQ ID NO:306. In some embodiments, the gRNA comprises the polynucleotide sequence of SEQ ID NO:307. In some embodiments, the gRNA comprises the polynucleotide sequence of SEQ ID NO:308. In some embodiments, the gRNA comprises the polynucleotide sequence of SEQ ID NO:309. In some embodiments, the gRNA comprises the polynucleotide sequence of SEQ ID NO:310. In some embodiments, the gRNA comprises the polynucleotide sequence of SEQ ID NO:311. In some embodiments, the gRNA comprises the polynucleotide sequence of SEQ ID NO:312. In some embodiments, the gRNA comprises the polynucleotide sequence of SEQ ID NO:313. In some embodiments, the gRNA comprises the polynucleotide sequence of SEQ ID NO:314. In some embodiments, the gRNA comprises the polynucleotide sequence of SEQ ID NO:315. In some embodiments, the gRNA comprises the polynucleotide sequence of SEQ ID NO:316. In some embodiments, the gRNA comprises the polynucleotide sequence of SEQ ID NO:317. In some embodiments, the gRNA comprises the polynucleotide sequence of SEQ ID NO:318. In some embodiments, the gRNA comprises the polynucleotide sequence of SEQ ID NO:319. In some embodiments, the gRNA comprises the polynucleotide sequence of SEQ ID NO:320. In some embodiments, the gRNA comprises the polynucleotide sequence of SEQ ID NO:321. In some embodiments, the gRNA comprises the polynucleotide sequence of SEQ ID NO:322. In some embodiments, the gRNA comprises the polynucleotide sequence of SEQ ID NO:323. In some embodiments, the gRNA comprises the polynucleotide sequence of SEQ ID NO:324. In some embodiments, the gRNA comprises the polynucleotide sequence of SEQ ID NO:325. In some embodiments, the gRNA comprises the polynucleotide sequence of SEQ ID NO:326. In some embodiments, the gRNA comprises the polynucleotide sequence of SEQ ID NO:327. In some embodiments, the gRNA comprises the polynucleotide sequence of SEQ ID NO:328. In some embodiments, the gRNA comprises the polynucleotide sequence of SEQ ID NO:329. In some embodiments, the gRNA comprises the polynucleotide sequence of SEQ ID NO:330. In some embodiments, the gRNA comprises the polynucleotide sequence of SEQ ID NO:331. In some embodiments, the gRNA comprises the polynucleotide sequence of SEQ ID NO:332. In some embodiments, the gRNA comprises the polynucleotide sequence of SEQ ID NO:333. In some embodiments, the gRNA comprises the polynucleotide sequence of SEQ ID NO:334. In some embodiments, the gRNA comprises the polynucleotide sequence of SEQ ID NO:335. In some embodiments, the gRNA comprises the polynucleotide sequence of SEQ ID NO:336. In some embodiments, the gRNA comprises the polynucleotide sequence of SEQ ID NO:337. In some embodiments, the gRNA comprises the polynucleotide sequence of SEQ ID NO:338. In some embodiments, the gRNA comprises the polynucleotide sequence of SEQ ID NO:339. In some embodiments, the gRNA comprises the polynucleotide sequence of SEQ ID NO:340. In some embodiments, the gRNA comprises the polynucleotide sequence of SEQ ID NO:341. In some embodiments, the gRNA comprises the polynucleotide sequence of SEQ ID NO:342. In some embodiments, the gRNA comprises the polynucleotide sequence of SEQ ID NO:343. In some embodiments, the gRNA comprises the polynucleotide sequence of SEQ ID NO:344. In some embodiments, the gRNA comprises the polynucleotide sequence of SEQ ID NO:345. In some embodiments, the gRNA comprises the polynucleotide sequence of SEQ ID NO:346. In some embodiments, the gRNA comprises the polynucleotide sequence of SEQ ID NO:347. In some embodiments, the gRNA comprises the polynucleotide sequence of SEQ ID NO: 481.

In some embodiments, the gRNA targeting the SOCS1 gene comprises modifications of the gRNA sequence (e.g., modifications of a gRNA sequence provided herein). In some cases, the gRNA (e.g., sgRNA) comprises a first polynucleotide sequence that is complementary to the target nucleic acid and a second polynucleotide sequence that interacts with a Cas polypeptide. In other instances, the gRNA (e.g., sgRNA) comprises one or more modified nucleotides. In some cases, one or more of the nucleotides in the first polynucleotide sequence and/or the second polynucleotide sequence are modified nucleotides.

In some embodiments, the modified nucleotides comprise a modification in a ribose group, a phosphate group, a nucleobase, or a combination thereof. In some instances, the modification in the ribose group comprises a modification at the 2′ position of the ribose group. In some cases, the modification at the 2′ position of the ribose group is selected from the group consisting of 2′-O-methyl, 2′-fluoro, 2′-deoxy, 2′-O-(2-methoxyethyl), and a combination thereof. In other instances, the modification in the phosphate group comprises a phosphorothioate modification. In other embodiments, the modified nucleotides are selected from the group consisting of a 2′-ribo 3′-phosphorothioate(S), 2′-O-methyl (M) nucleotide, a 2′-O-methyl 3′-phosphorothioate (MS) nucleotide, a 2′-O-methyl 3′-thioPACE (MSP) nucleotide, and a combination thereof.

Further, non-base modifiers (such as a C3 spacer propanediol group or a ZEN modifier napthyl-azo group) can be placed at one or both ends of the gRNA without loss of activity and also block exonuclease attack. Accordingly, in some embodiments, the gRNA comprises a C3 spacer propanediol group. In some embodiments, the gRNA comprises a ZEN modifier napthyl-azo group.

The gRNAs of the DNA targeting systems provided herein may be designed for use with a particular site-directed modifying polypeptide, such as a Cas9 polypeptide, a Cas12 polypeptide (e.g., Cas12a), or a base editor.

In one embodiment, the DNA targeting system comprises Cas9 (e.g., spCas9) as the site directed-modifying polypeptide and a guide RNA (gRNA), wherein the gRNA comprises a polynucleotide sequence comprising any one of SEQ ID NOs: 264, 266, 267, 270-346, and 481, or a variant thereof having at least 90%, 95%, 97%, 98%, or 99% sequence identity, wherein the variant is capable of directing the site-directed modifying polypeptide to the target site. In some embodiments, the gRNA targeting the SOCS1 gene targets the DNA sequence of any one of SEQ ID NOs: 180, 182, 183, 186-262, and 480, or a variant thereof having at least 90%, 95%, 97%, 98%, or 99% sequence identity. In some embodiments, the gRNA targeting the SOCS1 gene is encoded by an isolated polynucleotide comprising the polynucleotide sequence (DNA sequence) of any one of SEQ ID NOS: 348, 350, 351, 354-430, and 479, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the SOCS1 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the SOCS1 gene comprises a non-base modifier (such as a C3 spacer propanediol group or a ZEN modifier napthyl-azo group) at one or both ends of the gRNA.

In some embodiments, the DNA targeting system comprises Cas9 (e.g., spCas9) as the site directed-modifying polypeptide and a guide RNA (gRNA), wherein the gRNA comprises a polynucleotide sequence (e.g., gRNA spacer sequence) comprising any one of SEQ ID NOs: 307, 328, 299, 481, 270, 285, 277, or 297, or a variant thereof having at least 90%, 95%, 97%, 98%, or 99% sequence identity, wherein the variant is capable of directing the site-directed modifying polypeptide to the target site. In some embodiments, the gRNA targeting the SOCS1 gene targets the DNA sequence of any one of SEQ ID NOs: 223, 244, 215, 480, 186, 201, 193, and 213, or a variant thereof having at least 90%, 95%, 97%, 98%, or 99% sequence identity. In some embodiments, the gRNA targeting the SOCS1 gene comprises a polynucleotide sequence (e.g., a crispr RNA (crRNA) sequence) of any one of SEQ ID NOs: 455-462, or a variant thereof having at least 90%, 95%, 97%, 98%, or 99% sequence identity. In some embodiments, the gRNA targeting the SOCS1 gene comprises a polynucleotide sequence (e.g., a single guide RNA (sgRNA) sequence) of any one of SEQ ID NOs: 463-470, or a variant thereof having at least 90%, 95%, 97%, 98%, or 99% sequence identity.

In some embodiments, the gRNA targeting the SOCS1 gene is encoded by an isolated polynucleotide comprising the polynucleotide sequence (DNA sequence encoding crRNA) of any one of SEQ ID NOs: 471-478, or a variant thereof having at least 90%, 95%, 97%, 98%, or 99% sequence identity, wherein the variant encodes a gRNA capable of directing the site-directed modifying polypeptide to the target site.

In one embodiment, the DNA targeting system comprises Cas12 (e.g., Cas12a) as the site directed-modifying polypeptide and a guide RNA (gRNA), wherein the gRNA comprises a polynucleotide sequence comprising any one of SEQ ID NOs: 265, 268, and 269, or a variant thereof having at least 90%, 95%, 97%, 98%, or 99% sequence identity, wherein the variant is capable of directing the site-directed modifying polypeptide to the target site. In some embodiments, the gRNA targeting the SOCS1 gene targets the DNA sequence of any one of SEQ ID NOs: 181, 184, and 185, or a variant thereof having at least 90%, 95%, 97%, 98%, or 99% sequence identity. In some embodiments, the gRNA targeting the SOCS1 gene is encoded by an isolated polynucleotide comprising the polynucleotide sequence (DNA sequence) of any one of SEQ ID NOs: 349, 352, and 353, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the SOCS1 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the SOCS1 gene comprises a non-base modifier (such as a C3 spacer propanediol group or a ZEN modifier napthyl-azo group) at one or both ends of the gRNA.

In one embodiment, the DNA targeting system comprises a base editor (e.g., a cytosine base editor (CBE)) as the site directed-modifying polypeptide and a guide RNA (gRNA), wherein the gRNA comprises the polynucleotide sequence of SEQ ID NO: 347, or a variant thereof having at least 90%, 95%, 97%, 98%, or 99% sequence identity, wherein the variant is capable of directing the site-directed modifying polypeptide to the target site. In some embodiments, the gRNA targeting the SOCS1 gene targets the DNA sequence of any one of SEQ ID NOs: 263, or a variant thereof having at least 90%, 95%, 97%, 98%, or 99% sequence identity. In some embodiments, the gRNA targeting the SOCS1 gene is encoded by an isolated polynucleotide comprising the polynucleotide sequence (DNA sequence) of SEQ ID NO: 431, or a variant thereof having at least 90%, 95%, 97%, 98%, or 99% sequence identity, wherein the variant encodes a gRNA capable of directing the site-directed modifying polypeptide to the target site. In some embodiments, the gRNA targeting the SOCS1 gene comprises modifications of the gRNA sequence, such as a modification in a ribose group, a phosphate group, a nucleobase, or a combination thereof (e.g., a 2′-O-methyl or a phosphorothioate modification described herein). In some embodiments, the gRNA targeting the SOCS1 gene comprises a non-base modifier (such as a C3 spacer propanediol group or a ZEN modifier napthyl-azo group) at one or both ends of the gRNA.

The DNA targeting system may be used to target any exon in SOCS1, including exon 2. In some embodiments, the DNA targeting system comprises a site-directed modifying polypeptide (e.g., Cas9) and gRNA capable of targeting a 5′ region of the SOCS1 gene. gRNAs targeting earlier regions of the SOCS1 gene may induce a frameshift causing indel that lead to a loss of function protein. Alternatively, the DNA targeting system comprises a site-directed modifying polypeptide (e.g., Cas9) and gRNA capable of targeting a later region of the SOCS1 gene.

In some embodiments, the DNA targeting system comprises a site-directed modifying polypeptide (e.g., Cas9, Cas12a, or a base editor) and gRNA capable of targeting a region of the SOCS1 gene that encodes an SH2 domain of a human SOCS1 protein (e.g., corresponding to amino acid residues 79-174 of Uniprot Accession No. 015524); an SOCS box domain of a human SOCS1 protein (e.g., corresponding to amino acid residues 161-210 of Uniprot Accession No. 015524); a kinase inhibitory region of a human SOCS1 protein (e.g., corresponding to amino acid residues 55-66 of Uniprot Accession No. 015524); an extended SH2 subdomain of a human SOCS1 protein (e.g., corresponding to amino acid residues 67-78 of Uniprot Accession No. 015524); or an elongin BC complex (BC-box) interaction domain of a human SOCS1 protein (e.g., corresponding to amino acid residues 173-182 of Uniprot Accession No. 015524).

Further details regarding site-directed modifying polypeptides and gRNAs that may be used in the compositions and methods are provided herein (see, e.g., Table 1). Additionally, in some embodiments, the site-directed modifying polypeptide further comprises a cell targeting agent, thereby forming a targeted active gene editing (TAGE) agent, which are further described in section III.

TABLE 2
Human SOCS1 Guide RNAs (gRNAs)

| | | Target DNA | SEQ | | SEQ | DNA Sequence | SEQ |
|---|---|---|---|---|---|---|---|
| | Cas | Sequence | ID | gRNA sequence | ID | encoding gRNA | ID |
| gRNA Name | Enzyme | (5′ to 3′) | NO | (spacer; 5′ to 3′) | NO | (5′ to 3′) | NO |
| 11255284rev | SpCas9 | TCCGTTCGCAC | 180 | UCCGUUCGCAC | 264 | TCCGTTCGCAC | 348 |
| | | GCCGATTAC | | GCCGAUUAC | | GCCGATTACGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255073rev | Cas12a | ACCTGGATGG | 181 | ACCUGGAUGGC | 265 | TAATTTCTACTC | 349 |
| | | CAGCCGCGAG | | AGCCGCGAGA | | TTGTAGATACCT | |
| | | A | | | | GGATGGCAGCC | |
| | | | | | | GCGAGA | |
| 11255303forw | SpCas9 | GCCGGTAATC | 182 | GCCGGUAAUCG | 266 | GCCGGTAATCG | 350 |
| | | GGCGTGCGAA | | GCGUGCGAA | | GCGTGCGAAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255292forw | SpCas9 | GCGCGTGATG | 183 | GCGCGUGAUGC | 267 | GCGCGTGATGC | 351 |
| | | CGCCGGTAAT | | GCCGGUAAU | | GCCGGTAATGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255140rev | Cas12a | GCCCTTAGCGT | 184 | GCCCUUAGCGU | 268 | TAATTTCTACTC | 352 |
| | | GAAGATGGCC | | GAAGAUGGCC | | TTGTAGATGCC | |
| | | | | | | CTTAGCGTGAA | |
| | | | | | | GATGGCC | |
| 11255088rev | Cas12a | AGGCCGGCCG | 185 | AGGCCGGCCGC | 269 | TAATTTCTACTC | 353 |
| | | CTTTCACCTGG | | UUUCACCUGG | | TTGTAGATAGG | |
| | | | | | | CCGGCCGCTTT | |
| | | | | | | CACCTGG | |
| 11255312forw | SpCas9 | CGGCGTGCGA | 186 | CGGCGUGCGAA | 270 | CGGCGTGCGAA | 354 |
| | | ACGGAATGTG | | CGGAAUGUG | | CGGAATGTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254850forw | SpCas9 | CGGGCGCTGC | 187 | CGGGCGCUGC | 271 | CGGGCGCTGCC | 355 |
| | | CGGTCAAATC | | CGGUCAAAUC | | GGTCAAATCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255110forw | SpCas9 | AAGTGCACGC | 188 | AAGUGCACGCG | 272 | AAGTGCACGCG | 356 |
| | | GGATGCTCGT | | GAUGCUCGU | | GATGCTCGTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255235rev | SpCas9 | GGACGCCTGC | 189 | GGACGCCUGCG | 273 | GGACGCCTGCG | 357 |
| | | GGATTCTACT | | GAUUCUACU | | GATTCTACTGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255328forw | SpCas9 | TGTGCGGAAG | 190 | UGUGCGGAAGU | 274 | TGTGCGGAAGT | 358 |
| | | TGCGTGTCGC | | GCGUGUCGC | | GCGTGTCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255156forw | SpCas9 | GGGCGAAAAA | 191 | GGGCGAAAAAG | 275 | GGGCGAAAAAG | 359 |
| | | GCAGTTCCGC | | CAGUUCCGC | | CAGTTCCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255234rev | SpCas9 | GACGCCTGCG | 192 | GACGCCUGCGG | 276 | GACGCCTGCGG | 360 |
| | | GATTCTACTG | | AUUCUACUG | | ATTCTACTGGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255152rev | SpCas9 | TGGTGCGCGA | 193 | UGGUGCGCGAC | 277 | TGGTGCGCGAC | 361 |
| | | CAGCCGCCAG | | AGCCGCCAG | | AGCCGCCAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255109forw | SpCas9 | AAAGTGCACG | 194 | AAAGUGCACGC | 278 | AAAGTGCACGC | 362 |
| | | CGGATGCTCG | | GGAUGCUCG | | GGATGCTCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255236rev | SpCas9 | TGGACGCCTG | 195 | UGGACGCCUGC | 279 | TGGACGCCTGC | 363 |
| | | CGGATTCTAC | | GGAUUCUAC | | GGATTCTACGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255081rev | SpCas9 | ATCCGCGTGC | 196 | AUCCGCGUGCA | 280 | ATCCGCGTGCA | 364 |
| | | ACTTTCAGGC | | CUUUCAGGC | | CTTTCAGGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255258forw | SpCas9 | AGTAGAATCCG | 197 | AGUAGAAUCCG | 281 | AGTAGAATCCG | 365 |
| | | CAGGCGTCC | | CAGGCGUCC | | CAGGCGTCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255136forw | SpCas9 | CGAGGCCATC | 198 | CGAGGCCAUCU | 282 | CGAGGCCATCT | 366 |
| | | TTCACGCTAA | | UCACGCUAA | | TCACGCTAAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254854forw | SpCas9 | CGCTGCCGGT | 199 | CGCUGCCGGUC | 283 | CGCTGCCGGTC | 367 |
| | | CAAATCTGGA | | AAAUCUGGA | | AAATCTGGAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255121rev | SpCas9 | TTTCGCCCTTA | 200 | UUUCGCCCUUA | 284 | TTTCGCCCTTAG | 368 |
| | | GCGTGAAGA | | GCGUGAAGA | | CGTGAAGAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255159forw | SpCas9 | CGAAAAAGCA | 201 | CGAAAAAGCAG | 285 | CGAAAAAGCAG | 369 |
| | | GTTCCGCTGG | | UUCCGCUGG | | TTCCGCTGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255229forw | SpCas9 | GTGCGCCCCG | 202 | GUGCGCCCCGU | 286 | GTGCGCCCCGT | 370 |
| | | TGCACGCTCA | | GCACGCUCA | | GCACGCTCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255330forw | SpCas9 | TGCGGAAGTG | 203 | UGCGGAAGUGC | 287 | TGCGGAAGTGC | 371 |
| | | CGTGTCGCCG | | GUGUCGCCG | | GTGTCGCCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255331 forw | SpCas9 | GCGGAAGTGC | 204 | GCGGAAGUGCG | 288 | GCGGAAGTGCG | 372 |
| | | GTGTCGCCGG | | UGUCGCCGG | | TGTCGCCGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255250forw | SpCas9 | GGGCCCCCAG | 205 | GGGCCCCCAGU | 289 | GGGCCCCCAGT | 373 |
| | | TAGAATCCGC | | AGAAUCCGC | | AGAATCCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255285forw | SpCas9 | CGCTGGCGCG | 206 | CGCUGGCGCG | 290 | CGCTGGCGCGC | 374 |
| | | CGTGATGCGC | | CGUGAUGCGC | | GTGATGCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254886forw | SpCas9 | TCAGGTAGTCG | 207 | UCAGGUAGUCG | 291 | TCAGGTAGTCG | 375 |
| | | CGGAGGACG | | CGGAGGACG | | CGGAGGACGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255085rev | SpCas9 | GAGCATCCGC | 208 | GAGCAUCCGCG | 292 | GAGCATCCGCG | 376 |
| | | GTGCACTTTC | | UGCACUUUC | | TGCACTTTCGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254885forw | SpCas9 | CTCAGGTAGTC | 209 | CUCAGGUAGUC | 293 | CTCAGGTAGTC | 377 |
| | | GCGGAGGAC | | GCGGAGGAC | | GCGGAGGACGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255135forw | SpCas9 | CCGAGGCCAT | 210 | CCGAGGCCAUC | 294 | CCGAGGCCATC | 378 |
| | | CTTCACGCTA | | UUCACGCUA | | TTCACGCTAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254919rev | SpCas9 | CAGCGCATCG | 211 | CAGCGCAUCGU | 295 | CAGCGCATCGT | 379 |
| | | TGGCCACCGT | | GGCCACCGU | | GGCCACCGTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255228forw | SpCas9 | CGTGCGCCCC | 212 | CGUGCGCCCCG | 296 | CGTGCGCCCCG | 380 |
| | | GTGCACGCTC | | UGCACGCUC | | TGCACGCTCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255246rev | SpCas9 | AGCGCGCTCC | 213 | AGCGCGCUCCU | 297 | AGCGCGCTCCT | 381 |
| | | TGGACGCCTG | | GGACGCCUG | | GGACGCCTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254916forw | SpCas9 | GGATGCGAGC | 214 | GGAUGCGAGCC | 298 | GGATGCGAGCC | 382 |
| | | CAGGTTCTCG | | AGGUUCUCG | | AGGTTCTCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255230forw | SpCas9 | TGCGCCCCGT | 215 | UGCGCCCCGUG | 299 | TGCGCCCCGTG | 383 |
| | | GCACGCTCAG | | CACGCUCAG | | CACGCTCAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254905rev | SpCas9 | CACCGTGGGC | 216 | CACCGUGGGCC | 300 | CACCGTGGGCC | 384 |
| | | CGCGAGAACC | | GCGAGAACC | | GCGAGAACCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255203rev | SpCas9 | GCGTGCACGG | 217 | GCGUGCACGG | 301 | GCGTGCACGGG | 385 |
| | | GGCGCACGAG | | GGCGCACGAG | | GCGCACGAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255118forw | SpCas9 | GCGGATGCTC | 218 | GCGGAUGCUCG | 302 | GCGGATGCTCG | 386 |
| | | GTGGGTCCCG | | UGGGUCCCG | | TGGGTCCCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254855forw | SpCas9 | GCTGCCGGTC | 219 | GCUGCCGGUCA | 303 | GCTGCCGGTCA | 387 |
| | | AAATCTGGAA | | AAUCUGGAA | | AATCTGGAAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255099forw | SpCas9 | GGCCGGCCTG | 220 | GGCCGGCCUGA | 304 | GGCCGGCCTGA | 388 |
| | | AAAGTGCACG | | AAGUGCACG | | AAGTGCACGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254884forw | SpCas9 | GCTCAGGTAGT | 221 | GCUCAGGUAGU | 305 | GCTCAGGTAGT | 389 |
| | | CGCGGAGGA | | CGCGGAGGA | | CGCGGAGGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255067rev | SpCas9 | TCAGGCCGGC | 222 | UCAGGCCGGCC | 306 | TCAGGCCGGCC | 390 |
| | | CGCTTTCACC | | GCUUUCACC | | GCTTTCACCGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255460rev | SpCas9 | TAGGATGGTAG | 223 | UAGGAUGGUAG | 307 | TAGGATGGTAG | 391 |
| | | CACACAACC | | CACACAACC | | CACACAACCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254943forw | SpCas9 | CGGTGGCCAC | 224 | CGGUGGCCACG | 308 | CGGTGGCCACG | 392 |
| | | GATGCGCTGG | | AUGCGCUGG | | ATGCGCTGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255420forw | SpCas9 | AGGAAGGTTCT | 225 | AGGAAGGUUCU | 309 | AGGAAGGTTCT | 393 |
| | | GGCCGCCGT | | GGCCGCCGU | | GGCCGCCGTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254893forw | SpCas9 | GTCGCGGAGG | 226 | GUCGCGGAGGA | 310 | GTCGCGGAGGA | 394 |
| | | ACGGGGTTGA | | CGGGGUUGA | | CGGGGTTGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254926forw | SpCas9 | CAGGTTCTCGC | 227 | CAGGUUCUCGC | 311 | CAGGTTCTCGC | 395 |
| | | GGCCCACGG | | GGCCCACGG | | GGCCCACGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255487forw | SpCas9 | GTGTGCTACCA | 228 | GUGUGCUACCA | 312 | GTGTGCTACCA | 396 |
| | | TCCTACAGA | | UCCUACAGA | | TCCTACAGAGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255114rev | SpCas9 | CTTAGCGTGAA | 229 | CUUAGCGUGAA | 313 | CTTAGCGTGAA | 397 |
| | | GATGGCCTC | | GAUGGCCUC | | GATGGCCTCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254880forw | SpCas9 | AGGAGCTCAG | 230 | AGGAGCUCAGG | 314 | AGGAGCTCAGG | 398 |
| | | GTAGTCGCGG | | UAGUCGCGG | | TAGTCGCGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254963rev | SpCas9 | CGCTGCGCCA | 231 | CGCUGCGCCAG | 315 | CGCTGCGCCAG | 399 |
| | | GCGCCGCGTG | | CGCCGCGUG | | CGCCGCGTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254990rev | SpCas9 | CGGCGCCGCG | 232 | CGGCGCCGCG | 316 | CGGCGCCGCG | 400 |
| | | CCGCATGCTG | | CCGCAUGCUG | | CCGCATGCTGG | |
| | | | | | | TTTTAGAGCTAG | |
| | | | | | | AAATAGCAAGTT | |
| | | | | | | AAAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254923forw | SpCas9 | AGCCAGGTTCT | 233 | AGCCAGGUUCU | 317 | AGCCAGGTTCT | 401 |
| | | CGCGGCCCA | | CGCGGCCCA | | CGCGGCCCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255174forw | SpCas9 | GCTGGCGGCT | 234 | GCUGGCGGCU | 318 | GCTGGCGGCTG | 402 |
| | | GTCGCGCACC | | GUCGCGCACC | | TCGCGCACCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255183rev | SpCas9 | CGGCTGCGCG | 235 | CGGCUGCGCG | 319 | CGGCTGCGCGC | 403 |
| | | CCGAGCCCGT | | CCGAGCCCGU | | CGAGCCCGTGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255329forw | SpCas9 | GTGCGGAAGT | 236 | GUGCGGAAGUG | 320 | GTGCGGAAGTG | 404 |
| | | GCGTGTCGCC | | CGUGUCGCC | | CGTGTCGCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255057forw | SpCas9 | AGAGGCAGTC | 237 | AGAGGCAGUCG | 321 | AGAGGCAGTCG | 405 |
| | | GAAGCTCTCG | | AAGCUCUCG | | AAGCTCTCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254894forw | SpCas9 | TCGCGGAGGA | 238 | UCGCGGAGGAC | 322 | TCGCGGAGGAC | 406 |
| | | CGGGGTTGAG | | GGGGUUGAG | | GGGGTTGAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255336forw | SpCas9 | AGTGCGTGTC | 239 | AGUGCGUGUCG | 323 | AGTGCGTGTCG | 407 |
| | | GCCGGGGGCC | | CCGGGGGCC | | CCGGGGGCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254856forw | SpCas9 | CTGCCGGTCA | 240 | CUGCCGGUCAA | 324 | CTGCCGGTCAA | 408 |
| | | AATCTGGAAG | | AUCUGGAAG | | ATCTGGAAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255178forw | SpCas9 | GCGGCTGTCG | 241 | GCGGCUGUCG | 325 | GCGGCTGTCGC | 409 |
| | | CGCACCAGGA | | CGCACCAGGA | | GCACCAGGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255069forw | SpCas9 | AGCTCTCGCG | 242 | AGCUCUCGCGG | 326 | AGCTCTCGCGG | 410 |
| | | GCTGCCATCC | | CUGCCAUCC | | CTGCCATCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255063rev | SpCas9 | GCCGGCCGCT | 243 | GCCGGCCGCUU | 327 | GCCGGCCGCTT | 411 |
| | | TTCACCTGGA | | UCACCUGGA | | TCACCTGGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255457rev | SpCas9 | GATGGTAGCA | 244 | GAUGGUAGCAC | 328 | GATGGTAGCAC | 412 |
| | | CACAACCAGG | | ACAACCAGG | | ACAACCAGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255489forw | SpCas9 | GTGCTACCATC | 245 | GUGCUACCAUC | 329 | GTGCTACCATC | 413 |
| | | CTACAGAAG | | CUACAGAAG | | CTACAGAAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255462forw | SpCas9 | CTGCATTGTCG | 246 | CUGCAUUGUCG | 330 | CTGCATTGTCG | 414 |
| | | GCTGCCACC | | GCUGCCACC | | GCTGCCACCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255172rev | SpCas9 | CGAGCCCGTG | 247 | CGAGCCCGUGG | 331 | CGAGCCCGTGG | 415 |
| | | GGCACCTTCC | | GCACCUUCC | | GCACCTTCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255025rev | SpCas9 | CGACTGCCTCT | 248 | CGACUGCCUCU | 332 | CGACTGCCTCT | 416 |
| | | TCGAGCTGC | | UCGAGCUGC | | TCGAGCTGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254940forw | SpCas9 | CCACGGTGGC | 249 | CCACGGUGGCC | 333 | CCACGGTGGCC | 417 |
| | | CACGATGCGC | | ACGAUGCGC | | ACGATGCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254892forw | SpCas9 | AGTCGCGGAG | 250 | AGUCGCGGAGG | 334 | AGTCGCGGAGG | 418 |
| | | GACGGGGTTG | | ACGGGGUUG | | ACGGGGTTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254860forw | SpCas9 | CGGTCAAATCT | 251 | CGGUCAAAUCU | 335 | CGGTCAAATCT | 419 |
| | | GGAAGGGGA | | GGAAGGGGA | | GGAAGGGGAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255488forw | SpCas9 | TGTGCTACCAT | 252 | UGUGCUACCAU | 336 | TGTGCTACCAT | 420 |
| | | CCTACAGAA | | CCUACAGAA | | CCTACAGAAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255340rev | SpCas9 | GCGGCCGTGC | 253 | GCGGCCGUGC | 337 | GCGGCCGTGCC | 421 |
| | | CCCGCGGTCC | | CCCGCGGUCC | | CCGCGGTCCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255413rev | SpCas9 | CAGCAGCAGA | 254 | CAGCAGCAGAG | 338 | CAGCAGCAGAG | 422 |
| | | GCCCCGACGG | | CCCCGACGG | | CCCCGACGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254920rev | SpCas9 | CCAGCGCATC | 255 | CCAGCGCAUCG | 339 | CCAGCGCATCG | 423 |
| | | GTGGCCACCG | | UGGCCACCG | | TGGCCACCGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255436forw | SpCas9 | CCGTCGGGGC | 256 | CCGUCGGGGC | 340 | CCGTCGGGGCT | 424 |
| | | TCTGCTGCTG | | UCUGCUGCUG | | CTGCTGCTGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255187forw | SpCas9 | GCGCACCAGG | 257 | GCGCACCAGGA | 341 | GCGCACCAGGA | 425 |
| | | AAGGTGCCCA | | AGGUGCCCA | | AGGTGCCCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254992rev | SpCas9 | GGCGGCGCCG | 258 | GGCGGCGCCG | 342 | GGCGGCGCCG | 426 |
| | | CGCCGCATGC | | CGCCGCAUGC | | CGCCGCATGCG | |
| | | | | | | TTTTAGAGCTAG | |
| | | | | | | AAATAGCAAGTT | |
| | | | | | | AAAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255216rev | SpCas9 | TGGGGGCCCC | 259 | UGGGGGCCCC | 343 | TGGGGGCCCCT | 427 |
| | | TGAGCGTGCA | | UGAGCGUGCA | | GAGCGTGCAGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255346rev | SpCas9 | GCGCCCGCGG | 260 | GCGCCCGCGG | 344 | GCGCCCGCGG | 428 |
| | | CCGTGCCCCG | | CCGUGCCCCG | | CCGTGCCCCGG | |
| | | | | | | TTTTAGAGCTAG | |
| | | | | | | AAATAGCAAGTT | |
| | | | | | | AAAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255078forw | SpCas9 | GGCTGCCATC | 261 | GGCUGCCAUCC | 345 | GGCTGCCATCC | 429 |
| | | CAGGTGAAAG | | AGGUGAAAG | | AGGTGAAAGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11254895forw | SpCas9 | CGCGGAGGAC | 262 | CGCGGAGGACG | 346 | CGCGGAGGACG | 430 |
| | | GGGGTTGAGG | | GGGUUGAGG | | GGGTTGAGGGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255072forw | Base | GGGCCCCCAG | 263 | GGGCCCCCAGU | 347 | GGGCCCCCAGT | 431 |
| | Editor | TAGAATCCGC | | AGAAUCCGC | | AGAATCCGCGT | |
| | | | | | | TTTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |
| 11255233rev | Cas9 | ACGCCTGCGG | 480 | ACGCCUGCGGA | 481 | ACGCCTGCGGA | 479 |
| | | ATTCTACTGG | | UUCUACUGG | | TTCTACTGGGTT | |
| | | | | | | TTAGAGCTAGA | |
| | | | | | | AATAGCAAGTTA | |
| | | | | | | AAATAAGGCTA | |
| | | | | | | GTCCGTTATCAA | |
| | | | | | | CTTGAAAAAGT | |
| | | | | | | GGCACCGAGTC | |
| | | | | | | GGTGCTTTT | |

Site Directed Modifying Polypeptides and gRNAs

The site-directed modifying polypeptides used in the presently disclosed compositions and methods are site-specific, in that the polypeptide itself or an associated molecule recognizes and is targeted to a particular nucleic acid sequence or a set of similar sequences (i.e., target sequence(s), such as a target sequence in a TNFAIP3 gene or a SOCS1 gene). In some embodiments, the site-directed modifying polypeptide (or its associated molecule) recognizes sequences that are similar in sequence, comprising conserved bases or motifs that can be degenerate at one or more positions.

In particular embodiments, the site-directed modifying polypeptide modifies the polynucleotide (e.g., a TNFAIP3 gene or a SOCS1 gene) at particular location(s) (i.e., modification site(s)) outside of its target sequence. The modification site(s) modified by a particular site-directed modifying polypeptide are also generally specific to a particular sequence or set of similar sequences. In some of these embodiments, the site-directed modifying polypeptide modifies sequences that are similar in sequence, comprising conserved bases or motifs that can be degenerate at one or more positions. In other embodiments, the site-directed modifying polypeptide modifies sequences that are within a particular location relative to the target sequence(s). For example, the site-directed modifying polypeptide may modify sequences that are within a particular number of nucleic acids upstream or downstream from the target sequence(s).

As used herein with respect to site-directed modifying polypeptides, the term “modification” means any insertion, deletion, substitution, or chemical modification of at least one nucleotide the modification site or alternatively, a change in the expression of a gene that is adjacent to the target site. The substitution of at least one nucleotide in the modification site can be the result of the recruitment of a base editing domain, such as a cytidine deaminase or adenine deaminase domain (see, for example, Eid et al. (2018) Biochem J 475 (11): 1955-1964, which is herein incorporated in its entirety).

The change in expression of a gene adjacent to a target site can result from the recruitment of a transcriptional activation domain or transcriptional repression domain to the promoter region of the gene or the recruitment of an epigenetic modification domain that covalently modifies DNA or histone proteins to alter histone structure and/or chromosomal structure without altering the DNA sequence, leading to changes in gene expression of an adjacent gene. The term “modification” also encompasses the recruitment to a target site of a detectable label that can be conjugated to the site-directed modifying polypeptide or an associated molecule (e.g., gRNA) that allows for the detection of a specific nucleic acid sequence (e.g., a disease-associated sequence).

In some embodiments, the site-directed modifying polypeptide is a nuclease or variant thereof and the agent comprising the nuclease or variant thereof. As used herein a “nuclease” refers to an enzyme which cleaves a phosphodiester bond in the backbone of a polynucleotide chain. Suitable nucleases for the presently disclosed compositions and methods can have endonuclease and/or exonuclease activity. An exonuclease cleaves nucleotides one at a time from the end of a polynucleotide chain. An endonuclease cleaves a polynucleotide chain by cleaving phosphodiester bonds within a polynucleotide chain, other than those at the two ends of a polynucleotide chain. The nuclease can cleave RNA polynucleotide chains (i.e., ribonuclease) and/or DNA polynucleotide chains (i.e., deoxyribonuclease).

Nucleases cleave polynucleotide chains, resulting in a cleavage site. As used herein, the term “cleave” refers to the hydrolysis of phosphodiester bonds within the backbone of a polynucleotide chain. Cleavage by nucleases can be single-stranded or double-stranded. In some embodiments, a double-stranded cleavage of DNA is achieved via cleavage with two nucleases wherein each nuclease cleaves a single strand of the DNA. Cleavage by the nuclease can result in blunt ends or staggered ends.

Non-limiting examples of nucleases suitable for the presently disclosed compositions and methods include meganucleases, such as homing endonucleases; restriction endonucleases, such as Type IIS endonucleases (e.g., FokI)); zinc finger nucleases; transcription activator-like effector nucleases (TALENs), and nucleic acid-guided nucleases (e.g., RNA-guided endonuclease, DNA-guided endonuclease, or DNA/RNA-guided endonuclease).

As used herein, a “meganuclease” refers to an endonuclease that binds DNA at a target sequence that is greater than 12 base pairs in length. Meganucleases bind to double-stranded DNA as heterodimers. Suitable meganucleases for the presently disclosed compositions and methods include homing endonucleases, such as those of the LAGLIDADG (SEQ ID NO: 14) family comprising this amino acid motif or a variant thereof.

As used herein, a “zinc finger nuclease” or “ZFN” refers to a chimeric protein comprising a zinc finger DNA-binding domain fused to a nuclease domain from an exonuclease or endonuclease, such as a restriction endonuclease or meganuclease. The zinc finger DNA-binding domain is bound by a zinc ion that serves to stabilize the unique structure.

As used herein, a “transcription activator-like effector nuclease” or “TALEN” refers to a chimeric protein comprising a DNA-binding domain comprising multiple TAL domain repeats fused to a nuclease domain from an exonuclease or endonuclease, such as a restriction endonuclease or meganuclease. TAL domain repeats can be derived from the TALE family of proteins from the Xanthomonas genus of Proteobacteria. TAL domain repeats are 33-34 amino acid sequences with hypervariable 12th and 13th amino acids that are referred to as the repeat variable diresidue (RVD). The RVD imparts specificity of target sequence binding. The TAL domain repeats can be engineered through rational or experimental means to produce variant TALENs that have a specific target sequence specificity (see, for example, Boch et al. (2009) Science 326 (5959): 1509-1512 and Moscou and Bogdanove (2009) Science 326(5959): 1501, each of which is incorporated by reference in its entirety). DNA cleavage by a TALEN requires two DNA target sequences flanking a nonspecific spacer region, wherein each DNA target sequence is bound by a TALEN monomer. In some embodiments, the TALEN comprises a compact TALEN, which refers to an endonuclease comprising a DNA-binding domain with one or more TAL domain repeats fused in any orientation to any portion of a homing endonuclease (e.g., I-TevI, MmeI, EndA, End1, I-BasI, I-TevII, I-TevIII, I-TwoI, MspI, MvaI, NucA, and NucM). Compact TALENs are advantageous in that they do not require dimerization for DNA processing activity, thus only requiring a single target site.

As used herein, a “nucleic acid-guided nuclease” refers to a nuclease that is directed to a specific target sequence based on the complementarity (full or partial) between a guide nucleic acid (i.e., guide RNA or gRNA, guide DNA or gDNA, or guide DNA/RNA hybrid) that is associated with the nuclease and a target sequence. The binding between the guide RNA and the target sequence serves to recruit the nuclease to the vicinity of the target sequence. Non-limiting examples of nucleic acid-guided nucleases suitable for the presently disclosed compositions and methods include naturally-occurring Clustered Regularly Interspaced Short Palindromic Repeats (CRISPR)-associated (Cas) polypeptides from a prokaryotic organism (e.g., bacteria, archaea) or variants thereof. CRISPR sequences found within prokaryotic organisms are sequences that are derived from fragments of polynucleotides from invading viruses and are used to recognize similar viruses during subsequent infections and cleave viral polynucleotides via CRISPR-associated (Cas) polypeptides that function as an RNA-guided nuclease to cleave the viral polynucleotides. As used herein, a “CRISPR-associated polypeptide” or “Cas polypeptide” refers to a naturally-occurring polypeptide that is found within proximity to CRISPR sequences within a naturally-occurring CRISPR system. Certain Cas polypeptides function as RNA-guided nucleases.

There are at least two classes of naturally-occurring CRISPR systems, Class 1 and Class 2. In general, the nucleic acid-guided nucleases of the presently disclosed compositions and methods are Class 2 Cas polypeptides or variants thereof given that the Class 2 CRISPR systems comprise a single polypeptide with nucleic acid-guided nuclease activity, whereas Class 1 CRISPR systems require a complex of proteins for nuclease activity. There are at least three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). In general, Type II and Type V-B systems require a tracrRNA, in addition to crRNA, for activity. In contrast, Type V-A and Type VI only require a crRNA for activity. All known Type II and Type V RNA-guided nucleases target double-stranded DNA, whereas all known Type VI RNA-guided nucleases target single-stranded RNA. The RNA-guided nucleases of Type II CRISPR systems are referred to as Cas9 herein and in the literature. In some embodiments, the nucleic acid-guided nuclease of the presently disclosed compositions and methods is a Type II Cas9 protein or a variant thereof. Type V Cas polypeptides that function as RNA-guided nucleases do not require tracrRNA for targeting and cleavage of target sequences. The RNA-guided nuclease of Type VA CRISPR systems are referred to as Cpf1; of Type VB CRISPR systems are referred to as C2C1; of Type VC CRISPR systems are referred to as Cas12C or C2C3; of Type VIA CRISPR systems are referred to as C2C2 or Cas13A1; of Type VIB CRISPR systems are referred to as Cas13B; and of Type VIC CRISPR systems are referred to as Cas13A2 herein and in the literature. In certain embodiments, the nucleic acid-guided nuclease of the presently disclosed compositions and methods is a Type VA Cpf1 protein or a variant thereof. Naturally-occurring Cas polypeptides and variants thereof that function as nucleic acid-guided nucleases are known in the art and include, but are not limited to Streptococcus pyogenes Cas9 (SpCas9), Staphylococcus aureus Cas9 (SaCas9), Streptococcus thermophilus Cas9 (StCas9), Francisella novicida Cpf1, or those described in Shmakov et al. (2017) Nat Rev Microbiol 15 (3): 169-182; Makarova et al. (2015) Nat Rev Microbiol 13 (11): 722-736; and U.S. Pat. No. 9,790,490, each of which is incorporated herein in its entirety. Class 2 Type V CRISPR nucleases include Cas12 and any subtypes of Cas12, such as Cas12a, Cas12b, Cas12c, Cas12d, Cas12e, Cas12f, Cas12g, Cas12h, and Cas12i. Class 2 Type VI CRISPR nucleases including Cas13 can be used as the site-directed modifying polypeptide in order to cleave RNA target sequences.

The nucleic acid-guided nuclease of the presently disclosed compositions and methods can be a naturally-occurring nucleic acid-guided nuclease (e.g., S. pyogenes Cas9) or a variant thereof. Variant nucleic acid-guided nucleases can be engineered or naturally occurring variants that contain substitutions, deletions, or additions of amino acids that, for example, alter the activity of one or more of the nuclease domains, fuse the nucleic acid-guided nuclease to a heterologous domain that imparts a modifying property (e.g., transcriptional activation domain, epigenetic modification domain, detectable label), modify the stability of the nuclease, or modify the specificity of the nuclease.

In some embodiments, a nucleic acid-guided nuclease includes one or more mutations to improve specificity for a target site and/or stability in the intracellular microenvironment. For example, where the protein is Cas9 (e.g., SpCas9) or a modified Cas9, it may be beneficial to delete any or all residues from N175 to R307 (inclusive) of the Rec2 domain. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80A, C80L, C801, C80V, C80K, C574E, C574D, C574N, C574Q (in any combination) and in particular C80A. Substitutions may be included to reduce intracellular protein binding of the nuclease and/or increase target site specificity. Additionally, or alternatively, substitutions may be included to reduce off-target toxicity of the composition.

In some embodiments, the RNA-guided DNA endonuclease is a Cas9 nuclease. In some embodiments, the Cas9 nuclease is wildtype Cas9 nuclease (e.g., Streptococcus pyogenes Cas9, SEQ ID NO: 1). In some embodiments, the Cas9 nuclease comprises an amino acid sequence having at least 85%, 90%, 95%, 97%, 98%, 99%, or 100% identity to SEQ ID NO: 1. In certain embodiments, the Cas9 nuclease comprises the amino acid substitution C80A (e.g., SEQ ID NO: 1). In some embodiments, the Cas9 nuclease comprises an amino acid sequence having at least 85%, 90%, 95%, 97%, 98%, 99%, or 100% identity to SEQ ID NO: 1 (e.g., an engineered version of Cas9, such as variants of Cas9 described herein or otherwise known in the art).

In some embodiments, the RNA-guided DNA endonuclease is a nuclease other than Cas9 (e.g., such as a RNA-guided DNA endonuclease described herein). In some embodiments, the RNA-guided DNA endonuclease is a CRISPR Type V nuclease. In specific embodiments, the RNA-guided DNA endonuclease is a Cas12 nuclease. In some embodiments, the Cas12 nuclease is wildtype Cas12a nuclease (e.g., Acidaminococcus sp. Cas12a, SEQ ID NO: 2). In some embodiments, the Cas12 nuclease comprises an amino acid sequence having at least 85%, 90%, 95%, 97%, 98%, 99%, or 100% identity to SEQ ID NO: 2 (e.g., an engineered version of Cas12a, such as variants of Cas12a described herein or otherwise known in the art). Examples of Cas12a variants useful in the TAGE agents herein include, but are not limited to, Alt-R® Cas12a (Cpf1) Ultra (e.g., IDT Catalog No. 10001272) or Cas12a as described in Kleinstiver, et al. Nature biotechnology 37.3 (2019): 276-282, which is hereby incorporated by reference.

The nucleic acid-guided nuclease is directed to a particular target sequence through its association with a guide nucleic acid (e.g., guideRNA (gRNA), guideDNA (gDNA)). The nucleic acid-guided nuclease is bound to the guide nucleic acid via non-covalent interactions, thus forming a complex. The polynucleotide-targeting nucleic acid provides target specificity to the complex by comprising a nucleotide sequence that is complementary to a sequence of a target sequence. The nucleic acid-guided nuclease of the complex or a domain or label fused or otherwise conjugated thereto provides the site-specific activity. In other words, the nucleic acid-guided nuclease is guided to a target polynucleotide sequence (e.g. a target sequence in a chromosomal nucleic acid (e.g., a target sequence within a TNFAIP3 gene or a SOCS1 gene); a target sequence in an extrachromosomal nucleic acid, e.g. an episomal nucleic acid, a minicircle; a target sequence in a mitochondrial nucleic acid; a target sequence in a chloroplast nucleic acid; a target sequence in a plasmid) by virtue of its association with the protein-binding segment of the polynucleotide-targeting guide nucleic acid.

Thus, the guide nucleic acid comprises two segments, a “polynucleotide-targeting segment” and a “polypeptide-binding segment.” By “segment” it is meant a segment/section/region of a molecule (e.g., a contiguous stretch of nucleotides in an RNA). A segment can also refer to a region/section of a complex such that a segment may comprise regions of more than one molecule. For example, in some cases the polypeptide-binding segment (described below) of a polynucleotide-targeting nucleic acid comprises only one nucleic acid molecule and the polypeptide-binding segment therefore comprises a region of that nucleic acid molecule. In other cases, the polypeptide-binding segment (described below) of a DNA-targeting nucleic acid comprises two separate molecules that are hybridized along a region of complementarity.

The polynucleotide-targeting segment (or “polynucleotide-targeting sequence” or “guide sequence”) comprises a nucleotide sequence that is complementary (fully or partially) to a specific sequence within a target sequence, such as a target sequence within a TNFAIP3 gene or a SOCS1 gene (for example, the complementary strand of a target DNA sequence). The polypeptide-binding segment (or “polypeptide-binding sequence”) interacts with a nucleic acid-guided nuclease. In general, site-specific cleavage or modification of the target DNA (e.g., a target sequence within a TNFAIP3 gene or a SOCS1 gene) by a nucleic acid-guided nuclease occurs at locations determined by both (i) base-pairing complementarity between the polynucleotide-targeting sequence of the nucleic acid and the target DNA; and (ii) a short motif (referred to as the protospacer adjacent motif (PAM)) in the target DNA.

A protospacer adjacent motif can be of different lengths and can be a variable distance from the target sequence (e.g., a target sequence within a TNFAIP3 gene or a SOCS1 gene), although the PAM is generally within about 1 to about 10 nucleotides from the target sequence, including about 1, about 2, about 3, about 4, about 5, about 6, about 7, about 8, about 9, or about 10 nucleotides from the target sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given RNA-guided nuclease are known in the art and include, but are not limited to the PAM depletion assay described by Karvelis et al. (2015) Genome Biol 16:253, or the assay disclosed in Pattanayak et al. (2013) Nat Biotechnol 31 (9): 839-43, each of which is incorporated by reference in its entirety.

The polynucleotide-targeting sequence (i.e., guide sequence) is the nucleotide sequence that directly hybridizes with the target sequence of interest (e.g., a target sequence within a TNFAIP3 gene or a SOCS1 gene). The guide sequence is engineered to be fully or partially complementary with the target sequence of interest. In various embodiments, the guide sequence can comprise from about 8 nucleotides to about 30 nucleotides, or more. For example, the guide sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the guide sequence is about 10 to about 26 nucleotides in length, or about 12 to about 30 nucleotides in length. In particular embodiments, the guide sequence is about 30 nucleotides in length. In some embodiments, the degree of complementarity between a guide sequence and its corresponding target sequence (e.g., a target sequence within a TNFAIP3 gene or a SOCS1 gene), when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the guide sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106 (1): 23-24).

In some embodiments, a guide nucleic acid comprises two separate nucleic acid molecules (an “activator-nucleic acid” and a “targeter-nucleic acid”, see below) and is referred to herein as a “double-molecule guide nucleic acid” or a “two-molecule guide nucleic acid.” In other embodiments, the subject guide nucleic acid is a single nucleic acid molecule (single polynucleotide) and is referred to herein as a “single-molecule guide nucleic acid,” a “single-guide nucleic acid,” or an “sgNA.” The term “guide nucleic acid” or “gNA” is inclusive, referring both to double-molecule guide nucleic acids and to single-molecule guide nucleic acids (i.e., sgNAs). In those embodiments wherein the guide nucleic acid is an RNA, the gRNA can be a double-molecule guide RNA or a single-guide RNA (sgRNA). Likewise, in those embodiments wherein the guide nucleic acid is a DNA, the gDNA can be a double-molecule guide DNA or a single-guide DNA.

An exemplary two-molecule guide nucleic acid comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the polynucleotide-targeting segment (single stranded) of the guide RNA and a stretch (“duplex-forming segment”) of nucleotides that forms one half of the dsRNA duplex of the polypeptide-binding segment of the guide RNA, also referred to herein as the CRISPR repeat sequence.

The term “activator-nucleic acid” or “activator-NA” is used herein to mean a tracrRNA-like molecule of a double-molecule guide nucleic acid. The term “targeter-nucleic acid” or “targeter-NA” is used herein to mean a crRNA-like molecule of a double-molecule guide nucleic acid. The term “duplex-forming segment” is used herein to mean the stretch of nucleotides of an activator-NA or a targeter-NA that contributes to the formation of the dsRNA duplex by hybridizing to a stretch of nucleotides of a corresponding activator-NA or targeter-NA molecule. In other words, an activator-NA comprises a duplex-forming segment that is complementary to the duplex-forming segment of the corresponding targeter-NA. As such, an activator-NA comprises a duplex-forming segment while a targeter-NA comprises both a duplex-forming segment and the DNA-targeting segment of the guide nucleic acid. Therefore, a subject double-molecule guide nucleic acid can be comprised of any corresponding activator-NA and targeter-NA pair.

The activator-NA comprises a CRISPR repeat sequence comprising a nucleotide sequence that comprises a region with sufficient complementarity to hybridize to an activator-NA (the other part of the polypeptide-binding segment of the guide nucleic acid). In various embodiments, the CRISPR repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and the antirepeat region of its corresponding tracr sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

A corresponding tracrRNA-like molecule (i.e., activator-NA) comprises a stretch of nucleotides (duplex-forming segment) that forms the other part of the double-stranded duplex of the polypeptide-binding segment of the guide nucleic acid. In other words, a stretch of nucleotides of a crRNA-like molecule (i.e., the CRISPR repeat sequence) are complementary to and hybridize with a stretch of nucleotides of a tracrRNA-like molecule (i.e., the anti-repeat sequence) to form the double-stranded duplex of the polypeptide-binding domain of the guide nucleic acid. The crRNA-like molecule additionally provides the single stranded DNA-targeting segment. Thus, a crRNA-like and a tracrRNA-like molecule (as a corresponding pair) hybridize to form a guide nucleic acid. The exact sequence of a given crRNA or tracrRNA molecule is characteristic of the CRISPR system and species in which the RNA molecules are found. A subject double-molecule guide RNA can comprise any corresponding crRNA and tracrRNA pair.

A trans-activating-like CRISPR RNA or tracrRNA-like molecule (also referred to herein as an “activator-NA”) comprises a nucleotide sequence comprising a region that has sufficient complementarity to hybridize to a CRISPR repeat sequence of a crRNA, which is referred to herein as the anti-repeat region. In some embodiments, the tracrRNA-like molecule further comprises a region with secondary structure (e.g., stem-loop) or forms secondary structure upon hybridizing with its corresponding crRNA. In particular embodiments, the region of the tracrRNA-like molecule that is fully or partially complementary to a CRISPR repeat sequence is at the 5′ end of the molecule and the 3′ end of the tracrRNA-like molecule comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA-like molecule that is fully or partially complementary to the CRISPR repeat sequence comprises from about 8 nucleotides to about 30 nucleotides, or more. For example, the region of base pairing between the tracrRNA-like anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA-like anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA-like molecule can comprise from about 60 nucleotides to more than about 140 nucleotides. For example, the tracrRNA-like molecule can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA-like molecule is about 80 to about 100 nucleotides in length, including about 80, about 81, about 82, about 83, about 84, about 85, about 86, about 87, about 88, about 89, about 90, about 91, about 92, about 93, about 94, about 95, about 96, about 97, about 98, about 99, and about 100 nucleotides in length.

A subject single-molecule guide nucleic acid (i.e., sgNA, e.g., sgRNA) comprises two stretches of nucleotides (a targeter-NA and an activator-NA) that are complementary to one another, are covalently linked by intervening nucleotides (“linkers” or “linker nucleotides”), and hybridize to form the double stranded nucleic acid duplex of the protein-binding segment, thus resulting in a stem-loop structure. The targeter-NA and the activator-NA can be covalently linked via the 3′ end of the targeter-NA and the 5′ end of the activator-NA. Alternatively, the targeter-NA and the activator-NA can be covalently linked via the 5′ end of the targeter-NA and the 3′ end of the activator-NA.

The linker of a single-molecule DNA-targeting nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt, including but not limited to about 3, about 4, about 5, about 6, about 7, about 8, about 9, about 10, about 11, about 12, about 13, about 14, about 15, about 16, about 17, about 18, about 19, about 20, or more nucleotides. In some embodiments, the linker of a single-molecule DNA-targeting nucleic acid is 4 nt.

An exemplary single-molecule DNA-targeting nucleic acid comprises two complementary stretches of nucleotides that hybridize to form a double-stranded duplex, along with a guide sequence that hybridizes to a specific target sequence.

Appropriate naturally-occurring cognate pairs of crRNAs (and, in some embodiments, tracrRNAs) are known for most Cas proteins that function as nucleic acid-guided nucleases that have been discovered or can be determined for a specific naturally-occurring Cas protein that has nucleic acid-guided nuclease activity by sequencing and analyzing flanking sequences of the Cas nucleic acid-guided nuclease protein to identify tracrRNA-coding sequence, and thus, the tracrRNA sequence, by searching for known antirepeat-coding sequences or a variant thereof. Antirepeat regions of the tracrRNA comprise one-half of the ds protein-binding duplex. The complementary repeat sequence that comprises one-half of the ds protein-binding duplex is called the CRISPR repeat. CRISPR repeat and antirepeat sequences utilized by known CRISPR nucleic acid-guided nucleases are known in the art and can be found, for example, at the CRISPR database on the world wide web at crispr.i2bc.paris-saclay.fr/crispr/.

The single guide nucleic acid or dual-guide nucleic acid can be synthesized chemically or via in vitro transcription. Assays for determining sequence-specific binding between a nucleic acid-guided nuclease and a guide nucleic acid are known in the art and include, but are not limited to, in vitro binding assays between an expressed nucleic acid-guided nuclease and the guide nucleic acid, which can be tagged with a detectable label (e.g., biotin) and used in a pull-down detection assay in which the nucleoprotein complex is captured via the detectable label (e.g., with streptavidin beads). A control guide nucleic acid with an unrelated sequence or structure to the guide nucleic acid can be used as a negative control for non-specific binding of the nucleic acid-guided nuclease to nucleic acids.

In some embodiments, the DNA-targeting RNA, gRNA, or sgRNA or nucleotide sequence encoding the DNA-targeting RNA, gRNA, or sgRNA comprises modifications of the nucleotide sequence. In some cases, the sgRNA (e.g., truncated sgRNA) comprises a first nucleotide sequence that is complementary to the target nucleic acid and a second nucleotide sequence that interacts with a Cas polypeptide. In other instances, the sgRNA comprises one or more modified nucleotides. In some cases, one or more of the nucleotides in the first nucleotide sequence and/or the second nucleotide sequence are modified nucleotides.

In some embodiments, the modified nucleotides comprise a modification in a ribose group, a phosphate group, a nucleobase, or a combination thereof. In some instances, the modification in the ribose group comprises a modification at the 2′ position of the ribose group. In some cases, the modification at the 2′ position of the ribose group is selected from the group consisting of 2′-O-methyl, 2′-fluoro, 2′-deoxy, 2′-O-(2-methoxyethyl), and a combination thereof. In other instances, the modification in the phosphate group comprises a phosphorothioate modification. In other embodiments, the modified nucleotides are selected from the group consisting of a 2′-ribo 3′-phosphorothioate(S), 2′-O-methyl (M) nucleotide, a 2′-O-methyl 3′-phosphorothioate (MS) nucleotide, a 2′-O-methyl 3′-thioPACE (MSP) nucleotide, and a combination thereof.

In certain embodiments, the site-directed modifying polypeptide of the presently disclosed compositions and methods comprise a nuclease variant that functions as a nickase. In one embodiment, the nuclease comprises a mutation in comparison to the wild-type nuclease that results in the nuclease only being capable of cleaving a single strand of a double-stranded nucleic acid molecule, or lacks nuclease activity altogether (i.e., nuclease-dead).

A nuclease, such as a nucleic acid-guided nuclease, that functions as a nickase only comprises a single functioning nuclease domain. In some of these embodiments, additional nuclease domains have been mutated such that the nuclease activity of that particular domain is reduced or eliminated.

In other embodiments, the nuclease (e.g., RNA-guided nuclease) lacks nuclease activity completely and is referred to herein as nuclease-dead. In some of these embodiments, all nuclease domains within the nuclease have been mutated such that all nuclease activity of the polypeptide has been eliminated. Any method known in the art can be used to introduce mutations into one or more nuclease domains of a site-directed nuclease, including those set forth in U.S. Publ. Nos. 2014/0068797 and U.S. Pat. No. 9,790,490, each of which is incorporated by reference in its entirety.

Any mutation within a nuclease domain that reduces or eliminates the nuclease activity can be used to generate a nucleic acid-guided nuclease having nickase activity or a nuclease-dead nucleic acid-guided nuclease. Such mutations are known in the art and include, but are not limited to the D10A mutation within the RuvC domain or H840A mutation within the HNH domain of the S. pyogenes Cas9 or at similar position(s) within another nucleic acid-guided nuclease when aligned for maximal homology with the S. pyogenes Cas9. Other positions within the nuclease domains of S. pyogenes Cas9 that can be mutated to generate a nickase or nuclease-dead protein include G12, G17, E762, N854, N863, H982, H983, and D986. Other mutations within a nuclease domain of a nucleic acid-guided nuclease that can lead to nickase or nuclease-dead proteins include a D917A, E1006A, E1028A, D1227A, D1255A, N1257A, D917A, E1006A, E1028A, D1227A, D1255A, and N1257A of the Francisella novicida Cpf1 protein or at similar position(s) within another nucleic acid-guided nuclease when aligned for maximal homology with the F. novicida Cpf1 protein (U.S. Pat. No. 9,790,490, which is incorporated by reference in its entirety).

Site-directed modifying polypeptides comprising a nuclease-dead domain can further comprise a domain capable of modifying a polynucleotide. Non-limiting examples of modifying domains that may be fused to a nuclease-dead domain include but are not limited to, a transcriptional activation or repression domain, a base editing domain, and an epigenetic modification domain. In other embodiments, the site-directed modifying polypeptide comprising a nuclease-dead domain further comprises a detectable label that can aid in detecting the presence of the target sequence.

The epigenetic modification domain that can be fused to a nuclease-dead domain serves to covalently modify DNA or histone proteins to alter histone structure and/or chromosomal structure without altering the DNA sequence itself, leading to changes in gene expression (e.g., upregulation or downregulation of a TNFAIP3 gene or a SOCS1 gene). Non-limiting examples of epigenetic modifications that can be induced by site-directed modifying polypeptides include the following alterations in histone residues and the reverse reactions thereof: sumoylation, methylation of arginine or lysine residues, acetylation or ubiquitination of lysine residues, phosphorylation of serine and/or threonine residues; and the following alterations of DNA and the reverse reactions thereof: methylation or hydroxymethylation of cystosine residues. Non-limiting examples of epigenetic modification domains thus include histone acetyltransferase domains, histone deacetylation domains, histone methyltransferase domains, histone demethylase domains, DNA methyltransferase domains, and DNA demethylase domains.

In some embodiments, the site-directed polypeptide comprises a transcriptional activation domain that activates the transcription of at least one adjacent gene through the interaction with transcriptional control elements and/or transcriptional regulatory proteins, such as transcription factors or RNA polymerases. Suitable transcriptional activation domains are known in the art and include, but are not limited to, VP16 activation domains.

In other embodiments, the site-directed polypeptide comprises a transcriptional repressor domain, which can also interact with transcriptional control elements and/or transcriptional regulatory proteins, such as transcription factors or RNA polymerases, to reduce or terminate transcription of at least one adjacent gene. Suitable transcriptional repression domains are known in the art and include, but are not limited to, IκB and KRAB domains.

In still other embodiments, the site-directed modifying polypeptide comprising a nuclease-dead domain further comprises a detectable label that can aid in detecting the presence of the target sequence. A detectable label is a molecule that can be visualized or otherwise observed. The detectable label may be fused to the nucleic-acid guided nuclease as a fusion protein (e.g., fluorescent protein) or may be a small molecule conjugated to the nuclease polypeptide that can be detected visually or by other means. Detectable labels that can be fused to the presently disclosed nucleic-acid guided nucleases as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

In some embodiments, the site-directed modifying polypeptide comprises a base editor comprised of a nickase domain or a nuclease-dead domain and a deaminase domain. The deaminase domain is capable of catalyzing the deamination of a nucleobase, such as cytosine or adenine, and can thus be referred to as a nucleobase deaminase. Deamination of a nucleobase by a deaminase can lead to a point mutation. The deaminase domain may be active on single-stranded nucleic acids or on double-stranded nucleic acids, but in some embodiments, the deaminase is only capable of deaminating single-stranded nucleic acids, including a single-stranded region of a double-stranded nucleic acid molecule (e.g., dsDNA) that is present in an R-loop formed by the binding of a nucleic acid-guided nickase or nuclease-dead domain to the double-stranded nucleic acid molecule (e.g., dsDNA). In certain embodiments, the nickase cleaves one strand of a double-stranded molecule and the complementary, non-target strand is acted upon by the deaminase.

In some embodiments, the deaminase domain of the site-directed modifying polypeptide is an adenine deaminase and the site-directed modifying polypeptide is referred to herein as an “A-base editor,” “adenine base editor” or “ABE”. The adenine deaminase domain is capable of deaminating an adenine, adenosine, or deoxyadenosine, yielding inosine which is recognized as guanine by polymerases and allows for the incorporation of a cytosine on the complementary nucleic acid strand, resulting in an A: T to G: C base pair change. While there are no known naturally-occurring DNA adenine deaminases, methods known in the art can be used to evolve and optimize adenine deaminase acting on tRNA (ADAT) proteins to have activity on DNA molecules, including a bacterial selection assay (see, e.g. Gaudelli et al, 2017; Koblan, L. W. et al, 2018, Nat Biotechnol 36, 843-846; Richter, M. F. et al, 2020, Nat Biotechnol, doi: 10.1038/s41587-020-0562-8).

In other embodiments, the deaminase domain of the site-directed modifying polypeptide is a cytosine deaminase and the site-directed modifying polypeptide is referred to herein as a “C-base editor,” “cytosine base editor” or “CBE”. The cytosine deaminase domain is capable of deaminating a cytosine, cytidine, or deoxycytidine to uracil, which can then be subsequently converted to thymine through DNA replication or repair. In particular embodiments, the deamination of a cytosine can result in the conversion of the cytosine to a guanine or adenine due to the activity of a uracil DNA glycosylase during base excision repair of the uracil residue. Thus, in some embodiments wherein the site-directed modifying polypeptide is a C-base editor and the desired nucleotide modification is a C>T mutation, the site-directed modifying polypeptide further comprises an uracil DNA glycosylase inhibitor which prevents uracil DNA glycosylase from cleaving the N-glycosidic bond of uracil and initiating the base-excision repair pathway, leading to the stabilization of the uracil residue. Uracil glycosylase inhibitors are known in the art and include the small protein from Bacillus subtilis bacteriophage PBS1, the sequence of which is set forth as SEQ ID NO: 3
MTNLSDIIEKETGKQLVIQESILMLPEEVEEVIGNKPESDILVHTAYDESTDENVMLLT SDAPEYKPWALVIQDSNGENKIKML (SEQ ID NO: 3)

Cytosine deaminases are known in the art and include apolipoprotein B mRNA-editing complex (APOBEC) family deaminases, such as deaminases from the APOBEC1 family, the activation-induced cytidine deaminase (AID), and the ACF1/ASE deaminase. The cytosine deaminase may be a naturally-occurring protein or an active variant or fragment thereof.

The nucleic acid-guided nuclease can be delivered into a cell as a nucleoprotein complex comprising the nucleic acid-guided nuclease bound to its guide nucleic acid. Alternatively, the nucleic acid-guided nuclease is delivered and the guide nucleic acid is provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase III promoter), which can be a native promoter or heterologous to the guide RNA-encoding nucleotide sequence.

In certain embodiments, the site-directed polypeptide can comprise additional amino acid sequences, such as at least one nuclear localization sequence (NLS). Nuclear localization sequences enhance transport of the site-directed polypeptide into the nucleus of a cell. Proteins that are imported into the nucleus bind to one or more of the proteins within the nuclear pore complex, such as importin/karypherin proteins, which generally bind best to lysine and arginine residues. The best characterized pathway for nuclear localization involves short peptide sequence which binds to the importin-α protein. These nuclear localization sequences often comprise stretches of basic amino acids and given that there are two such binding sites on importin-α, two basic sequences separated by at least 10 amino acids can make up a bipartite NLS. The second most characterized pathway of nuclear import involves proteins that bind to the importin-β1 protein, such as the HIV-TAT and HIV-REV proteins, which use the sequences RKKRRQRRR (SEQ ID NO: 4) and RQARRNRRRRWR (SEQ ID NO: 5), respectively to bind to importin-β1. Other nuclear localization sequences are known in the art (see, e.g., Lange et al., J. Biol. Chem. (2007) 282:5101-5105). The NLS can be the naturally-occurring NLS of the site-directed polypeptide or a heterologous NLS. As used herein, “heterologous” in reference to a sequence is a sequence that originates from a foreign species, or, if from the same species, is substantially modified from its native form in composition and/or genomic locus by deliberate human intervention. Non-limiting examples of NLS sequences that can be used to enhance the nuclear localization of the site-directed polypeptides include the NLS of the SV40 Large T-antigen and c-Myc. In certain embodiments, the NLS comprises the amino acid sequence PKKKRKV (SEQ ID NO: 6).

The site-directed polypeptide can comprise more than one NLS, such as two, three, four, five, six, or more NLS sequences. Each of the multiple NLSs can be unique in sequence or there can be more than one of the same NLS sequence used. The NLS can be on the amino-terminal (N-terminal) end of the site-directed polypeptide, the carboxy-terminal (C-terminal) end, or both the N-terminal and C-terminal ends of the polypeptide. In certain embodiments, the site-directed polypeptide comprises four NLS sequences on its N-terminal end. In other embodiments, the site-directed polypeptide comprises two NLS sequences on the C-terminal end of the site-directed polypeptide. In still other embodiments, the site-directed polypeptide comprises four NLS sequences on its N-terminal end and two NLS sequences on its C-terminal end.

In certain embodiments, the site-directed polypeptide comprises a cell penetrating peptide (CPP), which induces the absorption of a linked protein or peptide through the plasma membrane of a cell. Generally, CPPs induce entry into the cell because of their general shape and tendency to either self-assemble into a membrane-spanning pore, or to have several positively charged residues, which interact with the negatively charged phospholipid outer membrane inducing curvature of the membrane, which in turn activates internalization. Exemplary permeable peptides include, but are not limited to, transportan, PEP1, MPG, p-VEC, MAP, CADY, polyR, HIV-TAT, HIV-REV, Penetratin, R6W3, P22N, DPV3, DPV6, K-FGF, and C105Y, and are reviewed in van den Berg and Dowdy (2011) Current Opinion in Biotechnology 22:888-893 and Farkhani et al. (2014) Peptides 57:78-94, each of which is herein incorporated by reference in its entirety.

Along with or as an alternative to an NLS, the site-directed polypeptide can comprise additional heterologous amino acid sequences, such as a detectable label (e.g., fluorescent protein) described elsewhere herein, or a purification tag, to form a fusion protein. A purification tag is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

The presently disclosed compositions and methods can be used to edit genomes (e.g., to edit a TNFAIP3 gene or a SOCS1 gene) through the introduction of a sequence-specific, double-stranded break that is repaired (via e.g., error-prone non-homologous end-joining (NHEJ), microhomology-mediated end joining (MMEJ), or alternative end-joining (alt-EJ) pathway) to introduce a mutation at a specific genomic location. Due to the error-prone nature of repair processes, repair of the double-stranded break can result in a modification to the target sequence. Alternatively, a donor template polynucleotide may be integrated into or exchanged with the target sequence (e.g., a target sequence within a TNFAIP3 gene or a SOCS1 gene) during the course of repair of the introduced double-stranded break, resulting in the introduction of the exogenous donor sequence. Accordingly, the compositions and methods can further comprise a donor template polynucleotide that may comprise flanking homologous ends. In some of these embodiments, the donor template polynucleotide is tethered to the site-directed modifying polypeptide via a linker as described elsewhere herein (e.g., the donor template polynucleotide is bound to the site-directed polypeptide via a cleavable linker).

In some embodiments, the donor sequence alters the original target sequence such that the newly integrated donor sequence will not be recognized and cleaved by the nucleic acid-guided nuclease. The donor sequence may comprise flanking sequences that have substantial sequence identity with the sequences flanking the target sequence (e.g., a target sequence in a TNFAIP3 gene or a SOCS1 gene) to enhance the integration of the donor sequence via homology-directed repair. In particular embodiments wherein the nucleic acid-guided nuclease generates double-stranded staggered breaks, the donor polynucleotide can be flanked by compatible overhangs, allowing for incorporation of the donor sequence via a non-homologous repair process during repair of the double-stranded break.

### Nanoparticles

DNA targeting systems herein (e.g., including a site-directed modifying polypeptide and a gRNA capable of targeting a TNFAIP3 gene or a SOCS1 gene) can alternatively be packaged into nanoparticles, such as lipid nanoparticles (LNP) (e.g., for delivery to a mammalian cell or a mammalian subject).

For example, in some embodiments, the LNP comprises a ribonucleoprotein (RNP) comprising a site-directed modifying polypeptide and a guide RNA capable of targeting a TNFAIP3 gene (e.g., a gRNA comprising a polynucleotide sequence set forth in any one of SEQ ID NOs: 70-124). In alternative embodiments, the LNP comprises an mRNA encoding the DNA targeting systems herein (e.g., a site-directed modifying polypeptide and a gRNA capable of targeting a TNFAIP3 gene (e.g., a gRNA comprising a polynucleotide sequence set forth in any one of SEQ ID NOs: 70-124). In some embodiments, the LNP comprises an mRNA encoding a gRNA capable of targeting a TNFAIP3 gene (e.g., a gRNA comprising a polynucleotide sequence set forth in any one of SEQ ID NOs: 70-124).

For example, in some embodiments, the LNP comprises a ribonucleoprotein (RNP) comprising a site-directed modifying polypeptide and a guide RNA capable of targeting a SOCS1 gene (e.g., a gRNA comprising a polynucleotide sequence set forth in any one of SEQ ID NOs: 264-347 and 481). In alternative embodiments, the LNP comprises an mRNA encoding the DNA targeting systems herein (e.g., a site-directed modifying polypeptide and a gRNA capable of targeting a SOCS1 gene (e.g., a gRNA comprising a polynucleotide sequence set forth in any one of SEQ ID NOs: 264-347 and 481). In some embodiments, the LNP comprises an mRNA encoding a gRNA capable of targeting a SOCS1 gene (e.g., a gRNA comprising a polynucleotide sequence set forth in any one of SEQ ID NOs: 264-347 and 481).

Accordingly, in some embodiments, RNPs or mRNA encoding the DNA targeting system are coupled covalently or non-covalently to a nanoparticle or encapsulated within such a nanoparticle using methods known in the art (Sharma, et al. (2014) Biomed Res Int. 2014). A nanoparticle is a nanoscale delivery system whose length scale is <1 mm, preferably <100 nm. Such nanoparticles may be designed using a core composed of metal, lipid, polymer, or biological macromolecule. RNPs or multiple copies of the mRNA can be attached to or encapsulated with the nanoparticle core. This increases the copy number of the mRNA that is delivered to each cell and, so, increases the intracellular expression of each engineered nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33 (30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell surface receptors and the natural ligands (or portions of the natural ligands) for cell surface receptors. In some embodiments, the nanoparticle is a lipid nanoparticle (LNP).

In some embodiments, RNPs or mRNA encoding the DNA targeting system are encapsulated within liposomes or complexed using cationic lipids (see, e.g., Lipofectamine™, Life Technologies Corp., Carlsbad, CA; Zuris et al. (2015) Nat Biotechnol. 33:73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the target cells.

In some embodiments, RNPs or mRNA encoding the DNA targeting system are encapsulated within polymeric scaffolds (e.g., PLGA) or complexed using cationic polymers (e.g., PEI, PLL) (Tamboli et al. (2011) Ther Deliv. 2 (4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, RNPS or mRNA encoding the DNA targeting system are combined with amphiphilic molecules that self-assemble into micelles (Tong et al. (2007) J Gene Med. 9 (11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, RNPs or mRNA encoding the DNA targeting system are formulated into an emulsion or a nanoemulsion (e.g., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, RNPS or mRNA encoding the DNA targeting system are covalently attached to, or non-covalently associated with, multifunctional polymer conjugates, DNA dendrimers, and polymeric dendrimers (Mastorakos et al. (2015) Nanoscale. 7 (9): 3845-56; Cheng et al. (2008) J Pharm Sci. 97 (1): 123-43). The dendrimer generation can control the payload capacity and size, and can provide a high drug payload capacity. Moreover, display of multiple surface groups can be leveraged to improve stability, reduce nonspecific interactions, and enhance cell-specific targeting and drug release.

### III. Targeted Active Gene Editing (TAGE) Agent

The present invention includes a targeted active gene editing (TAGE) agent that is useful for delivering a gene (e.g., an immunomodulatory gene selected from SOCS1 or editing polypeptide (i.e., a site-directed modifying polypeptide) to a target cell. In some embodiments, the TAGE agent can be a biologic. In particular embodiments, the site-directed modifying polypeptide contains a conjugation moiety that allows the protein to be conjugated to a cell targeting agent (e.g., an antigen binding protein, ligand, or cell penetrating peptide (CPP), or combinations thereof) that binds to an antigen associated with the extracellular region of a cell membrane or otherwise increases cellular or nuclear internalization of the site-directed modifying polypeptide.

In the case of TAGE agent that include a ligand or antigen-binding protein, this target specificity allows for delivery of the site-directed modifying polypeptide only to cells displaying the antigen (e.g., a cancer cell, a monocyte, a macrophage (e.g., M1 macrophage, M2 macrophage), an endothelial cell, an epithelial cell, a natural killer cell, a pericyte, hematopoietic stem cells (HSCs), hematopoietic progenitor stem cells (HPSCs), DC cells, non-DC myeloid cells, B cells, T cells (e.g., activated T cells), fibroblasts, or other cells). Such cells may be associated with a certain tissue or cell-type associated with a disease. The TAGE agent thus provides a means by which the genome of a target cell can be modified. TAGE agents are further described, for example, in International Publication Nos. WO2020/198151A1 and WO 2020/198160 A1, which are hereby incorporated by reference.

In one embodiment, a TAGE agent comprises a nucleic acid-guided endonuclease (e.g., RNA-guided endonuclease or DNA-guided endonuclease), such as Cas9, that recognizes a CRISPR sequence, and an antigen binding protein that specifically binds to an extracellular molecule (e.g., protein, glycan, lipid) localized on a target cell membrane. Examples of antigen binding proteins that can be used in the TAGE agent of the invention include, but are not limited to, an antibody, an antigen-binding portion of an antibody, or an antibody mimetic. The types of antigen binding proteins that can be used in the compositions and methods described herein are described in more detail in Section IV.

In another embodiment, a TAGE agent comprises a nucleic acid-guided endonuclease (e.g., RNA-guided endonuclease or DNA-guided endonuclease), such as Cas9, that recognizes a CRISPR sequence, and a ligand that specifically binds to an extracellular molecule (e.g., protein, glycan, lipid) localized on a target cell membrane. Examples of ligands that can be used in the compositions and methods described herein are described in more detail below.

In a further embodiment, a TAGE agent comprises a nucleic acid-guided endonuclease (e.g., RNA-guided endonuclease or DNA-guided endonuclease), such as Cas9, that recognizes a CRISPR sequence, and a CPP. Examples of CPPs that can be used in the compositions and methods described herein are described in more detail below.

Proteins within the TAGE agent (i.e., at least a site-directed polypeptide and a cell targeting agent) are stably associated such that the cell targeting agent directs the site-directed modifying polypeptide to the cell surface and the site-directed modifying polypeptide is internalized into the target cell. In certain embodiments, the cell targeting agent binds to the antigen on the cell surface such that the site-directed modifying polypeptide is internalized by the target cell but the cell targeting agent (e.g., antigen binding protein, ligand, or CPP) is not internalized. In some embodiments, the site-directed modifying polypeptide and a cell targeting agent are both internalized into the target cell.

Examples of a cell targeting agent include, but are not limited to, an antigen binding polypeptide such as an antibody or fragment thereof, a ligand, or a CPP. In certain embodiments, a TAGE agent includes a two or more cell membrane binding agents, e.g., a CPP and an antibody, a CPP and a ligand, or a ligand and antibody. Such class pairings can, in certain embodiments, improve internalization of the site-directed modifying polypeptide. For example, in certain embodiments, a class pairing includes a TAGE agent comprising a CPP, an antigen binding polypeptide (e.g., an antibody), and a site-directed modifying polypeptide, in any arrangement. Other combinations of class pairings of cell binding moieties include a ligand, CPP, and a site-directed modifying polypeptide, in any arrangement. In one embodiment, a TAGE agent comprises an antibody, a peptide cell surface TCR, and a site-directed modifying polypeptide, in any arrangement.

As described in more detail herein, in certain embodiments, when the site-directed modifying polypeptide is a nucleic acid-guided endonuclease, such as Cas9, the nucleic acid-guided endonuclease is associated with a guide nucleic acid to form a nucleoprotein. For example, the guide RNA (gRNA) binds to a RNA-guided nuclease to form a ribonucleoprotein (RNP) or a guide DNA binds to a DNA-guided nuclease to form a deoxyribonucleoprotein (DNP). In other embodiments, the nucleic acid-guided endonuclease is associated with a guide nucleic acid that comprises a DNA: RNA hybrid. In such instances, the ribonucleoprotein (i.e., the RNA-guided endonuclease and the guide RNA), deoxyribonucleoprotein (i.e., the DNA-guided endonuclease and the guide DNA), or the nucleic acid-guided endonuclease bound to a DNA: RNA hybrid guide are internalized into the target cell. In a separate embodiment, the guide nucleic acid (e.g., RNA, DNA, or DNA: RNA hybrid) is delivered to the target cell separately from the nucleic acid-guided endonuclease into the same cell. The guide nucleic acid (e.g., RNA, DNA, or DNA: RNA hybrid) may already be present in the target cell upon internalization of the nucleic acid-guided endonuclease upon contact with the TAGE agent.

A TAGE agent comprising a ligand or antigen binding protein specifically binds to an extracellular molecule (e.g., protein, glycan, lipid) localized on a target cell membrane. The target molecule can be, for example, an extracellular membrane-bound protein, but can also be a non-protein molecule such as a glycan or lipid. In one embodiment, the extracellular molecule is an extracellular protein that is expressed by the target cell, such as a ligand or a receptor. The extracellular target molecule may be associated with a specific disease condition or a specific tissue within in an organism. Examples of extracellular molecular targets associated with the cell membrane are described in the sections below.

The site-directed modifying polypeptide also comprises a conjugation moiety such that the cell targeting agent can stably associate with the site-directed modifying polypeptide (thus forming a TAGE agent). The conjugation moiety provides for either a covalent or a non-covalent linkage between the cell targeting agent and the site-directed modifying polypeptide.

In certain embodiments, the conjugation moiety useful for the present TAGE agents are stable extracellularly, prevent aggregation of TAGE molecules, and/or keep TAGE agents freely soluble in aqueous media and in a monomeric state. Before transport or delivery into a cell, the TAGE agent is stable and remains intact, e.g., the cell targeting agent remains linked to the nucleic acid-guided endonuclease.

In one embodiment, the conjugation moiety is Protein A, wherein the site-directed modifying polypeptide comprises Protein A and the cell targeting agent, e.g., an antigen binding protein, comprises an Fc region that can be bound by Protein A, e.g., an antibody comprising an Fc domain. In one embodiment, a site-directed modifying polypeptide comprises Protein A, or an Fc binding portion thereof.

In another embodiment, the conjugation moiety is a SpyCatcher/SpyTag peptide system. For example, in certain embodiments, the site-directed modifying polypeptide comprises SpyCatcher (e.g., at the N-terminus or C-terminus) and the cell targeting agent comprises a SpyTag. For example, in instances where the site-directed modifying polypeptide comprises Cas9, the Cas9 may be conjugated to SpyCatcher to form SpyCatcher-Cas9 or Cas9-SpyCatcher.

Other conjugation moieties useful in the TAGE agents provided herein include, but are not limited to, a Spycatcher tag, Snoop tag, Halo-tag (e.g., derived from haloalkane dehalogenase), Sortase, mono-avidin, ACP tag, a SNAP tag, or any other conjugation moieties known in the art. In one embodiment, the conjugation moiety is selected from Protein A, CBP, MBP, GST, poly (His), biotin/streptavidin, V5-tag, Myc-tag, HA-tag, NE-tag, His-tag, Flag tag, Halo-tag, Snap-tag, Fc-tag, Nus-tag, BCCP, thioredoxin, SnooprTag, SpyTag, SpyCatcher, Isopeptag, SBP-tag, S-tag, AviTag, and calmodulin.

In some embodiments, the conjugation moiety is a chemical tag. For example, a chemical tag may be SNAP tag, a CLIP tag, a HaloTag or a TMP-tag. In one example, the chemical tag is a SNAP-tag or a CLIP-tag. SNAP and CLIP fusion proteins enable the specific, covalent attachment of virtually any molecule to a protein of interest. In another example, the chemical tag is a HaloTag. HaloTag involves a modular protein tagging system that allows different molecules to be linked onto a single genetic fusion, either in solution, in living cells, or in chemically fixed cells. In another example, the chemical tag is a TMP-tag.

In some embodiments, the conjugation moiety is an epitope tag. For example, an epitope tag may be a poly-histidine tag such as a hexahistidine tag or a dodecahistidine, a FLAG tag, a Myc tag, a HA tag, a GST tag or a V5 tag.

Depending on the conjugation approach, the site-directed modifying polypeptide and the cell targeting agent may each be engineered to comprise complementary binding pairs that enable stable association upon contact. Exemplary binding moiety pairings include (i) streptavidin-binding peptide (streptavidin binding peptide; SBP) and streptavidin (STV), (ii) biotin and EMA (enhanced monomeric avidin), (iii) SpyTag (ST) and SpyCatcher (SC), (iv) Halo-tag and Halo-tag ligand, (v) and SNAP-Tag, (vi) Myc tag and anti-Myc immunoglobulins (vii) FLAG tag and anti-FLAG immunoglobulins, and (ix) ybbR tag and coenzyme A groups. In some embodiments, the conjugation moiety is selected from SBP, biotin, SpyTag, SpyCatcher, halo-tag, SNAP-tag, Myc tag, or FLAG tag.

In certain embodiments, the site-directed modifying polypeptide can alternatively be associated with a cell targeting agent, e.g., an antigen binding protein, ligand, or CPP, via one or more linkers as described herein wherein the linker is a conjugation moiety.

The term “linker” as used herein means a divalent chemical moiety comprising a covalent bond or a chain of atoms that covalently attaches a cell targeting agent to a site-directed modifying polypeptide to form a TAGE agent. Any known method of conjugation of peptides or macromolecules can be used in the context of the present disclosure. Generally, covalent attachment of the cell targeting agent and the site-directed modifying polypeptide requires the linker to have two reactive functional groups, i.e., bivalency in a reactive sense. Bivalent linker reagents which are useful to attach two or more functional or biologically active moieties, such as peptides, nucleic acids, drugs, toxins, antibodies, haptens, and reporter groups are known, and methods for such conjugation have been described in, for example, Hermanson, G. T. (1996) Bioconjugate Techniques; Academic Press: New York, p234-242, the disclosure of which is incorporated herein by reference as it pertains to linkers suitable for covalent conjugation. Further linkers are disclosed in, for example, Tsuchikama, K. and Zhiqiang, A. Protein and Cell, 9(1), p. 33-46, (2018), the disclosure of which is incorporated herein by reference as it pertains to linkers suitable for covalent conjugation.

Generally, linkers suitable for use in the compositions and methods disclosed are stable in circulation, but allow for release of the cell targeting agent and/or the site-directed modifying polypeptide in the target cell or, alternatively, in close proximity to the target cell. Linkers suitable for the present disclosure may be broadly categorized as non-cleavable or cleavable, as well as intracellular or extracellular, each of which is further described in International Publication No. WO2020/198151A1, which is hereby incorporated by reference.

The cell targeting agent of the TAGE agent can be produced using any method known in the art, e.g., expression in a suitable host cell from nucleic acid encoding the cell targeting agent. A number of methods are known in the art for producing proteins. For example, the proteins can be produced in and purified from yeast, bacteria, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the cell targeting agent (e.g. antigen binding protein, ligand, or CPPs) can be linked to a moiety that facilitates transfer into a cell, e.g., a lipid nanoparticle, optionally with a linker that is cleaved once the protein is inside the cell.

In some embodiments, the cell targeting agent (e.g., antigen binding protein, ligand, or CPP) may deliver a site-specific modifying polypeptide into a cell via an endocytic process. Examples of such a process might include macropinocytosis, clathrin-mediated endocytosis, caveolae/lipid raft-mediated endocytosis, and/or receptor mediated endocytosis mechanisms (e.g., scavenger receptor-mediated uptake, proteoglycan-mediated uptake).

### Cell Targeting Agent of the TAGE Agent

Examples of binding agents that can be used as the cell targeting agent of a TAGE agent include, but are not limited to, an antigen binding polypeptide, such as an antibody, a cell penetrating peptide (CPP), a ligand, or any combinations thereof. More detail regarding these binding agents is provided below. Further, cell targeting agents, such as ligands and antigen-binding polypeptides, not only allow for receptor-mediated entry of TAGE agents, but in certain instances, the moieties also mediate the biology of the cell (e.g., by altering intracellular signal transduction pathways), which can be exploited for therapeutic uses. Cell targeting agents (e.g., ligands, antigen binding polypeptides, and CPPs) are further described, for example, in International Publication No. WO 2020/198151 A1 and WO 2020/198160 A1, which are hereby incorporated by reference.

### (i) Antigen Binding Polypeptides

An antigen binding polypeptide targets an extracellular antigen associated with a cell membrane and provide specificity with which to deliver a site-directed modifying polypeptide. Examples of antigen binding polypeptides that may be included in the TAGE agent described herein include, but are not limited to, an antibody, an antigen-binding fragment of an antibody, or an antibody mimetic.

In certain embodiments, a TAGE agent as provided herein comprises an antigen binding polypeptide that is an antibody, or an antigen-binding fragment thereof, that specifically binds to an extracellular molecule (e.g., protein, glycan, lipid) localized on a target cell membrane or associated with a specific tissue.

Antigen binding polypeptides used in the TAGE agents described herein may also be specific to a certain cell type. For example, an antigen binding polypeptide, such as an antibody or antigen binding portion thereof, may bind to an antigen present on the cell surface of a cancer cell. Other cell types that may be bound by the antigen binding polypeptide via an antigen expressed or displayed on the cell's extracellular surface, and thus gene edited by the TAGE agent, include a monocyte, a macrophage (e.g., M1 macrophage, M2 macrophage), an endothelial cell, an epithelial cell, a natural killer cell, a pericyte, a neutrophil, a T cell, a B cell, a dendritic cell, or a fibroblast.

In one embodiment, a TAGE agent comprises a domain antibody and a site-directed modifying polypeptide. Domain antibodies (dAbs) are small functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Ser. No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

In one embodiment, a TAGE agent comprises a nanobody and a site-directed modifying polypeptide. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and may be produced in prokaryotic or eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The nanoclone method (see, e.g., WO 06/079372, which is herein incorporated by reference in its entirety) is a proprietary method for generating nanobodies against a desired target, based on automated high-throughput selection of B-cells and could be used in the context of the instant invention.

In one embodiment, a TAGE agent comprises a unibody and a site-directed modifying polypeptide. UniBodies are another antibody fragment technology, however this technology is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, unibodies may function to inhibit or silence, but not kill, the cells to which they are bound. Additionally, unibody binding to cancer cells do not stimulate them to proliferate. Furthermore, because unibodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

In one embodiment, a TAGE agent comprises an affibody and a site-directed modifying polypeptide. Affibody molecules represent a class of affinity proteins based on a 58-amino acid residue protein domain, derived from one of the IgG-binding domains of staphylococcal protein A. This three helix bundle domain has been used as a scaffold for the construction of combinatorial phagemid libraries, from which affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Harmon M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

In some embodiments, the antibody, antigen-binding fragment thereof, or antibody mimetic may specifically bind to an extracellular molecule (e.g., protein, glycan, lipid) localized on a target cell membrane or associated with a specific tissue with an Kd of at least about 1×10⁻⁴, 1×10⁻⁵, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to an antigen with an affinity that is at least two-fold greater than its affinity for a nonspecific antigen. Such binding can result in antigen-mediated surface interactions. It shall be understood, however, that the binding protein may be capable of specifically binding to two or more antigens which are related in sequence. For example, the binding polypeptides of the invention can specifically bind to both human and a non-human (e.g., mouse or non-human primate) orthologs of an antigen.

In some embodiments, the antibody, antigen-binding fragment thereof, or antibody mimetic binds to a hapten which in turn specifically binds an extracellular cell surface protein (e.g., a Cas9-antibody-hapten targeting a cell receptor).

Binding or affinity between an antigen and an antibody can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmuno assay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound in the presence of increasing amounts of an unlabeled second antibody.

The antibody or antigen-binding fragment thereof, described herein can be in the form of full-length antibodies, bispecific antibodies, dual variable domain antibodies, multiple chain or single chain antibodies, and/or binding fragments that specifically bind an extracellular molecule, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the antibody is an IgG (e.g. IgG1, IgG2, IgG3 or IgG4).

The antibody, or antigen-binding fragment thereof, described herein can be in the form of full-length antibodies, bispecific antibodies, dual variable domain antibodies, multiple chain or single chain antibodies, and/or binding fragments that specifically bind an extracellular molecule, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the antibody is an IgG (e.g. IgG1, IgG2, IgG3 or IgG4). In certain embodiments, the antigen binding polypeptide is a multispecific protein, such as a multispecific (e.g., bispecific) antibody.

In one embodiments, the antigen binding protein is a bispecific molecule comprising a first antigen binding site from a first antibody that binds to a target on the extracellular cell membrane of a cell and a second antigen binding site with a different binding specificity, such as a binding specificity for a second target on the extracellular cell membrane of the cell, i.e. a bispecific antibody wherein the first and second antigen binding sites do not cross-block each other for binding to either the first or the second antigen.

Exemplary bispecific antibody molecules comprise (i) two antibodies, one with a specificity to a first antigen and another to a second target that are conjugated together, (ii) a single antibody that has one chain or arm specific to a first antigen and a second chain or arm specific to a second antigen, (iii) a single chain antibody that has specificity to a first antigen and a second antigen, e.g., via two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)₂ fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fc-region; and (x) a diabody.

Examples of platforms useful for preparing bispecific antibodies include but are not limited to BITE (Micromet), DART (MacroGenics), Fcab and Mab.sup.2 (F-star), Fc-engineered IgG1 (Xencor) or DuoBody (based on Fab arm exchange, Genmab).

Examples of different classes of bispecific antibodies include but are not limited to asymmetric IgG-like molecules, wherein the one side of the molecule contains the Fab region or part of the Fab region of at least one antibody, and the other side of the molecule contains the Fab region or parts of the Fab region of at least one other antibody; in this class, asymmetry in the Fc region could also be present, and be used for specific linkage of the two parts of the molecule; symmetric IgG-like molecules, wherein the two sides of the molecule each contain the Fab region or part of the Fab region of at least two different antibodies; IgG fusion molecules, wherein full length IgG antibodies are fused to extra Fab regions or parts of Fab regions; Fc fusion molecules, wherein single chain Fv molecules or stabilized diabodies are fused to Fcgamma regions or parts thereof; Fab fusion molecules, wherein different Fab-fragments are fused together; ScFv- and diabody-based molecules wherein different single chain Fv molecules or different diabodies are fused to eachother or to another protein or carrier molecule.

Examples of asymmetric IgG-like molecules include but are not limited to the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (EMD Serono), the Biclonic (Merus) and the DuoBody (Genmab A/S).

Example of symmetric IgG-like molecules include but are not limited to Dual Targeting (DT)-Ig (GSK/Domantis), Two-in-one Antibody (Genentech), Cross-linked Mabs (Karmanos Cancer Center), mAb₂ (F-Star) and CovX-body (CovX/Pfizer).

Examples of IgG fusion molecules include but are not limited to Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

Examples of Fc fusion molecules include but are not limited to ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv) 2-Fab (National Research Center for Antibody Medicine-China).

Examples of class V bispecific antibodies include but are not limited to F(ab)₂ (Medarex/Amgen), Dual-Action or Bis-Fab (Genentech), Dock-and-Lock (DNL) (ImmunoMedics), Bivalent Bispecific (Biotecnoland Fab-Fv (UCB-Celltech). Examples of ScFv- and diabody-based molecules include but are not limited to Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COM BODY (Epigen Biotech).

Antibodies, antigen-binding fragments, or an antibody mimetic that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive antigen binding assay.

The antibodies or binding fragments described herein may also include modifications and/or mutations that alter the properties of the antibodies and/or fragments. Methods of engineering antibodies to include any modifications are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

Antibodies or fragments thereof, may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody (or antibody fragment), nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Antibody Mimetic

The TAGE agent may include an antibody mimetic capable of binding an antigen of interest. As detailed below, a wide variety of antibody fragment and antibody mimetic technologies have been developed and are widely known in the art. Generally, an antibody mimetic, described herein, are not structurally related to an antibody, and include adnectins, affibodies, DARPins, anticalins, avimers, versabodies, aptamers and SMIPS. An antibody mimetic uses binding structures that, while mimicking traditional antibody binding, are generated from and function via distinct mechanisms. Some of these alternative structures are reviewed in Gill and Damle (2006) 17:653-658.

In one embodiment, a TAGE agent comprises an adnectin molecule and a site-directed modifying polypeptide. Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluble form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium. As mentioned previously, fibronectin may function naturally as a cell adhesion molecule, or it may mediate the interaction of cells by making contacts in the extracellular matrix. Typically, fibronectin is made of three different protein modules, type I, type II, and type Ill modules. For a review of the structure of function of the fibronectin, see Pankov and Yamada (2002) J Cell Sci.; 115 (Pt 20):3861-3, Hohenester and Engel (2002) 21:115-128, and Lucena et al. (2007) Invest Clin. 48:249-262.

In one embodiment, adnectin molecules are derived from the fibronectin type Ill domain by altering the native protein which is composed of multiple beta strands distributed between two beta sheets. Depending on the originating tissue, fibronectin may contain multiple type Ill domains which may be denoted, e.g., 1Fn3, 2Fn3, 3Fn3, etc. The 10Fn3 domain contains an integrin binding motif and further contains three loops which connect the beta strands. These loops may be thought of as corresponding to the antigen binding loops of the IgG heavy chain, and they may be altered by methods discussed below to specifically bind a target of interest. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type Ill molecule which can be accessed from the Protein Data Bank (PDB, rosb.org/pdb/home/home.do) with the accession code: 1ttg. Adnectin molecules may also be derived from polymers of 10Fn3 related molecules rather than a simple monomeric 10Fn3 structure.

Although the native 10Fn3 domain typically binds to integrin, 10Fn3 proteins adapted to become adnectin molecules are altered so to bind antigens of interest. In one embodiment, the alteration to the 10Fn3 molecule comprises at least one mutation to a beta strand. In a preferred embodiment, the loop regions which connect the beta strands of the 10Fn3 molecule are altered to bind to the antigen of interest.

The alterations in the 10Fn3 may be made by any method known in the art including, but not limited to, error prone PCR, site-directed mutagenesis, DNA shuffling, or other types of recombinational mutagenesis which have been referenced herein. In one example, variants of the DNA encoding the 10Fn3 sequence may be directly synthesized in vitro, and later transcribed and translated in vitro or in vivo. Alternatively, a natural 10Fn3 sequence may be isolated or cloned from the genome using standard methods (as performed, e.g., in U.S. patent application No. 20070082365), and then mutated using mutagenesis methods known in the art.

In one embodiment, a target antigen may be immobilized on a solid support, such as a column resin or a well in a microtiter plate. The target is then contacted with a library of potential binding proteins. The library may comprise 10Fn3 clones or adnectin molecules derived from the wild type 10Fn3 by mutagenesis/randomization of the 10Fn3 sequence or by mutagenesis/randomization of the 10Fn3 loop regions (not the beta strands). In a preferred embodiment the library may be an RNA-protein fusion library generated by the techniques described in Szostak et al., U.S. Pat. Nos. 6,258,558 and 6,261,804; Szostak et al., WO989/31700; and Roberts & Szostak (1997) 94:12297-12302. The library may also be a DNA-protein library (e.g., as described in Lohse, U.S. Pat. No. 6,416,950, and WO 00/32823). The fusion library is then incubated with the immobilized target antigen and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created based on the techniques referenced above (e.g., Roberts & Szostak (1997) 94:12297-12302). Methods of generating libraries of altered 10Fn3 domains and selecting appropriate binders which may be used with the present invention are described fully in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. patents application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type Ill domains, such as 10Fn3, followed by a selection step may be accomplished using other methods known in the art such as phage display, ribosome display, or yeast surface display, e.g., Lipovsek et al. (2007) Journal of Molecular Biology 368:1024-1041; Sergeeva et al. (2006) Adv Drug Deliv Rev. 58:1622-1654; Petty et al. (2007) Trends Biotechnol. 25:7-15; Rothe et al. (2006) Expert Opin Biol Ther. 6:177-187; and Hoogenboom (2005) Nat Biotechnol. 23:1105-1116.

It should be appreciated by one of skill in the art that the methods references cited above may be used to derive antibody mimics from proteins other than the preferred 10Fn3 domain. Additional molecules which can be used to generate antibody mimics via the above referenced methods include, without limitation, human fibronectin modules 1Fn3-9Fn3 and 11Fn3-17Fn3 as well as related Fn3 modules from non-human animals and prokaryotes. In addition, Fn3 modules from other proteins with sequence homology to 10Fn3, such as tenascins and undulins, may also be used. Other exemplary proteins having immunoglobulin-like folds (but with sequences that are unrelated to the VH domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule PO, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create a adnecting like binding moiety. Such proteins may be identified, for example, using the program SCOP (Murzin et al., J. Mol. Biol. 247:536 (1995); Lo Conte et al., Nucleic Acids Res. 25:257 (2000).

In one embodiment, a TAGE agent comprises an aptamer and a site-directed modifying polypeptide. An “aptamer” used in the compositions and methods disclosed herein includes aptamer molecules made from either peptides or nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11 (7-8):334-41; and Bickle et al. 2006. Nat Protoc. 1 (3): 1066-91 which are incorporated herein by reference.

In certain embodiment, an aptamer is a small nucleotide polymer that binds to a specific molecular target. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers often form complex three-dimensional structures which determine their affinity for target molecules. Aptamers can offer many advantages over simple antibodies, primarily because they can be engineered and amplified almost entirely in vitro. Furthermore, aptamers often induce little or no immune response.

Aptamers may be generated using a variety of techniques, but were originally developed using in vitro selection (Ellington and Szostak. (1990) Nature. 346 (6287): 818-22) and the SELEX method (systematic evolution of ligands by exponential enrichment) (Schneider et al. 1992. J Mol Biol. 228 (3): 862-9) the contents of which are incorporated herein by reference. Other methods to make and uses of aptamers have been published including Klussmann. The Aptamer Handbook Functional Oligonucleotides and Their Applications. ISBN: 978-3-527-31059-3; Ulrich et al. 2006. Comb Chem High Throughput Screen 9 (8): 619-32; Cerchia and de Franciscis. 2007. Methods Mol Biol. 361:187-200; Ireson and Kelland. 2006. Mol Cancer Ther. 2006 5 (12):2957-62; U.S. Pat. Nos. 5,582,981; 5,840,867; 5,756,291; 6,261,783; 6,458,559; 5,792,613; 6,111,095; and U.S. patent application U.S. Pub. No. 20070009476A1; U.S. Pub. No. 20050260164A1; U.S. Pat. No. 7,960,102; and U.S. Pub. No. 20040110235A1, which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen which have higher and higher affinity for the target molecule. SELEX methods are described more fully in the following publications, which are incorporated herein by reference: Bugaut et al. 2006. 4 (22):4082-8; Stoltenburg et al. 2007 Biomol Eng. 2007 24 (4):381-403; and Gopinath. 2007. Anal Bioanal Chem. 2007. 387 (1):171-82.

In one embodiment, a TAGE agent comprises a DARPin and a site-directed modifying polypeptide. DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

In one embodiment, a TAGE agent comprises an anticalin and a site-directed modifying polypeptide. Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

In one embodiment, a TAGE agent comprises a lipocalin and a site-directed modifying polypeptide. Lipocalins are cloned and their loops are subjected to engineering in order to create anticalins. Libraries of structurally diverse anticalins have been generated and anticalin display allows the selection and screening of binding function, followed by the expression and production of soluble protein for further analysis in prokaryotic or eukaryotic systems. Studies have successfully demonstrated that anticalins can be developed that are specific for virtually any human target protein can be isolated and binding affinities in the nanomolar or higher range can be obtained.

Anticalins can also be formatted as dual targeting proteins, so-called duocalins. A duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as duocalins have significant potential in targeting cell surface molecules in disease, mediating agonistic effects on signal transduction pathways or inducing enhanced internalization effects via binding and clustering of cell surface receptors. Furthermore, the high intrinsic stability of duocalins is comparable to monomeric Anticalins, offering flexible formulation and delivery potential for Duocalins.

Additional information regarding anticalins can be found in U.S. Pat. No. 7,250,297 and International Patent Application Publication No. WO 99/16873, both of which are hereby incorporated by reference in their entirety.

Another antibody mimetic technology useful in the context of the instant invention are avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

In one embodiment, a TAGE agent comprises a versabody and a site-directed modifying polypeptide. Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of versabodies, these antibody mimetics offer a versatile format that includes multi-valency, multi-specificity, a diversity of half-life mechanisms, tissue targeting modules and the absence of the antibody Fc region. Furthermore, versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding versabodies can be found in U.S. Patent Application Publication No. 2007/0191272 which is hereby incorporated by reference in its entirety.

In one embodiment, a TAGE agent comprises an SMIP and a site-directed modifying polypeptide. SMIPs™ (Small Modular ImmunoPharmaceuticals-Trubion Pharmaceuticals) are engineered to maintain and optimize target binding, effector functions, in vivo half-life, and expression levels. SMIPS consist of three distinct modular domains. First they contain a binding domain which may consist of any protein which confers specificity (e.g., cell surface receptors, single chain antibodies, soluble proteins, etc.). Secondly, they contain a hinge domain which serves as a flexible linker between the binding domain and the effector domain, and also helps control multimerization of the SMIP drug. Finally, SMIPS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. patents application Nos. 20050238646; 20050202534; 20050202028; 20050202023; 20050202012; 20050186216; 20050180970; and 20050175614.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25 (8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### (ii) Ligands

A ligand binds to an extracellular molecule associated with a cell membrane and provides specificity with which to deliver a site-directed modifying polypeptide. Examples of ligands that may be included in the TAGE agent are described below.

In certain embodiments, the TAGE agent provided herein includes one or more ligands, which refers to a molecule that is capable of binding to another molecule on or in a cell, including one or more cell surface receptors, and includes molecules such as proteins, hormones, neurotransmitters, cytokines, growth factors, cell adhesion molecules, or nutrients. Also contemplated are binding fragments of the ligands described herein, e.g., where the fragment binds to the corresponding receptor of the ligand.

Ligands useful in TAGE agents herein include any molecules capable of binding a cell surface receptor. In certain embodiments, the ligand is a mammalian ligand, e.g., a human ligand, non-human primate ligand, or a mouse ligand.

Ligands useful in the TAGE agents herein include those capable of specifically binding cell surface receptors or cell surface molecules. In certain embodiments, the ligand binds surface molecules with demonstrated cytoplasmic release and/or nuclear trafficking.

A ligand suitable herein can alternatively comprise an amino acid sequence that is at least 90%, 91%, 92%, 93%, 94%, 95%, 96%, 97%, 98%, or 99% identical to any of the ligand amino acid sequences disclosed herein. Such a variant ligand protein should have ligand activity, such as the ability to retain binding activity to its corresponding receptor and/or the ability to mediate cellular uptake of molecular cargo (e.g., an amino acid sequence comprising one or more site-specific modifying polypeptide (e.g., nuclease)). Testing the activity of a variant ligand can be done any number of ways, such as by covalently linking it with a fluorescent protein (e.g., GFP) and measuring the degree of fluorescence emitted from a cell contacted with the ligand-fluorescent protein complex, or by testing for binding of the ligand to a receptor or cell using binding assays known in the art.

In some embodiments, the TAGE agent comprises a ligand that binds to a protein expressed on the surface of cells selected from cancer cells, monocytes, macrophages (e.g., M1 macrophage, M2 macrophage), endothelial cells, epithelial cells, natural killer cells, pericytes, DC cells, non-DC myeloid cells, B cells, T cells (e.g., activated T cells), fibroblasts, or other cells.

In addition, the TAGE agent can include a nuclear localization sequence, e.g., SV40 large T antigen NLS (PKKKRKV (SEQ ID NO: 6)) and nucleoplasmin NLS (KRPAATKKAGQAKKKK (SEQ ID NO: 7)). Other NLSs are known in the art; see, e.g., Cokol et al., EMBO Rep. 2000 Nov. 15; 1(5):411-415; Freitas and Cunha, Curr Genomics. 2009 December; 10 (8): 550-557. In some embodiments, the TAGE agent includes one or more NLS such as 1, 2, 3, 4, 5, 6, 7, 8, 9, 10, or more than 10 NLSs. In certain embodiments, the TAGE agent includes one or more (e.g., 1, 2, 3, 4, 5, 6, 7, 8, 9, 10, or more than 10 NLSs) C-terminal NLSs and one or more (e.g., 1, 2, 3, 4, 5, 6, 7, 8, 9, 10, or more than 10 NLSs) N-terminal NLSs.

One or more ligands can be located at the N-terminus or C-terminus of a site-specific modifying polypeptide to form a TAGE agent herein. Alternatively, one or more ligands can be located at both the N- and C-termini of the site-specific modifying polypeptide. Alternatively still, one or more ligands can be located within the amino acid sequence of the site-specific modifying polypeptide. Embodiments herein comprising more than one ligand can comprise at least 2, 3, 4, 5, 6, 7, 8, 9, 10, 11, 12, 13, 14, 15, 16, 17, 18, 19, or 20 ligands, or 5-10, 5-20, or 10-20 ligands. The ligands fused to the site-specific modifying polypeptide (e.g., nuclease) can be the same or different (e.g., 2, 3, 4, or more different types of ligands). One or more ligands can be fused directly to the amino acid sequence of a site-specific modifying polypeptide (e.g., nuclease), and/or can be fused to a heterologous domain(s) (e.g., NLS) that is fused with a site-specific modifying polypeptide (e.g., nuclease).

Ligands can be linked with a site-specific modifying polypeptide through covalent or non-covalent strategies. Methods for covalently joining a ligand and a site-specific modifying polypeptide are known in the art, e.g. chemical cross-linking or cloning a fusion protein, as further described herein. Non-covalent coupling between the cargo and short amphipathic ligands comprising polar and non-polar domains is established through electrostatic and hydrophobic interactions.

In one embodiment, a fusion between a ligand and a site-specific modifying polypeptide (e.g., nuclease) to form a TAGE agent herein can be directly through a peptide bond. Alternatively, a fusion between a ligand and a site-specific modifying polypeptide (e.g., nuclease) can be via an intermediary amino acid sequence. Examples of an intermediary amino acid sequence include suitable linker sequences comprising at least 1, 2, 3, 4, 5, 6, 7, 8, 9, 10, 11, 12, 13, 14, 15, 16, 17, 18, 19, 20, 30, 40, 50, 60, 70, 80, 90, or 100 amino acid residues such as glycine, serine, alanine and/or proline. Suitable amino acid linkers are disclosed in U.S. Pat. Nos. 8,580,922 and 5,990,275, for example, which are incorporated herein by reference. Other examples of intermediary amino acid sequences can comprise one or more other types of proteins and/or domains. For example, a marker protein (e.g., a fluorescent protein such as any of those disclosed herein) or NLS peptide can be comprised in an intermediary amino acid sequence.

Alternatively, a site-specific modifying polypeptide (e.g., nuclease) and at least one ligand can be covalently linked in a TAGE agent via crosslinking (chemical crosslinking). Crosslinking herein refers to a process of chemically joining two or more molecules (a site-specific modifying polypeptide (e.g., nuclease) and at least one ligand, in this case) by a covalent bond(s). Crosslinking can be performed using any number of processes known in the art, such as those disclosed in U.S. Patent Appl. Publ. No. 2011/0190813, U.S. Pat. No. 8,642,744, and Bioconjugate Techniques, 2nd Edition (G. T. Hermanson, Academic Press, 2008), which are all incorporated herein by reference. A ligand and/or a site-specific modifying polypeptide (e.g., nuclease) can be modified and/or synthesized to contain a suitable protein linking group at its N-terminus, C-terminus, and/or an amino acid side group, for the purpose of crosslinking the ligand to a site-specific modifying polypeptide (e.g., nuclease). Examples of chemical crosslinkers are further described herein.

A site-specific modifying polypeptide (e.g., nuclease) and at least one ligand herein can be non-covalently linked to each other in a TAGE agent using a variety of approaches known in the art. Though not intending to be held to any particular theory or mechanism, it is contemplated that a non-covalent linkage between the site-specific modifying polypeptide (e.g., nuclease) and at least one ligand can be due to electrostatic, Van der Waals, and/or hydrophobic forces.

A site-specific modifying polypeptide (e.g., nuclease) and at least one ligand herein can be non-covalently or covalently linked to each other in a TAGE agent using a variety of bioconjugation tools known in the art (see, e.g., Rabuka, David. Current opinion in chemical biology 14.6 (2010): 790-796, which is hereby incorporated by reference). To form the TAGE agent, a site-specific modifying polypeptide (e.g., nuclease) can be complexed with a ligand via a bio-conjugation molecule. Examples of bio-conjugation molecules include, without limitation, Spycatcher tag, Halo-tag, Sortase, mono-avidin, or a SNAP tag. In one embodiment, the bio-conjugation moleucle is selected from CBP, MBP, GST, poly (His), biotin/streptavidin, V5-tag, Myc-tag, HA-tag, NE-tag, His-tag, Flag tag, Halo-tag, Snap-tag, Fc-tag, Nus-tag, BCCP, Thioredoxin, SnooprTag, SpyTag, SpyCatcher, Isopeptag, SBP-tag, S-tag, AviTag, Calmodulin.

In some embodiments, the ligand or site-specific modifying polypeptide (e.g., nuclease) includes a chemical tag. For example, a chemical tag may be SNAP tag, a CLIP tag, a HaloTag or a TMP-tag. In one example, the chemical tag is a SNAP-tag or a CLIP-tag. SNAP and CLIP fusion proteins enable the specific, covalent attachment of virtually any molecule to a protein of interest. In another example, the chemical tag is a HaloTag. HaloTag involves a modular protein tagging system that allows different molecules to be linked onto a single genetic fusion, either in solution, in living cells, or in chemically fixed cells. In another example, the chemical tag is a TMP-tag.

In some embodiments, the ligand or site-specific modifying polypeptide (e.g., nuclease) includes an epitope tag. For example, an epitope tag may be a poly-histidine tag such as a hexahistidine tag or a dodecahistidine, a FLAG tag, a Myc tag, a HA tag, a GST tag or a V5 tag.

Depending on the conjugation approach, the ligand or site-specific modifying polypeptide (e.g., nuclease) may each be engineered to comprise complementary binding pairs that enable stable association of the ligand and site-specific modifying polypeptide (e.g., nuclease) upon contact. Exemplary conjugation moiety pairings include (i) streptavidin-binding peptide (streptavidin binding peptide; SBP) and streptavidin (STV), (ii) biotin and EMA (enhanced monomeric avidin), (iii) SpyTag (ST) and SpyCatcher (SC), (iv) Halo-tag and Halo-tag ligand, (v) and SNAP-Tag, (vi) Myc tag and anti-Myc immunoglobulins (vii) FLAG tag and anti-FLAG immunoglobulins, and (ix) ybbR tag and coenzyme A groups.

A site-specific modifying polypeptide (e.g., nuclease) and at least one ligand herein can be non-covalently linked to each other in a TAGE agent in certain aspects herein using a variety of approaches known in the art. Though not intending to be held to any particular theory or mechanism, it is contemplated that a non-covalent linkage between site-specific modifying polypeptide (e.g., nuclease) and at least one ligand can be due to electrostatic, Van der Waals, and/or hydrophobic forces.

In some embodiments, the ligand is delivered in trans with the site-directed modifying polypeptide.

More than one type of ligand (e.g., 2, 3, 4, or more different types of ligands) can be covalently or non-covalently linked to a site-specific modifying polypeptide (e.g., nuclease) in certain embodiments. The ratio (molar ratio) of ligand(s) to site-specific modifying polypeptide (e.g., nuclease) that can be used to prepare such a complex can be at least about 1:1, 2:1, 3:1, 4:1, 5:1, 6:1, 7:1, 8:1, 9:1, 10:1 15:1, 20:1, 30:1, 40:1, or 50:1, for example. In other aspects, the average number of ligands non-covalently linked to the site-specific modifying polypeptide (e.g., nuclease) protein may be at least 1, 2, 3, 4, 5, 6, 7, 8, 9, 10, 11, 12, 13, 14, 15, 16, 17, 18, 19, 20, 21, 22, 23, 24, or 25, or at least 5-10, 5-15, 5-20, or 5-25.

In another aspect, the provided herein is a method of modifying the genome of a target cell, the method comprising contacting the target cell with a targeted active gene editing (TAGE) agent comprising a ligand, as described herein. In certain embodiments, the target cell is a eukaryotic cell. In certain embodiments, the eukaryotic cell is a mammalian cell. In certain embodiments, the mammalian cell is a mouse cell, a non-human primate cell, or a human cell. In certain embodiments, the site-directed modifying polypeptide of the TAGE agent comprising the ligand produces a cleavage site at the target region of the genome, thereby modifying the genome. In certain embodiments, the target region of the genome is a target gene.

In certain embodiments, a method comprising the use of a TAGE agent (e.g., a TAGE agent comprising a ligand) described herein is effective to modify expression of the target gene. In certain embodiments, the method is effective to increase expression of the target gene relative to a reference level. In certain embodiments, the method is effective to decrease expression of the target gene relative to a reference level.

(iii) Cell Penetrating Peptides (CPPs)

The present invention includes a targeted active gene editing (TAGE) agent that is useful for delivering a gene editing polypeptide (i.e., a site-directed modifying polypeptide) to a target cell. In particular, the site-directed modifying polypeptide contains a conjugation moiety that allows the protein to be conjugated to a cell penetrating peptide (CPP) that internalizes within a target cell's extracellular membrane. This target specificity allows for delivery of the site-directed modifying polypeptide to cells. Such cells may be associated with a certain tissue or cell-type associated with a disease. The TAGE agent thus provides a means by which the genome of a target cell can be modified.

In one embodiment, a TAGE agent comprises a nucleic acid-guided endonuclease (e.g., RNA-guided endonuclease or DNA-guided endonuclease), such as Cas9, that recognizes a CRISPR sequence, and a CPP. Examples of CPPs that can be used in the TAGE agent of the invention are described further herein.

Proteins within the TAGE agent (i.e., at least a site-directed polypeptide and a CPP) are stably associated such that the CPP directs the site-directed modifying polypeptide to the cell surface and the site-directed modifying polypeptide is internalized into the target cell. In certain embodiments, the CPP binds to the cell surface such that the site-directed modifying polypeptide is internalized by the target cell as is the CPP.

In specific embodiments, internalization refers to at least 0.01%, at least 0.05%, at least 0.1%, at least 0.5%, at least 1%, at least 2%, at least 5% at least 10%, at least 15%, or at least 20% of the agents or compositions internalized localize a cell (e.g., within 10 minutes, 15 minutes, 20 minutes, 30 minutes, 40 minutes, 50 minutes, 1 hr, 2 hrs, 3 hrs, 4 hrs, or more). Internalization of an agent into a cell can refer to agents that have internalized, for example, into the cytoplasm of the cell or have been internalized as cargo in vesicles (e.g., endocytic vesicles).

A cell penetrating peptide (CPP) can facilitate cellular uptake of a conjugated molecule, such as one or more site-specific modifying polypeptides in a TAGE agent provided herein. A CPP can also be characterized in certain embodiments as being able to facilitate the movement or traversal of a molecular conjugate across or through one or more of a lipid bilayer, micelle, cell membrane, organelle membrane (e.g., nuclear membrane), vesicle membrane, or cell.

The TAGE agent provided herein includes one or more cell penetrating peptides (CPPs), which refers to a peptide, typically of about 5-60 amino acid residues in length, that can facilitate cellular uptake of molecular cargo, particularly one or more TAGE agents, or a site-specific modifying polypeptide thereof. Such site-specific modifying polypeptide can be associated with one or more CPPs through covalent or non-covalent linkage. A CPP can also be characterized in certain embodiments as being able to facilitate the movement or traversal of molecular cargo across/through one or more of a lipid bilayer, micelle, cell membrane, organelle membrane, vesicle membrane, or cell wall. A CPP herein can be cationic, amphipathic, or hydrophobic in certain embodiments. Examples of CPPs useful herein, and further description of CPPs in general, are disclosed in Ahmad et al. (2015). Biochimica et Biophysica Acta (BBA)-Biomembranes, 1848(2), 544-553; Becker-Hapak et al. (2001). Methods, 24(3), 247-256; Caron et al. (2004). Biochemical and biophysical research communications, 319(1), 12-20; Chauhan, A., Tikoo, A., Kapur, A. K., & Singh, M. (2007). Journal of controlled release, 117(2), 148-162; Choi, et al. (2010). PNAS, 107(43), 18575-18580; Del'Guidice et al. (2018). PloS one, 13(4), e0195558; Gautam et al. (2015). European Journal of Pharmaceutics and Biopharmaceutics, 89, 93-106; Gautam et al. (2016). Scientific reports, 6, 26278; Hatakeyama et al (2009). Journal of Controlled Release, 139(2), 127-132; Illien et al. (2016). Scientific reports, 6, 36938; Kosuge et al (2008). Bioconjugate chemistry, 19(3), 656-664; Lim et al. (2012). Molecules and cells, 34(6), 577-582; Matsui et al. (2003). Current Protein and Peptide Science, 4(2), 151-157; Salomone et al. (2012). Journal of controlled release, 163(3), 293-303; Sudo et al. (2017). Journal of Controlled Release, 255, 1-11; Komin et al. (2017). Advanced drug delivery reviews, 110, 52-64; Borrelli, Antonella, et al. Molecules 23.2 (2018): 295; Milletti, Francesca. Drug discovery today 17.15-16 (2012): 850-860, which are incorporated herein by reference. Further, there exists a database of experimentally validated CPPs (CPPsite, Gautam et al., 2012). The CPP of a TAGE agent of the invention can be any known CPP, such as a CPP shown in the CPPsite database.

CPPs useful in the TAGE agents herein include, but are not limited to, protein-derived CPPs, including the Tat protein and Penetratin; chimeric CPPs, such the Transportan derived from the binding of the neuropeptide galanin N-terminus to the Mastoparan toxin; and synthetic CPPs, including oligoarginines or peptide nucleic acids (PNAs) formed by synthetic nucleic acid analogues bound to pseudopeptide backbone.

In some embodiments, the CPP is an amphiphilic or amphipathic CPP. For example, an amphipathic or amphiphilic CPP may include an amino acid sequence containing an alternating pattern of polar/charged residues and non-polar, hydrophobic residues. An amphipathic CPP can alternatively be characterized as possessing both hydrophilic and lipophilic properties.

In some embodiments, the CPP is a cationic or polycationic CPP. For example, a cationic or polycationic CPP may include an amino acid sequence having a high relative abundance (at least 60%) of positively charged amino acids such as lysine (K), arginine (R), and/or histidine (H).

In some embodiments, the CPP is a hydrophobic or lipophilic CPP. For example, a hydrophobic or lipophilic CPP may include an amino acid sequence having mostly, or only, non-polar residues with low net charge and/or hydrophobic amino acid groups.

In some embodiments, the TAGE agents described herein can include one or more CPPS selected from NLS, Tat, Tat-NLS, His-Tat-NLS (HTN), Tat-HA, S19-Tat, CM18, CM18-Tat, hPH1, L17E, IMT-P8, IMT-P8 (C14S), TDP, TDP-KDEL (SEQ ID NO: 10; (“KDEL” disclosed as SEQ ID NO: 11), penetratin, polyR, Aurein, LAH4-L1, LMWP, Pardaxin, S10, S18, S19, S85, Vectofusin1, or ZF5.3. Additional examples of CPPs that can be used in TAGE agents, and their corresponding sequences, can be found for example, in International Publication No. WO2020/198151A1, which is hereby incorporated by reference.

In particular embodiments, the CPP is a TAT-related peptide comprising the transactivator of transcription (TAT) of human immunodeficiency virus (e.g., Tat, Tat-HA, S19-Tat). For example, a TAT-related peptide, or variant thereof, may comprise one or more (e.g., 2, 3, 4, 5, 6, 7, 8, 9, 10, 15, or more) additional amino acids at the N- or C-terminus of the sequence RKKRRQRRR (SEQ ID NO: 12). In some embodiments, the TAT-related peptide may include one or more (e.g., 1, 2, 3, 4, 5, or more) amino acid insertions, deletions, or substitutions (e.g., conservative amino acid substitutions) that do not disrupt the cell penetrating properties of the TAT sequence. Alternatively, the CPP may be a non-TAT-related peptide, such as NLS, hPH1, penetratin, TDP, TDP-KDEL (SEQ ID NO: 10; (“KDEL” disclosed as SEQ ID NO: 11), Aurein, IMT-P8, L17E, or a polyR CPP.

In certain embodiments, the TAGE agent includes a TAT peptide and one or more additional CPPs, such as an NLS. For example, the TAGE agent may include a TAT peptide and one or more NLS, optionally in combination with one or more His tags, thereby forming a HIS-TAT-NLS (HTN) fusion. In some embodiments, the TAGE agent comprises the HTN peptide of SEQ ID NO: 13.

In some embodiments, the CPP is an endosomal escape agent. For example, the endosomal escape agent may be TDP or TDP-KDEL (SEQ ID NO: 10; (“KDEL” disclosed as SEQ ID NO: 11).

Alternatively, or in addition, the TAGE agent can include CPPs that act as a nuclear localization sequence, e.g., SV40 large T antigen NLS (PKKKRKV (SEQ ID NO: 6)) and nucleoplasmin NLS (KRPAATKKAGQAKKKK (SEQ ID NO: 7)). Other NLSs are known in the art; see, e.g., Cokol et al., EMBO Rep. 2000 Nov. 15; 1(5): 411-415; Freitas and Cunha, Curr Genomics. 2009 December; 10 (8): 550-557. For example, in some embodiments, the NLS is c-Myc-NLS (PAAKRVKLD, SEQ ID NO: 8). In some embodiments, the TAGE agent includes one or more NLS such as 1, 2, 3, 4, 5, 6, 7, 8, 9, 10, or more than 10 NLSs. In certain embodiments, the TAGE agent includes one or more (e.g., 1, 2, 3, 4, 5, 6, 7, 8, 9, 10, or more than 10 NLSs) C-terminal NLSs and one or more (e.g., 1, 2, 3, 4, 5, 6, 7, 8, 9, 10, or more than 10 NLSs) N-terminal NLSs.

A CPP herein can be about 5-30, 5-25, 5-20, 10-30, 10-25, or 10-20 amino acid residues in length, for example. As other examples, a CPP can be about 5, 6, 7, 8, 9, 10, 11, 12, 13, 14, 15, 16, 17, 18, 19, 20, 21, 22, 23, 24, 25, 26, 27, 28, 29, or 30 amino acid residues in length. Yet in further aspects herein, a CPP can be up to about 35, 40, 45, 50, 55, or 60 amino acid residues in length.

A CPP suitable herein can alternatively comprise an amino acid sequence that is at least 90%, 91%, 92%, 93%, 94%, 95%, 96%, 97%, 98%, or 99% identical to any of the CPP amino acid sequences disclosed herein. Such a variant CPP protein should have CPP activity, such as the ability to mediate cellular uptake of molecular cargo (e.g., an amino acid sequence comprising one or more site-specific modifying polypeptides (e.g., nuclease)). Testing the activity of a variant CPP can be done any number of ways, such as by covalently linking it with a fluorescent protein (e.g., GFP) and measuring the degree of fluorescence emitted from a cell contacted with a the CPP-fluorescent protein complex.

One or more CPPs can be located at the N-terminus or C-terminus of a site-specific modifying polypeptide to form a TAGE agent herein. Alternatively, one or more CPPs can be located at both the N- and C-termini of the site-specific modifying polypeptide. Alternatively still, one or more CPPs can be located within the amino acid sequence of the site-specific modifying polypeptide. Embodiments herein comprising more than one CPP can comprise at least 2, 3, 4, 5, 6, 7, 8, 9, 10, 11, 12, 13, 14, 15, 16, 17, 18, 19, or 20 CPPs, or 5-10, 5-20, or 10-20 CPPs. The CPPs fused to the site-specific modifying polypeptide (e.g., nuclease) can be the same or different (e.g., 2, 3, 4, or more different types of CPPs). For example, in some embodiments, one or more TAT peptides and one or more NLS peptides is included in the TAGE agent described herein (e.g., as found in a His-Tat-NLS or HTN fusion described herein). One or more CPPs can be fused directly to the amino acid sequence of a site-specific modifying polypeptide (e.g., nuclease), and/or can be fused to a heterologous domain(s) (e.g., NLS) that is fused with a site-specific modifying polypeptide (e.g., nuclease).

CPPs can be linked with a site-specific modifying polypeptide through covalent or non-covalent strategies. Methods for covalently joining a CPP and a site-specific modifying polypeptide are known in the art, e.g. chemical cross-linking or cloning a fusion protein, as further described herein. Non-covalent coupling between the cargo and short amphipathic CPPs comprising polar and non-polar domains is established through electrostatic and hydrophobic interactions.

In one embodiment, a fusion between a CPP and a site-specific modifying polypeptide (e.g., nuclease) to form a TAGE agent herein can be directly through a peptide bond or an iso-peptide bond. Further, a fusion between a CPP and a conjugation moiety (e.g., SpyTag) of the TAGE agent herein can be directly through a peptide bond or an iso-peptide bond. Alternatively, a fusion between a CPP and a site-specific modifying polypeptide (e.g., nuclease) can be via an intermediary amino acid sequence. Examples of an intermediary amino acid sequence include suitable linker sequences comprising at least 1, 2, 3, 4, 5, 6, 7, 8, 9, 10, 11, 12, 13, 14, 15, 16, 17, 18, 19, 20, 30, 40, 50, 60, 70, 80, 90, or 100 amino acid residues such as glycine, serine, alanine and/or proline. Suitable amino acid linkers are disclosed in U.S. Pat. Nos. 8,580,922 and 5,990,275, for example, which are incorporated herein by reference. Other examples of intermediary amino acid sequences can comprise one or more other types of proteins and/or domains. For example, a marker protein (e.g., a fluorescent protein such as any of those disclosed herein) can be comprised in an intermediary amino acid sequence.

Alternatively, a site-specific modifying polypeptide (e.g., nuclease) and at least one CPP can be covalently linked in a TAGE agent via crosslinking (chemical crosslinking). Crosslinking herein refers to a process of chemically joining two or more molecules (a site-specific modifying polypeptide (e.g., nuclease) and at least one CPP, in this case) by a covalent bond(s). Crosslinking can be performed using any number of processes known in the art, such as those disclosed in U.S. Patent Appl. Publ. No. 2011/0190813, U.S. Pat. No. 8,642,744, and Bioconjugate Techniques, 2nd Edition (G. T. Hermanson, Academic Press, 2008), which are all incorporated herein by reference. A CPP and/or a site-specific modifying polypeptide (e.g., nuclease) can be modified and/or synthesized to contain a suitable protein linking group at its N-terminus, C-terminus, and/or an amino acid side group, for the purpose of crosslinking the CPP to a site-specific modifying polypeptide (e.g., nuclease). Examples of chemical crosslinkers are further described herein.

A site-specific modifying polypeptide (e.g., nuclease) and at least one CPP herein can be non-covalently linked to each other in a TAGE agent in certain aspects herein using a variety of approaches known in the art. Though not intending to be held to any particular theory or mechanism, it is contemplated that a non-covalent linkage between site-specific modifying polypeptide (e.g., nuclease) and at least one CPP can be due to electrostatic, Van der Waals, and/or hydrophobic forces.

More than one type of CPP (e.g., 2, 3, 4, or more different types of CPPs) can be covalently or non-covalently linked to a site-specific modifying polypeptide (e.g., nuclease) in certain embodiments. The ratio (molar ratio) of CPP(s) to site-specific modifying polypeptide (e.g., nuclease) that can be used to prepare such an agent can be at least about 1:1, 2:1, 3:1, 4:1, 5:1, 6:1, 7:1, 8:1, 9:1, 10:1 15:1, 20:1, 30:1, 40:1, or 50:1, for example. In other aspects, the average number of CPPs non-covalently linked to the site-specific modifying polypeptide (e.g., nuclease) protein may be at least 1, 2, 3, 4, 5, 6, 7, 8, 9, 10, 11, 12, 13, 14, 15, 16, 17, 18, 19, 20, 21, 22, 23, 24, or 25, or at least 5-10, 5-15, 5-20, or 5-25.

In another aspect, the provided herein is a method of modifying the genome of a target cell, the method comprising contacting the target cell with a targeted active gene editing (TAGE) agent comprising a CPP, as described herein. In certain embodiments, the target cell is a eukaryotic cell. In certain embodiments, the eukaryotic cell is a mammalian cell. In certain embodiments, the mammalian cell is a mouse cell, a non-human primate cell, or a human cell. In certain embodiments, the site-directed modifying polypeptide of the TAGE agent comprising the CPP produces a cleavage site at the target region of the genome, thereby modifying the genome. In certain embodiments, the target region of the genome is a target gene.

In certain embodiments, a method comprising the use of a TAGE agent (e.g., a TAGE agent comprising a CPP) described herein is effective to modify expression of the target gene. In certain embodiments, the method is effective to increase expression of the target gene relative to a reference level. In certain embodiments, the method is effective to decrease expression of the target gene relative to a reference level.

### Class Pairing

In certain embodiments, the TAGE includes a two or more cell targeting agents in addition to the conformation specific NP binding agent, e.g., a CPP and an antibody, a CPP and a ligand, or a ligand and antibody. Such pairings can include agents that bind to target molecules on the surface of a cell, e.g., an antibody/ligand pairing. Class pairings can, in certain embodiments, improve internalization of the site-directed modifying polypeptide. For example, in certain embodiments, a class pairing includes a TAGE agent comprising a CPP, an antigen binding polypeptide (e.g., an antibody), and a site-directed modifying polypeptide, in any arrangement. Other combinations of class pairings of cell binding moieties include a ligand, CPP, and a site-directed modifying polypeptide, in any arrangement. In one embodiment, a TAGE agent comprises an antibody, a peptide cell surface TCR, and a site-directed modifying polypeptide, in any arrangement.

In some embodiments, the TAGE agent comprises one or more CPPs and one or more antigen-binding polypeptides. In certain embodiments, the TAGE agent comprises two or more CPPs and one or more antigen-binding polypeptides. In other embodiments, the TAGE agent comprises four or more CPPs and one or more antigen-binding polypeptides. In some embodiments, the TAGE agent comprises six or more CPPs and one or more antigen-binding polypeptides. In some embodiments, the TAGE agent comprises eight or more CPPs and one or more antigen-binding polypeptides.

In some embodiments, the TAGE agent comprises one or more CPPs and one or more ligands. In certain embodiments, the TAGE agent comprises two or more CPPs and one or more ligands. In other embodiments, the TAGE agent comprises four or more CPPs and one or more ligands. In some embodiments, the TAGE agent comprises six or more CPPs and one or more ligands. In some embodiments, the TAGE agent comprises eight or more CPPs and one or more ligands.

In some embodiments, the TAGE agent comprises one or more antigen-binding polypeptides and one or more ligands.

In some embodiments, the TAGE agent comprises one or more antigen-binding polypeptides, one or more CPPs, and one or more ligands.

### III. Expression Vectors

In another aspect, provided herein is an expression vector comprising a nucleic acid encoding a guide RNA (gRNA), such as a gRNA capable of targeting an immunomodulatory gene selected from an TNFAIP3 gene or a SOCS1 gene. The expression vector may optionally further comprise a nucleic acid encoding a site-directed modifying polypeptide. In some embodiments, the expression construct(s) herein is an inducible co-expression system that co-expresses multiple components of a nucleoprotein, such as a guide nucleic acid (e.g., gRNA) and a nucleic acid-guided nuclease (e.g., a RNA-guided nuclease), which then assemble to form nucleoproteins in the host cell. The gRNA and nucleic acid-guided nuclease of the co-expression system can be encoded on the same construct or on separate constructs. In some embodiments, the gRNA targeting the TNFAIP3 gene is encoded by an expression vector (e.g., an isolated polynucleotide) comprising the DNA sequence of any one of SEQ ID NOs: 125-179 (see Table 1). In some embodiments, the gRNA targeting the SOCS1 gene is encoded by an expression vector (e.g., an isolated polynucleotide) comprising the DNA sequence of any one of SEQ ID NOs: 348-431 and 479 (see Table 2).

“Expression construct”, “construct” or “vector”, as used herein, refers to a polynucleotide vehicle that can be used to introduce genetic material into a cell. Constructs can be linear or circular. Constructs useful as expression constructs herein include plasmids, viral vectors (including phage), and integratable DNA fragments (i.e., fragments integratable into the host genome by homologous recombination). The four major types of vectors are plasmids, viral vectors, cosmids, and artificial chromosomes. Expression constructs can contain a replication sequence capable of effecting replication of the construct in a suitable host cell (i.e., an origin of replication). Typically, expression constructs comprise an origin of replication, a multicloning site, and/or a selectable marker. Upon transformation of a suitable host, the expression construct may replicate and function independently of the host genome or integrate into the host genome. For example, methods for integrating expression constructs for stable transformation of prokaryotes are known in the art (see, e.g., Heap, J. T., et al., “Integration of DNA into bacterial chromosomes from plasmids without a counter-selection marker,” Nucleic Acids Res. (2012) 40:e59). Construct design depends, among other things, on the intended use and host cell for the expression construct, and the design of an expression construct for a particular use and host cell is within the level of skill in the art.

General methods for construction of expression constructs are known in the art. Expression constructs for most host cells are commercially available. There are several commercial software products designed to facilitate selection of appropriate constructs and construction thereof, such as bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides.

Expression constructs typically comprise regulatory sequences that are involved in one or more of the following: regulation of transcription, post-transcriptional regulation, and regulation of translation. Expression constructs can be introduced into a wide variety of organisms including bacterial cells, yeast cells, mammalian cells, and plant cells. Expression constructs typically comprise functional regulatory sequences corresponding to the host cells or organism(s) into which they are being introduced. Further, expression constructs can include polynucleotides encoding protein tags (e.g., poly-His tags, hemagglutinin tags, fluorescent protein tags, quenchers, bioluminescent tags, nuclear localization tags). The coding sequences for such protein tags can be fused to the coding sequences (e.g., a sequence encoding a site directed modifying polypeptide). The expression construct can further comprise additional elements to promote nucleoprotein formation, such as 3′ hammerhead ribozyme or a ribozyme-guide-ribozyme system (see e.g., Gao, Y. and Yunde, Z. “Self-processing of ribozyme-flanked RNAs into guide RNAs in vitro and in vivo for CRISPR-mediated genome editing,” J. Integr. Plant Biol. (2013) 56:343-349), to produce uniform gRNA termini compatible with nucleoprotein formation. In further embodiments, the expression vector can include restrict site cassettes to facilitate insertion of additional sequences, such as spacer sequences on an expression construct encoding a gRNA.

Expression constructs can be designed for expression in prokaryotic or eukaryotic cells. Examples of regulatory elements and expression constructs suitable for use in a variety of host cells are described further herein.

### Prokaryotic Expression Constructs

In some embodiments, the expression construct comprising a gRNA (e.g., a gRNA capable of targeting a TNFAIP3 gene or a SOCS1 gene) is an expression construct that can be introduced into, propagated in, and/or expressed in a prokaryotic cell (i.e., a prokaryotic expression construct). Prokaryotic expression constructs are well known in the art. For example, expression constructs and co-expression systems for use in prokaryotic systems are described in, e.g., US20160160203A1, US20160376602A1, US20170159061A1, US20180282405A1, WO2017106583A1, and WO2014025663A1, which are hereby incorporated by reference.

To be maintained with the host cell as independently replicating polynucleotides, typically a prokaryotic expression construct comprises an origin of replication, also called a replicon, suitable for the target host cell (e.g., oriC derived from E. coli, pUC derived from pBR322, pSC101 derived from Salmonella), or 15A origin (derived from p15A) or bacterial artificial chromosomes). Origins of replication can be selected for use in expression constructs on the basis of incompatibility group, copy number, and/or host range, among other criteria.

The average number of copies of an expression construct in the cell, relative to the number of host chromosome molecules, is determined by the origin of replication contained in that expression construct. Copy number can range from a few copies per cell to several hundred.

Expression constructs can further include a selectable marker. A “selectable marker gene” refers to a gene that upon expression confers a phenotype by which successfully transformed cells carrying the expression construct can be identified. For example, the selectable marker can encode a protein necessary for the survival or growth of host cells in a selective culture medium. In such instances, host cells not containing the expression construct comprising the selection gene will not survive in the culture medium. Typical selection genes for prokaryotic expression systems encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmIR or CmR), kanamycin (KanR), spectinomycin (SpcR), streptomycin (StrR), tetracycline (TetR), gentamicin (GenR). The native promoter region for a selection gene is usually included, along with the coding sequence for its gene product, as part of a selectable marker portion of an expression construct. Alternatively, the coding sequence for the selection gene can be expressed from a constitutive promoter. As another example, Zeocin™ (Life Technologies, Grand Island, NY) can be used as a selection in bacteria, fungi (including yeast), plants and mammalian cell lines. In some embodiments, the selectable marker is a gene that upon expression confers an identifiable phenotype. For example, the selectable marker may be a fluorescent marker that confers fluorescence in cells carrying the expression construct that can be identified visually or by machine, e.g., flow cytometry.

Useful promoters are known for expression of proteins in prokaryotes, for example, include those inducible or regulated by rhamnose, arabinose, xylose, lactose, IPTG, or phosphate. In some embodiments, the inducible promoter is an L-arabinose-inducible promoter, a propionate-inducible promoter, a rhamnose-inducible promoter, a xylose-inducible promoter, a lactose-inducible promoter, an IPTG-inducible promoter, or a promoter inducible by phosphate depletion. In certain embodiments, the inducible promoter is the araBAD promoter (see, e.g., Guzman et al. J of bacteriology 177.14 (1995): 4121-4130), the T7 promoter, the T5 promoter, the pLac promoter, pTac promoter, the rhaBAD promoter (see, e.g., Kelly et al. ACS synthetic biology 5.10 (2016): 1136-1145), the prpBCDE promoter, the rhaSR promoter, or the xlyA promoter. For additional examples of promoters suitable for use in bacteria, see, for example, U.S. Publication No. 20160376602A1, which is hereby incorporated by reference in its entirety.

For example, in some embodiments the gRNA can be under a tunable promoter system such as AraC-ParaBAD or RhamS-PrhaBAD to generated optimal levels of expression for maximizing nucleoprotein formation. Alternatively, in other embodiments, the gRNA can be under a T7 promoter for maximum expression. In yet another embodiment the nucleic acid-guided nuclease or gRNA can be under an IPTG-inducible promoter such as T5, pLac, or pTac.

Expression constructs can also comprise coding sequences that are expressed from constitutive promoters. Unlike inducible promoters, constitutive promoters initiate continual gene product production under most growth conditions. Examples of constitutive promoters includes the promoter of the Tn3 bla gene, the promoter for the E. coli lipoprotein gene, Ipp; or the trpLEDCBA promoter. Constitutive promoters can be used in expression constructs for the expression of selectable markers, as described herein, and also for the constitutive expression of other gene products useful for the expression of the desired product. For example, transcriptional regulators of the inducible promoters, such as AraC, PrpR, RhaR, and XyIR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct.

Expression of proteins in prokaryotes is often carried out in bacteria, such as Escherichia coli with expression constructs containing constitutive or inducible promoters directing the expression of the expressed components of the expression construct. Examples of inducible promoters and related genes include those that function in Escherichia coli (E. coli) strain MG1655 (American Type Culture Collection deposit ATCC 700926), which is a substrain of E. coli K-12 (American Type Culture Collection deposit ATCC 10798). For example, Table 1 in US Patent Application No. US20160376602 lists the genomic locations, in E. coli MG1655, of the nucleotide sequences for these examples of inducible promoters and related genes, which is hereby incorporated by reference. Additional information about E. coli promoters, genes, and strains described herein can be found in many public sources, including the online EcoliWiki resource, located at ecoliwiki.net.

Prokaryotic expression constructs can also include ribosome binding sites of varying strength, and secretion signals (e.g., mal, see, tat, ompC, and pelB). For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. In some embodiments, the region of the expression construct that corresponds to the 5′ UTR comprises a polynucleotide sequence similar to the consensus ribosome binding site (RBS, also called the Shine-Dalgarno sequence) that is found in the species of the host cell. In prokaryotes, the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In some embodiments, the RBS sequence is at least 55% identical to the AGGAGGU consensus sequence, at least 70% identical, or at least 85% identical, and is separated from the initiation codon by 5 to 10 intervening nucleotides, by 6 to 9 intervening nucleotides, or by 6 or 7 intervening nucleotides.

### Eukaryotic Expression Constructs

In some embodiments, the expression construct comprising a gRNA (e.g., a gRNA capable of targeting a TNFAIP3 gene or a SOCS1 gene) is an expression construct that can be introduced into, propagated in, and/or expressed in a eukaryotic cell (i.e., a eukaryotic expression construct).

In one embodiment, the host cell is a yeast and the expression construct is a yeast expression construct. Examples of expression constructs for expression in Saccharomyces cerivisae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, CA). Typically, expression of protein-encoding genes in yeast requires a promoter operably linked to a coding region of interest plus a transcriptional terminator. Various yeast promoters can be used to construct expression cassettes for expression of genes in yeast. Examples of promoters include, but are not limited to, promoters of genes encoding the following yeast proteins: alcohol dehydrogenase 1 (ADH1) or alcohol dehydrogenase 2 (ADH2), phosphoglycerate kinase (PGK), triose phosphate isomerase (TPI), glyceraldehyde-3-phosphate dehydrogenase (GAPDH; also known as TDH3, or triose phosphate dehydrogenase), galactose-1-phosphate uridyl-transferase (GAL7), UDP-galactose epimerase (GAL10), cytochrome ci (CYC1), acid phosphatase (PHO5) and glycerol-3-phosphate dehydrogenase gene (GPD1). Hybrid promoters, such as the ADH2/GAPDH, CYC1/GAL10 and the ADH2/GAPDH promoter (which is induced at low cellular-glucose concentrations, e.g., about 0.1 percent to about 0.2 percent) also may be used. In S. pombe, suitable promoters include the thiamine-repressed nmtl promoter and the constitutive cytomegalovirus promoter in pTL2M.

Yeast RNA polymerase III promoters (e.g., promoters from 5S, U6 or RPR1 genes) as well as polymerase Ill termination sequences are known in the art (see, e.g., www.yeastgenome.org; Harismendy, O., et al., (2003) “Genome-wide location of yeast RNA polymerase Ill transcription machinery,” The EMBO Journal. 22 (18): 4738-4747.)

In addition to a promoter, several upstream activation sequences (UASs), also called enhancers, may be used to enhance polypeptide expression. Exemplary upstream activation sequences for expression in yeast include the UASs of genes encoding these proteins: CYC1, ADH2, GAL1, GAL7, GAL10, and ADH2. Exemplary transcription termination sequences for expression in yeast include the termination sequences of the α-factor, CYC1, GAPDH, and PGK genes. One or multiple termination sequences can be used.

Suitable promoters, terminators, and coding regions may be cloned into E. coli-yeast shuttle expression constructs and transformed into yeast cells. These expression constructs allow strain propagation in both yeast and E. coli strains. Typically, the expression construct contains a selectable marker and sequences enabling autonomous replication or chromosomal integration in each host. Examples of plasmids typically used in yeast are the shuttle vectors pRS423, pRS424, pRS425, and pRS426 (American Type Culture Collection, Manassas, VA). These plasmids contain a yeast 2 micron origin of replication, an E. coli replication origin (e.g., pMB1), and a selectable marker.

The various components can also be expressed in insects or insect cells. Suitable expression control sequences for use in such cells are well known in the art. In some aspects, it is desirable that the expression control sequence comprises a constitutive promoter. Examples of suitable strong promoters include, but are not limited to, the following: the baculovirus promoters for the piO, polyhedrin (polh), p 6.9, capsid, UAS (contains a Gal4 binding site), Ac5, cathepsin-like genes, the B. mori actin gene promoter; Drosophila melanogaster hsp70, actin, α-1-tubulin or ubiquitin gene promoters, RSV or MMTV promoters, copia promoter, gypsy promoter, and the cytomegalovirus IE gene promoter. Examples of weak promoters that can be used include, but are not limited to, the following: the baculovirus promoters for the ieI, ie2, ieO, etI, 39K (aka pp31), and gp64 genes. If it is desired to increase the amount of gene expression from a weak promoter, enhancer elements, such as the baculovirus enhancer element, hr5, may be used in conjunction with the promoter.

For the expression of some of the components disclosed herein in insects, RNA polymerase Ill promoters are known in the art, for example, the U6 promoter. Conserved features of RNA polymerase III promoters in insects are also known (see, e.g., Hernandez, G., (2007) “Insect small nuclear RNA gene promoters evolve rapidly yet retain conserved features involved in determining promoter activity and RNA polymerase specificity,” Nucleic Acids Res. 2007 January; 35 (1): 21-34).

In another aspect, the various components are incorporated into mammalian expression constructs for use in mammalian cells. A large number of mammalian expression constructs suitable for use with the systems of the present invention are commercially available (e.g., from Life Technologies, Grand Island, NY; NeoBiolab, Cambridge, MA; Promega, Madison, WI; DNA2.0, Menlo Park, CA; Addgene, Cambridge, MA).

Expression constructs derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., (2000) “Overview of vector design for mammalian gene expression,” Molecular Biotechnology, Volume 16, Issue 2, pp 151-160; Cooray S., et al., (2012) “Retrovirus and lentivirus vector design and methods of cell conditioning,” Methods Enzymol. 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act. and others known in the art (Khan, K. H. (2013) “Gene Expression in Mammalian Cells and its Applications,” Advanced Pharmaceutical Bulletin 3(2), 257-263). Further, mammalian RNA polymerase III promoters, including HI and U6, can be used.

### IV. Methods of Use

A DNA targeting system described herein (e.g., including a site-directed modifying polypeptide and a guide RNA (gRNA)) can be used to modify an immunomodulatory gene selected from a TNFAIP3 gene or a SOCS1 gene in the genome of a cell. Such methods may be used in an in vitro setting, ex vivo, or in vivo, including for therapeutic use where the modification of a TNFAIP3 gene or a SOCS1 gene in a subject in need thereof results in treatment of a disease or disorder.

The cell can be a eukaryotic cell, such as a mammalian cell (e.g., a human cell). Examples of mammalian cells that can be targeted (and have their genome's modified) by the DNA targeting systems herein include, but are not limited to, a mouse cell, a non-human primate cell, or a human cell.

The DNA targeting system, in certain instances, can be used to edit specific cell types ex vivo or in vivo, such as cancer cells, monocytes, macrophages (e.g., M1 macrophage, M2 macrophage, human macrophages), endothelial cells, epithelial cells, natural killer cells, pericytes, DC cells, non-DC myeloid cells, B cells, T cells (e.g., activated T cells, human T cells), fibroblasts, or other cells.

In some embodiments, the cells are cancer cells. In some embodiments, the cells are T cells. In some embodiments, the T cells are CD4 or CD8 T cells. In certain embodiments, the T cells are regulatory T cells (T regs) or effector T cells. In some embodiments, the T cells are tumor infiltrating T cells. In some embodiments, the cell is a hematopoietic stem cell (HSC) or a hematopoietic progenitor cells (HPSCs). In some embodiments, the cells are macrophages. In some embodiments, the macrophages are M0, M1, or M2 macrophages. In some embodiments, the cells are endothelial cells. In some embodiments, the cells are epithelial cells. In some embodiments, the cells are epithelial cells. In some embodiments, the cells are natural killer cells. In some embodiments, the cells are pericytes.

In some embodiments, the DNA targeting system is used to edit multiple (e.g., two or more) cell types selected from cancer cells, monocytes, macrophages (e.g., M1 macrophage, M2 macrophage), endothelial cells, epithelial cells, natural killer cells, pericytes, DC cells, non-DC myeloid cells, B cells, T cells (e.g., activated T cells), and fibroblasts.

The cell can be one that exists (i) in an organism/tissue in vivo, (ii) in a tissue or group of cells ex vivo, or (iii) in an in vitro state. In certain instances, the eukaryotic cell herein can be as it exists in an isolated state (e.g., in vitro cells, cultured cells) or a non-isolated state (e.g., in a subject, e.g., a mammal, such as a human, non-human primate, or a mouse).

The site-directed modifying polypeptide and gRNA of the DNA targeting system can be delivered into a cell as a nucleoprotein complex comprising the site-directed modifying polypeptide and the gRNA. Alternatively, the site-directed modifying polypeptide is delivered and the guide RNA is provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase III promoter), which can be a native promoter or heterologous to the guide RNA-encoding nucleotide sequence.

In certain embodiments, the site-directed modifying polypeptide of the DNA targeting system produces a cleavage site at the target region of the genome of the target cell, subsequently modifying the genome of the cell and impacting gene expression. Thus, in one embodiment, the target region of the genome is a target gene, such as a TNFAIP3 gene or a SOCS1 gene. The site-directed modifying polypeptide's ability to modify the genome of the target cell provides, in certain embodiments, a way to modify expression of the target gene (e.g., TNFAIP3 or SOCS1). Expression levels of a target nucleic acid, e.g., a gene (e.g., TNFAIP3 or SOCS1), can be determined according to standard methods. In certain circumstances, the method disclosed herein is effective to increase expression of the target gene relative to a reference level. Alternatively, in other circumstances, the method disclosed herein is able to decrease expression of the target gene (e.g., TNFAIP3 or SOCS1) relative to a reference level. Reference levels can be determined in standard assays using a non-specific guide RNA/site-directed modifying polypeptide, where increases or decreases in the target nucleic acid, e.g., gene (e.g., TNFAIP3 or SOCS1), may be measured relative to the control.

The ability of the DNA targeting system to modify a target nucleic acid, e.g., gene (e.g., TNFAIP3 or SOCS1), in a target cell can be determined according to methods known in the art, including, for example, phenotypic assays or sequencing assays. Such assays may determine the presence or absence of a modified gene (e.g., a modified TNFAIP3 gene or a modified SOCS1 gene), or a marker associated with the gene or nucleic acid, in a cell or in a population of cells that have been contacted by the site-directed modifying polypeptide and the gRNA.

The percentage of cells that include a modified gene of interest (e.g., a TNFAIP3 gene or a SOCS1 gene) may be used to assess the editing rate, as described in Examples 2 and 4. For example, the gene of interest (e.g., TNFAIP3 or SOCS1) may be modified in at least about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 2%, 4%, 5%, 10%, 15%, 20%, 30%, 40%, 50%, 60%, 70%, 80%, 90%, 95%, or more cells in the population of cells. In certain embodiments, the methods herein are effective to modify the gene of interest (e.g., TNFAIP3 or SOCS1) in at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of cells in a population of cells. Ranges of the foregoing percentages are also contemplated herein.

In some embodiments, editing of a gene of interest (e.g., TNFAIP3 or SOCS1) can be determined by measuring the levels of the corresponding mRNA transcript or protein, respectively, in a population of cells and compared to a control site-directed modifying polypeptide where a non-targeting guide RNA is used as a negative control in the same type of target cell. In some embodiments, decreases in the level of mRNA transcript or protein (e.g., TNFAIP3 or SOCS1), for example, relative to the control indicates editing of the corresponding gene (e.g., TNFAIP3 or SOCS1). In certain instances, a decrease in the level of mRNA transcript or protein (e.g., TNFAIP3 or SOCS1) of at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.3%, at least 0.4%, at least 0.5%, at least 0.6%, at least 0.7%, at least 0.8%, at least 0.9%, at least 1%, at least 2%, at least 5%, at least 10%, at least 15%, at least 20%, at least 25%, at least 30%, at least 35%, and so forth, relative to a control in a testing assay indicates editing of the corresponding gene (e.g., TNFAIP3 or SOCS1), respectively. Ranges of the foregoing percentages are also contemplated herein.

Other ways in which nucleic acid, e.g., gene (e.g., TNFAIP3 or SOCS1), editing activity of a site-directed modifying polypeptide can be determined include sequence based assays, e.g., amplicon sequencing, known in the art.

In some embodiments, the methods include use of a site-directed modifying polypeptide coupled with a cell targeting agent (e.g., antigen binding protein, ligand, or CPP) to form a TAGE. The TAGE can be produced using any method known in the art, e.g., through covalent or non-covalent linkages, or expression in a suitable host cell from nucleic acid encoding the variant protein. A number of methods are known in the art for producing proteins. For example, the proteins can be produced in and purified from yeast, bacteria, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the cell targeting agent (e.g. antigen binding protein, ligand, or CPPs) can be linked to a moiety that facilitates transfer into a cell, e.g., a lipid nanoparticle, optionally with a linker that is cleaved once the protein is inside the cell.

In some embodiments, the cell targeting agent may deliver a site-specific modifying polypeptide into a cell via an endocytic process. Examples of such a process might include macropinocytosis, clathrin-mediated endocytosis, caveolae/lipid raft-mediated endocytosis, and/or receptor mediated endocytosis mechanisms (e.g., scavenger receptor-mediated uptake, proteoglycan-mediated uptake).

Once a site-specific modifying polypeptide is inside a cell, it may traverse an organelle membrane such as a nuclear membrane or mitochondrial membrane, for example. In certain embodiments, the site-specific modifying polypeptide includes at least one (e.g., at least 1, 2, 3, 4, or more) nuclear-targeting sequence (e.g., NLS). In other embodiments, the ability to traverse an organelle membrane such as a nuclear membrane or mitochondrial membrane does not depend on the presence of a nuclear-targeting sequence. Accordingly, in some embodiments, the site-specific modifying polypeptide does not include an NLS.

In some embodiments, the site-directed modifying polypeptide comprising the gRNA targeting a TNFAIP3 gene or a SOCS1 gene is administered to cells ex vivo, such as T cells or other cells in the tumor microenvironment (e.g., macrophages, CD4+ T cells, CD8+ T cells, or fibroblasts).

A site-directed modifying polypeptide and the gRNA (e.g., gRNA targeting a TNFAIP3 gene or a SOCS1 gene) may be administered to a subject by a route in accordance with the therapeutic goal. A variety of routes may be used to deliver a site-directed modifying polypeptide comprising the gRNA to desired cells or tissues, including systemic or local delivery.

In certain embodiments, the site-directed modifying polypeptide and the gRNA (e.g., gRNA targeting a TNFAIP3 gene or a SOCS1 gene) are administered to a subject, e.g., by local administration. In some embodiments, the site-directed modifying polypeptide and the gRNA (e.g., gRNA targeting a TNFAIP3 gene or a SOCS1 gene) are administered to the subject transdermally, subcutaneously, intravenously, intramuscularly, intraocularly, intraosseously, peritumorally, or intratumorally (e.g., in a therapeutically effective amount). In one embodiment, the site-directed modifying polypeptide comprising the gRNA (e.g., gRNA targeting a TNFAIP3 gene or a SOCS1 gene) is administered to a subject intratumorally. In one embodiment, the site-directed modifying polypeptide comprising the gRNA (e.g., gRNA targeting TNFAIP3 or SOCS1) is administered to a subject peritumorally (e.g., in a therapeutically effective amount).

The site-directed modifying polypeptide and the gRNA (e.g., gRNA targeting a TNFAIP3 gene or a SOCS1 gene) may be administered to a subject in a therapeutically effective amount (e.g., in an amount to achieve a level of genome editing (e.g., editing of a TNFAIP3 gene or a SOCS1 gene) that treats or prevents a disease in a subject). For example, a therapeutically effective amount of a site-directed modifying polypeptide and the gRNA may be administered to a subject having a cancer. A therapeutically effective amount may depend on the mode of delivery, e.g., whether the site-directed modifying polypeptide and the gRNA are administered locally (e.g., by intradermal (e.g., via the flank or ear in the case of a mouse), intratumoral, peritumoral, intraosseous, intraocular, or intramuscular injection) or systemically.

The site-directed modifying polypeptide comprising the gRNA (e.g., gRNA targeting a TNFAIP3 gene or a SOCS1 gene) described herein may be formulated to be compatible with the intended route of administration, such as by intradermal, intratumoral, intraosseous, intraocular, or intramuscular injection. Solutions, suspensions, dispersions, or emulsions may be used for such administrations and may include a sterile diluent, such as water for injection, saline solution, polyethylene glycols, glycerine, propylene glycol or other synthetic solvents; anti-bacterial agents such as benzyl alcohol or methylparabens; antioxidants such as ascorbic acid or sodium bisulfate; buffers such as acetates, citrates or phosphates, and agents for the adjustment of tonicity such as sodium chloride or dextrose. The pH may be adjusted with acids or bases, such as hydrochloric acid or sodium hydroxide. Preparations may be enclosed in ampules, disposable syringes or multiple dose vials made of glass or plastic. In certain embodiments, a pharmaceutical composition comprises a site-directed modifying polypeptide comprising the gRNA (e.g., gRNA targeting a TNFAIP3 gene or a SOCS1 gene) and a pharmaceutically acceptable carrier.

The site-directed modifying polypeptide comprising the gRNA (e.g., gRNA targeting a TNFAIP3 gene or a SOCS1 gene) can be included in a kit, container, pack or dispenser, together with medical devices suitable for delivering the compositions to a subject, such as by intradermal, intratumoral, intraosseous, intraocular, or intramuscular injection. The compositions included in kits may be supplied in containers of any sort such that the life of the different components may be preserved and may not be adsorbed or altered by the materials of the container. For example, sealed glass ampules or vials may contain the compositions described herein that have been packaged under a neutral non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include bottles that are fabricated from similar substances as ampules, and envelopes that consist of foil-lined interiors, such as aluminum or an alloy. Other containers include test tubes, vials, flasks, bottles, syringes, etc. Some containers may have a sterile resealable access port, such as a bottle having a stopper that may be pierced repeatedly by a hypodermic injection needle.

In certain embodiments, a site-directed modifying polypeptide comprising the gRNA (e.g., gRNA targeting a TNFAIP3 gene or a SOCS1 gene) is administered to a subject having a cancer. In certain embodiments, the site-directed modifying polypeptide comprising the gRNA (e.g., gRNA targeting a TNFAIP3 gene or a SOCS1 gene) may be injected directly into a tumor (i.e., by intratumoral injection) in a subject, for instance, in an amount effective to edit one or more cell types in the tumor (e.g., macrophages, CD4+ T cells, CD8+ T cells, or fibroblasts). For example, site-directed modifying polypeptide comprising the gRNA (e.g., gRNA targeting a TNFAIP3 gene or a SOCS1 gene) may be used to treat a solid tumor in subject (e.g., a human) by administering the site-directed modifying polypeptide comprising the gRNA intratumorally.

In some embodiments, a site-directed modifying polypeptide comprising the gRNA (e.g., a gRNA targeting a TNFAIP3 gene or a SOCS1 gene) may be injected directly into a solid tumor with a needle, such as a Turner Biopsy Needle or a Chiba Biopsy Needle. When treating a solid tumor in the lung, for example, a site-directed modifying polypeptide comprising the gRNA may be administered within the thorax using a bronchoscope or other device capable of cannulating the bronchial. Masses accessible via the bronchial tree may be directly injected by using a widely available transbronchial aspiration needles. A site-directed modifying polypeptide comprising the gRNA may also be implanted within a solid tumor using any suitable method known to those skilled in the art of penetrating tumor tissue. Such techniques may include creating an opening into the tumor and administering a site-directed modifying polypeptide in the tumor.

In embodiments where the site-directed modifying polypeptide is a TAGE agent, a TAGE agent comprising a CPP, or a class paired TAGE agent containing a CPP and a ligand or an antigen binding polypeptide (e.g., antibody), may be administered to a human subject via local delivery. Local delivery refers to delivery to a specific location on a body where the TAGE agent will act within the region it is delivered to, and not systemically. Examples of local delivery for a TAGE agent include topical administration, ocular delivery, intra-articular delivery, intra-cardiac delivery, intradermal, intracutaneous delivery, intraosseous delivery, intrathecal delivery, or inhalation.

In one embodiment, a TAGE agent comprising a ligand or an antigen binding polypeptide (e.g., an antibody or an antigen-binding fragment thereof), or a class paired TAGE agent comprising a ligand or an antigen binding polypeptide (e.g., an antibody or an antigen-binding fragment thereof), is administered to a human subject via systemic administration. Examples of systemic delivery for a TAGE agent containing a ligand or an antigen binding polypeptide (e.g., an antibody or an antigen-binding fragment thereof), extracellular membrane binding moiety, include intravenous injection or intraperitoneal injection.

The site-directed modifying polypeptide comprising the gRNA (e.g., a gRNA targeting a TNFAIP3 gene or a SOCS1 gene) can optionally be administered to the subject in combination with an immune checkpoint blockade agent. In certain embodiments, the immune checkpoint blockade agent is a modulator (e.g., an inhibitor or an activator) of an immune checkpoint molecule selected from the group consisting of A2AR, B7-H3, B7-H4, BTLA, CD27, CD28, CD40, CD40L, CD47, CD70, CD80, CD86, CD112/PVRL2, CD122, CD137, CD137-L, CD155/PVR, CD160, CD226, CGEN-15049, CTLA-4, GITR, GITR-L, GALS, HVEM, ICOS, IDO, KIR, NOX2, OX40, OX40L, PD-1, PD-L1, PD-L2, PD-L3, PD-L4, SIGLEC7, SIGLEC15, SIRPa, TIGIT, TIM-3, VISTA, 2B4, and LAG-3. In some embodiments, the immune checkpoint blockade agent is an immune checkpoint binding protein (e.g., an antibody or antigen-binding fragment thereof). In other embodiments, the immune checkpoint blockade agent is a small molecule. In one embodiment, the immune checkpoint blockade agent is an anti-CTLA-4 antibody, an anti-PD-1 antibody, or an anti-PD-L1 antibody.

In some embodiments, the immune checkpoint blockade agent is a modulator (e.g., an inhibitor or an activator) of A2AR. In some embodiments, the immune checkpoint blockade agent is a modulator (e.g., an inhibitor or an activator) of B7-H3. In some embodiments, the immune checkpoint blockade agent is a modulator (e.g., an inhibitor or an activator) of B7-H4. In some embodiments, the immune checkpoint blockade agent is a modulator (e.g., an inhibitor or an activator) of BTLA. In some embodiments, the immune checkpoint blockade agent is a modulator (e.g., an activator) of CD27. In some embodiments, the immune checkpoint blockade agent is a modulator (e.g., an activator) of CD28. In some embodiments, the immune checkpoint blockade agent is a modulator (e.g., an inhibitor or an activator) of CD40. In some embodiments, the immune checkpoint blockade agent is a modulator (e.g., an inhibitor or an activator) of CD80. In some embodiments, the immune checkpoint blockade agent is a modulator (e.g., an inhibitor or an activator) of CD86. In some embodiments, the immune checkpoint blockade agent is a modulator (e.g., an inhibitor or an activator) of CD122. In some embodiments, the immune checkpoint blockade agent is a modulator (e.g., an activator) of CD137. In some embodiments, the immune checkpoint blockade agent is a modulator (e.g., an inhibitor or an activator) of CD137-L. In some embodiments, the immune checkpoint blockade agent is a modulator (e.g., an inhibitor or an activator) of CD160. In some embodiments, the immune checkpoint blockade agent is a modulator (e.g., an inhibitor or an activator) of CD226. In some embodiments, the immune checkpoint blockade agent is a modulator (e.g., an inhibitor or an activator) of CGEN-15049. In some embodiments, the immune checkpoint blockade agent is a modulator (e.g., an inhibitor or an activator) of CTLA-4. In some embodiments, the immune checkpoint blockade agent is a modulator (e.g., an inhibitor or an activator) of GITR. In some embodiments, the immune checkpoint blockade agent is a modulator (e.g., an inhibitor or an activator) of GITR-L. In some embodiments, the immune checkpoint blockade agent is a modulator (e.g., an inhibitor or an activator) of GALS. In some embodiments, the immune checkpoint blockade agent is a modulator (e.g., an inhibitor or an activator) of HVEM. In some embodiments, the immune checkpoint blockade agent is a modulator (e.g., an activator) of ICOS. In some embodiments, the immune checkpoint blockade agent is a modulator (e.g., an inhibitor or an activator) of IDO. In some embodiments, the immune checkpoint blockade agent is a modulator (e.g., an inhibitor or an activator) of KIR. In some embodiments, the immune checkpoint blockade agent is a modulator (e.g., an inhibitor or an activator) of NOX2. In some embodiments, the immune checkpoint blockade agent is a modulator (e.g., an activator) of OX40. In some embodiments, the immune checkpoint blockade agent is a modulator (e.g., an inhibitor or an activator) of PD-1. In some embodiments, the immune checkpoint blockade agent is a modulator (e.g., an inhibitor or an activator) of PD-L1. In some embodiments, the immune checkpoint blockade agent is a modulator (e.g., an inhibitor or an activator) of PD-L2. In some embodiments, the immune checkpoint blockade agent is a modulator (e.g., an inhibitor or an activator) of PD-L3. In some embodiments, the immune checkpoint blockade agent is a modulator (e.g., an inhibitor or an activator) of PD-L4. In some embodiments, the immune checkpoint blockade agent is a modulator (e.g., an inhibitor or an activator) of SIGLEC7. In some embodiments, the immune checkpoint blockade agent is a modulator (e.g., an inhibitor or an activator) of SIGLEC15. In some embodiments, the immune checkpoint blockade agent is a modulator (e.g., an inhibitor or an activator) of TIGIT. In some embodiments, the immune checkpoint blockade agent is a modulator (e.g., an inhibitor or an activator) of TIM-3. In some embodiments, the immune checkpoint blockade agent is a modulator (e.g., an inhibitor or an activator) of VISTA. In some embodiments, the immune checkpoint blockade agent is a modulator (e.g., an inhibitor or an activator) of 2B4. In some embodiments, the immune checkpoint blockade agent is a modulator (e.g., an inhibitor or an activator) of LAG-3.

### EXAMPLES

The invention will be more fully understood by reference to the following examples. They should not, however, be construed as limiting the scope of the invention. All literature and patent citations are incorporated herein by reference.

### Example 1. Target Gene Identification

This Example describes the identification of TNFAIP3 and SOCS-1 as target genes for modulating the tumor microenvironment and treating immuno-oncology indications.

An initial set of 311 genes were identified that are involved in T cell activation, macrophage polarization, T cell dysfunction, T cell activation, or T cell infiltration in tumors. From the initial set of 311 genes, 33 genes were selected for further analysis based on strategic and biological considerations, including the difficulty of targeting the candidate with known drugs and the likelihood that editing the gene in a small fraction of cells would confer an amplified biological effect (e.g., the likelihood that knockout of the gene can initiate a cascade effect that leads to anti-tumor immunity). The 33 candidate genes were then analyzed based on a scoring rubric that included biological, strategic, and feasibility criteria, leading to the identification of 15 candidate target genes, including TNFAIP3 and SOCS-1.

### Example 2. Identification of gRNAs Targeting Murine TNFAIP3

This Example describes a screen for guide RNAs (gRNAs) that target the mouse Tnfaip3 gene.

20 candidate cr:tracrRNAs were designed against the mouse Tnfaip3 gene and complexed with Streptococcus pyogenes Cas9 to form ribonucleoproteins (RNPs). RNPs including the candidate gRNAs were then introduced into mouse T cells by nucleofection. A benchmark gRNA targeting CD44 was also complexed with Cas9 and assessed for editing as a positive control. Editing at the target gene was detected by Next Generation Sequencing. Doench scores for each gRNA were calculated as a measure of predicted sgRNA activity (Doench et al., Nature Biotechnology 32, 1262-1267 (2014)).

Primary human T cells were electroporated with a dose titration of pool of Cas9 RNP complexed with indicated guide RNAs. Guide RNA were in two-part (crRNA:tracrRNA) format. Three days after electroporation, genomic DNA was isolated from the cells, and editing was measured by an amplicon-based Next-Generation Sequencing (NGS) assay to quantify insertion and deletion mutations created by the RNP at each of the target sites. The level of editing (% editing) as detected by NGS is shown for each of the indicated doses. Top candidate guide RNAs are indicated as shaded.

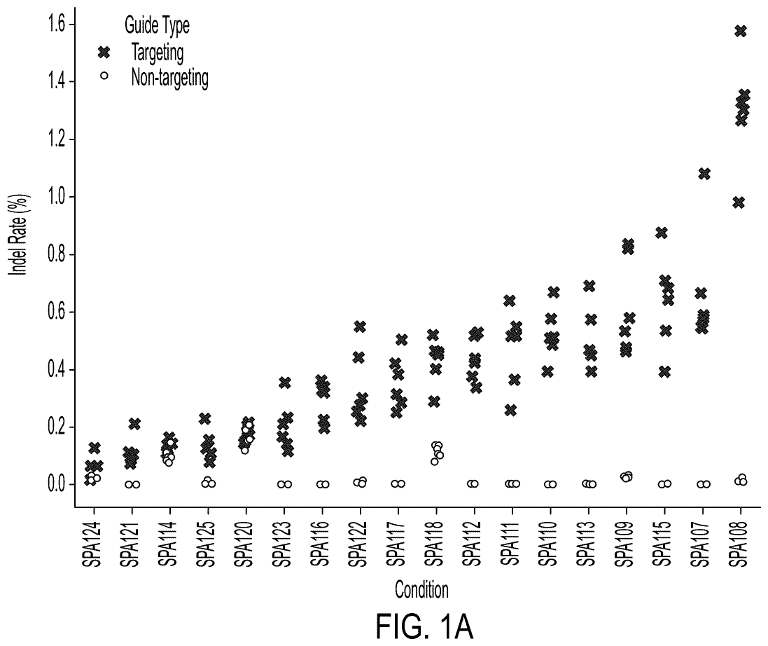

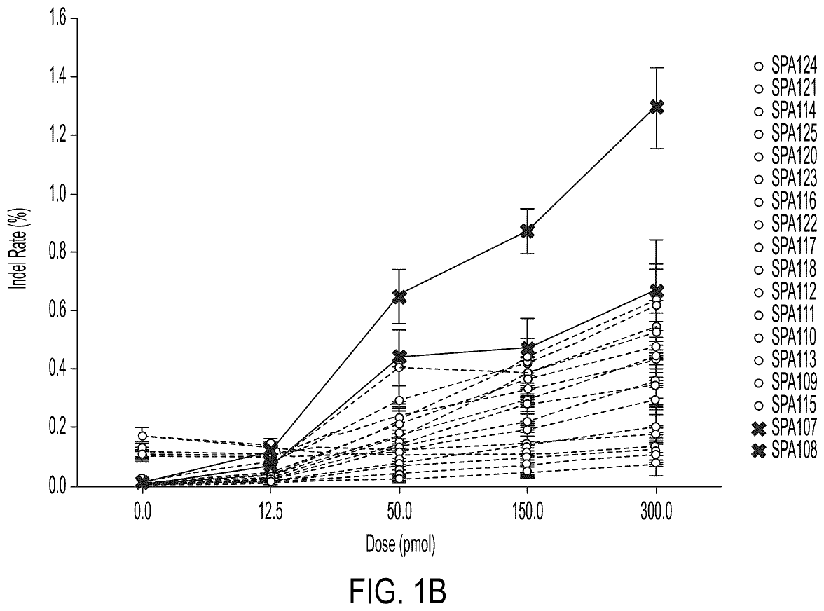

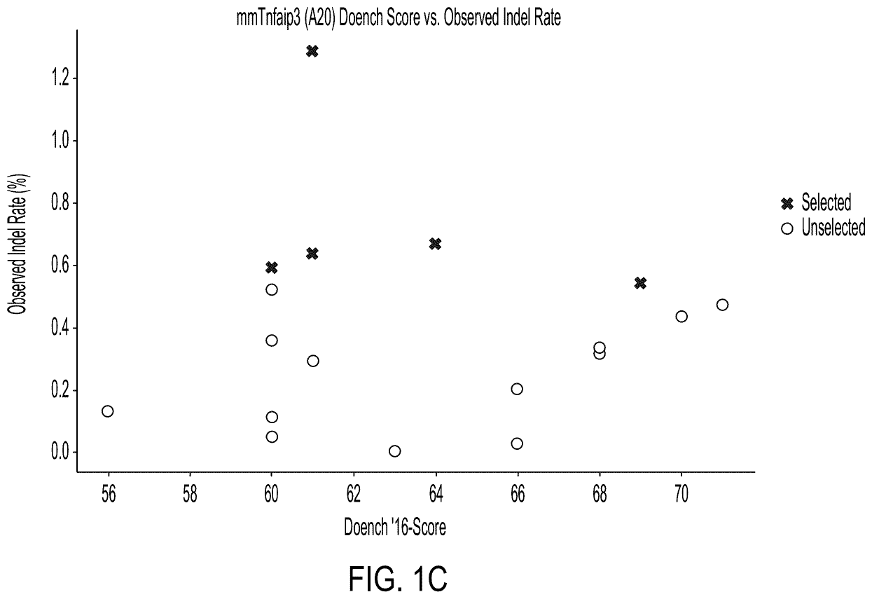

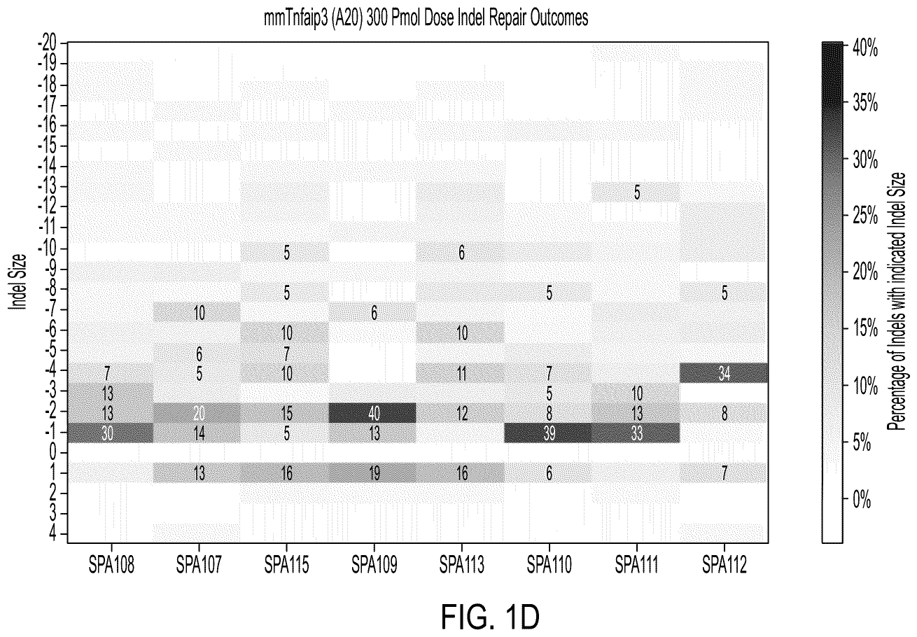

The percentage of edited cells (i.e., the editing rate) achieved with each gRNA was assessed as a function of the gRNA's Doench score. gRNAs having an increased editing rate relative to other gRNA candidates, had low variance in editing rate and high specificity in editing outcomes, and that fulfilled other biological criteria (e.g., based on isoform and protein domain mapping and/or guides with low variance in editing rate and high specificity scores) were selected for further validation. Additionally, each gRNA was assessed based on the indel repair outcomes observed following a 300 pmol dose of each gRNA (FIG. 1D). Selected guides predominantly led to indels that yielded frameshift mutations (e.g., −3 or −6 deletions are not preferred)

As shown in FIGS. 1A-1C, out of the 20 candidates screened, five Tnfaip3 gRNAs were identified that displayed an increased editing rate relative to other gRNA candidates assessed, had low variance in editing rate and high specificity in editing outcomes, and fulfilled other biological criteria. As shown in FIG. 1C, computational prediction of high activity was not sufficient to identify high efficiency guides. The data points corresponding to two of the top Tnfaip3 gRNAs selected for in vivo studies (RNA105 and RNA106) are notated in FIGS. 1B and 1C.

### Example 3. Ex Vivo Gene Editing of Tnfaip3 (A20) in Primary Murine T Cells Using Single gRNAs

This Example describes the results of an ex vivo study evaluating A20 (Tnfaip3) editing in primary murine T cells using single guide RNAs (sgRNAs). Spacers from the top candidate murine guide RNAs evaluated in two-part format (crRNA:tracrRNA) in Example 2 were evaluated in single gRNA format in the present Example. The sgRNAs and their corresponding spacer and sequences are outlined in Table 3.

TABLE 3
sgRNAs targeting murine Tnfaip3

| Spacer | sgRNA | | | | | Target |
|---|---|---|---|---|---|---|
| ID | ID | Name | Guide sequence + PAM | Protospacer | PAM | region |
| SPA108 | RNA105 | mmTnfaip3_ | TGATAGATGATCCCATTGG | TGATAGATGATCCC | AGG | Exon 2 |
| | | g3_SPA108 | TAGG (SEQ ID NO: 435) | ATTGGT | | |
| | | | | (SEQ ID NO: 440) | | |
| SPA107 | RNA106 | mmTnfaip3_ | TCGTGAAGTCAGGAAGCT | TCGTGAAGTCAGGA | TGG | Exon 2 |
| | | g2_SPA107 | CGTGG (SEQ ID NO: 436) | AGCTCG | | |
| | | | | (SEQ ID NO: 441) | | |
| SPA115 | RNA107 | mmTnfaip3_ | ATGCAGCTTGTCAGTACAT | ATGCAGCTTGTCAG | TGG | Exon 3 |
| | | g10_SPA115 | GTGG (SEQ ID NO: 437) | TACATG | | |
| | | | | (SEQ ID NO: 442) | | |
| SPA109 | RNA108 | mmTnfaip3_ | CTTTGTATTTGAGCAATAT | CTTTGTATTTGAGCA | CGG | Exon 2 |
| | | g4_SPA109 | GCGG (SEQ ID NO: 438) | ATATG | | |
| | | | | (SEQ ID NO: 443) | | |
| SPA113 | RNA109 | mmTnfaip3_ | GCAGCTTGTCAGTACATGT | GCAGCTTGTCAGTA | GGG | Exon 3 |
| | | g8_SPA113 | GGGG (SEQ ID NO: 439) | CATGTG | | |
| | | | | (SEQ ID NO: 444) | | |

Purified murine T cells were electroporated with 50 pmol of Cas9 ribonucleoprotein (RNP) using single guides targeting the A20 (Tnfaip3) gene. Three days after electroporation, genomic DNA was isolated from the cells, and editing was measured by a Next-Generation Sequencing (NGS) assay to quantify insertion and deletion mutations created by the RNP. The level of editing (% Indel) as detected by NGS is shown for each of the indicated conditions.

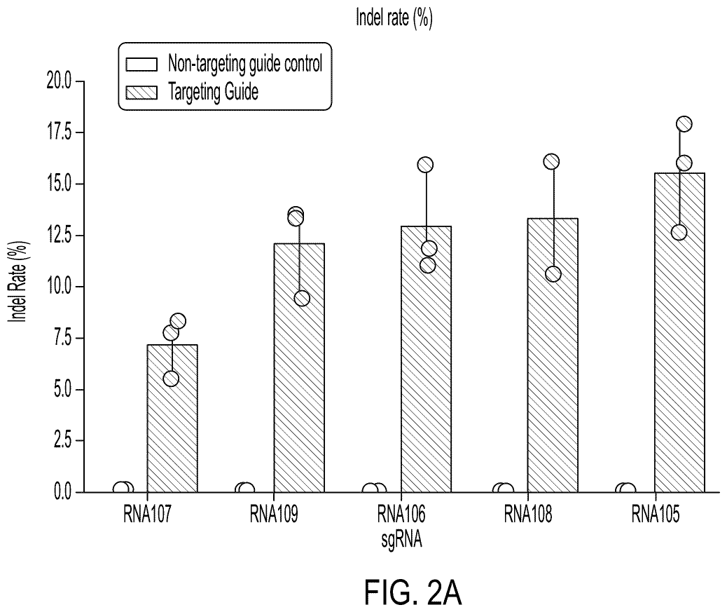

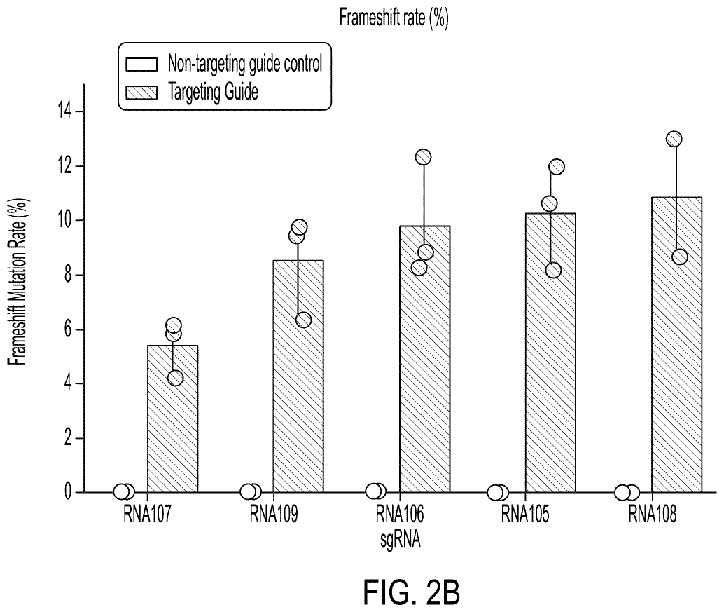

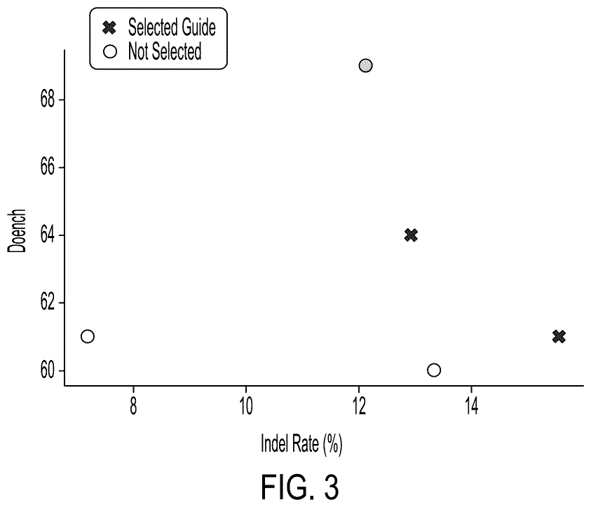

As shown in FIGS. 2A and 2B, editing activity was observed with murine guide RNAs in single guide RNA format targeting Tnfaip3. As shown in FIG. 3, computational on-target score did not correlate with observed editing rate, indicating that empirical evaluation is needed for identifying candidate guide RNAs.

### Example 4. Identification of gRNAs Targeting Human TNFAIP3

This Example describes the process of identifying gRNAs targeting the human TNFAIP3 gene.

Methodology of in Silico Analysis to Identify Human gRNAs

gRNAs targeting TNFAIP3 were identified based on a computational algorithm comprising multiple filtering steps. Potential gRNAs were first identified based on PAM recognition (NGG and TTTV for S. pyogenes Cas9 and AsCas12a, respectively). gRNAs within early exons were prioritized to enrich for guide sites in which a frameshift causing indel would lead to a loss of function protein (e.g., exons 2, 3, or 4 of TNFAIP3) or would otherwise target a functional domain of TNFAIP3. In addition, gRNAs targeting key domains (e.g., the OTU domain, a ubiquitin-interacting subdomain, a TRAF-binding domain, TNIP1-interaction domain, a RIPK1-interacting domain, a lysosomal localization domain, or a zinc-finger domain in human TNFAIP3) were prioritized. These potential gRNAs were then ranked based on on-target scoring prediction (Doench score) and by off-target profile. The off-target profile was generated by determining the number of potential genomic off-target sites with 0-4 mismatches in the protospacer sequence in both genic and intergenic sequences utilizing NAG and TTTN PAM sequences for S.Py. Cas9 and AsCas12a, respectively. Potential gRNAs were filtered out (excluded) when there was an off-target site with 0, 1, or 2 mismatches. Remaining guides were then rank ordered by mismatches (3-4 mismatches) and on-target scoring. gRNAs selected based on these criteria are summarized in Table 1.

### Example 5. Identification of gRNAs Targeting Murine SOCS1

This Example describes a screen for guide RNAs (gRNAs) that target the mouse Socs1 gene.

20 candidate cr:tracrRNAs were designed against the mouse Socs1 gene and complexed with Streptococcus pyogenes Cas9 to form ribonucleoproteins (RNPs). RNPs including the candidate gRNAs were then introduced into mouse T cells by nucleofection. A benchmark gRNA targeting CD44 was also complexed with Cas9 and assessed for editing as a positive control. Editing at the target gene was detected by Next Generation Sequencing. Doench scores for each gRNA were calculated as a measure of predicted sgRNA activity (Doench et al., Nature Biotechnology 32, 1262-1267 (2014)).

Primary human T cells were electroporated with a dose titration of pool of Cas9 RNP complexed with indicated guide RNAs. Guide RNA were in two-part (crRNA:tracrRNA) format. Three days after electroporation, genomic DNA was isolated from the cells, and editing was measured by an amplicon-based Next-Generation Sequencing (NGS) assay to quantify insertion and deletion mutations created by the RNP at each of the target sites. The level of editing (% editing) as detected by NGS is shown for each of the indicated doses. Top candidate guide RNAs are indicated as shaded.

The percentage of edited cells (i.e., the editing rate) achieved with each gRNA was assessed as a function of the gRNA's Doench score. gRNAs having an increased editing rate relative to other gRNA candidates assessed, had low variance in editing rate and high specificity in editing outcomes, and that fulfilled other biological criteria (e.g., based on isoform and protein domain mapping and/or guides with low variance in editing rate and high specificity scores) were selected for further validation. Additionally, each gRNA Was assessed based on the indel repair outcomes observed following a 300 pmol dose of each gRNA (FIG. 2D). Selected guides predominantly led to indels that yielded frameshift mutations (e.g., −3 or −6 deletions are not preferred).

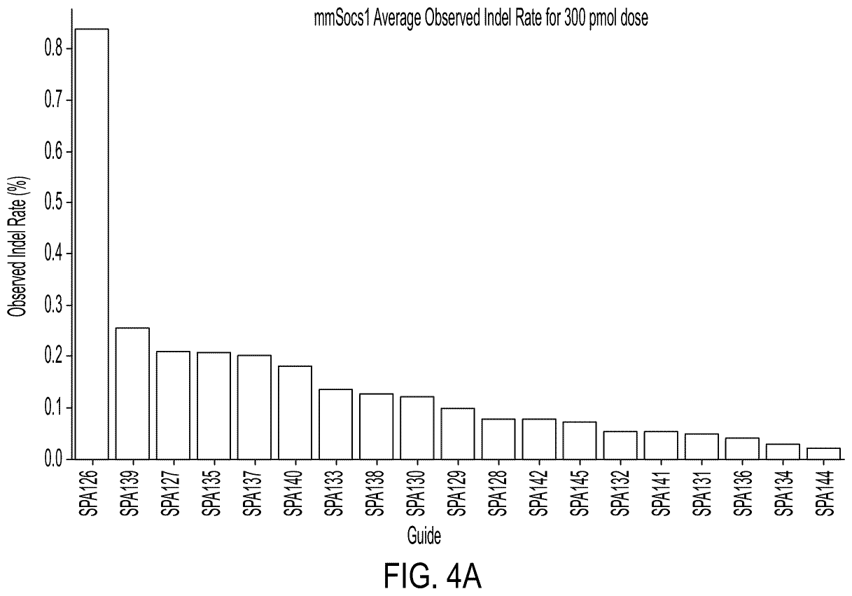

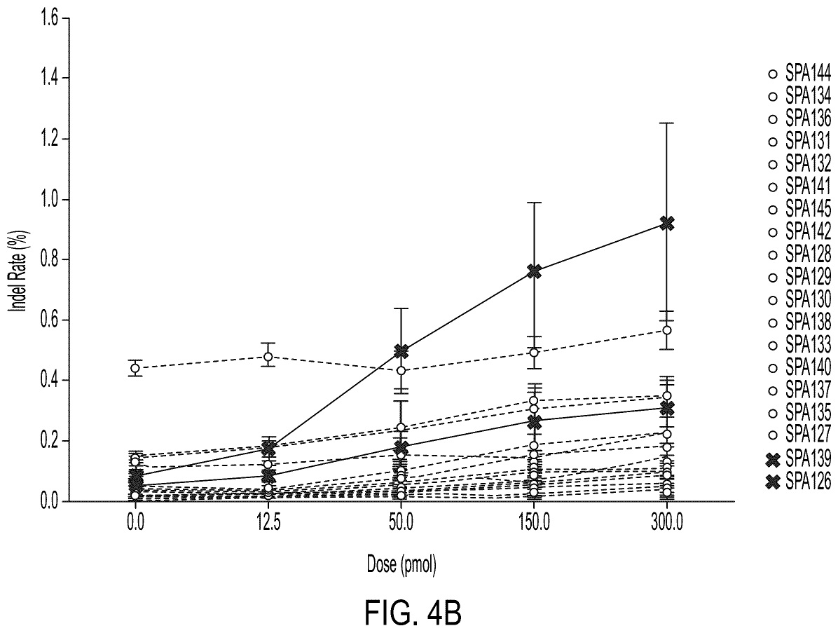

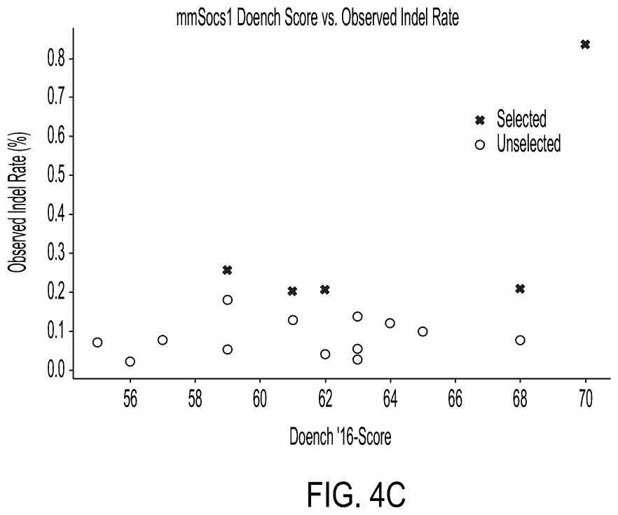

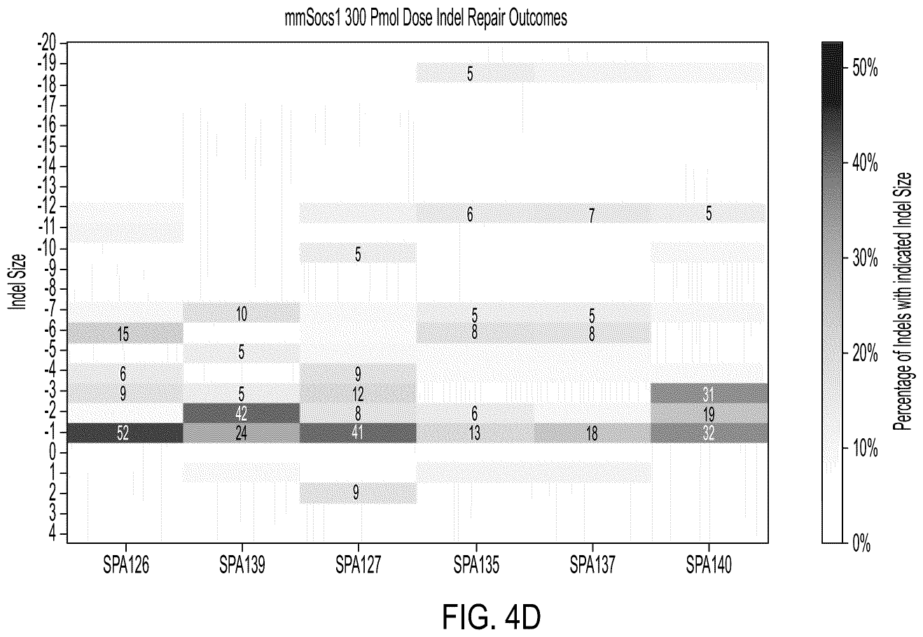

As shown in FIGS. 4A-4C, out of the 20 candidates screened, five Socs1 gRNAs were identified that displayed an increased editing rate relative to other gRNA candidates assessed, had low variance in editing rate and high specificity in editing outcomes, and fulfilled other biological criteria. As shown in FIG. 4C, computational prediction of high activity was not sufficient to identify high efficiency guides. The data points corresponding to the five selected Socs1 gRNAs are notated in FIGS. 4B and 4C.

### Example 6. Ex Vivo Gene Editing of SOCS1 in Primary Murine T Cells Using Single gRNAs

This Example describes the results of an ex vivo study evaluating SOCS1 editing in primary murine T cells using single guide RNAs (sgRNAs). Spacers from the top candidate murine guide RNAs evaluated in two-part format (crRNA:tracrRNA) in Example 5 were evaluated in sgRNA format in the present Example. The sgRNAs and their corresponding spacer and sequences are outlined in Table 4.

TABLE 4
sgRNAs targeting murine Socs1

| Spacer | sgRNA | | | | | Target |
|---|---|---|---|---|---|---|
| ID | ID | Name | Guide sequence + PAM | Protospacer | PAM | region |
| SPA126 | RNA110 | mmSocs1_ | GATGGTAGCACGCAACCA | GATGGTAGCACGCA | TGG | Exon 2 |
| | | g1_SPA126 | GGTGG (SEQ ID NO: 445) | ACCAGG | | first |
| | | | | (SEQ ID NO: 450) | | half |
| SPA139 | RNA111 | mmSocs1_ | AGCCGACAATGCGATCTC | AGCCGACAATGCGA | CGG | Exon 2 |
| | | g14_SPA139 | CCCGG (SEQ ID NO: 446) | TCTCCC | | first |
| | | | | (SEQ ID NO: 451) | | half |
| SPA137 | RNA112 | mmSocs1_ | GATGCGCCGGTAATCGGA | GATGCGCCGGTAAT | GGG | Exon 2 |
| | | g12_SPA137 | GTGGG (SEQ ID NO: 447) | CGGAGT | | first |
| | | | | (SEQ ID NO: 452) | | half |
| SPA135 | RNA113 | mmSocs1_ | TGATGCGCCGGTAATCGG | TGATGCGCCGGTAA | TGG | Exon 2 |
| | | g10_SPA135 | AGTGG (SEQ ID NO: 448) | TCGGAG | | first |
| | | | | (SEQ ID NO: 453) | | half |
| SPA127 | RNA114 | mmSocs1_ | GCGGAAGTGAGTGTCGCC | GCGGAAGTGAGTGTGGG | | Exon 2 |
| | | g2_SPA127 | AGGGG (SEQ ID NO: 449) | CGCCAG | | first |
| | | | | (SEQ ID NO: 454) | | half |

Purified murine T cells were electroporated with 50 pmol of Cas9 ribonucleoprotein (RNP) using single guides targeting the Socs1 gene. Three days after electroporation, genomic DNA was isolated from the cells, and editing was measured by a Next-Generation Sequencing (NGS) assay to quantify insertion and deletion mutations created by the RNP. The level of editing (% Indel) as detected by NGS is shown for each of the indicated conditions.

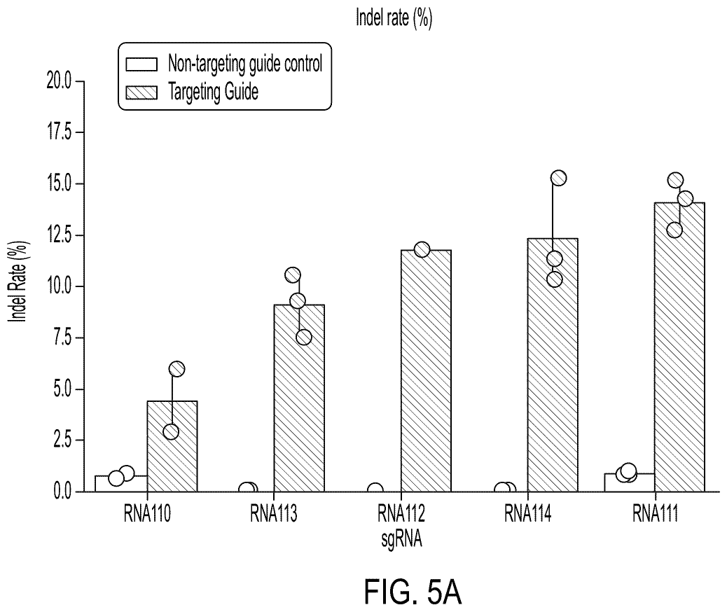

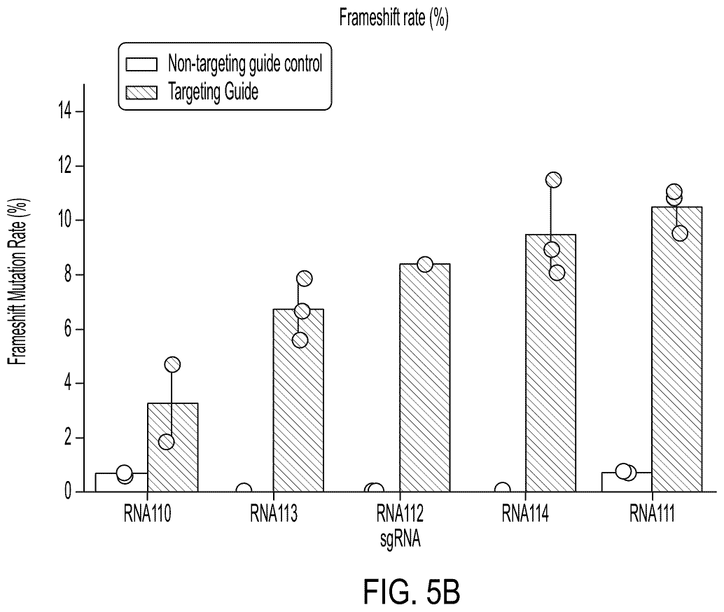

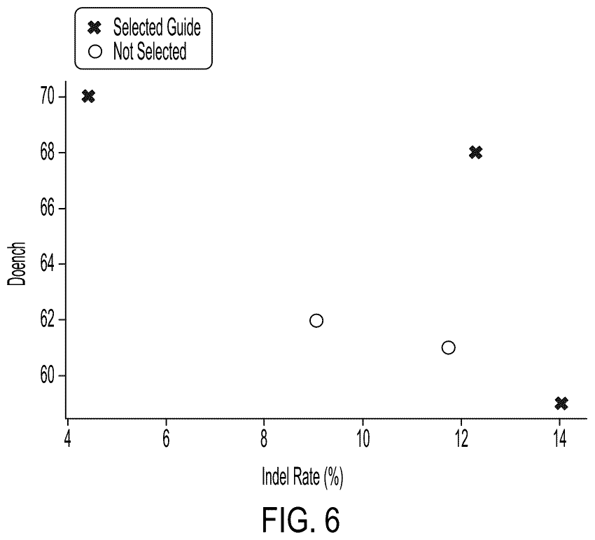

As shown in FIGS. 5A and 5B, editing activity was observed with murine guide RNAs in single guide RNA format targeting Socs1. As shown in FIG. 6, computational on-target score did not correlate with observed editing rate, indicating that empirical evaluation is needed for identifying candidate guide RNAs. RNA110 included the spacer SPA126, the top initial candidate in the screen for two-part gRNAs in Example 5; see also Table 4). Although the spacer in RNA111 (spacer SPA139) was not the top initial hit in the screen for two-part gRNAs in Example 5, RNA111 displayed similar or greater editing efficacy as a single guide RNA compared to other guide RNAs tested and in Example 8, RNA11 displayed more anti-tumor efficacy compared to other guide RNAs. These observations further indicate that empirical evaluation is needed for identifying candidate guide RNAs.

### Example 7. In Vivo Anti-Tumor Efficacy of Guide RNAs Targeting Tnfaip3 and Socs1 in Mice

This Example describes the results of an in vivo study evaluating the anti-tumor efficacy of treatment with a TAGE agent including a guide RNA that targets A20 (Tnfaip3) or Socs1 in combination with an immune checkpoint blockade.

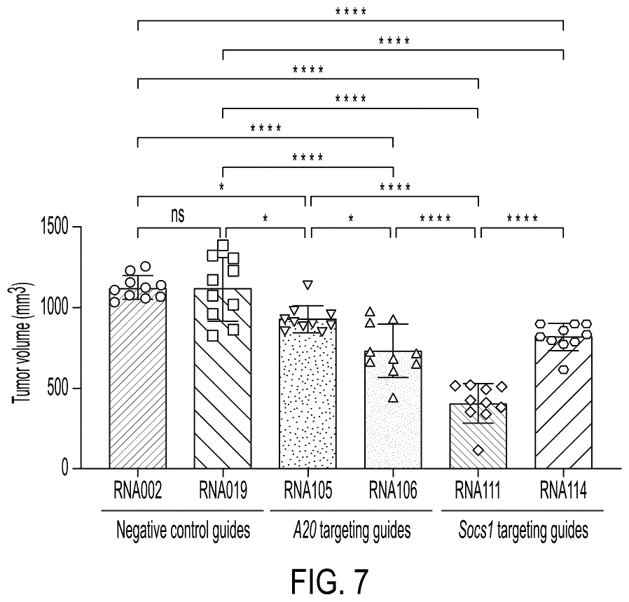

Mice were implanted with one flank subcutaneous CT26 tumors. TAGE including Cas9 were complexed with a Tnfaip3 (A20)-targeting guide RNA (RNA105 or RNA106), a Socs1-targeting guide RNA (RNA111 or RNA114) or a negative control guide (RNA002 that does not target a gene, or RNA019 which targets an irrelevant gene target) to form TAGE ribonucleoproteins (RNPs). The TAGE RNPs were administered via intratumoral injection. An immune checkpoint blockade was administered via intraperitoneal injection on days 0, 3, 6, and 9. Tumor measurements were performed 2-3 times per week with day 10 post TAGE treatment measurements displayed in FIG. 7. *>0.001, 1-way ANOVA with multiple testing correction.

As shown in FIG. 7, a significant reduction in tumor growth was observed following treatment with Tnfaip3 (A20) targeting guides (RNA105, RNA106) or Socs1 targeting guides (RNA111, RNA114) in comparison to negative control guides (RNA002, RNA019).

### Example 8. In Vivo Anti-Tumor Enestic and Anenestic Efficacy of Guide RNAs Targeting Socs1 in Mice

This Example describes the results of an in vivo study evaluating the enestic and anenestic anti-tumor efficacy of a TAGE agent including a guide RNA that targets Socs1 in combination with an immune checkpoint blockade.

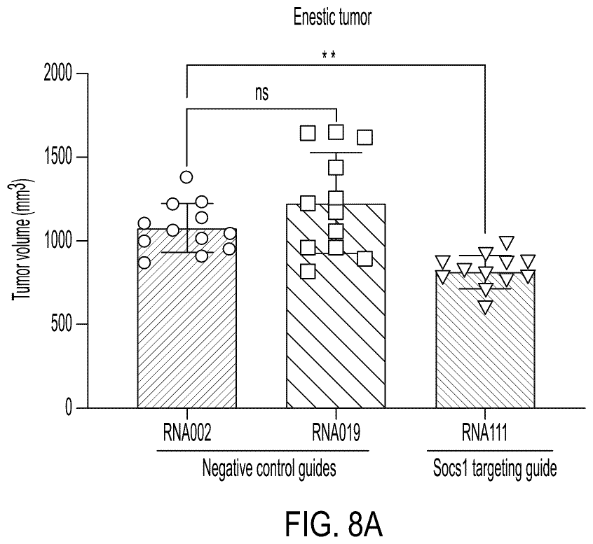

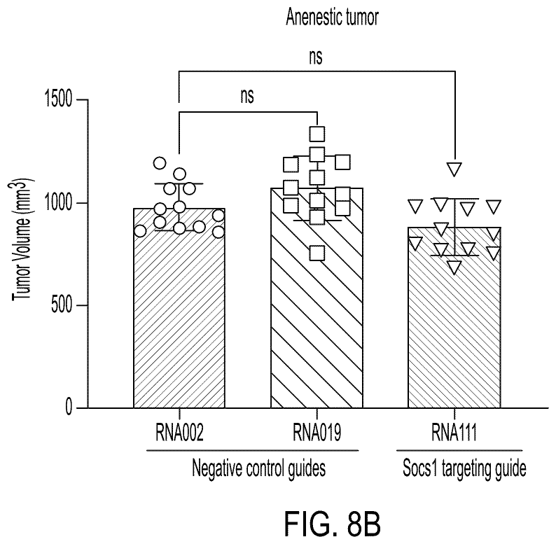

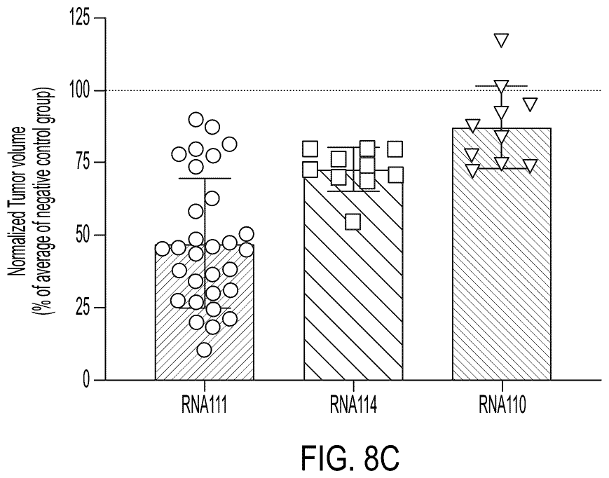

Mice were implanted with bilateral subcutaneous CT26 flank tumors. TAGE complexed with guides that targeted no gene target, an irrelevant gene target, or Socs1 were administered intratumorally into the right flank tumor. Immune checkpoint blockade was administered via intraperitoneal injection on days 0, 3, 6, and 9. Tumor measurements were performed 2-3 times per week. Day 10 post TAGE treatment measurements of the enestic (injected) and anenestic (non-TAGE injected) tumors are displayed in FIGS. 8A and 8B.

As shown in FIGS. 8A and 8B, significant reduction in enestic tumor growth in mice treated with a gRNA targeting Socs1 (RNA111) was observed in comparison to mice treated with negative control guides. *p>0.01, 1-way ANOVA with multiple testing correction.

Subsequently, a meta analysis of several experiments was performed in which the tumor volume was normalized to the average tumor volume of the negative control group at the latest timepoint with all mice alive in the study (prior to meeting of pre-defined study endpoints).

As shown in FIG. 8C, tumor reduction was observed with all Socs1 targeting guides, with the greatest tumor reduction observed in RNA111 (Cut site within the first 14 amino acids (40 nucleotides) from the translational start site). RNA110 included spacer SPA126 (Cut site within the first 6 amino acids (16 nucleotides) from the translational start site), which was one of the top initial hits in Example 5. In contrast, RNA111 displayed increased anti-tumor efficacy compared to RNA110 and RNA114 even though the spacer included in RNA111 (SPA139) was not a top candidate in the initial screen for two-part gRNAs in Example 5. RNA114 had similar editing rate in vitro (as shown in Example 5) but not similar in vivo anti-tumor efficacy (as shown in Example 8, FIG. 8C).

### Example 9. Identification of gRNAs Targeting Human SOCS1

This Example describes the process of identifying gRNAs targeting the human SOCS1 gene.

Methodology of in Silico Analysis to Identify Human gRNAs

gRNAs targeting SOCS1 were identified based on a computational algorithm comprising multiple filtering steps. Potential gRNAs were first identified based on PAM recognition (NGG and TTTV for S. pyogenes Cas9 and AsCas12a, respectively). gRNAs within early exons were prioritized to enrich for guide sites in which a frameshift causing indel would lead to a loss of function protein (e.g., exon 2 of SOCS1) or would otherwise target a functional domain in SOCS1. In addition, gRNAs targeting key domains (e.g., the SH2 (Src homology 2) domain, a SOCS box domain, a kinase inhibitory region, an extended SH2 subdomain, and an elongin BC complex interaction domain in human SOCS1) were prioritized. These potential gRNAs were then ranked based on on-target scoring prediction (Doench score) and by off-target profile. The off-target profile was generated by determining the number of potential genomic off-target sites with 0-4 mismatches in the protospacer sequence in both genic and intergenic sequences utilizing NAG and TTTN PAM sequences for S.Py. Cas9 and AsCas12a, respectively. Potential gRNAs were filtered out (excluded) when there was an off-target site with 0, 1, or 2 mismatches. Remaining guides were then rank ordered by mismatches (3-4 mismatches) and on-target scoring. gRNAs selected based on this criteria are summarized in Table 2.

### Example 10. Identification of gRNAs Targeting the Human SOCS1 Gene

This Example aimed to identify guide RNAs targeting the human SOCS1 gene.

RNP Complexation: Guide RNAs were used in crRNA:tracrRNA format. The components (crRNA and tracrRNA) were mixed at a concentration of 12.9 μM (each component). gRNAs were melted at 70 degrees Celsius, reannealed, and allowed to cool to room temperature. Cas9 was added to the annealed gRNA. The RNP was complexed at 37 degrees C. for 10 minutes. The final concentration of RNP was 10 μM.

T cell preparation: Purified human T cells were activated by culturing the cells with IL-2 and an anti-CD3/anti-CD28 stimulation reagent for 7 days prior to RNP nucleofection.

RNP Nucleofection: For each RNP, 5×10⁵ cells were mixed in a 25 μL volume with 50 pmol of RNP (2 μM final concentration of RNP with cells). Triplicate nucleofections were performed for each guide RNA. After nucleofection, cells were cultured in 96-well plates. Cells were maintained in media for 7 days after nucleofection. Genomic DNA was extracted from cells on Day 7 after nucleofection using QuickExtract reagent. Editing was measured using Next Generation Sequencing of PCR amplicons for the region targeted by each guide RNA.

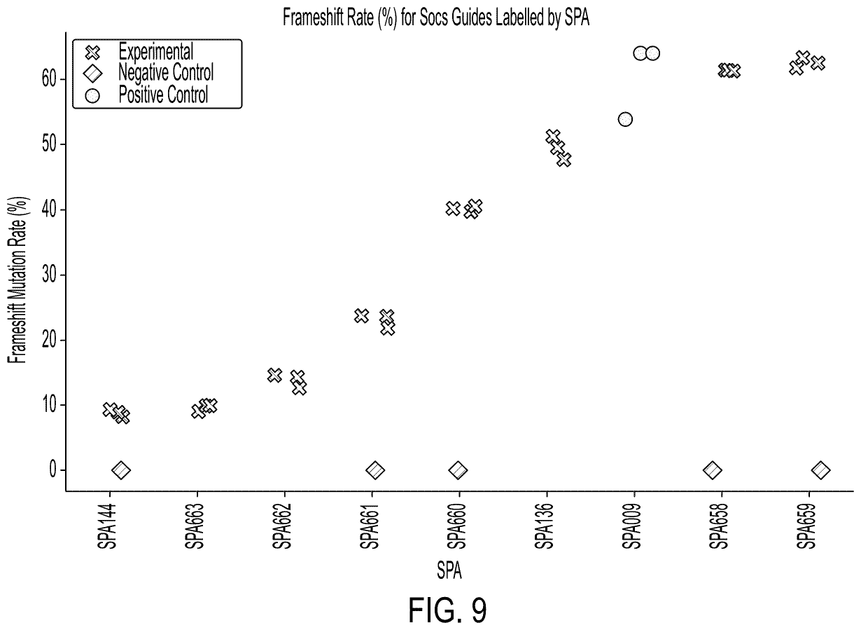

Results from the screen are summarized in FIG. 9 and Table 5, and indicate that high efficiency human SOCS1 guide RNAs could be identified. SPA144 was species cross-reactive and capable of binding the SOCS1 gene in multiple species (e.g., the SOCS1 gene in human and mice).

TABLE 5
Summary of select single guide RNAs targeting the human SOCS1 gene

| | | | | | | | | | DNA | | DNA | | |
|---|---|---|---|---|---|---|---|---|---|---|---|---|---|
| | Target | | gRNA | | | | | | Sequence | | Sequence | | Observed |
| | DNA | SEQ | spacer | SEQ | crRNA | SEQ | sgRNA | SEQ | encoding | SEQ | encoding | SEQ | Edit |
| | Sequence | ID | sequence | ID | Sequence | ID | Sequence | ID | crRNA | ID | sgRNA | ID | Rate |
| Name | (5′ to 3′) | NO | (5′ to 3′) | NO | (5′ to 3′) | NO | (5′ to 3′) | NO | (5′ to 3′) | NO | (5′ to 3′) | NO | (%) |
| 11255460rev | TAGGATGGTA | 223 | UAGGAUGGUAG | 307 | UAGGAUGGUAG | 455 | UAGGAUGGUAG | 463 | TAGGATGGTAGC | 471 | TAGGATGGTAGCA | 391 | 62.51333333 |
| | GCACACAACC | | CACACAACC | | CACACAACCGU | | CACACAACCGU | | ACACAACCGTTT | | CACAACCGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUU | | | | | | |
| | | | | | | | U | | | | | | |
| 1255457rev | GATGGTAGCA | 244 | GAUGGUAGCAC | 328 | GAUGGUAGCAC | 456 | GAUGGUAGCAC | 464 | GATGGTAGCACA | 472 | GATGGTAGCACAC | 412 | 61.35 |
| | CACAACCAGG | | ACAACCAGG | | ACAACCAGGGU | | ACAACCAGGGU | | CAACCAGGGTTT | | AACCAGGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255230forw | TGCGCCCCGT | 215 | UGCGCCCCGUG | 299 | UGCGCCCCGUG | 457 | UGCGCCCCGUG | 465 | TGCGCCCCGTGC | 473 | TGCGCCCCGTGCA | 383 | 49.47 |
| | GCACGCTCAG | | CACGCUCAG | | CACGCUCAGGU | | CACGCUCAGGU | | ACGCTCAGGTTT | | CGCTCAGGTTTTA | | |
| | | | | | UUUAGAGCUAUG | | UUUAGAGCUAG | | TAGAGCTATGCT | | GAGCTAGAAATAG | | |
| | | | | | CU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255233rev | ACGCCTGCGG | 480 | ACGCCUGCGGA | 481 | ACGCCUGCGGA | 458 | ACGCCUGCGGA | 466 | ACGCCTGCGGAT | 474 | ACGCCTGCGGATT | 479 | 40.09666667 |
| | ATTCTACTGG | | UUCUACUGG | | UUCUACUGGGU | | UUCUACUGGGU | | TCTACTGGGTTT | | CTACTGGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255312forw | CGGCGTGCGA | 186 | CGGCGUGCGAA | 270 | CGGCGUGCGAA | 459 | CGGCGUGCGAA | 467 | CGGCGTGCGAAC | 475 | CGGCGTGCGAACG | 354 | 23.03333333 |
| | ACGGAATGTG | | CGGAAUGUG | | CGGAAUGUGGU | | CGGAAUGUGGU | | GGAATGTGGTTT | | GAATGTGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255159forw | CGAAAAAGCA | 201 | CGAAAAAGCAG | 285 | CGAAAAAGCAG | 460 | CGAAAAAGCAG | 468 | CGAAAAAGCAGT | 476 | CGAAAAAGCAGTT | 369 | 13.87666667 |
| | GTTCCGCTGG | | UUCCGCUGG | | UUCCGCUGGGU | | UUCCGCUGGGU | | TCCGCTGGGTTT | | CCGCTGGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255152rev | TGGTGCGCGA | 193 | UGGUGCGCGAC | 277 | UGGUGCGCGAC | 461 | UGGUGCGCGAC | 469 | TGGTGCGCGACA | 477 | TGGTGCGCGACAG | 361 | 9.623333333 |
| | CAGCCGCCAG | | AGCCGCCAG | | AGCCGCCAGGU | | AGCCGCCAGGU | | GCCGCCAGGTTT | | CCGCCAGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |
| 11255246rev | AGCGCGCTCC | 213 | AGCGCGCUCCU | 297 | AGCGCGCUCCU | 462 | AGCGCGCUCCU | 470 | AGCGCGCTCCTG | 478 | AGCGCGCTCCTGG | 381 | 8.806666667 |
| | TGGACGCCTG | | GGACGCCUG | | GGACGCCUGGU | | GGACGCCUGGU | | GACGCCTGGTTT | | ACGCCTGGTTTTA | | |
| | | | | | UUUAGAGCUAU | | UUUAGAGCUAG | | TAGAGCTATGCT | | GAGCTAGAAATAG | | |
| | | | | | GCU | | AAAUAGCAAGU | | | | CAAGTTAAAATAA | | |
| | | | | | | | UAAAAUAAGGC | | | | GGCTAGTCCGTTA | | |
| | | | | | | | UAGUCCGUUAU | | | | TCAACTTGAAAAA | | |
| | | | | | | | CAACUUGAAAA | | | | GTGGCACCGAGTC | | |
| | | | | | | | AGUGGCACCGA | | | | GGTGCTTTT | | |
| | | | | | | | GUCGGUGCUUUU | | | | | | |

### Example 11. Ex Vivo Pharmacodynamic Assay in Primary Human T Cells to Assess gRNAs Targeting the Human SOCS1 Gene

This Example describes the results of an ex vivo pharmacodynamic assay in primary human T cells to assess gRNAs targeting the human SOCS1 gene.

Purified human T cells were activated by culturing IL-2 and an anti-CD3/anti-CD28 stimulation reagent for 7 days prior to RNP nucleofection. Cells were nucleofected (electroporated) with ribonucleoproteins including Cas9 complexed with either a guide RNA targeting SOCS1, a non-targeting control guide RNA, or a control guide RNA targeting an irrelevant gene control. For each RNP, 5×10⁵ cells in a 25 μL volume with 100 pmol of RNP (4 μM final concentration of RNP with cells).

Triplicate nucleofections were done for each guide RNA. After nucleofection, cells were cultured in 96-well plates. Cells were maintained in media for 3-5 days after nucleofection, then total cellular RNA was isolated. CXCL10 gene expression was measured by qRT-PCR, normalizing to GAPDH as a reference gene and normalizing all samples to the Irrelevant Target Gene Control #2 sample using the Delta Delta Ct method.

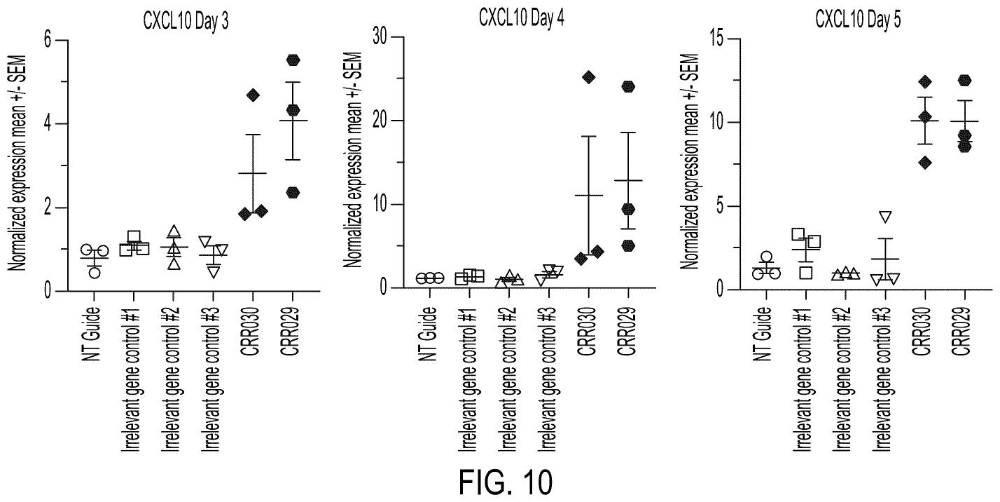

As shown in FIG. 10, there was increased expression of CXCL10 mRNA in human T cells 3, 4, or 5 days after SOCS1 editing. CXCL10 was assessed because it is a well characterized interferon response gene with increased CXCL10 indicative of induction of an interferon response. In addition, CXCL10 mRNA transcripts are more stable than interferon mRNA transcripts. CXCL10 gene expression was measured by qPCR and normalized to GAPDH as a reference gene. Points show biological replicates (individual electroporation samples), lines show mean±SEM.

TABLE 6
Summary of Sequences

| SEQ ID NO | Description | Sequence |
|---|---|---|
| 1 | Cas9(WT) | MDKKYSIGLDIGTNSVGWAVITDEYKVPSKKFKVLGNTD |
| | Streptococcus pyogenes | RHSIKKNLIGALLFDSGETAEATRLKRTARRRYTRRKNRI |
| | (amino acid sequence) | CYLQEIFSNEMAKVDDSFFHRLEESFLVEEDKKHERHPI |
| | | FGNIVDEVAYHEKYPTIYHLRKKLVDSTDKADLRLIYLAL |
| | | AHMIKFRGHFLIEGDLNPDNSDVDKLFIQLVQTYNQLFE |
| | | ENPINASGVDAKAILSARLSKSRRLENLIAQLPGEKKNGL |
| | | FGNLIALSLGLTPNFKSNFDLAEDAKLQLSKDTYDDDLD |
| | | NLLAQIGDQYADLFLAAKNLSDAILLSDILRVNTEITKAPL |
| | | SASMIKRYDEHHQDLTLLKALVRQQLPEKYKEIFFDQSK |
| | | NGYAGYIDGGASQEEFYKFIKPILEKMDGTEELLVKLNR |
| | | EDLLRKQRTFDNGSIPHQIHLGELHAILRRQEDFYPFLKD |
| | | NREKIEKILTFRIPYYVGPLARGNSRFAWMTRKSEETITP |
| | | WNFEEVVDKGASAQSFIERMTNFDKNLPNEKVLPKHSL |
| | | LYEYFTVYNELTKVKYVTEGMRKPAFLSGEQKKAIVDLL |
| | | FKTNRKVTVKQLKEDYFKKIECFDSVEISGVEDRFNASL |
| | | GTYHDLLKIIKDKDFLDNEENEDILEDIVLTLTLFEDREMI |
| | | EERLKTYAHLFDDKVMKQLKRRRYTGWGRLSRKLINGI |
| | | RDKQSGKTILDFLKSDGFANRNFMQLIHDDSLTFKEDIQ |
| | | KAQVSGQGDSLHEHIANLAGSPAIKKGILQTVKVVDELV |
| | | KVMGRHKPENIVIEMARENQTTQKGQKNSRERMKRIEE |
| | | GIKELGSQILKEHPVENTQLQNEKLYLYYLQNGRDMYVD |
| | | QELDINRLSDYDVDHIVPQSFLKDDSIDNKVLTRSDKNR |
| | | GKSDNVPSEEVVKKMKNYWRQLLNAKLITQRKFDNLTK |
| | | AERGGLSELDKAGFIKRQLVETRQITKHVAQILDSRMNT |
| | | KYDENDKLIREVKVITLKSKLVSDFRKDFQFYKVREINNY |
| | | HHAHDAYLNAVVGTALIKKYPKLESEFVYGDYKVYDVRK |
| | | MIAKSEQEIGKATAKYFFYSNIMNFFKTEITLANGEIRKRP |
| | | LIETNGETGEIVWDKGRDFATVRKVLSMPQVNIVKKTEV |
| | | QTGGFSKESILPKRNSDKLIARKKDWDPKKYGGFDSPT |
| | | VAYSVLVVAKVEKGKSKKLKSVKELLGITIMERSSFEKNP |
| | | IDFLEAKGYKEVKKDLIIKLPKYSLFELENGRKRMLASAG |
| | | ELQKGNELALPSKYVNFLYLASHYEKLKGSPEDNEQKQ |
| | | LFVEQHKHYLDEIIEQISEFSKRVILADANLDKVLSAYNKH |
| | | RDKPIREQAENIIHLFTLTNLGAPAAFKYFDTTIDRKRYTS |
| | | TKEVLDATLIHQSITGLYETRIDLSQLGGD |
| 2 | Cas12a | MTQFEGFTNLYQVSKTLRFELIPQGKTLKHIQEQGFIEE |
| | Acidaminococcus sp. | DKARNDHYKELKPIIDRIYKTYADQCLQLVQLDWENLSA |
| | (strain BV3L6) | AIDSYRKEKTEETRNALIEEQATYRNAIHDYFIGRTDNLT |
| | (amino acid sequence) | DAINKRHAEIYKGLFKAELFNGKVLKQLGTVTTTEHENAL |
| | | LRSFDKFTTYFSGFYENRKNVFSAEDISTAIPHRIVQDNF |
| | | PKFKENCHIFTRLITAVPSLREHFENVKKAIGIFVSTSIEE |
| | | VFSFPFYNQLLTQTQIDLYNQLLGGISREAGTEKIKGLNE |
| | | VLNLAIQKNDETAHIIASLPHRFIPLFKQILSDRNTLSFILE |
| | | EFKSDEEVIQSFCKYKTLLRNENVLETAEALFNELNSIDL |
| | | THIFISHKKLETISSALCDHWDTLRNALYERRISELTGKIT |
| | | KSAKEKVQRSLKHEDINLQEIISAAGKELSEAFKQKTSEI |
| | | LSHAHAALDQPLPTTLKKQEEKEILKSQLDSLLGLYHLLD |
| | | WFAVDESNEVDPEFSARLTGIKLEMEPSLSFYNKARNY |
| | | ATKKPYSVEKFKLNFQMPTLASGWDVNKEKNNGAILFV |
| | | KNGLYYLGIMPKQKGRYKALSFEPTEKTSEGFDKMYYD |
| | | YFPDAAKMIPKCSTQLKAVTAHFQTHTTPILLSNNFIEPL |
| | | EITKEIYDLNNPEKEPKKFQTAYAKKTGDQKGYREALCK |
| | | WIDFTRDFLSKYTKTTSIDLSSLRPSSQYKDLGEYYAEL |
| | | NPLLYHISFQRIAEKEIMDAVETGKLYLFQIYNKDFAKGH |
| | | HGKPNLHTLYWTGLFSPENLAKTSIKLNGQAELFYRPKS |
| | | RMKRMAHRLGEKMLNKKLKDQKTPIPDTLYQELYDYVN |
| | | HRLSHDLSDEARALLPNVITKEVSHEIIKDRRFTSDKFFF |
| | | HVPITLNYQAANSPSKFNQRVNAYLKEHPETPIIGIDRGE |
| | | RNLIYITVIDSTGKILEQRSLNTIQQFDYQKKLDNREKERV |
| | | AARQAWSVVGTIKDLKQGYLSQVIHEIVDLMIHYQAVVV |
| | | LENLNFGFKSKRTGIAEKAVYQQFEKMLIDKLNCLVLKD |
| | | YPAEKVGGVLNPYQLTDQFTSFAKMGTQSGFLFYVPAP |
| | | YTSKIDPLTGFVDPFVWKTIKNHESRKHFLEGFDFLHYD |
| | | VKTGDFILHFKMNRNLSFQRGLPGFMPAWDIVFEKNET |
| | | QFDAKGTPFIAGKRIVPVIENHRFTGRYRDLYPANELIAL |
| | | LEEKGIVFRDGSNILPKLLENDDSHAIDTMVALIRSVLQM |
| | | RNSNAATGEDYINSPVRDLNGVCFDSRFQNPEWPMDA |
| | | DANGAYHIALKGQLLLNHLKESKDLKLQNGISNQDWLAY |
| | | IQELRN |
| 3 | Uracil-Dna Glycosylase- | MTNLSDIIEKETGKQLVIQESILMLPEEVEEVIGNKPESDI |
| | Uracil Glycosylase | LVHTAYDESTDENVMLLTSDAPEYKPWALVIQDSNGEN |
| | Inhibitor | |
| | Protein Complex [Bacillus | KIKML |
| | virus PBS1] | |
| | (amino acid sequence) | |
| 4 | HIV-TAT | RKKRRQRRR |
| | (amino acid sequence) | |
| 5 | HIV-REV | RQARRNRRRRWR |
| | (amino acid sequence) | |
| 6 | SV40 NLS | PKKKRKV |
| | (amino acid sequence) | |
| 7 | Nucleoplasmin NLS | KRPAATKKAGQAKKKK |
| | (amino acid sequence) | |
| 8 | c-Myc-NLS | PAAKRVKLD |
| | (amino acid sequence) | |
| 9 | TDP | GDAHTGSSGSEFGGGSGGGSGGGSEGGSLAALTAHQ |
| | (amino acid sequence) | ACHLPLETFTRHRQPRGWEQLEQCGYPVQRLVALYLA |
| | | ARLSWNQVDQVIRNALASPGSGGDLGEAIREQPEQARL |
| | | ALTLAAAESERFVRQGTGNDEAGAANGGGSGGGSKLN |
| | | GSSGSELDK |
| 10 | TDP-KDEL | GDAHTGSSGSEFGGGSGGGSGGGSEGGSLAALTAHQ |
| | (″KDEL″ disclosed as | ACHLPLETFTRHRQPRGWEQLEQCGYPVQRLVALYLA |
| | SEQ ID NO: 11) | ARLSWNQVDQVIRNALASPGSGGDLGEAIREQPEQARL |
| | (amino acid sequence) | ALTLAAAESERFVRQGTGNDEAGAANGGGSGGGSKLN |
| | | GSSGSELDKKDEL |
| 11 | KDEL | KDEL |
| | (amino acid sequence) | |
| 12 | Tat | RKKRRQRRR |
| | (amino acid sequence) | |
| 13 | HIS-TAT-NLS (HTN) | HHHHHHGMGAAGRKKRRQRRRPPAGTSVSLKKKRKV |
| | (amino acid sequence) | |
| 14 | Homing Meganuclease Motif | LAGLIDADG |
| | (amino acid sequence) | |
| 15-179 | TNFAIP3 target DNA | See Table 1 |
| | sequences, gRNA sequences | |
| | (spacers); and DNA | |
| | sequences encoding said | |
| | gRNAs | |
| 180-431 | SOCS1 target DNA | See Table 2 |
| | sequences, gRNA sequences | |
| | (spacers); and DNA | |
| | sequences encoding said | |
| | gRNAs | |
| 432 | Subcomponent of Cas9 or | GUUUUAGAGCUAGAAAUAGCAAGUUAAAAUAAGGCU |
| | base editor single gRNA | AGUCCGUUAUCAACUUGAAAAAGUGGCACCGAGUC |
| | | GGUGCUUUU |
| 433 | Subcomponent of Cas9 or | GUUUUAGAGCUAUGCU |
| | base editor crRNA | |
| 434 | Subcomponent of Cas12 | UAAUUUCUACUCUUGUAGAU |
| | gRNA | |
| 435-444 | sgRNAs targeting murine | See Table 3 |
| | Tnfaip3 | |
| 445-454 | sgRNAs targeting murine | See Table 4 |
| | Socs1 | |
| 455-481 | Additional SOCS1 target | See Table 5 |
| | DNA sequences, gRNA | |
| | sequences (spacers, crRNA, | |
| | sgRNAs); and DNA | |
| | sequences encoding said | |
| | gRNAs | |

1. A method of reducing expression of a Suppressor of Cytokine Signaling 1 (SOCS1) gene in a mammalian cell, the method comprising contacting the mammalian cell with a site-directed modifying polypeptide and a guide RNA (gRNA) targeting the SOCS1 gene, thereby reducing expression of the SOCS1 gene in the mammalian cell.

2. The method of claim 1, wherein
the mammalian cell is a mouse cell, a non-human primate cell, or a human cell;
the mammalian cell is a cancer cell, a monocyte, a macrophage, an endothelial cell, an epithelial cell, a natural killer cell, a pericyte, a neutrophil, a T cell, a B cell, a dendritic cell, or a fibroblast;
the contacting step comprises delivering to the mammalian cell a nucleoprotein complex comprising the site-directed modifying polypeptide bound to the gRNA; or
the contacting step comprises delivering to the mammalian cell the site-directed modifying polypeptide and the gRNA separately.

3-5. (canceled)

6. A method of reducing expression of a Suppressor of Cytokine Signaling 1 (SOCS1) gene in a mammalian subject, the method comprising administering to the mammalian subject a therapeutically effective amount of a site-directed modifying polypeptide and a guide RNA (gRNA) targeting the (SOCS1) gene, thereby reducing expression of the SOCS1 gene in the mammalian subject.

7. The method of claim 6, wherein
the method comprises locally administering the site-directed modifying polypeptide and the gRNA to the mammalian subject;
the method comprises systemically administering the site-directed modifying polypeptide and the gRNA to the mammalian subject;
the mammalian subject is a human;
the mammalian subject has a cancer; and/or
the method further comprises administering an immune checkpoint blockade agent to the mammalian subject.

8-12. (canceled)

13. The method of claim 1, wherein
the gRNA comprises a single guide RNA (sgRNA); the gRNA comprises a crispr RNA (crRNA) and a trans-activating crispr RNA (trRNA); or the gRNA comprises a crRNA;
the site-directed modifying polypeptide is an RNA-guided nuclease or a base editor; and/or
the RNA-guided nuclease is a Class 2 Cas polypeptide.

14-17. (canceled)

18. The method of claim 13, wherein
the Class 2 Cas polypeptide is a Type II Cas polypeptide;
the Class 2 Cas polypeptide is a Type V Cas polypeptide; or
the base editor is a cytosine base editor or a adenine base editor.

19. The method of claim 18, wherein the Type II Cas polypeptide is Cas9 or the Type V Cas polypeptide is Cas 12.

20-24. (canceled)

25. The method of claim 1, wherein the site-directed modifying polypeptide further comprises a cell targeting agent, thereby forming a targeted active gene editing (TAGE) agent, wherein the cell targeting agent is a ligand, a cell penetrating peptide, or an antigen-binding polypeptide.

26-40. (canceled)

41. The method of claim 1, wherein
the gRNA targets a region of the SOCS1 gene that encodes an SH2 domain, a SOCS box domain, a kinase inhibitory region, extended SH2 subdomain, or an elongin BC complex (BC-box) interaction domain of a SOCS1 protein;
the gRNA targets a region within exon 2 of the SOCS1 gene;
the gRNA targets a region of the SOCS1 gene that encodes the first six amino acids of the SOCS1 protein;
the gRNA targets a region of the SOCS1 gene that encodes the first 14 amino acids of the SOCS1 protein;
the gRNA comprises a polynucleotide sequence set forth in any one of SEQ ID NOs: 264-347 and 481;
the site-directed modifying polypeptide comprises Cas9 and the gRNA comprises a polynucleotide sequence set forth in any one of SEQ ID NOs: 264, 266, 267, 270-346, and 481;
the site-directed modifying polypeptide comprises Cas12 and the gRNA comprises a polynucleotide sequence set forth in any one of SEQ ID NOs: 265, 268, and 269; or
the site-directed modifying polypeptide comprises a base editor and the comprises a polynucleotide sequence set forth in SEQ ID NO: 347.

42-48. (canceled)

49. A DNA targeting system for modifying a Suppressor of Cytokine Signaling 1 (SOCS1) gene, wherein the DNA targeting system comprises a site-directed modifying polypeptide and a guide RNA (gRNA) capable of targeting the SOCS1 gene gene.

50. The DNA targeting system of claim 49, wherein
the gRNA comprises a single guide RNA (sgRNA); a crispr RNA (crRNA) and a trans-activating crispr RNA (tracrRNA); or a crispr RNA (crRNA);
the site-directed modifying polypeptide is an RNA-guided nuclease or a base editor; and/or
the site-directed modifying polypeptide is a Class 2 Cas polypeptide.

51-54. (canceled)

55. The DNA targeting system of claim 50, wherein
the Class 2 Cas polypeptide is a Type II Cas polypeptide;
the Class 2 Cas polypeptide is a Type V Cas polypeptide; or
the base editor is a cytosine base editor or a adenine base editor.

56. The DNA targeting system of claim 55, wherein the Type II Cas polypeptide is Cas9 or the Type V Cas polypeptide is Cas12.

57-61. (canceled)

62. The DNA targeting system of claim 49, wherein the site-directed modifying polypeptide further comprises a cell targeting agent, thereby forming a targeted active gene editing (TAGE) agent, wherein the cell targeting agent is a ligand, a cell penetrating peptide, or an antigen-binding polypeptide.

63-77. (canceled)

78. The DNA targeting system of claim 49, wherein
the gRNA targets a region of the SOCS1 gene that encodes an SH2 domain, a SOCS box domain, a kinase inhibitory region, extended SH2 subdomain, or an elongin BC complex (BC-box) interaction domain of a SOCS1 protein;
the gRNA targets a region within exon 2 of the SOCS1 gene;
the gRNA targets a region of the SOCS1 gene that encodes the first six amino acids of the SOCS1 protein;
the gRNA targets a region of the SOCS1 gene that encodes the first 14 amino acids of the SOCS1 protein;
the gRNA comprises a polynucleotide sequence set forth in any one of SEQ ID NOs: 264-347 and 481;
the site-directed modifying polypeptide comprises Cas9 and the gRNA comprises a polynucleotide sequence set forth in any one of SEQ ID NOs: 264, 266, 267, 270-346, and 481;
the site-directed modifying polypeptide comprises Cas12 and the gRNA comprises a polynucleotide sequence set forth in any one of SEQ ID NOs: 265, 268, and 269; or
the site-directed modifying polypeptide comprises a base editor and the gRNA comprises a polynucleotide sequence set forth in SEQ ID NO: 347.

79-91. (canceled)

92. An isolated polynucleotide encoding the DNA targeting system of claim 49.

93-95. (canceled)

96. An isolated polynucleotide encoding a gRNA, wherein the gRNA comprises a polynucleotide sequence set forth in any one of SEQ ID NOs: 264-347 and 481.

97. A vector comprising the isolated polynucleotide of claim 92.

98. A cell comprising the isolated polynucleotide of claim 92.

99. A lipid nanoparticle (LNP) comprising an mRNA encoding the DNA targeting system of claim 49.

100. (canceled)

101. A lipid nanoparticle (LNP) comprising the isolated polynucleotide of claim 96, wherein the isolated polynucleotide is an mRNA.

102-105. (canceled)

106. A vector comprising the isolated polynucleotide of claim 96.

107. A cell comprising the isolated polynucleotide of claim 96.
